(19) United States
(12) Patent Application Publication
(10) Pub. No.: US 2026/0041761 A1
(43) Pub. Date: Feb. 12, 2026

(54) PHARMACEUTICAL COMPOSITION FOR RESISTING INFECTION WITH SARS-COV-2 OR MUTANT THEREOF, AND COMBINED DRUG THEREOF

(71) Applicant: WESTVAC BIOPHARMA CO., LTD., Chengdu, Sichuan (CN)

(72) Inventors: Xiawei WEI, Chengdu, Sichuan (CN); Guangwen LU, Chengdu, Sichuan (CN); Ping CHENG, Chengdu, Sichuan (CN); Li YANG, Chengdu, Sichuan (CN); Jiong LI, Chengdu, Sichuan (CN); Jingyun YANG, Chengdu, Sichuan (CN); Wei WANG, Chengdu, Sichuan (CN); Yuquan WEI, Chengdu, Sichuan (CN); Zhenling WANG, Chengdu, Sichuan (CN); Guobo SHEN, Chengdu, Sichuan (CN); Zhiwei ZHAO, Chengdu, Sichuan (CN); Jinliang YANG, Chengdu, Sichuan (CN)

(21) Appl. No.: 18/881,413

(22) PCT Filed: Jun. 30, 2023
(86) PCT No.: PCT/CN2023/105178

§ 371 Date: Jan. 6, 2025

(30) Foreign Application Priority Data
Jul. 7, 2022 (CN) 202210802239.5
Dec. 15, 2022 (CN) 202211618137.4

(51) Int. Cl. A61K 39/215; A61K 39/00; A61K 39/235; A61P 37/04; C07K 14/005

(52) U.S. Cl. CPC A61K 39/215; A61K 39/235; A61P 37/04; C07K 14/005; A61K 2039/543; C12N 2710/10022; C12N 2710/10034; C12N 2710/10071; C12N 2770/20022; C12N 2770/20034; C12N 2770/20071

(57) ABSTRACT

Provided are a pharmaceutical composition for resisting infection with SARS-COV-2 or a mutant thereof, and a combined drug thereof. To solve the problem of the lack of effective prevention and treatment drugs for infection with SARS-COV-2 or a mutant virus thereof, provided are a recombinant protein vaccine and/or an adenovirus vaccine for preventing and/or treating an infection with SARS-COV-2 or a mutant thereof, and in particular, provided are a nasal spray administration compound formulation containing active ingredients of two vaccines, i.e., a recombinant protein vaccine and an adenovirus vaccine, and a combination of the two vaccines for nasal spray administration, which can induce generation of strong antibody and cellular immune responses in vivo and block the binding of a protein S of SARS-COV-2 to an ACE2 receptor of a host cell, thus enabling a host to resist coronavirus infection. Particularly, the present invention has good prevention and treatment effects on various mutant viruses.

### TECHNICAL FIELD

The present invention relates to a pharmaceutical composition for resisting infection with SARS-COV-2 or a mutant thereof and a combined drug thereof, and in particular, to a broad-spectrum nasal spray of compound preparation for resisting infection with SARS-COV-2 or a mutant thereof and a combined drug thereof, which belongs to the medical field.

### BACKGROUND

SARS-COV-2 is a novel betacoronavirus named by the World Health Organization. The virus has an envelope and the particles are round or oval, and often polymorphic, with a diameter of 60 nm-140 nm. Its genetic characteristics are significantly different from those of SARS-COV and MERS-COV, and SARS-COV-2 is a branch of novel coronavirus that has not been previously found in humans. At present, there are five major types of mutated novel coronavirus: Alpha, Beta, Gamma, Delta, and Omicron. Omicron variant strains are further divided into several substrains, such as BA.1, BA.2, BA.2.12.1, BA.4, and BA.5. Up to now, the novel coronavirus SARS-COV-2 has infected 545 million people worldwide, including more than 6.3 million of fatality.

The main structural proteins of SARS-COV-2 include Spike(S), Envelop (E), Membrane (M) and Nucleocapsid (N). Among them, S protein plays a key role in virus infection and virulence. It is often used as an antigen in vaccines. As the SARS-COV-2 variant strain contains multiple mutation sites and the S protein of the virus also contains multiple mutation sites, the variant strain can escape antibodies stimulated by the SARS-COV-2 prototype strain (namely, “novel coronavirus wild strain”) vaccine to a certain extent, resulting in the ineffectiveness or reduced protection of the SARS-COV-2 vaccine, which brings great pressure to the prevention and control of the SARS-COV-2 pandemic. Therefore, the development of a vaccine against various variants of SARS-COV-2 virus, especially a broad spectrum vaccine against various variants of SARS-COV-2 virus, is very important for the prevention and treatment of novel coronavirus pneumonia.

At present, hundreds of companies and units around the world are developing SARS-COV-2 vaccines, which mainly involve in five technical routes of inactivated vaccines, recombinant protein vaccines, adenovirus vector vaccines, attenuated influenza virus vector vaccines, and nucleic acid vaccines (including mRNA vaccines and DNA vaccines). Meanwhile, a variety of vaccines have been launched. Vaccination routes mainly include subcutaneous injection, intradermal injection, intramuscular injection, oral administration and inhalation (including nasal spray). Many viruses such as influenza virus and novel coronavirus are mainly diffused through a respiratory tract, and nasal spray or inhalation vaccines are developed through simulation of the natural infection process of human body. Therefore, it is important to develop vaccines that can induce strong mucosal immunity to effectively prevent the invasion and infection of these viruses from the respiratory tract.

### SUMMARY

The present invention is aimed to solve one of the technical problems existing in the prior art. For this purpose, the present invention is to provide a nasal spray of compound preparation containing a recombinant protein vaccine and an adenovirus vaccine for resisting infection with SARS-COV-2 or a mutant thereof, and a combined nasal spray.

The present invention provides a pharmaceutical composition for preventing and/or treating infection with SARS-COV-2 or a mutant thereof, where the pharmaceutical composition is a compound preparation containing active ingredients of a recombinant protein vaccine and/or an adenovirus vaccine for resisting infection with SARS-COV-2 or the mutant thereof. Further, the compound preparation also contains other pharmaceutically acceptable excipients or complementary ingredients.

The present invention further provides a combined drug for preventing and/or treating infection with SARS-COV-2 or a mutant thereof, where the combined drug contains a recombinant protein vaccine and an adenovirus vaccine for resisting infection with SARS-COV-2 or the mutant thereof, and the recombinant protein vaccine and the adenovirus vaccine are administered separately or simultaneously.

Further, the pharmaceutical composition or the combined drug is an intramuscular injection, a nasal drop, a spray, a nasal spray or inhalation; and preferably, the pharmaceutical composition or the combined drug is a nasal spray.

Further, the recombinant protein vaccine and/or the adenovirus vaccine contain/contains a protein and/or a protein precursor for resisting infection with SARS-COV-2 or the mutant thereof.

Further, the protein and/or the protein precursor contain/contains a full-length S protein or a protein formed by at least one RBD sequence and/or at least one HR sequence in the S protein of SARS-COV-2 or the mutant thereof;

preferably, the RBD sequence is shown in SEQ ID NO: 1; or the RBD sequence is a variant that has homology and same or similar biological activity with SEQ ID NO:1 and is obtained by substitution and/or deletion and/or insertion of at least one amino acid in the SEQ ID NO: 1 sequence; preferably, the RBD sequence is a variant obtained by substitution and/or deletion and/or insertion of 1-400 amino acids in the SEQ ID NO: 1 sequence; and preferably, the RBD sequence is a variant obtained by substitution and/or deletion and/or insertion of 5-30 amino acids in the SEQ ID NO: 1 sequence.

Further, the protein formed by the RBD sequence and the HR sequence in the S protein are capable of spontaneously forming a trimer.

Further, the homologous amino acid sequence is selected from at least one of RBD sequences of Alpha, Beta, Gamma, Delta or Omicron.

Further, protein precursor is that a signal peptide and/or a protein tag are/is bonded to the protein for resisting infection with SARS-COV-2 or the mutant thereof; preferably, the signal peptide comprises the signal peptide of the S protein or the mutant thereof and/or a human tPA signal peptide additionally provided outside the forehead of the self-provided signal peptide; preferably, the protein tag is selected from at least one of a histidine tag, a thioredoxin tag, a glutathione transferase tag, a ubiquitin-like modified protein tag, a maltose-binding protein tag, a c-Myc protein tag or a Avi tag protein tag; and more preferably, the protein tag is a Trx tag and/or a 6His tag.

Further, the protein for resisting infection with SARS-COV-2 or the mutant thereof is further bonded to a protease recognition zone for removing the protein tag; and preferably, the protease is selected from at least one of enterokinase, TEV protease, thrombin, coagulation factor Xa, carboxypeptidase A or rhinovirus 3c protease.

Further, the amino acid sequence of the protein and/or the protein precursor are/is selected from at least one of SEQ ID NO: 1, SEQ ID NO:2, SEQ ID NO:3, SEQ ID NO:4, SEQ ID NO: 5, SEQ ID NO:6, SEQ ID NO: 7, SEQ ID NO: 8, SEQ ID NO:9, SEQ ID NO: 18, SEQ ID NO: 20, SEQ ID NO:22, SEQ ID NO: 40, SEQ ID NO: 43, SEQ ID NO:46, SEQ ID NO:49, SEQ ID NO: 52, SEQ ID NO:55, SEQ ID NO:58, SEQ ID NO:61, SEQ ID NO:64, SEQ ID NO:67, SEQ ID NO:70, or SEQ ID NO:73.

Further, a nucleotide sequence for encoding the amino acid sequence is shown in SEQ ID NO:10, SEQ ID NO:11, SEQ ID NO:12, SEQ ID NO:13, SEQ ID NO:14, SEQ ID NO: 15, SEQ ID NO:16, SEQ ID NO:17, SEQ ID NO:19, SEQ ID NO:21, SEQ ID NO:23, SEQ ID NO: 41, SEQ ID NO:42, SEQ ID NO:44, SEQ ID NO:45, SEQ ID NO:47, SEQ ID NO:48, SEQ ID NO:50, SEQ ID NO:51, SEQ ID NO:53, SEQ ID NO:54, SEQ ID NO:56, SEQ ID NO: 57, SEQ ID NO:59, SEQ ID NO:60, SEQ ID NO: 62, SEQ ID NO: 63, SEQ ID NO: 65, SEQ ID NO: 66, SEQ ID NO: 68, SEQ ID NO: 69, SEQ ID NO: 71, SEQ ID NO: 72, SEQ ID NO: 74, or SEQ ID NO:75.

Further, the recombinant protein vaccine and/or the adenovirus vaccine contain/contains a nucleic acid for resisting infection with SARS-COV-2 or the mutant thereof.

Further, the nucleotide sequence of the nucleic acid is shown in SEQ ID NO:24, SEQ ID NO:26, SEQ ID NO:28, SEQ ID NO:30, SEQ ID NO:32, SEQ ID NO:34, SEQ ID NO: 36, SEQ ID NO:38, SEQ ID NO: 41, SEQ ID NO: 42, SEQ ID NO: 44, SEQ ID NO:45, SEQ ID NO: 47, SEQ ID NO:48, SEQ ID NO:50, SEQ ID NO:51, SEQ ID NO:53, SEQ ID NO:54, SEQ ID NO:56, SEQ ID NO:57, SEQ ID NO:59, SEQ ID NO:60, SEQ ID NO:62, SEQ ID NO: 63, SEQ ID NO: 65, SEQ ID NO: 66, SEQ ID NO: 68, SEQ ID NO: 69, SEQ ID NO: 71, SEQ ID NO: 72, SEQ ID NO: 74, SEQ ID NO:75, or SEQ ID NO:76.

Further, the nucleotide sequence is obtained based on the encoded amino acid sequence SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:40, SEQ ID NO:43, SEQ ID NO: 46, SEQ ID NO:49, SEQ ID NO: 52, SEQ ID NO: 55, SEQ ID NO: 58, SEQ ID NO: 61, SEQ ID NO: 64, SEQ ID NO:67, SEQ ID NO: 70, SEQ ID NO:73, or SEQ ID NO:77 through optimization of codons or cells; and further, the cells are mammalian cells CHO or insect cells.

The present invention further provides a recombinant vector or an adenovirus vector, where the recombinant vector or the adenovirus vector contains a polynucleotide sequence in the recombinant protein vaccine or the adenovirus vaccine in the pharmaceutical composition or the combined drug, and the polynucleotide sequence is selected from at least one of SEQ ID NO: 10, SEQ ID NO:11, SEQ ID NO: 12, SEQ ID NO: 13, SEQ ID NO: 14, SEQ ID NO: 15, SEQ ID NO: 16, SEQ ID NO: 17, SEQ ID NO: 19, SEQ ID NO:21, SEQ ID NO:23, SEQ ID NO:24, SEQ ID NO:26, SEQ ID NO:28, SEQ ID NO:30, SEQ ID NO:32, SEQ ID NO:34, SEQ ID NO: 36, SEQ ID NO:38, SEQ ID NO:41, SEQ ID NO: 42, SEQ ID NO: 44, SEQ ID NO:45, SEQ ID NO: 47, SEQ ID NO:48, SEQ ID NO: 50, SEQ ID NO:51, SEQ ID NO:53, SEQ ID NO: 54, SEQ ID NO:56, SEQ ID NO:57, SEQ ID NO:59, SEQ ID NO:60, SEQ ID NO:62, SEQ ID NO: 63, SEQ ID NO: 65, SEQ ID NO: 66, SEQ ID NO: 68, SEQ ID NO: 69, SEQ ID NO: 71, SEQ ID NO: 72, SEQ ID NO: 74, SEQ ID NO: 75, or SEQ ID NO:76.

Further, the recombinant vector is selected from at least one of an insect baculovirus expression vector, a mammalian cell expression vector, an Escherichia coli expression vector or a yeast expression vector; preferably, the insect baculovirus expression vector is pFastBac1; preferably, the mammalian cell expression vector is an CHO cell expression vector; further preferably, the CHO cell expression vector is pTT5 or FTP-002; preferably, the Escherichia coli expression vector is pET32a; and preferably, the yeast expression vector is pPICZaA.

Further, the adenovirus vector is a human replication-deficient recombinant adenovirus vector; preferably, the adenovirus vector is selected from a human type 5, type 35 or type 26 replication-deficient adenovirus or/and a chimpanzee type AdC68 or AdC7 replication-deficient adenovirus, and more preferably, is selected from a human type 5 replication-deficient adenovirus with combined deletion of E1 and E3.

The present invention also provides a host cell containing the recombinant vector or the adenovirus vector described above.

Further, the host cell is selected from at least one of insect cells, mammalian cells, Escherichia coli or yeasts; preferably, the insect cells are selected from at least one of sf9 cells, sf21 cells or Hi5 cells; and preferably, the mammalian cells are CHO cells or HEK293 cells.

Further, the recombinant protein vaccine and/or the adenovirus vaccine further comprise/comprises a pharmaceutically acceptable excipient or a complementary ingredient.

Further, the complementary ingredient is an immunologic adjuvant; preferably, the immunologic adjuvant is selected from at least one of a squalene oil-in-water emulsion, aluminum salt, calcium salt, plant saponin, plant polysaccharide, monophosphoryl lipid A, muramyl dipeptide, muramyl tripeptide, bacterial toxin, GM-CSF cytokines, lipid or cationic liposome materials;

further, the immunologic adjuvant satisfies at least one the squalene oil-in-water emulsion being MF59, the aluminum salt being selected from at least one of aluminum hydroxide or alum, the calcium salt being tricalcium phosphate, the plant saponin being QS-21 or ISCOM, the plant polysaccharide being astragalus polysaccharide, the bacterial toxin being selected from at least one of recombinant cholera toxin or diphtheria toxin, the lipid being selected from at least one of phosphatidylethanolamine, phosphatidylcholine, cholesterol or dioleoyl phosphatidylethanolamine, or the cationic liposome material being selected from at least one of (2,3-dioleoxypropyl) trimethyl ammonium chloride, N-[1-(2,3-dioleoyl chloride) propyl]-N,N,N-trimethylamine chloride, cationic cholesterol, dimethyl-2,3-dioleoxypropyl-2-(2-arginine formyl amino) ethyl ammonium trimethyldodecylammonium bromide, dodecyltrimethylammonium bromide, tetradecy ltrimethylammonium bromide, hexadecyltrimethylammonium bromide, dimethyldioctadecylammonium bromide or CpGODN.

Further, the vaccine preparations of the recombinant protein vaccine and the adenovirus vaccine are an intramuscular injection, an injection, a nasal drop, a spray, a nasal spray or inhalation; and preferably, the vaccine is a nasal spray.

The present invention further provides use of the pharmaceutical composition or the combined drug in preparation of a drug for preventing and/or treating infection with SARS-CoV-2 or a mutant thereof.

The present invention further provides use of the pharmaceutical composition or the combined drug in preparation of a drug for treating and/or preventing SARS-COV-2 variant strain infection or pathopoiesis.

Further, the SARS-COV-2 variant strain comprises at least one of Alpha, Beta, Gamma, Delta or Omicron.

The present invention provides use of the pharmaceutical composition or the combined drug in preparation of a drug for treating and/or preventing influenza virus or other respiratory virus infection or pathopoiesis.

The present invention further provides a preparation method of the protein, and the method comprises the following steps: culturing the host cell to express the required protein or precursor, and then recovering the required protein.

The present invention further provides a preparation method of adenovirus, and the method comprises the following steps: constructing a plasmid vector containing the polynucleotide; transfecting the host cell and culturing the transfected host cell; obtaining the recombinant adenovirus; extending the culture of the recombinant adenovirus; and purifying the recombinant adenovirus.

The polynucleotide sequence is selected from at least one of SEQ ID NO: 10, SEQ ID NO: 11, SEQ ID NO:12, SEQ ID NO:13, SEQ ID NO:14, SEQ ID NO: 15, SEQ ID NO: 16, SEQ ID NO: 17, SEQ ID NO: 19, SEQ ID NO:21, SEQ ID NO:23, SEQ ID NO:24, SEQ ID NO:26, SEQ ID NO:28, SEQ ID NO:30, SEQ ID NO:32, SEQ ID NO:34, SEQ ID NO:36, SEQ ID NO: 38, SEQ ID NO:41, SEQ ID NO: 42, SEQ ID NO: 44, SEQ ID NO:45, SEQ ID NO: 47, SEQ ID NO: 48, SEQ ID NO:50, SEQ ID NO:51, SEQ ID NO:53, SEQ ID NO: 54, SEQ ID NO:56, SEQ ID NO:57, SEQ ID NO:59, SEQ ID NO:60, SEQ ID NO:62, SEQ ID NO:63, SEQ ID NO: 65, SEQ ID NO:66, SEQ ID NO: 68, SEQ ID NO: 69, SEQ ID NO: 71, SEQ ID NO: 72, SEQ ID NO: 74, SEQ ID NO:75, or SEQ ID NO:76.

The present invention further provides a preparation method of the recombinant protein, and the method comprises the following steps: constructing a recombinant vector containing the polynucleotide, and immunizing a human body to produce the protein.

Further, the vector is selected from at least one of mRNA, DNA vaccine, adenovirus, vaccinia Ankara virus, or adeno-associated virus.

The present invention further provides a second pharmaceutical composition that contains the recombinant protein vaccine and an inactivated virus vaccine as active ingredients. Further, the inactivated virus vaccine is an inactivated influenza virus vaccine.

The present invention further provides a third pharmaceutical composition that contains the adenovirus vaccine and an influenza virus recombinant protein vaccine as active ingredients.

The present invention further provides use of the second or third pharmaceutical composition in preparation of a drug for treating and/or preventing respiratory virus infection or pathopoiesis.

In the three pharmaceutical compositions of the present invention, the active ingredients are combined in any combination ratio.

Further, in the pharmaceutical compositions, the volume ratio of the recombinant protein vaccine to the adenovirus vaccine is 1-5:1-5.

The present invention further provides a second combined drug, where the recombinant protein vaccine and the inactivated virus vaccine are administered separately or simultaneously. Further, the inactivated virus vaccine is an inactivated influenza virus vaccine.

The present invention further provides a third combined drug, where the adenovirus vaccine and the influenza virus recombinant protein vaccine are administered separately or simultaneously.

Preferably, the influenza virus is selected from influenza A virus and/or influenza B virus.

Further preferably, the influenza A virus is influenza A (H1N1) virus.

SEQ ID NO: 1: original strain (Wuhan strain)
VQPTESIVRFPNITNLCPFGEVFNATRFASVYAWNRKRISNCVADYSVL
YNSASFSTFKCYGVSPTKLNDLCFTNVYADSFVIRGDEVRQIAPGQTGK
IADYNYKLPDDFTGCVIAWNSNNLDSKVGGNYNYLYRLFRKSNLKPFER
DISTEIYQAGSTPCNGVEGENCYFPLQSYGFQPTNGVGYQPYRVVVLSF
ELLHAPATVCGPKKSTNLVKNKCVNFNFNG
SEQ ID NO: 1 shares a same amino acid sequence
with sites 320-545 in the S protein of
SARS-COV-2.
SEQ ID NO: 2: RBD-HR-Beta sequence
MLLVNQSHQGFNKEHTSKMVSAIVLYVLLAAAAHSAFAADSIHIKDSDD
LKNRLAEAGDKLVVIDFMATWCGPCKMIGPKLDEMANEMSDSIVVLKVD
VDECEDIATEYNINSMPTFVFVKNSKKIEEFSGANVDKLRNTIIKLKLA
GSGSGHMHHHHHHSSGDDDDKVQPTESIVRFPNITNLCPFGEVFNATRF
ASVYAWNRKRISNCVADYSVLYNSASFSTFKCYGVSPTKLNDLCFTNVY
ADSFVIRGDEVRQIAPGQTGNIADYNYKLPDDFTGCVIAWNSNNLDSKV
GGNYNYRYRLFRKSNLKPFERDISTEIYQAGSTPCNGVKGFNCYFPLQS
YGFQPTYGVGYQPYRVVVLSFELLHAPATVCGPKKSTNLVKNKSVNFNF
NGLYENQKLIANQFNSAIGKIQDSLSSTASALGKLQDVVNQNAQALNTL
VKQLKNHTSPDVDLGDISGINASVVNIQKEIDRLNEVAKNLNESLIDLQ
EL

The above sequence consists of signal peptide-Trx tag-6His tag-EK restriction enzyme cutting site-RBD sequence-HR1 sequence-HR2 sequence. For the RBD sequence, the amino acid at the site 98 of the SEQ ID NO: 1 sequence is replaced by K with N, the amino acid at the site 133 is replaced by L with R, the amino acid at the site 165 is replaced by E with K, the amino acid at the site 182 is replaced by N with Y, and the amino acid at the site 219 is replaced by C with S.

SEQ ID NO: 3: RBD-HR-Delta sequence
MLLVNQSHQGFNKEHTSKMVSAIVLYVLLAAAAHSAFAADSIHIKDSDD
LKNRLAEAGDKLVVIDFMATWCGPCKMIGPKLDEMANEMSDSIVVLKVD
VDECEDIATEYNINSMPTFVFVKNSKKIEEFSGANVDKLRNTIIKLKLA
GSGSGHMHHHHHHSSGDDDDKVQPTESIVRFPNITNLCPFGEVFNATRF
ASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKV
GGNYNYRYRLFRKSNLKPFERDISTEIYQAGSKPCNGVEGENCYFPLQS
YGFQPTNGVGYQPYRVVVLSFELLHAPATVCGPKKSTNLVKNKSVNFNF
NGLYENQKLIANQFNSAIGKIQDSLSSTASALGKLQDVVNQNAQALNTL
VKQLKNHTSPDVDLGDISGINASVVNIQKEIDRLNEVAKNLNESLIDLQ
EL

The above sequence consists of signal peptide-Trx tag-6His tag-EK restriction enzyme cutting site-RBD sequence-HR1 sequence-HR2 sequence. For the RBD sequence, the amino acid at the site 133 of the SEQ ID NO: 1 sequence is replaced by L with R, the amino acid at the site 159 is replaced by T with K, and the amino acid at the site 219 is replaced by C with S.

SEQ ID NO: 4: RBD-HR-Omicron BA.1
MLLVNQSHQGFNKEHTSKMVSAIVLYVLLAAAAHSAFAADSIHIKDSDD
LKNRLAEAGDKLVVIDFMATWCGPCKMIGPKLDEMANEMSDSIVVLKVD
VDECEDIATEYNINSMPTFVFVKNSKKIEEFSGANVDKLRNTIIKLKLA
GSGSGHMHHHHHHSSGDDDDKVQPTESIVRFPNITNLCPFDEVFNATRF
ASVYAWNRKRISNCVADYSVLYNLAPFFTFKCYGVSPTKLNDLCFTNVY
ADSFVIRGDEVRQIAPGQTGNIADYNYKLPDDFTGCVIAWNSNKLDSKV
SGNYNYLYRLFRKSNLKPFERDISTEIYQAGNKPCNGVAGENCYFPLRS
YSFRPTYGVGHQPYRVVVLSFELLHAPATVCGPKKSTNLVKNKSVNFNF
NGLYENQKLIANQFNSAIGKIQDSLSSTASALGKLQDVVNQNAQALNTL
VKQLKNHTSPDVDLGDISGINASVVNIQKEIDRLNEVAKNLNESLIDLQ
EL

The above sequence consists of signal peptide-Trx tag-6His tag-EK restriction enzyme cutting site-RBD sequence-HR1 sequence-HR2 sequence. The RBD sequence is obtained by mutating five or more amino acid sequences in the SEQ ID NO: 1 sequence.

SEQ ID NO: 5: RBD-HR-Omicron BA.2
MLLVNQSHQGFNKEHTSKMVSAIVLYVLLAAAAHSAFAADSIHIKDSDD
LKNRLAEAGDKLVVIDFMATWCGPCKMIGPKLDEMANEMSDSIVVLKVD
VDECEDIATEYNINSMPTFVFVKNSKKIEEFSGANVDKLRNTIIKLKLA
GSGSGHMHHHHHHSSGDDDDKVQPTESIVRFPNITNLCPFDEVFNATRF
ASVYAWNRKRISNCVADYSVLYNFAPFFAFKCYGVSPTKLNDLCFTNVY
ADSFVIRGNEVSQIAPGQTGNIADYNYKLPDDFTGCVIAWNSNKLDSKV
GGNYNYLYRLFRKSNLKPFERDISTEIYQAGNKPCNGVAGENCYFPLRS
YGFRPTYGVGHQPYRVVVLSFELLHAPATVCGPKKSTNLVKNKSVNFNF
NGLYENQKLIANQFNSAIGKIQDSLSSTASALGKLQDVVNQNAQALNTL
VKQLKNHTSPDVDLGDISGINASVVNIQKEIDRLNEVAKNLNESLIDLQ
EL

The above sequence consists of signal peptide-Trx tag-6His tag-EK restriction enzyme cutting site-RBD sequence-HR1 sequence-HR2 sequence. The RBD sequence is obtained by mutating five or more amino acid sequences in the SEQ ID NO: 1 sequence.

SEQ ID NO: 6: RBD-HR-Omicron BA.4/.5
MLLVNQSHQGFNKEHTSKMVSAIVLYVLLAAAAHSAFAADSIHIKDSDD
LKNRLAEAGDKLVVIDFMATWCGPCKMIGPKLDEMANEMSDSIVVLKVD
VDECEDIATEYNINSMPTFVFVKNSKKIEEFSGANVDKLRNTIIKLKLA
GSGSGHMHHHHHHSSGDDDDKVQPTESIVRFPNITNLCPFDEVFNATRF
ASVYAWNRKRISNCVADYSVLYNFAPFFAFKCYGVSPTKLNDLCFTNVY
ADSFVIRGNEVSQIAPGQTGNIADYNYKLPDDFTGCVIAWNSNKLDSKV
GGNYNYRYRLFRKSNLKPFERDISTEIYQAGNKPCNGVAGVNCYFPLQS
YGFRPTYGVGHQPYRVVVLSFELLHAPATVCGPKKSTNLVKNKSVNFNF
NGLYENQKLIANQFNSAIGKIQDSLSSTASALGKLQDVVNQNAQALNTL
VKQLKNHTSPDVDLGDISGINASVVNIQKEIDRLNEVAKNLNESLIDLQ
EL

The above sequence consists of signal peptide-Trx tag-6His tag-EK restriction enzyme cutting site-RBD sequence-HR1 sequence-HR2 sequence. The RBD sequence is obtained by mutating five or more amino acid sequences in the SEQ ID NO: 1 sequence.

SEQ ID NO: 7: RBD-HR-Omicron BA2.12.1
MLLVNQSHQGFNKEHTSKMVSAIVLYVLLAAAAHSAFAADSIHIKDSDD
LKNRLAEAGDKLVVIDFMATWCGPCKMIGPKLDEMANEMSDSIVVLKVD
VDECEDIATEYNINSMPTFVFVKNSKKIEEFSGANVDKLRNTIIKLKLA
GSGSGHMHHHHHHSSGDDDDKVQPTESIVRFPNITNLCPFDEVFNATRF
ASVYAWNRKRISNCVADYSVLYNFAPFFAFKCYGVSPTKLNDLCFTNVY
ADSFVIRGNEVSQIAPGQTGNIADYNYKLPDDFTGCVIAWNSNKLDSKV
GGNYNYQYRLFRKSNLKPFERDISTEIYQAGNKPCNGVAGENCYFPLRS
YGFRPTYGVGHQPYRVVVLSFELLHAPATVCGPKKSTNLVKNKSVNFNF
NGLYENQKLIANQFNSAIGKIQDSLSSTASALGKLQDVVNQNAQALNTL
VKQLKNHTSPDVDLGDISGINASVVNIQKEIDRLNEVAKNLNESLIDLQ
EL

The above sequence consists of signal peptide-Trx tag-6His tag-EK restriction enzyme cutting site-RBD sequence-HR1 sequence-HR2 sequence. The RBD sequence is obtained by mutating five or more amino acid sequences in the SEQ ID NO: 1 sequence.

SEQ ID NO: 8: RBD-HR- BF.7
MLLVNQSHQGFNKEHTSKMVSAIVLYVLLAAAAHSAFAADSIHIKDSDD
LKNRLAEAGDKLVVIDFMATWCGPCKMIGPKLDEMANEMSDSIVVLKVD
VDECEDIATEYNINSMPTFVFVKNSKKIEEFSGANVDKLRNTIIKLKLA
GSGSGHMHHHHHHSSGDDDDKVQPTESIVRFPNITNLCPFDEVFNATTF
ASVYAWNRKRISNCVADYSVLYNFAPFFAFKCYGVSPTKLNDLCFTNVY
ADSFVIRGNEVSQIAPGQTGNIADYNYKLPDDFTGCVIAWNSNKLDSKV
GGNYNYRYRLFRKSNLKPFERDISTEIYQAGNKPCNGVAGVNCYFPLQS
YGFRPTYGVGHQPYRVVVLSFELLHAPATVCGPKKSTNLVKNKSVNFNF
NGLYENQKLIANQFNSAIGKIQDSLSSTASALGKLQDVVNQNAQALNTL
VKQLKNHTSPDVDLGDISGINASVVNIQKEIDRLNEVAKNLNESLIDLQ
EL

The above sequence consists of signal peptide-Trx tag-6His tag-EK restriction enzyme cutting site-RBD sequence-HR1 sequence-HR2 sequence. The RBD sequence is obtained by mutating five or more amino acid sequences in the SEQ ID NO: 1 sequence.

SEQ ID NO: 9: RBD-HR-BQ.1
MLLVNQSHQGFNKEHTSKMVSAIVLYVLLAAAAHSAFAADSIHIKDSDD
LKNRLAEAGDKLVVIDFMATWCGPCKMIGPKLDEMANEMSDSIVVLKVD
VDECEDIATEYNINSMPTFVFVKNSKKIEEFSGANVDKLRNTIIKLKLA
GSGSGHMHHHHHHSSGDDDDKVQPTESIVRFPNITNLCPFDEVFNATRF
ASVYAWNRKRISNCVADYSVLYNFAPFFAFKCYGVSPTKLNDLCFTNVY
ADSFVIRGNEVSQIAPGQTGNIADYNYKLPDDFTGCVIAWNSNKLDSTV
GGNYNYRYRLFRKSKLKPFERDISTEIYQAGNKPCNGVAGVNCYFPLQS
YGFRPTYGVGHQPYRVVVLSFELLHAPATVCGPKKSTNLVKNKSVNFNF
NGLYENQKLIANQFNSAIGKIQDSLSSTASALGKLQDVVNQNAQALNTL
VKQLKNHTSPDVDLGDISGINASVVNIQKEIDRLNEVAKNLNESLIDLQ
EL

The above sequence consists of signal peptide-Trx tag-6His tag-EK restriction enzyme cutting site-RBD sequence-HR1 sequence-HR2 sequence. The RBD sequence is obtained by mutating five or more amino acid sequences in the SEQ ID NO: 1 sequence.

SEQ ID NO: 10 (nucleotide sequence encoding
SEQ ID NO: 2):
ATGCTACTAGTAAATCAGTCACACCAAGGCTTCAATAAGGAACACACAA
GCAAGATGGTGAGCGCTATCGTGCTGTACGTCCTGCTGGCCGCTGCTGC
TCACAGCGCTTTCGCTGCTGACTCCATCCACATCAAGGACAGCGACGAC
CTGAAGAACCGTCTGGCCGAGGCCGGTGACAAGCTGGTCGTCATCGACT
TCATGGCCACTTGGTGCGGTCCTTGCAAGATGATCGGCCCTAAGCTGGA
CGAGATGGCTAACGAGATGTCCGACAGCATCGTGGTCCTGAAGGTGGAC
GTCGACGAGTGCGAGGACATCGCCACCGAATACAACATCAACAGCATGC
CCACCTTCGTGTTCGTGAAGAACAGCAAGAAGATCGAGGAATTTTCCGG
CGCTAACGTCGACAAGCTGCGTAACACCATCATCAAGCTGAAGCTGGCC
GGCTCCGGCTCCGGCCACATGCATCACCACCACCACCATTCCTCCGGTG
ACGACGACGACAAGGTGCAGCCAACCGAATCTATCGTCAGATTCCCAAA
CATCACTAACCTGTGCCCTTTCGGAGAGGTGTTCAACGCTACCAGGTTC
GCCAGCGTCTACGCTTGGAACCGCAAGCGTATCAGCAACTGCGTCGCCG
ACTACTCTGTGCTGTACAACTCCGCTAGCTTCTCTACTTTCAAGTGCTA
CGGCGTGTCACCTACCAAGCTGAACGACCTGTGCTTCACTAACGTCTAC
GCCGACTCCTTCGTGATCCGCGGAGACGAAGTCCGTCAGATCGCTCCTG
GACAGACCGGAAATATCGCTGACTACAACTACAAGCTGCCAGACGACTT
CACTGGCTGCGTGATCGCTTGGAACTCAAACAACCTGGACTCCAAGGTC
GGTGGCAACTACAACTACCGGTACAGGCTGTTCAGAAAGTCAAACCTGA
AGCCTTTCGAGCGCGACATCTCAACCGAAATCTACCAGGCTGGTTCCAC
TCCCTGCAACGGTGTGAAGGGCTTCAACTGCTACTTCCCCCTGCAGTCC
TACGGTTTCCAGCCAACCTATGGAGTCGGTTACCAGCCTTACCGTGTGG
TCGTGCTGAGCTTCGAACTGCTCCACGCTCCTGCTACTGTGTGCGGTCC
CAAGAAGTCTACTAACCTGGTCAAAAACAAAAGCGTCAACTTCAACTTC
AACGGTCTGTACGAGAACCAGAAGCTGATCGCTAACCAGTTCAACAGCG
CCATCGGCAAGATCCAGGACTCACTGTCATCCACTGCCTCCGCTCTGGG
AAAGCTGCAGGACGTCGTGAACCAGAACGCCCAGGCTCTGAACACCCTG
GTCAAGCAGCTGAAGAACCACACCTCTCCTGACGTCGACCTGGGCGACA
TCAGCGGAATCAACGCTTCTGTCGTGAACATCCAGAAGGAGATCGACCG
CCTGAACGAAGTGGCCAAGAACCTGAACGAATCCCTGATTGACCTCCAA
GAACTCTAA

Signal peptide coding sequence-Trx tag coding sequence-6His tag coding sequence-EK restriction enzyme cutting site coding sequence-RBD coding sequence-HR1

SEQ ID NO: 11 (nucleotide sequence encoding
SEQ ID NO: 3):
ATGCTACTAGTAAATCAGTCACACCAAGGCTTCAATAAGGAACACACAA
GCAAGATGGTGAGCGCTATCGTGCTGTACGTCCTGCTGGCCGCTGCTGC
TCACAGCGCTTTCGCTGCTGACTCCATCCACATCAAGGACAGCGACGAC
CTGAAGAACCGTCTGGCCGAGGCCGGTGACAAGCTGGTCGTCATCGACT
TCATGGCCACTTGGTGCGGTCCTTGCAAGATGATCGGCCCTAAGCTGGA
CGAGATGGCTAACGAGATGTCCGACAGCATCGTGGTCCTGAAGGTGGAC
GTCGACGAGTGCGAGGACATCGCCACCGAATACAACATCAACAGCATGC
CCACCTTCGTGTTCGTGAAGAACAGCAAGAAGATCGAGGAATTTTCCGG
CGCTAACGTCGACAAGCTGCGTAACACCATCATCAAGCTGAAGCTGGCC
GGCTCCGGCTCCGGCCACATGCATCACCACCACCACCATTCCTCCGGTG
ACGACGACGACAAGGTGCAGCCAACCGAATCTATCGTCAGATTCCCAAA
CATCACTAACCTGTGCCCTTTCGGAGAGGTGTTCAACGCTACCAGGTTC
GCCAGCGTCTACGCTTGGAACCGCAAGCGTATCAGCAACTGCGTCGCCG
ACTACTCTGTGCTGTACAACTCCGCTAGCTTCTCTACTTTCAAGTGCTA
CGGCGTGTCACCTACCAAGCTGAACGACCTGTGCTTCACTAACGTCTAC
GCCGACTCCTTCGTGATCCGCGGAGACGAAGTCCGTCAGATCGCTCCTG
GACAGACCGGAAAGATCGCTGACTACAACTACAAGCTGCCAGACGACTT
CACTGGCTGCGTGATCGCTTGGAACTCAAACAACCTGGACTCCAAGGTC
GGTGGCAACTACAACTACCGGTACAGGCTGTTCAGAAAGTCAAACCTGA
AGCCTTTCGAGCGCGACATCTCAACCGAAATCTACCAGGCTGGTTCCAA
GCCCTGCAACGGTGTGGAGGGCTTCAACTGCTACTTCCCCCTGCAGTCC
TACGGTTTCCAGCCAACCAACGGAGTCGGTTACCAGCCTTACCGTGTGG
TCGTGCTGAGCTTCGAACTGCTCCACGCTCCTGCTACTGTGTGCGGTCC
CAAGAAGTCTACTAACCTGGTCAAAAACAAAAGCGTCAACTTCAACTTC
AACGGTCTGTACGAGAACCAGAAGCTGATCGCTAACCAGTTCAACAGCG
CCATCGGCAAGATCCAGGACTCACTGTCATCCACTGCCTCCGCTCTGGG
AAAGCTGCAGGACGTCGTGAACCAGAACGCCCAGGCTCTGAACACCCTG
GTCAAGCAGCTGAAGAACCACACCTCTCCTGACGTCGACCTGGGCGACA
TCAGCGGAATCAACGCTTCTGTCGTGAACATCCAGAAGGAGATCGACCG
CCTGAACGAAGTGGCCAAGAACCTGAACGAATCCCTGATTGACCTCCAA
GAACTCTAA

Signal peptide coding sequence-Trx tag coding sequence-6His tag coding sequence-EK restriction enzyme cutting site coding sequence-RBD coding sequence-HR1

SEQ ID NO: 12 (nucleotide sequence encoding
SEQ ID NO: 4):
ATGCTACTAGTAAATCAGTCACACCAAGGCTTCAATAAGGAACACACAA
GCAAGATGGTGAGCGCTATCGTGCTGTACGTCCTGCTGGCCGCTGCTGC
TCACAGCGCTTTCGCTGCTGACTCCATCCACATCAAGGACAGCGACGAC
CTGAAGAACCGTCTGGCCGAGGCCGGTGACAAGCTGGTCGTCATCGACT
TCATGGCCACTTGGTGCGGTCCTTGCAAGATGATCGGCCCTAAGCTGGA
CGAGATGGCTAACGAGATGTCCGACAGCATCGTGGTCCTGAAGGTGGAC
GTCGACGAGTGCGAGGACATCGCCACCGAATACAACATCAACAGCATGC
CCACCTTCGTGTTCGTGAAGAACAGCAAGAAGATCGAGGAATTTTCCGG
CGCTAACGTCGACAAGCTGCGTAACACCATCATCAAGCTGAAGCTGGCC
GGCTCCGGCTCCGGCCACATGCATCACCACCACCACCATTCCTCCGGTG
ACGACGACGACAAGGTGCAGCCAACCGAATCTATCGTCAGATTCCCAAA
CATCACTAACCTGTGCCCTTTCGATGAGGTGTTCAACGCTACCAGGTTC
GCCAGCGTCTACGCTTGGAACCGCAAGCGTATCAGCAACTGCGTCGCCG
ACTACTCTGTGCTGTACAACCTCGCTCCTTTCTTCACTTTCAAGTGCTA
CGGCGTGTCACCTACCAAGCTGAACGACCTGTGCTTCACTAACGTCTAC
GCCGACTCCTTCGTGATCCGCGGAGACGAAGTCCGTCAGATCGCTCCTG
GACAGACCGGAAATATCGCTGACTACAACTACAAGCTGCCAGACGACTT
CACTGGCTGCGTGATCGCTTGGAACTCAAACAAGCTGGACTCCAAGGTC
AGTGGCAACTACAACTACCTGTACAGGCTGTTCAGAAAGTCAAACCTGA
AGCCTTTCGAGCGCGACATCTCAACCGAAATCTACCAGGCTGGTAATAA
GCCCTGCAACGGTGTGGCTGGCTTCAACTGCTACTTCCCCCTGAGATCC
TACTCTTTCCGACCAACCTACGGAGTCGGTCATCAGCCTTACCGTGTGG
TCGTGCTGAGCTTCGAACTGCTCCACGCTCCTGCTACTGTGTGCGGTCC
CAAGAAGTCTACTAACCTGGTCAAAAACAAAAGCGTCAACTTCAACTTC
AACGGTCTGTACGAGAACCAGAAGCTGATCGCTAACCAGTTCAACAGCG
CCATCGGCAAGATCCAGGACTCACTGTCATCCACTGCCTCCGCTCTGGG
AAAGCTGCAGGACGTCGTGAACCAGAACGCCCAGGCTCTGAACACCCTG
GTCAAGCAGCTGAAGAACCACACCTCTCCTGACGTCGACCTGGGCGACA
TCAGCGGAATCAACGCTTCTGTCGTGAACATCCAGAAGGAGATCGACCG
CCTGAACGAAGTGGCCAAGAACCTGAACGAATCCCTGATTGACCTCCAA
GAACTCTAA

Signal peptide coding sequence-Trx tag coding sequence-6His tag coding sequence-EK restriction enzyme cutting site coding sequence-RBD coding sequence-HR1

SEQ ID NO: 13 (nucleotide sequence encoding
SEQ ID NO: 5):
ATGCTGCTGGTGAACCAGAGCCACCAGGGTTTCAACAAGGAACACACTA
GCAAGATGGTGTCTGCTATCGTCCTCTACGTGCTGCTCGCCGCCGCTGC
TCACTCAGCTTTCGCCGCTGACTCTATCCACATCAAGGACTCCGACGAC
CTCAAGAACCGTTTGGCCGAAGCCGGCGACAAGCTGGTCGTTATCGACT
TCATGGCTACCTGGTGTGGCCCTTGCAAGATGATCGGACCTAAGCTGGA
CGAAATGGCTAACGAGATGAGCGACAGCATCGTCGTTCTGAAGGTGGAC
GTCGACGAATGTGAGGACATCGCCACTGAGTACAACATCAACAGCATGC
CTACCTTCGTTTTCGTGAAGAACTCTAAGAAGATCGAGGAGTTCTCTGG
TGCCAACGTGGACAAGCTCCGTAACACCATCATCAAGCTCAAGCTGGCC
GGCAGCGGTAGCGGTCACATGCACCACCACCACCACCATTCAAGCGGCG
ACGACGACGACAAGGTGCAGCCAACCGAATCAATCGTCCGCTTCCCAAA
CATCACTAACCTGTGCCCATTCGACGAGGTGTTCAACGCCACCCGTTTC
GCCTCTGTCTACGCTTGGAACCGCAAGCGCATCTCCAACTGTGTCGCCG
ACTACAGCGTGCTGTACAACTTCGCTCCTTTCTTCGCATTCAAGTGTTA
CGGAGTGTCCCCTACCAAGCTCAACGATTTGTGCTTCACAAACGTGTAC
GCTGACAGCTTCGTGATCCGTGGTAACGAGGTTTCTCAAATCGCCCCTG
GTCAGACAGGTAACATCGCTGACTACAACTACAAGCTCCCAGACGACTT
CACTGGCTGTGTTATCGCTTGGAACTCAAACAAGCTGGACTCTAAGGTG
GGTGGTAACTACAACTACCTCTACCGTCTGTTCCGCAAGAGCAACCTCA
AGCCATTCGAACGTGACATCAGCACAGAGATCTACCAAGCCGGTAACAA
GCCCTGCAACGGCGTTGCCGGATTCAACTGCTACTTCCCCCTCCGTTCC
TACGGATTCCGCCCAACCTACGGCGTGGGTCACCAGCCATACCGCGTGG
TTGTGCTGAGCTTCGAACTGCTGCACGCCCCCGCTACCGTGTGTGGCCC
TAAGAAGAGCACTAACCTCGTTAAGAACAAGTCCGTGAACTTCAACTTC
AACGGTCTCTACGAAAACCAGAAGCTGATCGCTAACCAGTTCAACTCCG
CCATCGGCAAGATCCAGGACTCCCTGTCCTCCACAGCTTCCGCTCTGGG
CAAGCTGCAGGACGTTGTGAACCAAAACGCTCAGGCCCTGAACACACTG
GTGAAGCAACTGAAGAACCACACCAGCCCCGACGTTGACCTGGGTGACA
TCAGCGGTATCAACGCTAGCGTGGTGAACATCCAGAAGGAGATCGACAG
GCTGAACGAAGTGGCTAAGAACTTGAACGAGAGCCTCATCGACCTGCAG
GAACTGTAA

Signal peptide coding sequence-Trx tag coding sequence-6His tag coding sequence-EK restriction enzyme cutting site coding sequence-RBD coding sequence-HR1

SEQ ID NO: 14 (nucleotide sequence encoding
SEQ ID NO: 6):
ATGCTGCTGGTGAACCAGAGCCACCAGGGTTTCAACAAGGAACACACTA
GCAAGATGGTGTCTGCTATCGTCCTCTACGTGCTGCTCGCCGCCGCTGC
TCACTCAGCTTTCGCCGCTGACTCTATCCACATCAAGGACTCCGACGAC
CTCAAGAACCGTTTGGCCGAAGCCGGCGACAAGCTGGTCGTTATCGACT
TCATGGCTACCTGGTGTGGCCCTTGCAAGATGATCGGACCTAAGCTGGA
CGAAATGGCTAACGAGATGAGCGACAGCATCGTCGTTCTGAAGGTGGAC
GTCGACGAATGTGAGGACATCGCCACTGAGTACAACATCAACAGCATGC
CTACCTTCGTTTTCGTGAAGAACTCTAAGAAGATCGAGGAGTTCTCTGG
TGCCAACGTGGACAAGCTCCGTAACACCATCATCAAGCTCAAGCTGGCC
GGCAGCGGTAGCGGTCACATGCACCACCACCACCACCATTCAAGCGGCG
ACGACGACGACAAGGTGCAGCCAACCGAATCAATCGTCCGCTTCCCAAA
CATCACTAACCTGTGCCCATTCGACGAGGTGTTCAACGCCACCCGTTTC
GCCTCTGTCTACGCTTGGAACCGCAAGCGCATCTCCAACTGTGTCGCCG
ACTACAGCGTGCTGTACAACTTCGCTCCTTTCTTCGCATTCAAGTGTTA
CGGAGTGTCCCCTACCAAGCTCAACGATTTGTGCTTCACAAACGTGTAC
GCTGACAGCTTCGTGATCCGTGGTAACGAGGTTTCTCAAATCGCCCCTG
GTCAGACAGGTAACATCGCTGACTACAACTACAAGCTCCCAGACGACTT
CACTGGCTGTGTTATCGCTTGGAACTCAAACAAGCTGGACTCTAAGGTG
GGTGGTAACTACAACTACCGGTACCGTCTGTTCCGCAAGAGCAACCTCA
AGCCATTCGAACGTGACATCAGCACAGAGATCTACCAAGCCGGTAACAA
GCCCTGCAACGGCGTTGCCGGAGTCAACTGCTACTTCCCCCTCCAGTCC
TACGGATTCCGCCCAACCTACGGCGTGGGTCACCAGCCATACCGCGTGG
TTGTGCTGAGCTTCGAACTGCTGCACGCCCCCGCTACCGTGTGTGGCCC
TAAGAAGAGCACTAACCTCGTTAAGAACAAGTCCGTGAACTTCAACTTC
AACGGTCTCTACGAAAACCAGAAGCTGATCGCTAACCAGTTCAACTCCG
CCATCGGCAAGATCCAGGACTCCCTGTCCTCCACAGCTTCCGCTCTGGG
CAAGCTGCAGGACGTTGTGAACCAAAACGCTCAGGCCCTGAACACACTG
GTGAAGCAACTGAAGAACCACACCAGCCCCGACGTTGACCTGGGTGACA
TCAGCGGTATCAACGCTAGCGTGGTGAACATCCAGAAGGAGATCGACAG
GCTGAACGAAGTGGCTAAGAACTTGAACGAGAGCCTCATCGACCTGCAG
GAACTGTAA

Signal peptide coding sequence-Trx tag coding sequence-6His tag coding sequence-EK restriction enzyme cutting site coding sequence-RBD coding sequence-HR1

SEQ ID NO: 15 (nucleotide sequence encoding
SEQ ID NO: 7):
ATGCTGCTGGTGAACCAGAGCCACCAGGGTTTCAACAAGGAACACACTA
GCAAGATGGTGTCTGCTATCGTCCTCTACGTGCTGCTCGCCGCCGCTGC
TCACTCAGCTTTCGCCGCTGACTCTATCCACATCAAGGACTCCGACGAC
CTCAAGAACCGTTTGGCCGAAGCCGGCGACAAGCTGGTCGTTATCGACT
TCATGGCTACCTGGTGTGGCCCTTGCAAGATGATCGGACCTAAGCTGGA
CGAAATGGCTAACGAGATGAGCGACAGCATCGTCGTTCTGAAGGTGGAC
GTCGACGAATGTGAGGACATCGCCACTGAGTACAACATCAACAGCATGC
CTACCTTCGTTTTCGTGAAGAACTCTAAGAAGATCGAGGAGTTCTCTGG
TGCCAACGTGGACAAGCTCCGTAACACCATCATCAAGCTCAAGCTGGCC
GGCAGCGGTAGCGGTCACATGCACCACCACCACCACCATTCAAGCGGCG
ACGACGACGACAAGGTGCAGCCAACCGAATCAATCGTCCGCTTCCCAAA
CATCACTAACCTGTGCCCATTCGACGAGGTGTTCAACGCCACCCGTTTC
GCCTCTGTCTACGCTTGGAACCGCAAGCGCATCTCCAACTGTGTCGCCG
ACTACAGCGTGCTGTACAACTTCGCTCCTTTCTTCGCATTCAAGTGTTA
CGGAGTGTCCCCTACCAAGCTCAACGATTTGTGCTTCACAAACGTGTAC
GCTGACAGCTTCGTGATCCGTGGTAACGAGGTTTCTCAAATCGCCCCTG
GTCAGACAGGTAACATCGCTGACTACAACTACAAGCTCCCAGACGACTT
CACTGGCTGTGTTATCGCTTGGAACTCAAACAAGCTGGACTCTAAGGTG
GGTGGTAACTACAACTACCAGTACCGTCTGTTCCGCAAGAGCAACCTCA
AGCCATTCGAACGTGACATCAGCACAGAGATCTACCAAGCCGGTAACAA
GCCCTGCAACGGCGTTGCCGGATTCAACTGCTACTTCCCCCTCCGTTCC
TACGGATTCCGCCCAACCTACGGCGTGGGTCACCAGCCATACCGCGTGG
TTGTGCTGAGCTTCGAACTGCTGCACGCCCCCGCTACCGTGTGTGGCCC
TAAGAAGAGCACTAACCTCGTTAAGAACAAGTCCGTGAACTTCAACTTC
AACGGTCTCTACGAAAACCAGAAGCTGATCGCTAACCAGTTCAACTCCG
CCATCGGCAAGATCCAGGACTCCCTGTCCTCCACAGCTTCCGCTCTGGG
CAAGCTGCAGGACGTTGTGAACCAAAACGCTCAGGCCCTGAACACACTG
GTGAAGCAACTGAAGAACCACACCAGCCCCGACGTTGACCTGGGTGACA
TCAGCGGTATCAACGCTAGCGTGGTGAACATCCAGAAGGAGATCGACAG
GCTGAACGAAGTGGCTAAGAACTTGAACGAGAGCCTCATCGACCTGCAG
GAACTGTAA

Signal peptide coding sequence-Trx tag coding sequence-6His tag coding sequence-EK restriction enzyme cutting site coding sequence-RBD coding sequence-HR1

SEQ ID NO: 16 (nucleotide sequence encoding
SEQ ID NO: 8):
ATGCTGCTGGTGAACCAGAGCCACCAGGGTTTCAACAAGGAACACACTA
GCAAGATGGTGTCTGCTATCGTCCTCTACGTGCTGCTCGCCGCCGCTGC
TCACTCAGCTTTCGCCGCTGACTCTATCCACATCAAGGACTCCGACGAC
CTCAAGAACCGTTTGGCCGAAGCCGGCGACAAGCTGGTCGTTATCGACT
TCATGGCTACCTGGTGTGGCCCTTGCAAGATGATCGGACCTAAGCTGGA
CGAAATGGCTAACGAGATGAGCGACAGCATCGTCGTTCTGAAGGTGGAC
GTCGACGAATGTGAGGACATCGCCACTGAGTACAACATCAACAGCATGC
CTACCTTCGTTTTCGTGAAGAACTCTAAGAAGATCGAGGAGTTCTCTGG
TGCCAACGTGGACAAGCTCCGTAACACCATCATCAAGCTCAAGCTGGCC
GGCAGCGGTAGCGGTCACATGCACCACCACCACCACCATTCAAGCGGCG
ACGACGACGACAAGGTGCAGCCAACCGAATCAATCGTCCGCTTCCCAAA
CATCACTAACCTGTGCCCATTCGACGAGGTGTTCAACGCCACCCGTTTC
GCCTCTGTCTACGCTTGGAACCGCAAGCGCATCTCCAACTGTGTCGCCG
ACTACAGCGTGCTGTACAACTTCGCTCCTTTCTTCGCATTCAAGTGTTA
CGGAGTGTCCCCTACCAAGCTCAACGATTTGTGCTTCACAAACGTGTAC
GCTGACAGCTTCGTGATCCGTGGTAACGAGGTTTCTCAAATCGCCCCTG
GTCAGACAGGTAACATCGCTGACTACAACTACAAGCTCCCAGACGACTT
CACTGGCTGTGTTATCGCTTGGAACTCAAACAAGCTGGACTCTAAGGTG
GGTGGTAACTACAACTACCGGTACCGTCTGTTCCGCAAGAGCAACCTCA
AGCCATTCGAACGTGACATCAGCACAGAGATCTACCAAGCCGGTAACAA
GCCCTGCAACGGCGTTGCCGGAGTCAACTGCTACTTCCCCCTCCAGTCC
TACGGATTCCGCCCAACCTACGGCGTGGGTCACCAGCCATACCGCGTGG
TTGTGCTGAGCTTCGAACTGCTGCACGCCCCCGCTACCGTGTGTGGCCC
TAAGAAGAGCACTAACCTCGTTAAGAACAAGTCCGTGAACTTCAACTTC
AACGGTCTCTACGAAAACCAGAAGCTGATCGCTAACCAGTTCAACTCCG
CCATCGGCAAGATCCAGGACTCCCTGTCCTCCACAGCTTCCGCTCTGGG
CAAGCTGCAGGACGTTGTGAACCAAAACGCTCAGGCCCTGAACACACTG
GTGAAGCAACTGAAGAACCACACCAGCCCCGACGTTGACCTGGGTGACA
TCAGCGGTATCAACGCTAGCGTGGTGAACATCCAGAAGGAGATCGACAG
GCTGAACGAAGTGGCTAAGAACTTGAACGAGAGCCTCATCGACCTGCAG
GAACTGTAA

Signal peptide coding sequence-Trx tag coding sequence-6His tag coding sequence-EK restriction enzyme cutting site coding sequence-RBD coding sequence-HR1

SEQ ID NO: 17 (nucleotide sequence encoding
SEQ ID NO: 9):
ATGCTGCTGGTGAACCAGAGCCACCAGGGTTTCAACAAGGAACACACTA
GCAAGATGGTGTCTGCTATCGTCCTCTACGTGCTGCTCGCCGCCGCTGC
TCACTCAGCTTTCGCCGCTGACTCTATCCACATCAAGGACTCCGACGAC
CTCAAGAACCGTTTGGCCGAAGCCGGCGACAAGCTGGTCGTTATCGACT
TCATGGCTACCTGGTGTGGCCCTTGCAAGATGATCGGACCTAAGCTGGA
CGAAATGGCTAACGAGATGAGCGACAGCATCGTCGTTCTGAAGGTGGAC
GTCGACGAATGTGAGGACATCGCCACTGAGTACAACATCAACAGCATGC
CTACCTTCGTTTTCGTGAAGAACTCTAAGAAGATCGAGGAGTTCTCTGG
TGCCAACGTGGACAAGCTCCGTAACACCATCATCAAGCTCAAGCTGGCC
GGCAGCGGTAGCGGTCACATGCACCACCACCACCACCATTCAAGCGGCG
ACGACGACGACAAGGTGCAGCCAACCGAATCAATCGTCCGCTTCCCAAA
CATCACTAACCTGTGCCCATTCGACGAGGTGTTCAACGCCACCCGTTTC
GCCTCTGTCTACGCTTGGAACCGCAAGCGCATCTCCAACTGTGTCGCCG
ACTACAGCGTGCTGTACAACTTCGCTCCTTTCTTCGCATTCAAGTGTTA
CGGAGTGTCCCCTACCAAGCTCAACGATTTGTGCTTCACAAACGTGTAC
GCTGACAGCTTCGTGATCCGTGGTAACGAGGTTTCTCAAATCGCCCCTG
GTCAGACAGGTAACATCGCTGACTACAACTACAAGCTCCCAGACGACTT
CACTGGCTGTGTTATCGCTTGGAACTCAAACAAGCTGGACTCTACGGTG
GGTGGTAACTACAACTACCGGTACCGTCTGTTCCGCAAGAGCAAGCTCA
AGCCATTCGAACGTGACATCAGCACAGAGATCTACCAAGCCGGTAACAA
GCCCTGCAACGGCGTTGCCGGAGTCAACTGCTACTTCCCCCTCCAGTCC
TACGGATTCCGCCCAACCTACGGCGTGGGTCACCAGCCATACCGCGTGG
TTGTGCTGAGCTTCGAACTGCTGCACGCCCCCGCTACCGTGTGTGGCCC
TAAGAAGAGCACTAACCTCGTTAAGAACAAGTCCGTGAACTTCAACTTC
AACGGTCTCTACGAAAACCAGAAGCTGATCGCTAACCAGTTCAACTCCG
CCATCGGCAAGATCCAGGACTCCCTGTCCTCCACAGCTTCCGCTCTGGG
CAAGCTGCAGGACGTTGTGAACCAAAACGCTCAGGCCCTGAACACACTG
GTGAAGCAACTGAAGAACCACACCAGCCCCGACGTTGACCTGGGTGACA
TCAGCGGTATCAACGCTAGCGTGGTGAACATCCAGAAGGAGATCGACAG
GCTGAACGAAGTGGCTAAGAACTTGAACGAGAGCCTCATCGACCTGCAG
GAACTGTAA

Signal peptide coding sequence-Trx tag coding sequence-6His tag coding sequence-EK restriction enzyme cutting site coding sequence-RBD coding sequence-HR1 coding sequence-HR2 coding sequence

Construction mode of M65: RBD (BA.1)-HR1-RBD (Delta)-HR2-RBD (Beta)

The amino acid sequence constructed for M65 is:

SEQ ID NO: 18: RBD (BA.1)-HR1-RBD (Delta)-HR2-RBD
(Beta)
MDAMKRGLCCVLLLCGAVFVSPVQPTESIVRFPNITNLCPFDEVFNATR
FASVYAWNRKRISNCVADYSVLYNLAPFFTFKCYGVSPTKLNDLCFTNV
YADSFVIRGDEVRQIAPGQTGNIADYNYKLPDDFTGCVIAWNSNKLDSK
VSGNYNYLYRLFRKSNLKPFERDISTEIYQAGNKPCNGVAGFNCYFPLR
SYSFRPTYGVGHQPYRVVVLSFELLHAPATVCGPKKSTNLVKNKSVNFN
FNGLYENQKLIANQFNSAIGKIQDSLSSTASALGKLQDVVNQNAQALNT
LVKQLVQPTESIVRFPNITNLCPFGEVFNATRFASVYAWNRKRISNCVA
DYSVLYNSASFSTFKCYGVSPTKLNDLCFTNVYADSFVIRGDEVRQIAP
GQTGKIADYNYKLPDDFTGCVIAWNSNNLDSKVGGNYNYRYRLFRKSNL
KPFERDISTEIYQAGSKPCNGVEGFNCYFPLQSYGFQPTNGVGYQPYRV
VVLSFELLHAPATVCGPKKSTNLVKNKSVNFNFNGKNHTSPDVDLGDIS
GINASVVNIQKEIDRLNEVAKNLNESLIDLQELGKYVQPTESIVRFPNI
TNLCPFGEVFNATRFASVYAWNRKRISNCVADYSVLYNSASFSTFKCYG
VSPTKLNDLCFTNVYADSFVIRGDEVRQIAPGQTGNIADYNYKLPDDFT
GCVIAWNSNNLDSKVGGNYNYLYRLFRKSNLKPFERDISTEIYQAGSTP
CNGVKGFNCYFPLQSYGFQPTYGVGYQPYRVVVLSFELLHAPATVCGPK
KSTNLVKNKSVNFNFNG

tPA signal peptide—RBD sequence of OmicronBA.1-HR1 sequence-RBD sequence of Delta-HR2 sequence-RBD sequence of Beta

SEQ ID NO: 19 (nucleotide sequence encoding
SEQ ID NO: 18):
ATGGACGCCATGAAGAGAGGCCTGTGCTGCGTGCTGCTCCTGTGCGGCG
CCGTGTTCGTGAGCCCCGTGCAACCCACCGAAAGCATTGTGCGGTTTCC
CAATATCACCAACCTCTGTCCTTTCGACGAGGTGTTCAACGCCACAAGA
TTTGCTAGCGTGTACGCCTGGAACCGGAAGAGAATCTCCAATTGTGTCG
CCGACTATAGCGTCCTCTATAACCTGGCCCCCTTCTTCACATTCAAGTG
CTACGGCGTGTCCCCCACAAAGCTGAACGACCTGTGCTTTACCAATGTG
TACGCCGACAGCTTCGTCATCAGAGGGGACGAGGTCCGGCAGATCGCTC
CTGGGCAGACCGGCAATATCGCCGACTACAACTATAAGCTCCCTGACGA
CTTCACCGGGTGCGTCATTGCTTGGAACTCCAACAAGCTGGATTCCAAA
GTGAGCGGGAACTACAACTATCTCTACAGACTGTTCAGAAAGAGCAACC
TGAAACCTTTCGAAAGAGACATCTCCACAGAGATCTACCAAGCCGGCAA
CAAGCCCTGCAACGGCGTGGCCGGCTTTAATTGTTACTTCCCCCTCAGA
AGCTACAGCTTCAGACCCACCTACGGCGTGGGCCATCAGCCCTATAGAG
TCGTGGTGCTGTCCTTCGAACTGCTGCACGCCCCCGCCACCGTGTGTGG
CCCCAAGAAAAGCACAAATCTGGTCAAGAATAAGAGCGTCAATTTTAAT
TTCAACGGCCTGTACGAGAATCAGAAGCTGATCGCCAATCAGTTCAACA
GCGCCATCGGCAAGATCCAAGACAGCCTGAGCAGCACCGCTAGCGCCCT
GGGCAAGCTGCAAGACGTGGTGAATCAGAACGCCCAAGCCCTGAACACC
CTGGTGAAGCAGCTGGTGCAGCCCACCGAATCCATTGTCAGATTCCCCA
ACATCACCAACCTGTGCCCCTTTGGCGAAGTGTTTAACGCCACCCGGTT
CGCTAGCGTGTATGCCTGGAACCGGAAGAGAATTAGCAACTGCGTGGCC
GATTACAGCGTGCTCTATAACAGCGCTAGCTTCAGCACCTTTAAGTGCT
ATGGCGTGAGCCCTACCAAGCTGAATGACCTCTGCTTCACCAACGTGTA
TGCTGATAGCTTCGTGATCAGAGGCGACGAAGTGAGACAGATCGCCCCT
GGGCAGACCGGCAAGATTGCCGACTACAATTATAAGCTGCCTGATGATT
TCACCGGCTGCGTGATCGCCTGGAACAGCAACAACCTGGATTCCAAGGT
CGGGGGCAATTACAATTACAGATACAGACTCTTCAGAAAGTCCAACCTG
AAGCCCTTCGAGAGAGACATCAGCACCGAGATCTATCAAGCCGGGAGCA
AGCCCTGTAACGGCGTGGAGGGCTTCAATTGCTACTTTCCCCTGCAGTC
CTATGGGTTTCAGCCCACCAACGGGGTGGGCTATCAGCCCTATAGAGTC
GTCGTCCTCAGCTTCGAACTGCTGCACGCTCCTGCTACAGTGTGCGGGC
CCAAGAAGAGCACCAATCTCGTCAAGAATAAAAGCGTCAACTTCAACTT
CAACGGCAAGAACCACACAAGCCCCGACGTGGACCTGGGCGACATCAGC
GGCATCAACGCTAGCGTGGTGAACATTCAGAAGGAGATCGACAGACTGA
ACGAGGTGGCCAAGAACCTGAACGAGAGCCTGATCGACCTGCAAGAGCT
GGGCAAGTACGTGCAGCCCACCGAGAGCATCGTGAGATTCCCCAATATC
ACAAATCTCTGCCCTTTTGGCGAGGTGTTTAACGCTACAAGATTCGCTA
GCGTCTACGCCTGGAACAGAAAACGGATCAGCAATTGTGTGGCCGACTA
TAGCGTGCTGTACAATTCCGCTAGCTTCAGCACATTCAAATGCTACGGG
GTGTCCCCCACCAAGCTGAACGATCTCTGCTTCACCAACGTCTACGCCG
ACAGCTTCGTGATTAGAGGCGACGAGGTCAGACAAATCGCCCCTGGGCA
GACCGGCAACATCGCTGACTATAATTACAAGCTGCCTGACGATTTCACC
GGGTGTGTGATCGCCTGGAACTCCAACAATCTGGACTCCAAGGTGGGGG
GCAACTATAACTACCTGTACAGACTCTTTAGAAAGAGCAATCTCAAGCC
CTTCGAACGGGATATTAGCACCGAAATTTACCAAGCCGGCTCCACACCC
TGTAACGGCGTGAAGGGGTTCAACTGCTATTTCCCCCTGCAGAGCTACG
GCTTTCAGCCCACCTACGGCGTGGGGTATCAGCCTTACCGGGTGGTCGT
GCTCAGCTTCGAGCTGCTGCATGCTCCTGCCACCGTCTGTGGGCCTAAG
AAGAGCACCAACCTGGTGAAGAACAAATCCGTGAACTTCAACTTTAACG
GCTGA

Construction of M65-BA.4/5: replacement of BA.1RBD in M65 with RBD sequence of BA.4/5, and construction mode thereof: RBD (BA.4/5)-HR1-RBD (Delta)-HR2-RBD (WT)

The amino acid sequence constructed for M65-BA.4/5 is:

EQ ID No. 20: RBD (BA.4/5)-HR1-RBD (Delta)-HR2-
RBD (WT)
MDAMKRGLCCVLLLCGAVFVSPVQPTESIVRFPNITNLCPFDEVFNATR
FASVYAWNRKRISNCVADYSVLYNFAPFFAFKCYGVSPTKLNDLCFTNV
YADSFVIRGNEVSQIAPGQTGNIADYNYKLPDDFTGCVIAWNSNKLDSK
VGGNYNYRYRLFRKSNLKPFERDISTEIYQAGNKPCNGVAGVNCYFPLQ
SYGFRPTYGVGHQPYRVVVLSFELLHAPATVCGPKKSTNLVKNKSVNFN
FNGLYENQKLIANQFNSAIGKIQDSLSSTASALGKLQDVVNQNAQALNT
LVKQLVQPTESIVRFPNITNLCPFGEVFNATRFASVYAWNRKRISNCVA
DYSVLYNSASFSTFKCYGVSPTKLNDLCFTNVYADSFVIRGDEVRQIAP
GQTGKIADYNYKLPDDFTGCVIAWNSNNLDSKVGGNYNYRYRLFRKSNL
KPFERDISTEIYQAGSKPCNGVEGFNCYFPLQSYGFQPTNGVGYQPYRV
VVLSFELLHAPATVCGPKKSTNLVKNKSVNFNFNGKNHTSPDVDLGDIS
GINASVVNIQKEIDRLNEVAKNLNESLIDLQELGKYVQPTESIVRFPNI
TNLCPFGEVENATRFASVYAWNRKRISNCVADYSVLYNSASFSTFKCYG
VSPTKLNDLCFTNVYADSFVIRGDEVRQIAPGQTGKIADYNYKLPDDFT
GCVIAWNSNNLDSKVGGNYNYLYRLFRKSNLKPFERDISTEIYQAGSTP
CNGVEGENCYFPLQSYGFQPTNGVGYQPYRVVVLSFELLHAPATVCGPK
KSTNLVKNKSVNFNFNG

tPA signal peptide-RBD sequence of OmicronBA.4/5-HR1 sequence-RBD sequence of Delta-HR2 sequence-RBD sequence of WT.

SEQ ID NO: 21 (nucleotide sequence encoding
SEQ ID NO: 20):
ATGGACGCCATGAAGAGAGGCCTGTGCTGCGTGCTGCTCCTGTGCGGCG
CCGTGTTCGTGAGCCCCGTGCAACCCACCGAGAGCATTGTGAGATTCCC
CAATATCACCAACCTCTGCCCCTTCGACGAAGTGTTTAACGCCACAAGA
TTCGCTAGCGTCTACGCTTGGAATAGAAAGCGGATCAGCAACTGCGTCG
CTGATTACTCCGTGCTGTACAACTTCGCCCCCTTCTTCGCTTTCAAGTG
TTACGGGGTGAGCCCTACCAAGCTGAACGACCTCTGTTTCACCAATGTG
TACGCCGATAGCTTCGTGATCCGGGGCAACGAAGTGAGCCAAATCGCCC
CCGGCCAAACCGGCAATATCGCTGACTACAACTACAAGCTCCCTGATGA
CTTCACCGGCTGTGTGATCGCTTGGAACAGCAACAAGCTGGACAGCAAA
GTGGGCGGGAATTATAACTACAGATACCGGCTCTTCCGGAAGAGCAACC
TCAAGCCTTTTGAGCGGGACATCTCCACCGAAATCTACCAAGCCGGCAA
TAAACCCTGCAATGGCGTGGCCGGCGTGAACTGCTACTTCCCTCTGCAA
AGCTACGGCTTTAGACCCACCTACGGCGTGGGCCATCAGCCCTACAGAG
TGGTCGTGCTCAGCTTTGAGCTGCTCCACGCTCCTGCTACAGTCTGCGG
CCCTAAAAAGAGCACAAACCTCGTCAAGAACAAAAGCGTGAACTTCAAC
TTTAACGGCCTGTATGAGAATCAGAAGCTGATCGCCAATCAGTTCAACA
GCGCCATCGGCAAGATCCAAGACAGCCTGAGCAGCACCGCTAGCGCCCT
GGGCAAGCTGCAAGACGTGGTGAATCAGAACGCCCAAGCCCTGAACACC
CTGGTGAAGCAGCTGGTGCAGCCTACCGAGTCCATCGTGCGGTTCCCCA
ACATCACAAACCTGTGTCCCTTTGGGGAAGTCTTTAACGCCACCCGGTT
CGCTAGCGTGTATGCCTGGAATAGAAAGAGAATTAGCAATTGCGTGGCT
GACTATAGCGTGCTGTACAACAGCGCCTCCTTCTCCACATTTAAGTGCT
ACGGCGTGAGCCCCACAAAGCTGAATGATCTCTGCTTCACCAACGTGTA
CGCCGACAGCTTCGTCATTAGAGGCGATGAAGTGAGACAGATCGCTCCT
GGGCAGACCGGCAAAATTGCTGACTACAACTATAAGCTGCCCGACGATT
TCACCGGCTGCGTGATTGCCTGGAACAGCAATAACCTGGACAGCAAGGT
CGGCGGCAACTATAACTACAGATACAGACTGTTCCGGAAGTCCAACCTG
AAGCCCTTCGAGAGAGACATCTCCACCGAGATCTATCAAGCCGGCAGCA
AGCCCTGCAACGGCGTGGAGGGCTTTAACTGCTATTTCCCCCTGCAGAG
CTACGGCTTTCAGCCTACAAACGGCGTGGGGTATCAGCCCTACAGAGTC
GTGGTCCTGAGCTTTGAACTGCTCCATGCTCCCGCTACCGTGTGTGGCC
CCAAGAAGAGCACCAACCTCGTGAAAAACAAGTCCGTGAATTTCAACTT
CAACGGGAAGAACCACACAAGCCCCGACGTGGACCTGGGCGACATCAGC
GGCATCAACGCTAGCGTGGTGAACATTCAGAAGGAGATCGACAGACTGA
ACGAGGTGGCCAAGAACCTGAACGAGAGCCTGATCGACCTGCAAGAGCT
GGGCAAGTACGTGCAGCCCACAGAGAGCATCGTGCGGTTCCCCAACATC
ACCAATCTGTGTCCCTTCGGCGAGGTCTTCAATGCCACCCGGTTCGCTA
GCGTGTATGCTTGGAACAGAAAACGGATCAGCAATTGCGTGGCCGACTA
CAGCGTGCTGTATAATAGCGCTAGCTTCAGCACCTTCAAATGTTATGGC
GTCTCCCCCACCAAGCTGAATGACCTGTGTTTCACAAACGTGTATGCCG
ACTCCTTCGTGATCAGAGGCGACGAGGTCAGACAGATTGCCCCTGGGCA
GACCGGCAAGATCGCCGACTACAACTACAAACTGCCCGATGACTTCACC
GGCTGCGTGATCGCCTGGAATTCCAACAATCTGGACTCCAAAGTGGGCG
GGAACTACAACTACCTGTACCGGCTGTTTAGAAAAAGCAACCTGAAACC
TTTCGAGCGGGATATTAGCACAGAAATCTATCAAGCCGGGAGCACCCCC
TGCAATGGGGTGGAGGGCTTCAATTGCTACTTCCCCCTGCAATCCTATG
GGTTTCAGCCCACCAACGGCGTGGGCTACCAACCCTATAGAGTGGTGGT
CCTCAGCTTTGAGCTGCTGCACGCCCCCGCCACCGTGTGCGGGCCCAAA
AAGAGCACCAACCTGGTCAAGAACAAGTCCGTCAACTTTAACTTTAACG
GCTGA

Construction of M65-HR-opt: further optimization of HR1 and HR2 sequences in M65, and construction mode thereof: RBD (BA.4/5)-HR1 (opt)-RBD (Delta)-HR2 (opt)-RBD (WT)

The amino acid sequence constructed for M65-HR-opt is:

SEQ ID NO: 22: RBD (BA.4/5)-HR1 (opt)-RBD
(Delta)-HR2 (opt)-RBD (WT)
MDAMKRGLCCVLLLCGAVFVSPVQPTESIVRFPNITNLCPFDEVFNATR
FASVYAWNRKRISNCVADYSVLYNFAPFFAFKCYGVSPTKLNDLCFTNV
YADSFVIRGNEVSQIAPGQTGNIADYNYKLPDDFTGCVIAWNSNKLDSK
VGGNYNYRYRLFRKSNLKPFERDISTEIYQAGNKPCNGVAGVNCYFPLQ
SYGFRPTYGVGHQPYRVVVLSFELLHAPATVCGPKKSTNLVKNKSVNFN
FNGTQNVLYENQKLIANQFNSAIGKIQDSLSSTASALGKLQDVVNQNAQ
ALNTLVKQLSSNFGAISSVLNDILSRLDKVEVQPTESIVRFPNITNLCP
FGEVFNATRFASVYAWNRKRISNCVADYSVLYNSASFSTFKCYGVSPTK
LNDLCFTNVYADSFVIRGDEVRQIAPGQTGKIADYNYKLPDDFTGCVIA
WNSNNLDSKVGGNYNYRYRLFRKSNLKPFERDISTEIYQAGSKPCNGVE
GENCYFPLQSYGFQPTNGVGYQPYRVVVLSFELLHAPATVCGPKKSTNL
VKNKSVNFNFNGVYDPLQPELDSFKEELDKYFKNHTSPDVDLGDISGIN
ASVVNIQKEIDRLNEVAKNLNESLIDLQELGKYGKYEQYIKWPWYIWLG
FIAGVQPTESIVRFPNITNLCPFGEVFNATRFASVYAWNRKRISNCVAD
YSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLK
PFERDISTEIYQAGSTPCNGVEGENCYFPLQSYGFQPTNGVGYQPYRVV
VLSFELLHAPATVCGPKKSTNLVKNKSVNFNFNG

tPA signal peptide—RBD sequence of OmicronBA.4/5-HR1 sequence-RBD sequence of Delta-HR2 sequence-RBD sequence of WT.

SEQ ID NO: 23 (nucleotide sequence encoding SEQ ID NO: 22):
ATGGACGCCATGAAGAGAGGCCTGTGCTGCGTGCTGCTCCTGTGCGGCGCCGTGTTCGTG
AGCCCCGTGCAACCTACCGAGAGCATTGTCCGGTTCCCCAATATTACCAACCTCTGCCCC
TTCGACGAGGTCTTTAACGCTACAAGATTCGCCTCCGTGTACGCCTGGAACCGGAAGAGA
ATCAGCAATTGCGTCGCCGACTACTCCGTGCTCTACAACTTTGCCCCCTTCTTCGCTTTC
AAGTGCTACGGCGTGAGCCCCACAAAGCTCAACGACCTGTGCTTCACCAACGTGTACGCC
GACAGCTTTGTCATCAGAGGCAACGAGGTGAGCCAAATTGCCCCTGGCCAAACCGGCAAC
ATCGCCGATTACAACTACAAGCTCCCCGACGACTTCACCGGCTGTGTGATTGCTTGGAAC
AGCAATAAGCTCGACAGCAAGGTGGGGGGCAACTACAATTACCGGTACCGGCTGTTCCGG
AAGTCCAATCTGAAGCCTTTCGAGAGAGACATCAGCACCGAGATCTACCAAGCCGGGAAC
AAACCCTGCAATGGCGTGGCCGGCGTGAACTGCTACTTCCCTCTGCAGAGCTACGGGTTT
CGGCCCACCTACGGCGTGGGCCACCAACCCTACAGAGTGGTGGTCCTGAGCTTCGAGCTG
CTGCACGCCCCTGCTACAGTGTGTGGCCCCAAGAAAAGCACCAATCTCGTGAAGAACAAG
AGCGTCAACTTCAATTTTAACGGCACACAGAACGTGCTGTACGAGAATCAGAAGCTGATC
GCCAATCAGTTCAACAGCGCCATCGGCAAGATCCAAGACAGCCTGAGCAGCACCGCTAGC
GCCCTGGGCAAGCTGCAAGACGTGGTGAATCAGAACGCCCAAGCCCTGAACACCCTGGTG
AAGCAGCTGAGCAGCAACTTCGGCGCCATCAGCAGCGTGCTGAACGACATCCTGAGCAGA
CTGGACAAGGTGGAGGTGCAGCCTACAGAGTCCATCGTCAGATTCCCCAACATCACCAAC
CTGTGCCCCTTCGGCGAGGTGTTTAATGCCACACGGTTCGCTAGCGTGTACGCCTGGAAT
CGGAAGCGGATCTCCAACTGCGTCGCTGACTATTCCGTGCTCTATAACTCCGCTAGCTTC
AGCACATTTAAGTGCTATGGGGTGTCCCCCACCAAGCTGAATGACCTGTGCTTCACAAAC
GTCTACGCCGACTCCTTTGTCATCCGGGGCGACGAAGTCCGGCAGATCGCCCCTGGGCAG
ACCGGCAAAATCGCTGACTACAACTACAAACTGCCCGATGACTTCACCGGCTGTGTGATC
GCCTGGAACTCCAACAATCTGGACTCCAAGGTCGGCGGCAATTACAATTACAGATACAGA
CTGTTCAGAAAATCCAACCTGAAACCCTTCGAGCGGGACATTTCCACAGAGATCTATCAA
GCCGGCAGCAAACCTTGCAACGGCGTGGAGGGGTTCAACTGCTATTTTCCCCTGCAGAGC
TACGGGTTTCAGCCCACCAACGGCGTCGGCTACCAACCCTATCGGGTGGTCGTGCTGAGC
TTCGAACTCCTGCACGCTCCCGCCACAGTCTGCGGGCCTAAAAAGAGCACCAACCTGGTG
AAGAATAAATCCGTCAATTTCAACTTCAACGGCGTGTACGACCCCCTGCAGCCCGAGCTG
GACAGCTTCAAGGAGGAGCTGGACAAGTACTTCAAGAACCACACAAGCCCCGACGTGGAC
CTGGGCGACATCAGCGGCATCAACGCTAGCGTGGTGAACATTCAGAAGGAGATCGACAGA
CTGAACGAGGTGGCCAAGAACCTGAACGAGAGCCTGATCGACCTGCAAGAGCTGGGCAAG
TACGGCAAGTACGAGCAGTACATCAAGTGGCCCTGGTACATCTGGCTGGGCTTCATCGCC
GGCGTGCAGCCCACCGAGAGCATCGTCCGGTTTCCCAACATTACCAACCTGTGTCCCTTC
GGCGAGGTCTTCAATGCCACACGGTTCGCTAGCGTCTATGCCTGGAACCGGAAGAGAATT
AGCAACTGCGTGGCCGACTATAGCGTCCTGTACAACAGCGCTAGCTTCTCCACCTTCAAG
TGTTATGGCGTGTCCCCCACCAAGCTGAATGACCTCTGTTTTACCAATGTCTATGCCGAC
AGCTTCGTGATCAGAGGCGATGAAGTGAGACAGATCGCCCCTGGGCAGACCGGGAAGATC
GCCGACTACAACTATAAGCTCCCCGATGACTTCACCGGCTGCGTCATTGCTTGGAACTCC
AATAATCTGGACAGCAAGGTGGGGGGCAATTATAACTACCTGTATCGGCTGTTCAGAAAG
AGCAACCTGAAGCCTTTTGAGAGAGATATCAGCACCGAAATTTATCAAGCCGGGAGCACC
CCCTGCAACGGGGTGGAAGGCTTCAACTGTTACTTCCCCCTGCAGAGCTACGGCTTTCAG
CCTACCAACGGGGTGGGGTATCAGCCTTATAGAGTCGTGGTCCTGTCCTTCGAGCTGCTG
CACGCCCCCGCCACAGTGTGCGGGCCCAAGAAGAGCACCAATCTGGTGAAAAACAAAAGC
GTCAACTTCAATTTCAACGGCTGA
SEQ ID NO: 24:
Nucleotide sequence of S antigen of wild Ad-S vaccine
ATGTTCGTGTTCCTGGTGCTGCTGCCCCTGGTGAGCAGCCAGTGTGTGAACCTGACAACA
AGGACCCAGCTGCCTCCCGCCTACACCAACAGCTTCACAAGGGGCGTGTACTACCCTGAC
AAGGTGTTCAGGAGCAGCGTGCTGCACTCCACACAGGACCTGTTCCTGCCCTTCTTCTCC
AACGTGACATGGTTCCACGCCATCCACGTGTCCGGCACCAACGGCACAAAGAGATTCGAC
AACCCCGTGCTGCCTTTCAACGACGGCGTGTACTTCGCCTCCACAGAGAAGTCCAACATC
ATCAGGGGCTGGATCTTCGGCACAACACTGGACAGCAAGACACAGAGCCTGCTGATCGTG
AACAACGCCACAAACGTGGTCATTAAGGTGTGTGAGTTCCAGTTCTGCAACGACCCCTTC
CTGGGCGTGTACTATCACAAGAACAACAAGAGCTGGATGGAGAGCGAGTTCAGGGTGTAC
TCCAGCGCCAACAACTGTACATTCGAGTACGTGTCCCAGCCTTTCCTGATGGACCTGGAG
GGCAAGCAGGGCAACTTCAAGAACCTGAGGGAGTTCGTGTTCAAGAACATCGACGGCTAC
TTCAAGATCTACTCCAAGCACACCCCCATCAACCTGGTGAGAGACCTGCCTCAGGGCTTC
TCCGCCCTGGAGCCTCTGGTGGACCTGCCCATCGGCATCAACATCACAAGATTCCAGACA
CTGCTGGCCCTGCACAGAAGCTACCTGACCCCCGGCGATTCCAGCAGCGGCTGGACCGCT
GGCGCCGCTGCTTACTACGTGGGCTACCTGCAGCCCAGGACATTCCTGCTGAAGTACAAC
GAGAACGGCACAATCACCGATGCCGTGGATTGCGCCCTGGATCCTCTGAGCGAGACCAAG
TGTACACTGAAGTCCTTCACAGTGGAGAAGGGCATCTACCAGACCTCCAACTTCAGAGTG
CAGCCCACAGAGAGCATCGTGAGATTCCCTAACATCACAAACCTGTGCCCTTTCGGCGAG
GTGTTCAACGCCACAAGGTTCGCCAGCGTGTACGCCTGGAACAGAAAGAGGATCAGCAAC
TGCGTGGCCGATTACAGCGTGCTGTACAACTCCGCCTCCTTCTCCACATTCAAGTGCTAC
GGCGTGTCCCCCACCAAGCTGAACGATCTGTGTTTCACCAACGTGTACGCCGACTCCTTC
GTGATCAGAGGCGACGAGGTGAGGCAGATCGCCCCCGGACAGACCGGCAAGATCGCCGAT
TACAACTACAAGCTGCCCGATGACTTCACCGGCTGTGTGATCGCCTGGAACTCCAACAAC
CTGGATTCCAAGGTGGGCGGCAACTACAACTACCTGTACAGACTGTTCAGAAAGTCCAAC
CTGAAGCCCTTCGAGAGAGATATCTCCACCGAGATCTACCAGGCCGGCTCCACACCTTGT
AACGGCGTGGAGGGCTTCAACTGCTACTTCCCCCTGCAGAGCTACGGCTTCCAGCCTACC
AACGGCGTGGGCTACCAGCCTTACAGAGTGGTGGTGCTGTCCTTCGAGCTGCTGCACGCC
CCCGCCACAGTGTGTGGCCCAAAGAAGTCCACCAACCTGGTGAAGAACAAGTGTGTGAAC
TTCAACTTCAACGGCCTGACCGGCACAGGCGTGCTGACCGAGAGCAACAAGAAGTTCCTG
CCTTTCCAGCAGTTCGGCAGAGACATCGCCGATACCACAGACGCCGTGAGAGACCCTCAG
ACCCTGGAGATCCTGGATATCACCCCTTGCAGCTTCGGCGGCGTGTCCGTGATCACCCCT
GGCACAAACACCAGCAACCAGGTGGCCGTGCTGTACCAGGACGTGAACTGTACAGAGGTG
CCCGTGGCCATCCACGCCGACCAGCTGACCCCCACATGGAGAGTGTACTCCACCGGCTCC
AACGTGTTCCAGACAAGGGCCGGCTGCCTGATCGGCGCCGAGCATGTGAACAACAGCTAC
GAGTGTGACATCCCTATCGGCGCCGGCATCTGCGCCAGCTACCAGACCCAGACCAACAGC
CCCAGGAGGGCCAGATCCGTGGCCAGCCAGAGCATCATCGCCTACACCATGTCCCTGGGC
GCCGAGAACAGCGTGGCCTACTCCAACAACAGCATCGCCATCCCCACCAACTTCACAATC
TCCGTGACCACCGAGATCCTGCCCGTGAGCATGACCAAGACATCCGTGGATTGTACCATG
TACATCTGCGGCGACAGCACAGAGTGCTCCAACCTGCTGCTGCAGTACGGCTCCTTCTGT
ACCCAGCTGAACAGGGCCCTGACAGGCATCGCCGTGGAGCAGGACAAGAACACCCAGGAG
GTGTTCGCCCAGGTGAAGCAGATCTACAAGACACCTCCCATCAAGGACTTCGGCGGCTTC
AACTTCTCCCAGATCCTGCCCGACCCTTCCAAGCCCTCCAAGAGATCCTTCATCGAGGAC
CTGCTGTTCAACAAGGTGACCCTGGCCGATGCCGGCTTCATCAAGCAGTACGGCGACTGC
CTGGGCGACATCGCCGCCAGAGACCTGATCTGCGCCCAGAAGTTCAACGGCCTCACCGTG
CTGCCCCCTCTGCTGACCGATGAGATGATCGCCCAGTACACCAGCGCCCTGCTGGCCGGA
ACAATCACCAGCGGCTGGACATTCGGCGCCGGCGCTGCTCTGCAGATCCCTTTCGCCATG
CAGATGGCCTACAGGTTCAACGGCATCGGCGTGACCCAGAACGTGCTGTACGAGAACCAG
AAGCTGATCGCCAACCAGTTCAACAGCGCCATCGGCAAGATCCAGGATTCCCTGAGCAGC
ACAGCCAGCGCCCTGGGCAAGCTGCAGGATGTGGTGAACCAGAACGCCCAGGCCCTGAAC
ACACTGGTGAAGCAGCTGAGCAGCAACTTCGGCGCCATCAGCAGCGTGCTCAACGACATC
CTGAGCAGACTGGATAAGGTGGAGGCCGAGGTGCAGATCGATAGACTGATCACCGGCAGG
CTGCAGTCCCTGCAGACATACGTGACCCAGCAGCTGATCAGAGCCGCCGAGATCAGGGCC
AGCGCCAACCTGGCCGCCACCAAGATGTCCGAGTGTGTGCTGGGCCAGAGCAAGAGGGTG
GACTTCTGTGGCAAGGGCTACCACCTGATGTCCTTCCCTCAGTCCGCCCCCCACGGCGTG
GTGTTCCTGCACGTGACATACGTGCCTGCCCAGGAGAAGAACTTCACAACAGCCCCTGCC
ATCTGTCACGACGGCAAGGCCCACTTCCCCAGGGAGGGCGTGTTCGTGAGCAACGGCACC
CACTGGTTCGTGACCCAGAGGAACTTCTACGAGCCTCAGATCATCACAACCGATAACACA
TTCGTGAGCGGCAACTGTGATGTGGTCATTGGCATCGTGAACAATACCGTGTACGATCCC
CTGCAGCCTGAGCTGGACTCCTTCAAGGAGGAGCTGGATAAGTACTTCAAGAACCACACC
TCCCCCGACGTGGATCTGGGCGACATTAGCGGCATCAACGCCTCCGTGGTGAACATCCAG
AAGGAGATCGACAGGCTGAACGAGGTGGCCAAGAACCTGAACGAGTCCCTGATCGATCTG
CAGGAGCTGGGCAAGTACGAGCAGTACATCAAGTGGCCCTGGTACATCTGGCTGGGCTTC
ATCGCCGGCCTGATCGCCATCGTGATGGTGACAATCATGCTGTGTTGCATGACATCCTGT
TGTTCCTGTCTGAAGGGCTGTTGTAGCTGTGGCTCCTGTTGCAAGTTCGACGAGGATGAC
TCCGAGCCCGTGCTGAAGGGCGTGAAGCTGCACTACACCTGA
SEQ ID NO: 25:
Amino acid sequence of S antigen of wild Ad-S vaccine
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLENKVTLADAGFIKQYGDC
LGDIAARDLICAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDD
SEPVLKGVKLHYT*
SEQ ID NO: 26:
Nucleotide sequence of S antigen of Ad-S-Beta vaccine
ATGTTCGTGTTCCTGGTGCTGCTGCCCCTGGTGAGCAGCCAATGCGTGAACCTGACCACA
AGAACACAGCTGCCCCCCGCCTACACCAACAGCTTCACAAGAGGCGTGTATTATCCCGAC
AAGGTGTTCAGAAGCAGCGTGCTGCACAGCACCCAAGACCTGTTTCTGCCTTTTTTCAGC
AACGTGACCTGGTTCCACGCCATCCACGTGAGCGGCACCAACGGCACCAAGAGATTCGCC
AACCCCGTGCTGCCCTTCAACGACGGCGTGTACTTCGCTAGCACCGAGAAGAGCAACATC
ATCAGAGGCTGGATCTTCGGCACCACCCTGGATAGCAAGACACAGAGCCTGCTGATCGTG
AACAACGCCACCAACGTGGTGATCAAGGTGTGCGAGTTTCAGTTCTGCAACGACCCCTTC
CTGGGCGTGTATTACCACAAGAACAACAAGAGCTGGATGGAGAGCGAGTTCAGAGTCTAC
AGCAGCGCCAACAACTGCACCTTCGAGTACGTGAGCCAACCCTTCCTGATGGACCTGGAG
GGCAAGCAAGGCAATTTCAAGAACCTGAGAGAGTTCGTGTTCAAGAACATCGACGGCTAC
TTCAAGATCTACAGCAAGCACACCCCCATCAACCTGGTGAGAGGCCTGCCCCAAGGCTTC
AGCGCCCTGGAGCCCCTGGTGGACCTGCCCATCGGCATCAACATCACAAGATTTCAGACA
CTGCACCGGTCCTATCTCACCCCTGGCGACAGCAGCAGCGGGTGGACCGCTGGCGCCGCC
GCTTACTACGTGGGCTACCTGCAGCCTAGAACCTTCCTGCTGAAGTACAACGAGAACGGC
ACCATTACCGACGCCGTGGACTGCGCCCTGGACCCCCTGAGCGAGACCAAGTGCACCCTG
AAGAGCTTCACCGTGGAGAAGGGCATCTATCAGACAAGCAACTTCAGAGTGCAGCCCACC
GAGAGCATCGTGAGATTCCCCAACATCACCAACCTGTGCCCCTTCGGCGAGGTGTTCAAC
GCCACAAGATTCGCTAGCGTGTACGCCTGGAACCGGAAAAGAATCAGCAACTGCGTGGCC
GACTACAGCGTGCTGTACAACAGCGCTAGCTTCAGCACCTTCAAGTGCTACGGCGTGAGC
CCCACCAAGCTGAACGACCTGTGCTTCACCAACGTGTACGCCGACAGCTTCGTGATCAGA
GGCGACGAGGTGAGACAGATCGCCCCCGGGCAGACCGGCAACATCGCCGACTACAACTAC
AAGCTGCCCGACGACTTCACCGGCTGCGTGATCGCCTGGAACAGCAATAACCTGGACAGC
AAGGTGGGCGGCAACTACAACTACCTGTACAGACTGTTCAGAAAGAGCAACCTGAAGCCC
TTCGAGAGAGACATCAGCACCGAGATCTACCAAGCCGGCAGCACCCCCTGCAACGGCGTG
AAGGGCTTCAACTGCTACTTCCCCCTGCAGAGCTACGGCTTTCAGCCCACCTACGGCGTG
GGCTATCAGCCCTACAGAGTGGTCGTGCTGAGCTTCGAGCTGCTGCACGCCCCTGCCACC
GTGTGTGGCCCCAAGAAGAGCACCAACCTGGTGAAGAACAAGTGCGTGAACTTCAACTTC
AACGGGCTGACCGGGACCGGCGTGCTGACCGAGAGCAACAAGAAGTTTCTGCCCTTTCAA
CAGTTCGGCAGAGACATCGCCGACACCACCGACGCCGTCCGGGACCCTCAGACCCTGGAG
ATCCTGGACATCACCCCCTGCTCCTTCGGCGGCGTGAGCGTGATCACCCCCGGCACCAAC
ACAAGCAACCAAGTGGCCGTGCTGTACCAAGGCGTGAACTGCACCGAGGTGCCCGTGGCC
ATCCACGCCGATCAGCTGACCCCCACCTGGAGAGTGTACAGCACCGGCAGCAACGTGTTT
CAGACAAGAGCCGGCTGCCTGATCGGCGCCGAGCACGTGAACAACAGCTACGAGTGCGAC
ATCCCCATCGGCGCCGGCATCTGCGCTAGCTATCAGACACAGACCAACAGCCCTAGAAGA
GCTAGAAGCGTGGCTAGCCAAAGCATCATCGCCTACACCATGAGCCTGGGCGTGGAGAAC
AGCGTGGCCTACAGCAACAACAGCATCGCCATCCCCACCAACTTCACCATCAGCGTGACC
ACCGAAATCCTGCCCGTGAGCATGACCAAGACAAGCGTGGACTGCACCATGTACATCTGC
GGCGACAGCACCGAGTGCAGCAACCTGCTCCTGCAGTACGGCAGCTTCTGCACACAGCTG
AACAGAGCCCTGACCGGCATCGCCGTGGAGCAAGACAAGAACACCCAAGAGGTGTTCGCC
CAAGTGAAGCAGATCTACAAGACCCCCCCCATCAAGGACTTCGGCGGCTTCAACTTCAGC
CAAATTCTGCCCGACCCTAGCAAGCCTAGCAAGAGAAGCTTCATCGAGGACCTGCTGTTC
AACAAGGTGACCCTGGCCGACGCCGGCTTCATCAAGCAGTACGGCGACTGCCTGGGCGAT
ATCGCTGCTAGAGACCTGATCTGCGCTCAGAAGTTCAACGGCCTGACCGTGCTCCCCCCC
CTGCTGACCGACGAGATGATCGCTCAGTACACAAGCGCTCTGCTCGCCGGGACCATCACA
TCCGGGTGGACATTCGGGGCCGGCGCTGCCCTGCAGATCCCCTTCGCCATGCAGATGGCC
TACAGATTCAACGGCATCGGCGTGACACAGAACGTGCTGTACGAGAATCAGAAGCTGATC
GCCAATCAGTTCAACAGCGCCATCGGCAAGATCCAAGACAGCCTGAGCAGCACCGCTAGC
GCCCTGGGCAAGCTGCAAGACGTGGTGAATCAGAACGCCCAAGCCCTGAACACCCTGGTG
AAGCAGCTGAGCAGCAACTTCGGCGCCATCAGCTCCGTGCTGAACGACATCCTGAGCAGA
CTGGACCCCCCCGAGGCCGAGGTGCAGATTGACAGACTGATCACCGGCAGACTGCAGAGC
CTGCAGACCTACGTGACACAGCAGCTGATCAGAGCCGCCGAGATCAGAGCTAGCGCCAAC
CTGGCCGCCACCAAGATGAGCGAGTGCGTGCTGGGGCAGAGCAAGAGAGTGGACTTCTGC
GGCAAGGGCTACCACCTGATGAGCTTCCCTCAGAGCGCCCCCCACGGCGTGGTGTTCCTG
CACGTGACCTACGTGCCCGCCCAAGAGAAGAACTTCACCACCGCCCCCGCCATCTGCCAC
GACGGCAAGGCCCACTTCCCTAGAGAGGGCGTGTTCGTGAGCAACGGCACCCACTGGTTC
GTGACACAGAGAAACTTCTACGAGCCTCAGATCATCACCACCGACAACACCTTCGTGAGC
GGCAACTGCGACGTGGTGATCGGCATTGTGAACAATACCGTGTACGACCCCCTGCAGCCC
GAGCTGGACAGCTTCAAGGAGGAGCTGGACAAGTACTTCAAGAACCACACAAGCCCCGAC
GTGGACCTGGGCGACATTAGCGGCATCAACGCTAGCGTGGTGAACATTCAGAAGGAGATC
GACAGACTGAACGAGGTGGCCAAGAACCTGAACGAGAGCCTGATCGACCTGCAAGAGCTG
GGCAAGTACGAGCAGTACATCAAGTGGCCCTGGTACATCTGGCTGGGCTTCATCGCCGGC
CTGATCGCCATCGTGATGGTGACCATCATGCTGTGCTGCATGACAAGCTGTTGCAGCTGC
CTGAAGGGCTGCTGCAGCTGCGGGAGCTGCTGCAAGTTCGACGAGGACGACAGCGAGCCC
GTGCTGAAGGGCGTGAAGCTGCACTACACCTGA
SEQ ID NO: 27:
Amino acid sequence of S antigen of Ad-S-Beta vaccine
MFVFLVLLPLVSSQCVNLTTRTQLPPAYTNSFTRGVYYPDKVFRSSVLHSTQDLFLPFFS
NVTWFHAIHVSGTNGTKRFANPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRGLPQGFSALEPLVDLPIGINITRFQT
LHRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGNIADYNY
KLPDDFTGCVIAWNSNNLDSKVGGNYNYLYRLFRKSNLKPFERDISTEIYQAGSTPCNGV
KGFNCYFPLQSYGFQPTYGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEHVNNSYECD
IPIGAGICASYQTQTNSPRRARSVASQSIIAYTMSLGVENSVAYSNNSIAIPTNFTISVT
TEILPVSMTKTSVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAGFIKQYGDCLGD
IAARDLICAQKFNGLTVLPPLLTDEMIAQYTSALLAGTITSGWTFGAGAALQIPFAMQMA
YRENGIGVTQNVLYENQKLIANQFNSAIGKIQDSLSSTASALGKLQDVVNQNAQALNTLV
KQLSSNFGAISSVLNDILSRLDPPEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEP
VLKGVKLHYT*
SEQ ID NO: 28:
Nucleotide sequence of S antigen of Ad-S-Delta vaccine
ATGTTCGTGTTCCTGGTGCTGCTGCCCCTGGTGAGCAGCCAATGCGTGAACCTGAGAACA
AGAACACAGCTGCCCCCCGCCTACACCAACAGCTTCACAAGAGGGGTCTACTACCCCGAC
AAGGTGTTCAGAAGCAGCGTGCTGCACAGCACCCAAGACCTGTTCCTGCCCTTCTTCTCC
AACGTGACCTGGTTCCACGCCATCCACGTGAGCGGCACCAACGGCACCAAGAGATTCGAC
AACCCCGTGCTGCCCTTCAACGACGGCGTGTACTTCGCTAGCACCGAGAAGAGCAACATC
ATCAGAGGCTGGATCTTCGGCACCACCCTGGACAGCAAGACACAGAGCCTGCTGATTGTG
AACAACGCTACCAACGTGGTGATCAAGGTGTGCGAGTTTCAGTTCTGCAACGACCCCTTC
CTGGGGGTGTACTACCACAAAAACAACAAGAGCTGGATGGAGAGCGGCGTGTACAGCAGC
GCCAACAACTGCACCTTCGAGTACGTGAGCCAACCCTTCCTGATGGACCTGGAGGGCAAG
CAAGGCAACTTCAAGAACCTCAGAGAGTTCGTGTTCAAGAACATCGACGGCTACTTCAAG
ATCTACAGCAAGCACACCCCCATCAACCTGGTGAGAGACCTGCCCCAAGGCTTCAGCGCC
CTGGAGCCCCTGGTGGACCTGCCCATCGGCATCAACATCACAAGATTTCAGACCCTGCTG
GCCCTGCACAGAAGCTACCTGACACCCGGCGACAGCAGCAGCGGGTGGACAGCCGGCGCT
GCCGCTTACTACGTGGGCTACCTGCAGCCTAGAACCTTCCTGCTGAAGTACAACGAGAAC
GGCACCATTACCGACGCCGTCGACTGCGCCCTGGACCCCCTGAGCGAGACCAAGTGCACC
CTGAAGAGCTTCACCGTGGAGAAGGGCATCTATCAGACAAGCAACTTCAGAGTGCAGCCC
ACCGAGAGCATCGTGAGATTCCCCAACATCACCAACCTGTGCCCCTTCGGCGAGGTGTTC
AACGCCACAAGATTCGCTAGCGTGTACGCCTGGAACAGAAAGAGAATCAGCAACTGCGTG
GCCGACTACAGCGTGCTGTACAACAGCGCTAGCTTCAGCACCTTCAAATGCTACGGGGTC
AGCCCCACCAAGCTGAACGACCTGTGCTTCACCAACGTGTACGCCGACAGCTTCGTGATC
AGAGGCGACGAGGTGAGACAGATCGCCCCCGGGCAGACCGGCAAGATCGCCGACTACAAC
TACAAGCTGCCCGACGACTTCACCGGCTGCGTGATCGCCTGGAACTCCAACAACCTGGAT
AGCAAGGTGGGCGGCAACTACAACTATAGATACAGACTGTTCAGAAAGAGCAACCTGAAG
CCCTTCGAGAGAGACATCAGCACCGAGATCTACCAAGCCGGCAGCAAGCCCTGCAACGGC
GTGGAGGGCTTCAACTGCTACTTCCCCCTGCAGAGCTACGGCTTTCAGCCCACCAACGGC
GTGGGCTATCAGCCCTACAGAGTGGTCGTGCTGAGCTTCGAGCTGCTGCACGCCCCTGCC
ACCGTGTGCGGCCCCAAGAAGAGCACCAACCTGGTGAAGAACAAGTGCGTGAACTTCAAC
TTCAACGGCCTGACCGGCACCGGCGTGCTGACCGAGAGCAACAAGAAGTTCCTCCCTTTT
CAGCAGTTCGGCAGAGACATCGCCGACACCACCGATGCCGTGAGAGACCCTCAGACCCTG
GAGATCCTGGACATCACCCCCTGCAGCTTTGGCGGCGTGAGCGTGATCACCCCCGGCACC
AACACAAGCAACCAAGTGGCCGTGCTGTACCAAGGCGTGAACTGCACCGAGGTGCCCGTG
GCCATCCACGCCGATCAGCTGACCCCCACCTGGAGAGTGTACAGCACCGGCAGCAACGTG
TTTCAGACAAGAGCCGGCTGCCTGATCGGCGCCGAGCACGTGAACAACAGCTACGAGTGC
GACATCCCCATCGGCGCCGGCATCTGCGCTAGCTATCAGACACAGACCAACAGCAGACGG
AGAGCTAGAAGCGTGGCTAGCCAAAGCATCATCGCCTACACCATGAGCCTGGGCGCCGAG
AACAGCGTGGCCTACAGCAACAACAGCATCGCCATCCCCACCAACTTCACCATCAGCGTG
ACCACCGAGATCCTGCCCGTCTCCATGACCAAGACAAGCGTGGACTGCACCATGTACATC
TGCGGCGACAGCACCGAGTGCAGCAACCTGCTCCTGCAGTACGGCAGCTTCTGCACACAG
CTGAACAGAGCCCTGACCGGCATCGCCGTGGAGCAAGACAAGAACACCCAAGAGGTGTTC
GCCCAAGTGAAGCAGATCTACAAGACCCCCCCCATCAAGGACTTCGGCGGCTTCAACTTC
AGCCAAATCCTGCCCGACCCTAGCAAGCCTAGCAAGAGAAGCTTCATCGAGGACCTGCTG
TTCAACAAGGTGACCCTGGCCGACGCCGGCTTCATCAAGCAGTACGGCGACTGCCTGGGC
GACATCGCCGCTAGAGACCTGATCTGCGCTCAGAAGTTCAATGGCCTGACCGTCCTGCCC
CCCCTGCTGACCGACGAGATGATCGCTCAGTACACAAGCGCCCTGCTGGCCGGCACAATT
ACAAGCGGCTGGACATTCGGCGCTGGCGCCGCCCTGCAAATCCCCTTCGCCATGCAGATG
GCCTACAGATTCAACGGCATCGGCGTGACACAGAACGTGCTGTACGAGAATCAGAAGCTG
ATCGCCAATCAGTTCAACAGCGCCATCGGCAAGATCCAAGACAGCCTGAGCAGCACCGCT
AGCGCCCTGGGCAAGCTGCAGAACGTGGTGAATCAGAACGCCCAAGCCCTGAACACCCTG
GTGAAGCAGCTGAGCAGCAACTTCGGCGCCATCAGCTCCGTGCTGAACGATATCCTGAGC
AGACTGGACCCCCCCGAGGCCGAGGTGCAGATCGACCGGCTGATCACCGGCAGACTGCAG
AGCCTGCAGACCTACGTGACACAGCAGCTGATCAGAGCCGCCGAGATCAGAGCTAGCGCC
AACCTGGCCGCCACCAAGATGAGCGAGTGCGTGCTGGGGCAGAGCAAGAGAGTGGACTTC
TGCGGCAAGGGCTACCACCTGATGAGCTTCCCTCAGAGCGCCCCCCACGGCGTGGTGTTC
CTGCACGTGACCTACGTGCCCGCCCAAGAGAAGAACTTCACCACCGCCCCCGCCATCTGC
CACGACGGCAAGGCCCACTTCCCTAGAGAGGGCGTGTTCGTGAGCAACGGCACCCACTGG
TTCGTGACACAGAGAAACTTCTACGAGCCTCAGATCATCACCACCGACAACACCTTCGTG
AGCGGCAACTGCGACGTGGTGATCGGCATTGTGAACAATACAGTCTACGACCCTCTGCAG
CCCGAGCTGGACAGCTTCAAGGAGGAGCTGGACAAGTACTTCAAGAATCACACAAGCCCC
GACGTGGACCTGGGGGACATCAGCGGCATCAACGCTAGCGTGGTGAACATTCAGAAGGAG
ATCGACAGACTGAACGAGGTGGCCAAGAACCTGAACGAGAGCCTGATCGACCTGCAAGAG
CTGGGCAAGTACGAGCAGTACATCAAGTGGCCCTGGTACATCTGGCTGGGCTTCATCGCC
GGCCTGATCGCCATCGTGATGGTGACCATCATGCTGTGCTGCATGACAAGCTGCTGCAGC
TGCCTGAAGGGCTGCTGCTCCTGCGGCAGCTGCTGCAAGTTCGACGAGGACGACAGCGAG
CCCGTGCTGAAGGGCGTGAAGCTGCACTACACCTGA
SEQ ID NO: 29:
Amino acid sequence of S antigen of Ad-S-Delta vaccine
MFVFLVLLPLVSSQCVNLRTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESGVYSSANNCTFEYVSQPFLMDLEGK
QGNFKNLREFVFKNIDGYFKIYSKHTPINLVRDLPQGFSALEPLVDLPIGINITRFQTLL
ALHRSYLTPGDSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYAWNRKRISNCV
ADYSVLYNSASFSTFKCYGVSPTKLNDLCFTNVYADSFVIRGDEVRQIAPGQTGKIADYN
YKLPDDFTGCVIAWNSNNLDSKVGGNYNYRYRLFRKSNLKPFERDISTEIYQAGSKPCNG
VEGFNCYFPLQSYGFQPTNGVGYQPYRVVVLSFELLHAPATVCGPKKSTNLVKNKCVNFN
FNGLTGTGVLTESNKKFLPFQQFGRDIADTTDAVRDPQTLEILDITPCSFGGVSVITPGT
NTSNQVAVLYQGVNCTEVPVAIHADQLTPTWRVYSTGSNVFQTRAGCLIGAEHVNNSYEC
DIPIGAGICASYQTQTNSRRRARSVASQSIIAYTMSLGAENSVAYSNNSIAIPTNFTISV
TTEILPVSMTKTSVDCTMYICGDSTECSNLLLQYGSFCTQLNRALTGIAVEQDKNTQEVF
AQVKQIYKTPPIKDFGGFNFSQILPDPSKPSKRSFIEDLLFNKVTLADAGFIKQYGDCLG
DIAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQNVVNQNAQALNTL
VKQLSSNFGAISSVLNDILSRLDPPEAEVQIDRLITGRLQSLQTYVTQQLIRAAEIRASA
NLAATKMSECVLGQSKRVDFCGKGYHLMSFPQSAPHGVVFLHVTYVPAQEKNFTTAPAIC
HDGKAHFPREGVFVSNGTHWFVTQRNFYEPQIITTDNTFVSGNCDVVIGIVNNTVYDPLQ
PELDSFKEELDKYFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSCGSCCKFDEDDSE
PVLKGVKLHYT*
SEQ ID NO: 30:
Nucleotide sequence of S antigen of Ad-S-Beta-Delta vaccine
ATGTTCGTGTTCCTGGTGCTGCTGCCCCTGGTGAGCAGCCAATGCGTGAACCTGACCACA
AGAACACAGCTGCCCCCCGCCTACACCAACAGCTTCACAAGAGGCGTGTATTATCCCGAC
AAGGTGTTCAGAAGCAGCGTGCTGCACAGCACCCAAGACCTGTTTCTGCCCTTCTTCAGC
AACGTGACCTGGTTCCACGCCATCCACGTGAGCGGCACCAACGGCACCAAGAGATTCGCC
AACCCCGTGCTGCCCTTCAACGACGGCGTGTACTTCGCTAGCACCGAGAAGAGCAACATC
ATCAGAGGCTGGATCTTCGGCACCACCCTGGATAGCAAGACACAGAGCCTGCTGATCGTG
AACAACGCCACCAACGTGGTGATCAAGGTGTGCGAGTTTCAGTTCTGCAACGACCCCTTC
CTGGGCGTGTATTACCACAAGAACAACAAGAGCTGGATGGAGAGCGAGTTCAGAGTCTAC
AGCAGCGCCAACAACTGCACCTTCGAGTACGTGAGCCAACCCTTCCTGATGGACCTGGAG
GGCAAGCAAGGCAATTTCAAGAACCTGAGAGAGTTCGTGTTCAAGAACATCGACGGCTAC
TTCAAGATCTACAGCAAGCACACCCCCATCAACCTGGTGAGAGGCCTGCCCCAAGGCTTC
AGCGCCCTGGAGCCCCTGGTGGACCTGCCCATCGGCATCAACATCACAAGATTTCAGACC
CTGCACCGGAGCTATCTGACCCCTGGCGACTCCTCCTCCGGGTGGACCGCTGGCGCCGCC
GCTTACTACGTGGGCTACCTGCAGCCTAGAACCTTCCTGCTGAAGTACAACGAGAACGGC
ACCATTACAGACGCCGTGGATTGCGCCCTGGACCCCCTGAGCGAGACCAAGTGCACCCTG
AAGAGCTTCACCGTGGAGAAGGGCATCTATCAGACAAGCAACTTCAGAGTGCAGCCCACC
GAGAGCATCGTGAGATTCCCCAACATCACCAACCTGTGCCCCTTCGGCGAGGTGTTCAAC
GCCACAAGATTCGCTAGCGTGTACGCCTGGAACAGAAAAAGAATCAGCAACTGCGTGGCC
GACTACAGCGTGCTGTACAACAGCGCTAGCTTCAGCACCTTCAAGTGCTACGGCGTGAGC
CCCACCAAGCTGAACGACCTGTGCTTCACCAACGTGTACGCCGACAGCTTCGTGATCAGA
GGCGACGAGGTGAGACAGATCGCCCCCGGGCAGACCGGCAACATCGCCGACTACAACTAT
AAGCTCCCCGACGACTTCACCGGCTGCGTGATCGCTTGGAACTCCAACAACCTGGACAGC
AAGGTGGGCGGCAACTACAACTACAGATATAGACTGTTCAGAAAGAGCAACCTGAAGCCC
TTCGAGAGAGACATCAGCACCGAGATCTACCAAGCCGGCAGCAAGCCCTGCAACGGCGTG
AAGGGCTTCAACTGCTACTTCCCCCTGCAGAGCTACGGCTTTCAGCCCACCTACGGCGTG
GGCTATCAGCCCTACAGAGTGGTCGTGCTGAGCTTCGAGCTGCTGCACGCCCCCGCCACC
GTCTGCGGCCCCAAGAAGAGCACCAACCTGGTGAAGAACAAGTGCGTGAACTTCAACTTC
AACGGGCTGACCGGCACCGGCGTGCTGACCGAGAGCAACAAGAAGTTCCTGCCCTTTCAG
CAGTTCGGCAGAGACATCGCCGACACCACCGACGCCGTGAGAGACCCTCAGACCCTGGAG
ATCCTGGACATCACACCCTGCAGCTTCGGCGGCGTGAGCGTGATCACCCCCGGCACCAAC
ACAAGCAACCAAGTGGCCGTGCTGTACCAAGGCGTGAACTGCACCGAGGTGCCCGTGGCC
ATCCACGCCGATCAGCTGACCCCCACCTGGAGAGTCTATAGCACCGGCAGCAACGTGTTT
CAGACAAGAGCCGGCTGCCTGATCGGCGCCGAGCACGTGAACAACAGCTACGAGTGCGAC
ATCCCCATCGGCGCCGGCATCTGCGCTAGCTATCAGACACAGACCAACAGCCCTAGAAGA
GCTAGAAGCGTGGCTAGCCAAAGCATCATCGCCTACACCATGAGCCTGGGCGTGGAGAAC
AGCGTGGCCTACAGCAACAACAGCATCGCCATCCCCACCAACTTCACCATCAGCGTGACC
ACCGAGATCCTCCCTGTGAGCATGACCAAGACAAGCGTGGACTGCACCATGTACATCTGC
GGCGACAGCACCGAGTGCAGCAACCTGCTCCTGCAGTACGGCAGCTTCTGCACACAGCTG
AACAGAGCCCTGACCGGCATCGCCGTGGAGCAAGACAAGAACACCCAAGAGGTGTTCGCC
CAAGTGAAGCAGATCTACAAGACCCCCCCCATCAAGGACTTCGGCGGCTTCAACTTCAGC
CAAATCCTGCCCGACCCTAGCAAGCCTAGCAAGAGAAGCTTCATCGAGGACCTGCTGTTC
AACAAGGTGACCCTGGCCGACGCCGGCTTCATCAAGCAGTACGGCGACTGCCTGGGCGAC
ATCGCCGCTAGAGACCTGATCTGCGCTCAGAAGTTCAACGGCCTGACCGTGCTGCCCCCC
CTGCTGACCGACGAGATGATCGCTCAATACACAAGCGCCCTCCTGGCCGGGACAATCACA
TCCGGCTGGACCTTCGGCGCCGGGGCTGCCCTGCAGATCCCCTTCGCCATGCAGATGGCC
TACAGATTCAACGGCATCGGCGTGACACAGAACGTGCTGTACGAGAATCAGAAGCTGATC
GCCAATCAGTTCAACAGCGCCATCGGCAAGATCCAAGACAGCCTGAGCAGCACCGCTAGC
GCCCTGGGCAAGCTGCAAGACGTGGTGAATCAGAACGCCCAAGCCCTGAACACCCTGGTG
AAGCAGCTGAGCAGCAACTTCGGCGCCATCAGCAGCGTGCTGAACGACATCCTGAGCAGA
CTGGACCCCCCCGAGGCCGAGGTGCAGATCGATAGACTGATCACCGGCAGACTGCAGAGC
CTGCAGACCTACGTGACACAGCAGCTGATCAGAGCCGCCGAGATCAGAGCTAGCGCCAAC
CTGGCCGCCACCAAGATGAGCGAGTGCGTGCTGGGGCAGAGCAAGAGAGTGGACTTCTGC
GGCAAGGGCTACCACCTGATGAGCTTCCCTCAGAGCGCCCCCCACGGCGTGGTGTTCCTG
CACGTGACCTACGTGCCCGCCCAAGAGAAGAACTTCACCACCGCTCCCGCCATCTGCCAC
GACGGCAAGGCCCACTTCCCTAGAGAGGGCGTGTTCGTGAGCAACGGCACCCACTGGTTC
GTGACACAGAGAAACTTCTACGAGCCTCAGATCATCACCACCGACAACACCTTCGTGAGC
GGCAACTGCGACGTGGTGATCGGCATCGTGAACAATACCGTCTACGACCCCCTGCAACCC
GAGCTGGACAGCTTCAAGGAGGAGCTGGACAAGTACTTCAAGAACCACACAAGCCCCGAC
GTGGACCTGGGCGATATCAGCGGCATCAACGCTAGCGTGGTGAACATTCAGAAGGAAATC
GATCGGCTGAATGAGGTGGCCAAGAACCTGAACGAGAGCCTGATCGACCTGCAAGAGCTG
GGCAAGTACGAGCAGTACATCAAGTGGCCCTGGTACATCTGGCTGGGCTTCATCGCCGGC
CTGATCGCCATCGTGATGGTGACCATCATGCTGTGCTGCATGACAAGCTGCTGCAGCTGC
CTGAAGGGCTGCTGTAGCTGTGGCAGCTGCTGCAAGTTCGACGAGGACGACAGCGAGCCC
GTGCTGAAGGGCGTGAAGCTGCACTACACCTGA
SEQ ID NO: 31:
Amino acid sequence of S antigen of Ad-S-Beta-Delta vaccine
MFVFLVLLPLVSSQCVNLTTRTQLPPAYTNSFTRGVYYPDKVFRSSVLHSTQDLFLPFFS
NVTWFHAIHVSGTNGTKRFANPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRGLPQGFSALEPLVDLPIGINITRFQT
LHRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGNIADYNY
KLPDDFTGCVIAWNSNNLDSKVGGNYNYRYRLFRKSNLKPFERDISTEIYQAGSKPCNGV
KGFNCYFPLQSYGFQPTYGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEHVNNSYECD
IPIGAGICASYQTQTNSPRRARSVASQSIIAYTMSLGVENSVAYSNNSIAIPTNFTISVT
TEILPVSMTKTSVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAGFIKQYGDCLGD
IAARDLICAQKFNGLTVLPPLLTDEMIAQYTSALLAGTITSGWTFGAGAALQIPFAMQMA
YRENGIGVTQNVLYENQKLIANQFNSAIGKIQDSLSSTASALGKLQDVVNQNAQALNTLV
KQLSSNFGAISSVLNDILSRLDPPEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEP
VLKGVKLHYT*
SEQ ID NO: 32:
Nucleotide sequence of S antigen of Ad-S-Omicron-BA. 1 vaccine
ATGTTCGTGTTCCTGGTGCTGCTGCCCCTGGTGAGCAGCCAATGCGTGAACCTGACCACA
AGAACACAGCTGCCCCCCGCCTACACCAACAGCTTCACAAGAGGCGTGTACTACCCCGAC
AAGGTGTTCAGAAGCAGCGTCCTCCACAGCACCCAAGACCTGTTCCTGCCCTTCTTCAGC
AACGTGACCTGGTTCCACGTGATCAGCGGCACCAACGGCACCAAGAGATTCGACAACCCC
GTGCTGCCCTTCAACGACGGCGTGTACTTCGCTAGCATCGAGAAGAGCAACATCATCAGA
GGCTGGATCTTCGGCACCACCCTGGACAGCAAGACACAGAGCCTGCTGATCGTCAACAAC
GCCACCAACGTGGTGATCAAGGTGTGCGAGTTTCAGTTCTGCAACGACCCCTTCCTGGAC
CACAAGAACAACAAGAGCTGGATGGAGAGCGAGTTCAGAGTGTATAGCAGCGCTAACAAC
TGCACCTTCGAGTACGTGAGCCAACCCTTCCTGATGGACCTGGAGGGCAAGCAAGGCAAC
TTCAAAAACCTGAGAGAGTTCGTGTTCAAGAACATCGACGGCTACTTCAAGATCTACAGC
AAGCACACCCCCATCATCGTGAGAGAGCCCGAGGACCTGCCCCAAGGCTTCAGCGCCCTG
GAGCCCCTGGTGGACCTGCCCATCGGCATCAACATCACAAGATTTCAGACCCTGCTGGCC
CTGCACAGATCCTATCTGACCCCTGGGGACAGCAGCAGCGGCTGGACCGCTGGCGCTGCC
GCCTACTACGTGGGCTACCTGCAGCCTAGAACCTTCCTGCTGAAGTACAACGAGAACGGC
ACAATCACCGATGCCGTCGACTGCGCCCTGGACCCCCTGAGCGAGACCAAGTGCACCCTG
AAGAGCTTCACCGTGGAGAAGGGCATCTATCAGACAAGCAACTTCAGAGTGCAGCCCACC
GAGAGCATCGTGAGATTCCCCAACATCACCAACCTGTGCCCCTTCGACGAGGTGTTCAAC
GCCACAAGATTCGCTAGCGTGTACGCCTGGAATAGAAAGAGAATCAGCAACTGCGTGGCC
GACTACAGCGTGCTGTACAACCTGGCCCCCTTCTTCACCTTCAAATGCTACGGCGTGAGC
CCCACCAAGCTGAACGACCTGTGCTTCACCAACGTGTACGCCGACAGCTTCGTGATCAGA
GGCGACGAGGTGAGACAGATCGCCCCCGGGCAGACCGGCAACATCGCCGACTACAACTAC
AAGCTGCCCGACGACTTCACCGGCTGCGTGATCGCCTGGAACAGCAACAAGCTCGACAGC
AAGGTGAGCGGCAACTACAACTACCTGTACAGACTGTTCAGAAAGAGCAACCTGAAGCCC
TTCGAGAGAGACATCAGCACCGAGATCTACCAAGCCGGCAACAAGCCCTGCAACGGCGTG
GCCGGCTTCAACTGCTACTTCCCCCTGAGAAGCTACAGCTTCAGACCCACCTACGGCGTG
GGCCATCAGCCCTACAGAGTGGTCGTGCTGAGCTTCGAGCTGCTGCACGCCCCCGCCACC
GTGTGCGGCCCCAAGAAGAGCACCAACCTGGTGAAGAACAAGTGCGTGAACTTCAACTTC
AACGGCCTGAAGGGCACCGGCGTGCTGACCGAGAGCAACAAGAAGTTTCTGCCCTTTCAG
CAGTTCGGCAGAGACATCGCCGACACCACCGACGCCGTGAGAGACCCTCAGACCCTGGAG
ATCCTGGACATCACCCCCTGCTCCTTCGGCGGCGTGAGCGTGATCACCCCCGGCACCAAC
ACAAGCAACCAAGTGGCCGTGCTGTACCAAGGCGTGAACTGCACCGAGGTGCCCGTGGCC
ATCCACGCCGATCAGCTGACCCCCACCTGGAGAGTGTACAGCACCGGCAGCAACGTGTTT
CAGACAAGAGCCGGCTGCCTGATCGGCGCCGAGTACGTGAACAACAGCTACGAGTGCGAC
ATCCCCATCGGCGCCGGCATCTGCGCTAGCTATCAGACACAGACCAAGAGCCACAGAAGA
GCTAGAAGCGTGGCTAGCCAAAGCATCATCGCCTACACCATGAGCCTGGGCGCCGAGAAC
AGCGTGGCCTACAGCAACAACAGCATCGCCATCCCCACCAACTTCACCATCAGCGTGACC
ACCGAAATTCTGCCCGTGAGCATGACCAAGACAAGCGTGGACTGCACCATGTACATCTGC
GGCGACAGCACCGAGTGCAGCAACCTGCTCCTGCAGTACGGCAGCTTCTGCACACAGCTG
AAGAGAGCCCTGACCGGCATCGCCGTGGAGCAAGACAAGAACACCCAAGAGGTGTTCGCC
CAAGTGAAGCAGATCTACAAGACCCCCCCCATCAAGTACTTCGGCGGCTTCAACTTCAGC
CAAATCCTGCCCGACCCTAGCAAGCCTAGCAAGAGAAGCTTCATCGAGGACCTGCTGTTC
AACAAGGTGACCCTGGCCGACGCCGGCTTCATCAAGCAGTACGGCGACTGCCTGGGCGAT
ATTGCCGCTAGAGACCTGATCTGCGCTCAGAAGTTCAAGGGCCTGACCGTGCTGCCCCCC
CTGCTGACCGACGAGATGATCGCTCAGTATACAAGCGCCCTCCTCGCTGGGACCATCACA
AGCGGCTGGACATTCGGGGCCGGCGCTGCCCTGCAGATCCCCTTCGCCATGCAGATGGCC
TACAGATTCAACGGCATCGGCGTGACACAGAACGTGCTGTACGAGAATCAGAAGCTGATC
GCCAATCAGTTCAACAGCGCCATCGGCAAGATCCAAGACAGCCTGAGCAGCACCGCTAGC
GCCCTGGGCAAGCTGCAAGACGTGGTGAACCACAACGCCCAAGCCCTGAACACCCTGGTG
AAGCAGCTGAGCAGCAAGTTCGGCGCCATCAGCAGCGTGCTGAACGACATCTTCAGCAGA
CTGGACCCCCCCGAGGCCGAGGTGCAGATCGACCGGCTCATCACCGGCAGACTGCAGAGC
CTGCAGACCTACGTGACACAGCAGCTGATCAGAGCCGCCGAGATCAGAGCTAGCGCCAAC
CTGGCCGCCACCAAGATGAGCGAGTGCGTGCTGGGGCAGAGCAAGAGAGTGGACTTCTGC
GGCAAGGGCTACCACCTGATGAGCTTCCCTCAGAGCGCCCCCCACGGCGTGGTGTTCCTG
CACGTGACCTACGTGCCCGCCCAAGAGAAGAACTTCACCACCGCTCCCGCCATTTGCCAC
GACGGCAAGGCCCACTTCCCTAGAGAGGGCGTGTTCGTGAGCAACGGCACCCACTGGTTC
GTGACACAGAGAAACTTCTACGAGCCTCAGATCATCACCACCGACAACACCTTCGTCAGC
GGCAACTGTGACGTGGTGATCGGCATCGTGAACAATACCGTGTACGACCCCCTGCAGCCC
GAACTGGACAGCTTCAAGGAGGAGCTGGACAAGTACTTCAAGAACCACACAAGCCCCGAC
GTGGACCTGGGCGACATTAGCGGCATCAACGCTAGCGTGGTGAACATTCAGAAGGAGATT
GACAGACTGAACGAGGTGGCCAAGAACCTGAACGAGAGCCTGATCGACCTGCAAGAGCTG
GGCAAGTACGAGCAGTACATCAAGTGGCCCTGGTACATCTGGCTGGGCTTCATCGCCGGC
CTGATCGCCATCGTGATGGTGACCATCATGCTGTGCTGCATGACAAGCTGCTGTAGCTGC
CTGAAAGGCTGCTGCAGCTGCGGCAGCTGCTGCAAGTTCGACGAGGACGACAGCGAGCCC
GTGCTGAAGGGCGTGAAGCTGCACTACACCTGA
SEQ ID NO: 33:
Amino acid sequence of S antigen of Ad-S-Omicron-BA. 1 vaccine
MFVFLVLLPLVSSQCVNLTTRTQLPPAYTNSFTRGVYYPDKVFRSSVLHSTQDLFLPFFS
NVTWFHVISGTNGTKRFDNPVLPFNDGVYFASIEKSNIIRGWIFGTTLDSKTQSLLIVNN
ATNVVIKVCEFQFCNDPFLDHKNNKSWMESEFRVYSSANNCTFEYVSQPFLMDLEGKQGN
FKNLREFVFKNIDGYFKIYSKHTPIIVREPEDLPQGFSALEPLVDLPIGINITRFQTLLA
LHRSYLTPGDSSSGWTAGAAAYYVGYLQPRTFLLKYNENGTITDAVDCALDPLSETKCTL
KSFTVEKGIYQTSNFRVQPTESIVRFPNITNLCPFDEVFNATRFASVYAWNRKRISNCVA
DYSVLYNLAPFFTFKCYGVSPTKLNDLCFTNVYADSFVIRGDEVRQIAPGQTGNIADYNY
KLPDDFTGCVIAWNSNKLDSKVSGNYNYLYRLFRKSNLKPFERDISTEIYQAGNKPCNGV
AGFNCYFPLRSYSFRPTYGVGHQPYRVVVLSFELLHAPATVCGPKKSTNLVKNKCVNFNF
NGLKGTGVLTESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEYVNNSYECD
IPIGAGICASYQTQTKSHRRARSVASQSIIAYTMSLGAENSVAYSNNSIAIPTNFTISVT
TEILPVSMTKTSVDCTMYICGDSTECSNLLLQYGSFCTQLKRALTGIAVEQDKNTQEVFA
QVKQIYKTPPIKYFGGFNFSQILPDPSKPSKRSFIEDLLFNKVTLADAGFIKQYGDCLGD
IAARDLICAQKFKGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVNHNAQALNTLV
KQLSSKFGAISSVLNDIFSRLDPPEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEP
VLKGVKLHYT*
SEQ ID NO: 34:
Nucleotide sequence of S antigen of Ad-S-Omicron-BA.2 vaccine
ATGTTCGTGTTCCTGGTGCTGCTGCCCCTGGTGAGCAGCCAATGCGTGAACCTGATCACA
AGAACACAGAGCTACACCAACAGCTTCACAAGAGGCGTGTACTACCCCGACAAGGTGTTC
AGAAGCAGCGTCCTCCACAGCACCCAAGACCTGTTCCTGCCTTTCTTTAGCAACGTGACC
TGGTTCCACGCCATCCACGTGAGCGGCACCAACGGCACCAAGAGATTCGACAACCCCGTG
CTGCCCTTCAACGACGGCGTGTACTTCGCTAGCACCGAGAAGAGCAACATCATCAGAGGC
TGGATCTTCGGCACCACCCTGGACAGCAAGACACAGAGCCTGCTGATCGTCAACAACGCC
ACCAACGTGGTGATCAAGGTGTGCGAGTTTCAGTTCTGCAACGACCCCTTCCTGGACGTG
TACTACCACAAGAACAACAAGAGCTGGATGGAGAGCGAGTTCAGAGTGTACAGCAGCGCC
AACAACTGCACCTTCGAGTACGTGAGCCAACCCTTCCTGATGGACCTGGAGGGCAAGCAA
GGCAACTTCAAAAACCTGAGAGAGTTCGTGTTCAAGAACATCGACGGCTACTTCAAGATC
TACAGCAAGCACACCCCCATCAACCTGGGCAGAGACCTGCCCCAAGGCTTCAGCGCCCTG
GAGCCCCTGGTGGACCTGCCCATCGGCATCAACATCACAAGATTTCAGACCCTGCTGGCC
CTGCACAGATCCTACCTCACACCTGGCGACAGCAGCTCCGGCTGGACCGCTGGCGCTGCC
GCTTACTACGTGGGCTACCTGCAGCCTAGAACCTTCCTGCTGAAGTACAACGAGAACGGC
ACAATCACCGATGCCGTCGACTGCGCCCTGGACCCCCTGAGCGAGACCAAGTGCACCCTG
AAGAGCTTCACCGTGGAGAAGGGCATCTATCAGACAAGCAACTTCAGAGTGCAGCCCACC
GAGAGCATCGTGAGATTCCCCAACATCACCAACCTGTGCCCCTTCGACGAGGTGTTCAAC
GCCACAAGATTCGCTAGCGTGTACGCCTGGAACAGAAAAAGAATCAGCAACTGCGTGGCC
GACTACAGCGTGCTGTACAACTTCGCCCCCTTCTTCGCCTTCAAATGCTACGGCGTGAGC
CCCACCAAGCTGAACGACCTGTGCTTCACCAACGTGTACGCCGACAGCTTCGTGATCAGA
GGCAACGAGGTGAGCCAAATCGCCCCCGGGCAGACCGGCAACATCGCCGACTACAACTAC
AAGCTGCCCGACGACTTCACCGGCTGCGTGATCGCCTGGAACTCCAACAAGCTCGACAGC
AAGGTGGGCGGCAACTACAACTACCTGTACAGACTGTTCAGAAAGAGCAACCTGAAGCCC
TTCGAGAGAGACATCAGCACCGAGATCTACCAAGCCGGCAACAAGCCCTGCAACGGCGTG
GCCGGCTTCAACTGCTACTTCCCCCTGAGAAGCTACGGCTTCAGACCCACCTACGGCGTG
GGCCATCAGCCCTACAGAGTGGTCGTGCTGAGCTTCGAGCTGCTGCACGCCCCCGCCACC
GTGTGCGGCCCCAAGAAGAGCACCAACCTGGTGAAGAACAAGTGCGTGAACTTCAACTTC
AACGGCCTGACCGGCACCGGCGTGCTGACCGAGAGCAACAAGAAGTTCCTCCCCTTTCAG
CAGTTCGGCAGAGACATCGCCGACACCACCGACGCCGTGAGAGACCCTCAGACCCTGGAG
ATCCTGGACATCACCCCCTGCAGCTTCGGCGGCGTGAGCGTGATCACCCCCGGCACCAAC
ACAAGCAACCAAGTGGCCGTGCTGTACCAAGGCGTGAACTGCACCGAGGTGCCCGTGGCC
ATCCACGCCGATCAGCTGACCCCCACCTGGAGAGTGTACTCCACCGGCAGCAACGTGTTT
CAGACAAGAGCCGGCTGCCTGATCGGCGCCGAGTACGTGAACAACAGCTACGAGTGCGAC
ATCCCCATCGGCGCCGGCATCTGCGCTAGCTATCAGACACAGACCAAGAGCCACAGAAGA
GCTAGAAGCGTGGCTAGCCAAAGCATCATCGCCTACACCATGAGCCTGGGCGCCGAGAAC
AGCGTGGCCTACAGCAACAACAGCATCGCCATCCCCACCAACTTCACCATCAGCGTGACC
ACCGAGATCCTGCCTGTGAGCATGACCAAGACAAGCGTGGACTGCACCATGTACATCTGC
GGCGACAGCACCGAGTGCAGCAACCTGCTCCTGCAGTACGGCAGCTTCTGCACACAGCTG
AAGAGAGCCCTGACCGGCATCGCCGTGGAGCAAGACAAGAACACCCAAGAGGTGTTCGCC
CAAGTGAAGCAGATCTACAAGACCCCCCCCATCAAGTACTTCGGCGGCTTCAACTTCAGC
CAAATCCTGCCCGACCCTAGCAAGCCTAGCAAGCGGAGCTTCATCGAGGACCTGCTGTTC
AACAAGGTGACCCTGGCCGACGCCGGCTTCATCAAGCAGTACGGCGACTGCCTCGGCGAC
ATCGCCGCTAGAGACCTGATCTGCGCTCAGAAGTTCAATGGCCTGACCGTGCTGCCCCCC
CTGCTGACCGACGAGATGATCGCTCAGTATACCTCCGCCCTGCTCGCCGGGACCATCACA
AGCGGCTGGACCTTCGGCGCCGGCGCCGCCCTCCAAATCCCCTTCGCCATGCAGATGGCC
TACAGATTCAACGGCATCGGCGTGACACAGAACGTGCTGTACGAGAATCAGAAGCTGATC
GCCAATCAGTTCAACAGCGCCATCGGCAAGATCCAAGACAGCCTGAGCAGCACCGCTAGC
GCCCTGGGCAAGCTGCAAGACGTGGTGAACCACAACGCCCAAGCCCTGAACACCCTGGTG
AAGCAGCTGAGCAGCAAGTTCGGCGCCATCAGCAGCGTGCTGAACGATATCCTGAGCAGA
CTGGACCCCCCCGAGGCCGAGGTGCAGATCGATAGACTGATCACCGGCAGACTGCAGAGC
CTGCAGACCTACGTGACACAGCAGCTGATCAGAGCCGCCGAGATCAGAGCTAGCGCCAAC
CTGGCCGCCACCAAGATGAGCGAGTGCGTGCTGGGGCAGAGCAAGAGAGTGGACTTCTGC
GGCAAGGGCTACCACCTGATGAGCTTCCCTCAGAGCGCCCCCCACGGCGTGGTGTTCCTG
CACGTGACCTACGTGCCCGCCCAAGAGAAGAACTTCACAACCGCCCCCGCCATCTGCCAC
GACGGCAAGGCCCACTTCCCTAGAGAGGGCGTGTTCGTGAGCAACGGCACCCACTGGTTC
GTGACACAGAGAAACTTCTACGAGCCTCAGATCATCACCACCGACAACACCTTCGTGAGC
GGCAACTGCGACGTGGTGATCGGGATCGTGAACAACACCGTGTACGATCCCCTGCAGCCC
GAGCTGGACAGCTTCAAGGAGGAGCTGGACAAGTACTTCAAGAACCACACAAGCCCCGAC
GTGGACCTGGGCGACATTAGCGGCATCAACGCTAGCGTGGTGAACATTCAGAAGGAGATC
GACAGACTGAATGAGGTGGCCAAGAACCTGAACGAGAGCCTGATCGACCTGCAAGAGCTG
GGCAAGTACGAGCAGTACATCAAGTGGCCCTGGTACATCTGGCTGGGCTTCATCGCCGGC
CTGATCGCCATCGTGATGGTGACCATCATGCTGTGCTGCATGACAAGCTGCTGTAGCTGC
CTGAAGGGCTGCTGCAGCTGCGGCTCCTGCTGCAAGTTCGACGAGGACGACAGCGAGCCC
GTGCTGAAGGGCGTGAAGCTGCACTACACCTGA
SEQ ID NO: 35:
Amino acid sequence of S antigen of Ad-S-Omicron-BA.2 vaccine
MFVFLVLLPLVSSQCVNLITRTQSYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNA
TNVVIKVCEFQFCNDPFLDVYYHKNNKSWMESEFRVYSSANNCTFEYVSQPFLMDLEGKQ
GNFKNLREFVFKNIDGYFKIYSKHTPINLGRDLPQGFSALEPLVDLPIGINITRFQTLLA
LHRSYLTPGDSSSGWTAGAAAYYVGYLQPRTFLLKYNENGTITDAVDCALDPLSETKCTL
KSFTVEKGIYQTSNFRVQPTESIVRFPNITNLCPFDEVFNATRFASVYAWNRKRISNCVA
DYSVLYNFAPFFAFKCYGVSPTKLNDLCFTNVYADSFVIRGNEVSQIAPGQTGNIADYNY
KLPDDFTGCVIAWNSNKLDSKVGGNYNYLYRLFRKSNLKPFERDISTEIYQAGNKPCNGV
AGFNCYFPLRSYGFRPTYGVGHQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEYVNNSYECD
IPIGAGICASYQTQTKSHRRARSVASQSIIAYTMSLGAENSVAYSNNSIAIPTNFTISVT
TEILPVSMTKTSVDCTMYICGDSTECSNLLLQYGSFCTQLKRALTGIAVEQDKNTQEVFA
QVKQIYKTPPIKYFGGFNFSQILPDPSKPSKRSFIEDLLFNKVTLADAGFIKQYGDCLGD
IAARDLICAQKFNGLTVLPPLLTDEMIAQYTSALLAGTITSGWTFGAGAALQIPFAMQMA
YRENGIGVTQNVLYENQKLIANQFNSAIGKIQDSLSSTASALGKLQDVVNHNAQALNTLV
KQLSSKFGAISSVLNDILSRLDPPEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEP
VLKGVKLHYT*
SEQ ID NO: 36:
Nucleotide sequence of S antigen of Ad-S-Omicron-BA.2.12.1
vaccine
ATGTTCGTGTTCCTGGTGCTGCTGCCCCTGGTGAGCAGCCAATGCGTGAACCTGATCACA
AGAACACAGAGCTACACCAACAGCTTCACAAGAGGCGTGTACTACCCCGACAAGGTGTTC
AGAAGCAGCGTCCTCCACAGCACCCAAGACCTGTTCCTGCCTTTCTTTAGCAACGTGACC
TGGTTCCACGCCATCCACGTGAGCGGCACCAACGGCACCAAGAGATTCGACAACCCCGTG
CTGCCCTTCAACGACGGCGTGTACTTCGCTAGCACCGAGAAGAGCAACATCATCAGAGGC
TGGATCTTCGGCACCACCCTGGACAGCAAGACACAGAGCCTGCTGATCGTCAACAACGCC
ACCAACGTGGTGATCAAGGTGTGCGAGTTTCAGTTCTGCAACGACCCCTTCCTGGACGTG
TACTACCACAAGAACAACAAGAGCTGGATGGAGAGCGAGTTCAGAGTGTACAGCAGCGCC
AACAACTGCACCTTCGAGTACGTGAGCCAACCCTTCCTGATGGACCTGGAGGGCAAGCAA
GGCAACTTCAAAAACCTGAGAGAGTTCGTGTTCAAGAACATCGACGGCTACTTCAAGATC
TACAGCAAGCACACCCCCATCAACCTGGGCAGAGACCTGCCCCAAGGCTTCAGCGCCCTG
GAGCCCCTGGTGGACCTGCCCATCGGCATCAACATCACAAGATTTCAGACCCTGCTGGCC
CTGCACAGATCCTACCTCACACCTGGCGACAGCAGCTCCGGCTGGACCGCTGGCGCTGCC
GCTTACTACGTGGGCTACCTGCAGCCTAGAACCTTCCTGCTGAAGTACAACGAGAACGGC
ACAATCACCGATGCCGTCGACTGCGCCCTGGACCCCCTGAGCGAGACCAAGTGCACCCTG
AAGAGCTTCACCGTGGAGAAGGGCATCTATCAGACAAGCAACTTCAGAGTGCAGCCCACC
GAGAGCATCGTGAGATTCCCCAACATCACCAACCTGTGCCCCTTCGACGAGGTGTTCAAC
GCCACAAGATTCGCTAGCGTGTACGCCTGGAACAGAAAAAGAATCAGCAACTGCGTGGCC
GACTACAGCGTGCTGTACAACTTCGCCCCCTTCTTCGCCTTCAAATGCTACGGCGTGAGC
CCCACCAAGCTGAACGACCTGTGCTTCACCAACGTGTACGCCGACAGCTTCGTGATCAGA
GGCAACGAGGTGAGCCAAATCGCCCCCGGGCAGACCGGCAACATCGCCGACTACAACTAC
AAGCTGCCCGACGACTTCACCGGCTGCGTGATCGCCTGGAACTCCAACAAGCTCGACAGC
AAGGTGGGCGGCAACTACAACTATCAGTACAGACTGTTCAGAAAGAGCAACCTGAAGCCC
TTCGAGAGAGACATCAGCACCGAGATCTACCAAGCCGGCAACAAGCCCTGCAACGGCGTG
GCCGGCTTCAACTGCTACTTCCCCCTGAGAAGCTACGGCTTCAGACCCACCTACGGCGTG
GGCCATCAGCCCTACAGAGTGGTCGTGCTGAGCTTCGAGCTGCTGCACGCCCCCGCCACC
GTGTGCGGCCCCAAGAAGAGCACCAACCTGGTGAAGAACAAGTGCGTGAACTTCAACTTC
AACGGCCTGACCGGCACCGGCGTGCTGACCGAGAGCAACAAGAAGTTCCTCCCCTTTCAG
CAGTTCGGCAGAGACATCGCCGACACCACCGACGCCGTGAGAGACCCTCAGACCCTGGAG
ATCCTGGACATCACCCCCTGCAGCTTCGGCGGCGTGAGCGTGATCACCCCCGGCACCAAC
ACAAGCAACCAAGTGGCCGTGCTGTACCAAGGCGTGAACTGCACCGAGGTGCCCGTGGCC
ATCCACGCCGATCAGCTGACCCCCACCTGGAGAGTGTACTCCACCGGCAGCAACGTGTTT
CAGACAAGAGCCGGCTGCCTGATCGGCGCCGAGTACGTGAACAACAGCTACGAGTGCGAC
ATCCCCATCGGCGCCGGCATCTGCGCTAGCTATCAGACACAGACCAAGAGCCACAGAAGA
GCTAGAAGCGTGGCTAGCCAAAGCATCATCGCCTACACCATGAGCCTGGGCGCCGAGAAC
CTGGTGGCCTACAGCAACAACAGCATCGCCATCCCCACCAACTTCACCATCAGCGTGACC
ACCGAGATCCTGCCTGTGAGCATGACCAAGACAAGCGTGGACTGCACCATGTACATCTGC
GGCGACAGCACCGAGTGCAGCAACCTGCTCCTGCAGTACGGCAGCTTCTGCACACAGCTG
AAGAGAGCCCTGACCGGCATCGCCGTGGAGCAAGACAAGAACACCCAAGAGGTGTTCGCC
CAAGTGAAGCAGATCTACAAGACCCCCCCCATCAAGTACTTCGGCGGCTTCAACTTCAGC
CAAATCCTGCCCGACCCTAGCAAGCCTAGCAAGCGGAGCTTCATCGAGGACCTGCTGTTC
AACAAGGTGACCCTGGCCGACGCCGGCTTCATCAAGCAGTACGGCGACTGCCTCGGCGAC
ATCGCCGCTAGAGACCTGATCTGCGCTCAGAAGTTCAATGGCCTGACCGTGCTGCCCCCC
CTGCTGACCGACGAGATGATCGCTCAGTATACCTCCGCCCTGCTCGCCGGGACCATCACA
AGCGGCTGGACCTTCGGCGCCGGCGCCGCCCTCCAAATCCCCTTCGCCATGCAGATGGCC
TACAGATTCAACGGCATCGGCGTGACACAGAACGTGCTGTACGAGAATCAGAAGCTGATC
GCCAATCAGTTCAACAGCGCCATCGGCAAGATCCAAGACAGCCTGAGCAGCACCGCTAGC
GCCCTGGGCAAGCTGCAAGACGTGGTGAACCACAACGCCCAAGCCCTGAACACCCTGGTG
AAGCAGCTGAGCAGCAAGTTCGGCGCCATCAGCAGCGTGCTGAACGATATCCTGAGCAGA
CTGGACCCCCCCGAGGCCGAGGTGCAGATCGATAGACTGATCACCGGCAGACTGCAGAGC
CTGCAGACCTACGTGACACAGCAGCTGATCAGAGCCGCCGAGATCAGAGCTAGCGCCAAC
CTGGCCGCCACCAAGATGAGCGAGTGCGTGCTGGGGCAGAGCAAGAGAGTGGACTTCTGC
GGCAAGGGCTACCACCTGATGAGCTTCCCTCAGAGCGCCCCCCACGGCGTGGTGTTCCTG
CACGTGACCTACGTGCCCGCCCAAGAGAAGAACTTCACAACCGCCCCCGCCATCTGCCAC
GACGGCAAGGCCCACTTCCCTAGAGAGGGCGTGTTCGTGAGCAACGGCACCCACTGGTTC
GTGACACAGAGAAACTTCTACGAGCCTCAGATCATCACCACCGACAACACCTTCGTGAGC
GGCAACTGCGACGTGGTGATCGGGATCGTGAACAACACCGTGTACGATCCCCTGCAGCCC
GAGCTGGACAGCTTCAAGGAGGAGCTGGACAAGTACTTCAAGAACCACACAAGCCCCGAC
GTGGACCTGGGCGACATTAGCGGCATCAACGCTAGCGTGGTGAACATTCAGAAGGAGATC
GACAGACTGAATGAGGTGGCCAAGAACCTGAACGAGAGCCTGATCGACCTGCAAGAGCTG
GGCAAGTACGAGCAGTACATCAAGTGGCCCTGGTACATCTGGCTGGGCTTCATCGCCGGC
CTGATCGCCATCGTGATGGTGACCATCATGCTGTGCTGCATGACAAGCTGCTGTAGCTGC
CTGAAGGGCTGCTGCAGCTGCGGCTCCTGCTGCAAGTTCGACGAGGACGACAGCGAGCCC
GTGCTGAAGGGCGTGAAGCTGCACTACACCTGA
SEQ ID NO: 37:
Amino acid sequence of S antigen of Ad-S-Omicron-BA.2.12.1
vaccine
MFVFLVLLPLVSSQCVNLITRTQSYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNA
TNVVIKVCEFQFCNDPFLDVYYHKNNKSWMESEFRVYSSANNCTFEYVSQPFLMDLEGKQ
GNFKNLREFVFKNIDGYFKIYSKHTPINLGRDLPQGFSALEPLVDLPIGINITRFQTLLA
LHRSYLTPGDSSSGWTAGAAAYYVGYLQPRTFLLKYNENGTITDAVDCALDPLSETKCTL
KSFTVEKGIYQTSNFRVQPTESIVRFPNITNLCPFDEVFNATRFASVYAWNRKRISNCVA
DYSVLYNFAPFFAFKCYGVSPTKLNDLCFTNVYADSFVIRGNEVSQIAPGQTGNIADYNY
KLPDDFTGCVIAWNSNKLDSKVGGNYNYQYRLFRKSNLKPFERDISTEIYQAGNKPCNGV
AGFNCYFPLRSYGFRPTYGVGHQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEYVNNSYECD
IPIGAGICASYQTQTKSHRRARSVASQSIIAYTMSLGAENLVAYSNNSIAIPTNFTISVT
TEILPVSMTKTSVDCTMYICGDSTECSNLLLQYGSFCTQLKRALTGIAVEQDKNTQEVFA
QVKQIYKTPPIKYFGGFNFSQILPDPSKPSKRSFIEDLLFNKVTLADAGFIKQYGDCLGD
IAARDLICAQKFNGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVNHNAQALNTLV
KQLSSKFGAISSVLNDILSRLDPPEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEP
VLKGVKLHYT*
SEQ ID NO: 38:
Nucleotide sequence of S antigen of Ad-S-Omicron-BA.4/5 vaccine
ATGTTCGTGTTCCTGGTGCTGCTGCCCCTGGTGAGCAGCCAATGCGTGAACCTGATCACA
AGAACACAGAGCTACACCAACAGCTTCACAAGAGGCGTGTACTACCCCGACAAGGTGTTC
AGAAGCAGCGTCCTGCATTCCACCCAAGACCTGTTTCTCCCCTTCTTCAGCAACGTGACC
TGGTTCCACGCCATCAGCGGCACCAACGGCACCAAGAGATTCGACAACCCCGTGCTGCCC
TTCAACGACGGCGTGTACTTCGCTAGCACCGAGAAGAGCAACATCATCAGAGGCTGGATC
TTCGGCACCACCCTGGACAGCAAGACACAGAGCCTGCTGATCGTGAACAACGCCACAAAC
GTGGTGATCAAGGTGTGCGAGTTTCAGTTCTGCAACGACCCCTTCCTGGACGTGTACTAC
CACAAGAACAACAAGAGCTGGATGGAGAGCGAGTTCCGGGTGTACAGCAGCGCCAACAAC
TGCACCTTCGAGTACGTGAGCCAACCCTTCCTGATGGACCTGGAGGGCAAGCAAGGCAAC
TTCAAGAACCTGAGAGAGTTCGTGTTCAAGAACATCGACGGCTACTTCAAGATCTACAGC
AAGCACACCCCCATCAACCTGGGCAGAGACCTGCCCCAAGGCTTCAGCGCCCTGGAGCCC
CTGGTGGACCTGCCCATCGGCATCAACATCACAAGATTTCAGACACTGCTCGCCCTGCAC
CGGAGCTACCTCACCCCTGGCGACAGCAGCTCCGGCTGGACCGCTGGGGCTGCCGCCTAC
TACGTGGGCTACCTGCAGCCTAGAACCTTCCTGCTGAAGTACAACGAGAACGGCACCATC
ACCGACGCTGTGGACTGTGCCCTGGACCCCCTGAGCGAGACCAAGTGCACCCTGAAGAGC
TTCACCGTGGAGAAGGGCATCTATCAGACAAGCAACTTCAGAGTGCAGCCCACCGAGAGC
ATCGTGAGATTCCCCAACATCACCAACCTGTGCCCCTTCGACGAGGTGTTCAACGCCACA
AGATTCGCTAGCGTGTACGCCTGGAACAGAAAGAGAATCAGCAACTGCGTGGCCGACTAC
AGCGTGCTGTACAACTTCGCCCCCTTCTTCGCCTTCAAATGCTACGGCGTCAGCCCCACC
AAGCTGAACGACCTGTGCTTCACCAACGTGTACGCCGACAGCTTCGTGATCAGAGGCAAC
GAGGTGAGCCAAATCGCCCCCGGGCAGACCGGCAACATCGCCGACTATAACTACAAACTG
CCCGACGACTTCACCGGCTGCGTGATCGCCTGGAACTCCAACAAGCTGGACAGCAAAGTG
GGCGGCAACTACAACTACAGATACAGACTGTTCAGAAAGAGCAACCTGAAGCCCTTCGAG
AGAGACATCAGCACCGAGATCTACCAAGCCGGCAACAAGCCCTGCAACGGCGTGGCCGGC
GTGAACTGCTACTTCCCCCTGCAGAGCTACGGCTTCAGACCCACCTACGGCGTGGGCCAT
CAGCCCTACAGAGTGGTCGTGCTGAGCTTCGAGCTGCTGCACGCCCCTGCCACAGTGTGC
GGCCCCAAGAAGAGCACCAACCTGGTGAAGAACAAGTGCGTGAACTTCAACTTCAACGGC
CTGACCGGCACCGGCGTGCTGACCGAGAGCAACAAGAAGTTCCTGCCCTTTCAGCAGTTC
GGCAGAGACATCGCCGACACCACCGATGCTGTGAGAGACCCTCAGACCCTGGAGATCCTG
GACATCACCCCTTGCAGCTTCGGCGGCGTGAGCGTGATCACCCCCGGCACCAACACAAGC
AACCAAGTGGCCGTGCTGTACCAAGGCGTCAACTGCACAGAGGTGCCCGTGGCCATCCAC
GCCGATCAGCTGACCCCCACCTGGAGAGTGTACTCCACCGGCAGCAACGTGTTTCAGACA
AGAGCCGGCTGCCTGATCGGCGCCGAGTACGTGAACAACAGCTACGAGTGCGACATCCCC
ATCGGCGCCGGCATCTGCGCTAGCTATCAGACACAGACCAAGAGCCACAGAAGAGCTAGA
AGCGTGGCTAGCCAAAGCATCATCGCCTACACCATGAGCCTGGGCGCCGAGAACAGCGTG
GCCTACAGCAACAACAGCATCGCCATCCCCACCAACTTCACCATCAGCGTGACCACCGAG
ATCCTGCCTGTGAGCATGACCAAGACAAGCGTGGACTGCACCATGTACATCTGCGGCGAC
AGCACCGAGTGCAGCAACCTGCTCCTGCAGTACGGCAGCTTCTGCACACAGCTGAAGAGA
GCCCTGACCGGCATCGCCGTGGAGCAAGACAAGAACACCCAAGAGGTGTTCGCCCAAGTG
AAGCAGATCTACAAGACCCCCCCCATCAAGTACTTCGGCGGCTTCAACTTCAGCCAAATC
CTGCCCGACCCTAGCAAGCCTAGCAAGCGGAGCTTCATCGAGGACCTGCTGTTCAACAAG
GTGACCCTGGCCGACGCCGGCTTCATCAAGCAGTACGGCGACTGCCTCGGCGACATCGCT
GCTAGAGACCTGATCTGCGCTCAGAAGTTCAACGGCCTCACAGTGCTGCCCCCCCTGCTG
ACCGACGAGATGATCGCTCAGTACACCTCCGCTCTGCTGGCCGGCACAATTACATCCGGC
TGGACCTTCGGCGCCGGCGCCGCCCTGCAAATCCCCTTCGCCATGCAGATGGCCTACAGA
TTCAACGGCATCGGCGTGACACAGAACGTGCTGTACGAGAATCAGAAGCTGATCGCCAAT
CAGTTCAACAGCGCCATCGGCAAGATCCAAGACAGCCTGAGCAGCACCGCTAGCGCCCTG
GGCAAGCTGCAAGACGTGGTGAACCACAACGCCCAAGCCCTGAACACCCTGGTGAAGCAG
CTGAGCAGCAAGTTCGGCGCCATCTCCTCCGTCCTGAACGACATCCTGAGCAGACTGGAC
CCCCCCGAGGCCGAGGTGCAGATTGACAGACTGATTACCGGCAGACTGCAGAGCCTGCAG
ACCTACGTGACACAGCAGCTGATCAGAGCCGCCGAGATCAGAGCTAGCGCCAACCTGGCC
GCCACCAAGATGAGCGAGTGCGTGCTGGGGCAGAGCAAGAGAGTGGACTTCTGCGGCAAG
GGCTACCACCTGATGAGCTTCCCTCAGAGCGCCCCCCACGGCGTGGTGTTCCTGCACGTG
ACCTACGTGCCCGCCCAAGAGAAGAACTTCACCACCGCTCCCGCCATCTGCCACGACGGC
AAGGCCCACTTCCCTAGAGAGGGCGTGTTCGTGAGCAACGGCACCCACTGGTTCGTGACA
CAGAGAAACTTCTACGAGCCTCAGATCATCACCACCGACAACACCTTCGTGAGCGGCAAC
TGCGACGTGGTGATCGGCATCGTCAACAACACCGTGTACGACCCCCTGCAACCCGAGCTG
GACAGCTTCAAGGAGGAGCTGGACAAGTACTTTAAGAACCACACAAGCCCCGACGTGGAC
CTGGGGGACATCTCCGGCATCAACGCTAGCGTGGTGAACATTCAGAAGGAAATTGACAGA
CTGAATGAGGTGGCCAAGAACCTGAACGAGAGCCTGATCGACCTGCAAGAGCTGGGCAAG
TACGAGCAGTACATCAAGTGGCCCTGGTACATCTGGCTGGGCTTCATCGCCGGCCTGATC
GCCATCGTGATGGTGACCATCATGCTGTGCTGCATGACAAGCTGCTGCAGCTGTCTGAAG
GGCTGCTGCTCCTGCGGCAGCTGCTGCAAGTTCGACGAGGACGACAGCGAGCCCGTGCTG
AAGGGCGTGAAGCTGCACTACACCTGA
SEQ ID NO: 39:
Amino acid sequence of S antigen of Ad-S-Omicron-BA.4/5
vaccine
MFVFLVLLPLVSSQCVNLITRTQSYTNSFTRGVYYPDKVFRSSVLHSTQDLFLPFFSNVT
WFHAISGTNGTKRFDNPVLPFNDGVYFASTEKSNIIRGWIFGTTLDSKTQSLLIVNNATN
VVIKVCEFQFCNDPFLDVYYHKNNKSWMESEFRVYSSANNCTFEYVSQPFLMDLEGKQGN
FKNLREFVFKNIDGYFKIYSKHTPINLGRDLPQGFSALEPLVDLPIGINITRFQTLLALH
RSYLTPGDSSSGWTAGAAAYYVGYLQPRTFLLKYNENGTITDAVDCALDPLSETKCTLKS
FTVEKGIYQTSNFRVQPTESIVRFPNITNLCPFDEVFNATRFASVYAWNRKRISNCVADY
SVLYNFAPFFAFKCYGVSPTKLNDLCFTNVYADSFVIRGNEVSQIAPGQTGNIADYNYKL
PDDFTGCVIAWNSNKLDSKVGGNYNYRYRLFRKSNLKPFERDISTEIYQAGNKPCNGVAG
VNCYFPLQSYGFRPTYGVGHQPYRVVVLSFELLHAPATVCGPKKSTNLVKNKCVNFNFNG
LTGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEYVNNSYECDIP
IGAGICASYQTQTKSHRRARSVASQSIIAYTMSLGAENSVAYSNNSIAIPTNFTISVTTE
ILPVSMTKTSVDCTMYICGDSTECSNLLLQYGSFCTQLKRALTGIAVEQDKNTQEVFAQV
KQIYKTPPIKYFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNHNAQALNTLVKQ
LSSKFGAISSVLNDILSRLDPPEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK
YEQYIKWPWYIWLGFIAGLIAIVMVTIMLCCMTSCCSCLKGCCSCGSCCKFDEDDSEPVL
KGVKLHYT*
SEQ ID NO: 40:
Full-length amino acid sequence of wild S antigen:
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLENKVTLADAGFIKQYGDC
LGDIAARDLICAQKFNGLTVLPPLLTDEMIAQYTSALLAGTITSGWTFGAGAALQIPFAM
QMAYRENGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDD
SEPVLKGVKLHYT
Note:
the above sequence consists of signal peptide-extracellular domain-
transmembrane domain-intracellular domain.
SEQ ID NO: 41:
full-length nucleic acid sequence of wild S antigen (optimization of
CHO cells):
ATGTTCGTGTTCCTGGTGCTGCTGCCCCTGGTGAGCAGCCAGTGCGTGAACCTGACCACC
AGAACCCAGCTGCCCCCCGCCTACACCAACAGCTTCACCAGAGGCGTGTACTACCCCGAC
AAGGTGTTCAGAAGCAGCGTGCTGCACAGCACCCAGGACCTGTTCCTGCCCTTCTTCAGC
AACGTGACCTGGTTCCACGCCATCCACGTGAGCGGCACCAACGGCACCAAGAGATTCGAC
AACCCCGTGCTGCCCTTCAACGACGGCGTGTACTTCGCCAGCACCGAGAAGAGCAACATC
ATCAGAGGCTGGATCTTCGGCACCACCCTGGACAGCAAGACCCAGAGCCTGCTGATCGTG
AACAACGCCACCAACGTGGTGATCAAGGTGTGCGAGTTCCAGTTCTGCAACGACCCCTTC
CTGGGCGTGTACTACCACAAGAACAACAAGAGCTGGATGGAGAGCGAGTTCAGAGTGTAC
AGCAGCGCCAACAACTGCACCTTCGAGTACGTGAGCCAGCCCTTCCTGATGGACCTGGAG
GGCAAGCAGGGCAACTTCAAGAACCTGAGAGAGTTCGTGTTCAAGAACATCGACGGCTAC
TTCAAGATCTACAGCAAGCACACCCCCATCAACCTGGTGAGAGACCTGCCCCAGGGCTTC
AGCGCCCTGGAGCCCCTGGTGGACCTGCCCATCGGCATCAACATCACCAGATTCCAGACC
CTGCTGGCCCTGCACAGAAGCTACCTGACCCCCGGCGACAGCAGCAGCGGCTGGACCGCC
GGCGCCGCCGCCTACTACGTGGGCTACCTGCAGCCCAGAACCTTCCTGCTGAAGTACAAC
GAGAACGGCACCATCACCGACGCCGTGGACTGCGCCCTGGACCCCCTGAGCGAGACCAAG
TGCACCCTGAAGAGCTTCACCGTGGAGAAGGGCATCTACCAGACCAGCAACTTCAGAGTG
CAGCCCACCGAGAGCATCGTGAGATTCCCCAACATCACCAACCTGTGCCCCTTCGGCGAG
GTGTTCAACGCCACCAGATTCGCCAGCGTGTACGCCTGGAACAGAAAGAGAATCAGCAAC
TGCGTGGCCGACTACAGCGTGCTGTACAACAGCGCCAGCTTCAGCACCTTCAAGTGCTAC
GGCGTGAGCCCCACCAAGCTGAACGACCTGTGCTTCACCAACGTGTACGCCGACAGCTTC
GTGATCAGAGGCGACGAGGTGAGACAGATCGCCCCCGGCCAGACCGGCAAGATCGCCGAC
TACAACTACAAGCTGCCCGACGACTTCACCGGCTGCGTGATCGCCTGGAACAGCAACAAC
CTGGACAGCAAGGTGGGCGGCAACTACAACTACCTGTACAGACTGTTCAGAAAGAGCAAC
CTGAAGCCCTTCGAGAGAGACATCAGCACCGAGATCTACCAGGCCGGCAGCACCCCCTGC
AACGGCGTGGAGGGCTTCAACTGCTACTTCCCCCTGCAGAGCTACGGCTTCCAGCCCACC
AACGGCGTGGGCTACCAGCCCTACAGAGTGGTGGTGCTGAGCTTCGAGCTGCTGCACGCC
CCCGCCACCGTGTGCGGCCCCAAGAAGAGCACCAACCTGGTGAAGAACAAGTGCGTGAAC
TTCAACTTCAACGGCCTGACCGGCACCGGCGTGCTGACCGAGAGCAACAAGAAGTTCCTG
CCCTTCCAGCAGTTCGGCAGAGACATCGCCGACACCACCGACGCCGTGAGAGACCCCCAG
ACCCTGGAGATCCTGGACATCACCCCCTGCAGCTTCGGCGGCGTGAGCGTGATCACCCCC
GGCACCAACACCAGCAACCAGGTGGCCGTGCTGTACCAGGACGTGAACTGCACCGAGGTG
CCCGTGGCCATCCACGCCGACCAGCTGACCCCCACCTGGAGAGTGTACAGCACCGGCAGC
AACGTGTTCCAGACCAGAGCCGGCTGCCTGATCGGCGCCGAGCACGTGAACAACAGCTAC
GAGTGCGACATCCCCATCGGCGCCGGCATCTGCGCCAGCTACCAGACCCAGACCAACAGC
CCCAGAAGAGCCAGAAGCGTGGCCAGCCAGAGCATCATCGCCTACACCATGAGCCTGGGC
GCCGAGAACAGCGTGGCCTACAGCAACAACAGCATCGCCATCCCCACCAACTTCACCATC
AGCGTGACCACCGAGATCCTGCCCGTGAGCATGACCAAGACCAGCGTGGACTGCACCATG
TACATCTGCGGCGACAGCACCGAGTGCAGCAACCTGCTGCTGCAGTACGGCAGCTTCTGC
ACCCAGCTGAACAGAGCCCTGACCGGCATCGCCGTGGAGCAGGACAAGAACACCCAGGAG
GTGTTCGCCCAGGTGAAGCAGATCTACAAGACCCCCCCCATCAAGGACTTCGGCGGCTTC
AACTTCAGCCAGATCCTGCCCGACCCCAGCAAGCCCAGCAAGAGAAGCTTCATCGAGGAC
CTGCTGTTCAACAAGGTGACCCTGGCCGACGCCGGCTTCATCAAGCAGTACGGCGACTGC
CTGGGCGACATCGCCGCCAGAGACCTGATCTGCGCCCAGAAGTTCAACGGCCTGACCGTG
CTGCCCCCCCTGCTGACCGACGAGATGATCGCCCAGTACACCAGCGCCCTGCTGGCCGGC
ACCATCACCAGCGGCTGGACCTTCGGCGCCGGCGCCGCCCTGCAGATCCCCTTCGCCATG
CAGATGGCCTACAGATTCAACGGCATCGGCGTGACCCAGAACGTGCTGTACGAGAACCAG
AAGCTGATCGCCAACCAGTTCAACAGCGCCATCGGCAAGATCCAGGACAGCCTGAGCAGC
ACCGCCAGCGCCCTGGGCAAGCTGCAGGACGTGGTGAACCAGAACGCCCAGGCCCTGAAC
ACCCTGGTGAAGCAGCTGAGCAGCAACTTCGGCGCCATCAGCAGCGTGCTGAACGACATC
CTGAGCAGACTGGACAAGGTGGAGGCCGAGGTGCAGATCGACAGACTGATCACCGGCAGA
CTGCAGAGCCTGCAGACCTACGTGACCCAGCAGCTGATCAGAGCCGCCGAGATCAGAGCC
AGCGCCAACCTGGCCGCCACCAAGATGAGCGAGTGCGTGCTGGGCCAGAGCAAGAGAGTG
GACTTCTGCGGCAAGGGCTACCACCTGATGAGCTTCCCCCAGAGCGCCCCCCACGGCGTG
GTGTTCCTGCACGTGACCTACGTGCCCGCCCAGGAGAAGAACTTCACCACCGCCCCCGCC
ATCTGCCACGACGGCAAGGCCCACTTCCCCAGAGAGGGCGTGTTCGTGAGCAACGGCACC
CACTGGTTCGTGACCCAGAGAAACTTCTACGAGCCCCAGATCATCACCACCGACAACACC
TTCGTGAGCGGCAACTGCGACGTGGTGATCGGCATCGTGAACAACACCGTGTACGACCCC
CTGCAGCCCGAGCTGGACAGCTTCAAGGAGGAGCTGGACAAGTACTTCAAGAACCACACC
AGCCCCGACGTGGACCTGGGCGACATCAGCGGCATCAACGCCAGCGTGGTGAACATCCAG
AAGGAGATCGACAGACTGAACGAGGTGGCCAAGAACCTGAACGAGAGCCTGATCGACCTG
CAGGAGCTGGGCAAGTACGAGCAGTACATCAAGTGGCCCTGGTACATCTGGCTGGGCTTC
ATCGCCGGCCTGATCGCCATCGTGATGGTGACCATCATGCTGTGCTGCATGACCAGCTGC
TGCAGCTGCCTGAAGGGCTGCTGCAGCTGCGGCAGCTGCTGCAAGTTCGACGAGGACGAC
AGCGAGCCCGTGCTGAAGGGCGTGAAGCTGCACTACACCTAG
Note:
the above sequence consists of signal peptide-extracellular domain-
transmembrane domain-intracellular domain.
SEQ ID NO: 42:
Full-length nucleic acid sequence of wild S antigen (optimization of
insect cells):
ATGTTCGTGTTCCTGGTGCTGCTGCCCCTGGTGTCCTCCCAGTGCGTGAACCTGACCACC
CGTACCCAGCTGCCCCCCGCTTACACCAACTCCTTCACCCGTGGTGTGTACTACCCCGAC
AAGGTGTTCCGTTCCTCCGTGCTGCACTCCACCCAGGACCTGTTCCTGCCCTTCTTCTCC
AACGTGACCTGGTTCCACGCTATCCACGTGTCCGGTACCAACGGTACCAAGCGTTTCGAC
AACCCCGTGCTGCCCTTCAACGACGGTGTGTACTTCGCTTCCACCGAGAAGTCCAACATC
ATCCGTGGTTGGATCTTCGGTACCACCCTGGACTCCAAGACCCAGTCCCTGCTGATCGTG
AACAACGCTACCAACGTGGTGATCAAGGTGTGCGAGTTCCAGTTCTGCAACGACCCCTTC
CTGGGTGTGTACTACCACAAGAACAACAAGTCCTGGATGGAGTCCGAGTTCCGTGTGTAC
TCCTCCGCTAACAACTGCACCTTCGAGTACGTGTCCCAGCCCTTCCTGATGGACCTGGAG
GGTAAGCAGGGTAACTTCAAGAACCTGCGTGAGTTCGTGTTCAAGAACATCGACGGTTAC
TTCAAGATCTACTCCAAGCACACCCCCATCAACCTGGTGCGTGACCTGCCCCAGGGTTTC
TCCGCTCTGGAGCCCCTGGTGGACCTGCCCATCGGTATCAACATCACCCGTTTCCAGACC
CTGCTGGCTCTGCACCGTTCCTACCTGACCCCCGGTGACTCCTCCTCCGGTTGGACCGCT
GGTGCTGCTGCTTACTACGTGGGTTACCTGCAGCCCCGTACCTTCCTGCTGAAGTACAAC
GAGAACGGTACCATCACCGACGCTGTGGACTGCGCTCTGGACCCCCTGTCCGAGACCAAG
TGCACCCTGAAGTCCTTCACCGTGGAGAAGGGTATCTACCAGACCTCCAACTTCCGTGTG
CAGCCCACCGAGTCCATCGTGCGTTTCCCCAACATCACCAACCTGTGCCCCTTCGGTGAG
GTGTTCAACGCTACCCGTTTCGCTTCCGTGTACGCTTGGAACCGTAAGCGTATCTCCAAC
TGCGTGGCTGACTACTCCGTGCTGTACAACTCCGCTTCCTTCTCCACCTTCAAGTGCTAC
GGTGTGTCCCCCACCAAGCTGAACGACCTGTGCTTCACCAACGTGTACGCTGACTCCTTC
GTGATCCGTGGTGACGAGGTGCGTCAGATCGCTCCCGGTCAGACCGGTAAGATCGCTGAC
TACAACTACAAGCTGCCCGACGACTTCACCGGTTGCGTGATCGCTTGGAACTCCAACAAC
CTGGACTCCAAGGTGGGTGGTAACTACAACTACCTGTACCGTCTGTTCCGTAAGTCCAAC
CTGAAGCCCTTCGAGCGTGACATCTCCACCGAGATCTACCAGGCTGGTTCCACCCCCTGC
AACGGTGTGGAGGGTTTCAACTGCTACTTCCCCCTGCAGTCCTACGGTTTCCAGCCCACC
AACGGTGTGGGTTACCAGCCCTACCGTGTGGTGGTGCTGTCCTTCGAGCTGCTGCACGCT
CCCGCTACCGTGTGCGGTCCCAAGAAGTCCACCAACCTGGTGAAGAACAAGTGCGTGAAC
TTCAACTTCAACGGTCTGACCGGTACCGGTGTGCTGACCGAGTCCAACAAGAAGTTCCTG
CCCTTCCAGCAGTTCGGTCGTGACATCGCTGACACCACCGACGCTGTGCGTGACCCCCAG
ACCCTGGAGATCCTGGACATCACCCCCTGCTCCTTCGGTGGTGTGTCCGTGATCACCCCC
GGTACCAACACCTCCAACCAGGTGGCTGTGCTGTACCAGGACGTGAACTGCACCGAGGTG
CCCGTGGCTATCCACGCTGACCAGCTGACCCCCACCTGGCGTGTGTACTCCACCGGTTCC
AACGTGTTCCAGACCCGTGCTGGTTGCCTGATCGGTGCTGAGCACGTGAACAACTCCTAC
GAGTGCGACATCCCCATCGGTGCTGGTATCTGCGCTTCCTACCAGACCCAGACCAACTCC
CCCCGTCGTGCTCGTTCCGTGGCTTCCCAGTCCATCATCGCTTACACCATGTCCCTGGGT
GCTGAGAACTCCGTGGCTTACTCCAACAACTCCATCGCTATCCCCACCAACTTCACCATC
TCCGTGACCACCGAGATCCTGCCCGTGTCCATGACCAAGACCTCCGTGGACTGCACCATG
TACATCTGCGGTGACTCCACCGAGTGCTCCAACCTGCTGCTGCAGTACGGTTCCTTCTGC
ACCCAGCTGAACCGTGCTCTGACCGGTATCGCTGTGGAGCAGGACAAGAACACCCAGGAG
GTGTTCGCTCAGGTGAAGCAGATCTACAAGACCCCCCCCATCAAGGACTTCGGTGGTTTC
AACTTCTCCCAGATCCTGCCCGACCCCTCCAAGCCCTCCAAGCGTTCCTTCATCGAGGAC
CTGCTGTTCAACAAGGTGACCCTGGCTGACGCTGGTTTCATCAAGCAGTACGGTGACTGC
CTGGGTGACATCGCTGCTCGTGACCTGATCTGCGCTCAGAAGTTCAACGGTCTGACCGTG
CTGCCCCCCCTGCTGACCGACGAGATGATCGCTCAGTACACCTCCGCTCTGCTGGCTGGT
ACCATCACCTCCGGTTGGACCTTCGGTGCTGGTGCTGCTCTGCAGATCCCCTTCGCTATG
CAGATGGCTTACCGTTTCAACGGTATCGGTGTGACCCAGAACGTGCTGTACGAGAACCAG
AAGCTGATCGCTAACCAGTTCAACTCCGCTATCGGTAAGATCCAGGACTCCCTGTCCTCC
ACCGCTTCCGCTCTGGGTAAGCTGCAGGACGTGGTGAACCAGAACGCTCAGGCTCTGAAC
ACCCTGGTGAAGCAGCTGTCCTCCAACTTCGGTGCTATCTCCTCCGTGCTGAACGACATC
CTGTCCCGTCTGGACAAGGTGGAGGCTGAGGTGCAGATCGACCGTCTGATCACCGGTCGT
CTGCAGTCCCTGCAGACCTACGTGACCCAGCAGCTGATCCGTGCTGCTGAGATCCGTGCT
TCCGCTAACCTGGCTGCTACCAAGATGTCCGAGTGCGTGCTGGGTCAGTCCAAGCGTGTG
GACTTCTGCGGTAAGGGTTACCACCTGATGTCCTTCCCCCAGTCCGCTCCCCACGGTGTG
GTGTTCCTGCACGTGACCTACGTGCCCGCTCAGGAGAAGAACTTCACCACCGCTCCCGCT
ATCTGCCACGACGGTAAGGCTCACTTCCCCCGTGAGGGTGTGTTCGTGTCCAACGGTACC
CACTGGTTCGTGACCCAGCGTAACTTCTACGAGCCCCAGATCATCACCACCGACAACACC
TTCGTGTCCGGTAACTGCGACGTGGTGATCGGTATCGTGAACAACACCGTGTACGACCCC
CTGCAGCCCGAGCTGGACTCCTTCAAGGAGGAGCTGGACAAGTACTTCAAGAACCACACC
TCCCCCGACGTGGACCTGGGTGACATCTCCGGTATCAACGCTTCCGTGGTGAACATCCAG
AAGGAGATCGACCGTCTGAACGAGGTGGCTAAGAACCTGAACGAGTCCCTGATCGACCTG
CAGGAGCTGGGTAAGTACGAGCAGTACATCAAGTGGCCCTGGTACATCTGGCTGGGTTTC
ATCGCTGGTCTGATCGCTATCGTGATGGTGACCATCATGCTGTGCTGCATGACCTCCTGC
TGCTCCTGCCTGAAGGGTTGCTGCTCCTGCGGTTCCTGCTGCAAGTTCGACGAGGACGAC
TCCGAGCCCGTGCTGAAGGGTGTGAAGCTGCACTACACCTAG
Note:
the above sequence consists of signal peptide-extracellular domain-
transmembrane domain-intracellular domain.
SEQ ID NO: 43:
Extracellular amino acid sequence of wild S antigen:
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGENCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLENKVTLADAGFIKQYGDC
LGDIAARDLICAQKFNGLTVLPPLLTDEMIAQYTSALLAGTITSGWTFGAGAALQIPFAM
QMAYRENGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWP
Note:
the above sequence consists of signal peptide-extracellular domain.
SEQ ID NO: 44:
Extracellular nucleic acid sequence of wild S antigen (optimization
of CHO cells):
ATGTTCGTGTTCCTGGTGCTGCTGCCCCTGGTGAGCAGCCAGTGCGTGAACCTGACCACC
AGAACCCAGCTGCCCCCCGCCTACACCAACAGCTTCACCAGAGGCGTGTACTACCCCGAC
AAGGTGTTCAGAAGCAGCGTGCTGCACAGCACCCAGGACCTGTTCCTGCCCTTCTTCAGC
AACGTGACCTGGTTCCACGCCATCCACGTGAGCGGCACCAACGGCACCAAGAGATTCGAC
AACCCCGTGCTGCCCTTCAACGACGGCGTGTACTTCGCCAGCACCGAGAAGAGCAACATC
ATCAGAGGCTGGATCTTCGGCACCACCCTGGACAGCAAGACCCAGAGCCTGCTGATCGTG
AACAACGCCACCAACGTGGTGATCAAGGTGTGCGAGTTCCAGTTCTGCAACGACCCCTTC
CTGGGCGTGTACTACCACAAGAACAACAAGAGCTGGATGGAGAGCGAGTTCAGAGTGTAC
AGCAGCGCCAACAACTGCACCTTCGAGTACGTGAGCCAGCCCTTCCTGATGGACCTGGAG
GGCAAGCAGGGCAACTTCAAGAACCTGAGAGAGTTCGTGTTCAAGAACATCGACGGCTAC
TTCAAGATCTACAGCAAGCACACCCCCATCAACCTGGTGAGAGACCTGCCCCAGGGCTTC
AGCGCCCTGGAGCCCCTGGTGGACCTGCCCATCGGCATCAACATCACCAGATTCCAGACC
CTGCTGGCCCTGCACAGAAGCTACCTGACCCCCGGCGACAGCAGCAGCGGCTGGACCGCC
GGCGCCGCCGCCTACTACGTGGGCTACCTGCAGCCCAGAACCTTCCTGCTGAAGTACAAC
GAGAACGGCACCATCACCGACGCCGTGGACTGCGCCCTGGACCCCCTGAGCGAGACCAAG
TGCACCCTGAAGAGCTTCACCGTGGAGAAGGGCATCTACCAGACCAGCAACTTCAGAGTG
CAGCCCACCGAGAGCATCGTGAGATTCCCCAACATCACCAACCTGTGCCCCTTCGGCGAG
GTGTTCAACGCCACCAGATTCGCCAGCGTGTACGCCTGGAACAGAAAGAGAATCAGCAAC
TGCGTGGCCGACTACAGCGTGCTGTACAACAGCGCCAGCTTCAGCACCTTCAAGTGCTAC
GGCGTGAGCCCCACCAAGCTGAACGACCTGTGCTTCACCAACGTGTACGCCGACAGCTTC
GTGATCAGAGGCGACGAGGTGAGACAGATCGCCCCCGGCCAGACCGGCAAGATCGCCGAC
TACAACTACAAGCTGCCCGACGACTTCACCGGCTGCGTGATCGCCTGGAACAGCAACAAC
CTGGACAGCAAGGTGGGCGGCAACTACAACTACCTGTACAGACTGTTCAGAAAGAGCAAC
CTGAAGCCCTTCGAGAGAGACATCAGCACCGAGATCTACCAGGCCGGCAGCACCCCCTGC
AACGGCGTGGAGGGCTTCAACTGCTACTTCCCCCTGCAGAGCTACGGCTTCCAGCCCACC
AACGGCGTGGGCTACCAGCCCTACAGAGTGGTGGTGCTGAGCTTCGAGCTGCTGCACGCC
CCCGCCACCGTGTGCGGCCCCAAGAAGAGCACCAACCTGGTGAAGAACAAGTGCGTGAAC
TTCAACTTCAACGGCCTGACCGGCACCGGCGTGCTGACCGAGAGCAACAAGAAGTTCCTG
CCCTTCCAGCAGTTCGGCAGAGACATCGCCGACACCACCGACGCCGTGAGAGACCCCCAG
ACCCTGGAGATCCTGGACATCACCCCCTGCAGCTTCGGCGGCGTGAGCGTGATCACCCCC
GGCACCAACACCAGCAACCAGGTGGCCGTGCTGTACCAGGACGTGAACTGCACCGAGGTG
CCCGTGGCCATCCACGCCGACCAGCTGACCCCCACCTGGAGAGTGTACAGCACCGGCAGC
AACGTGTTCCAGACCAGAGCCGGCTGCCTGATCGGCGCCGAGCACGTGAACAACAGCTAC
GAGTGCGACATCCCCATCGGCGCCGGCATCTGCGCCAGCTACCAGACCCAGACCAACAGC
CCCAGAAGAGCCAGAAGCGTGGCCAGCCAGAGCATCATCGCCTACACCATGAGCCTGGGC
GCCGAGAACAGCGTGGCCTACAGCAACAACAGCATCGCCATCCCCACCAACTTCACCATC
AGCGTGACCACCGAGATCCTGCCCGTGAGCATGACCAAGACCAGCGTGGACTGCACCATG
TACATCTGCGGCGACAGCACCGAGTGCAGCAACCTGCTGCTGCAGTACGGCAGCTTCTGC
ACCCAGCTGAACAGAGCCCTGACCGGCATCGCCGTGGAGCAGGACAAGAACACCCAGGAG
GTGTTCGCCCAGGTGAAGCAGATCTACAAGACCCCCCCCATCAAGGACTTCGGCGGCTTC
AACTTCAGCCAGATCCTGCCCGACCCCAGCAAGCCCAGCAAGAGAAGCTTCATCGAGGAC
CTGCTGTTCAACAAGGTGACCCTGGCCGACGCCGGCTTCATCAAGCAGTACGGCGACTGC
CTGGGCGACATCGCCGCCAGAGACCTGATCTGCGCCCAGAAGTTCAACGGCCTGACCGTG
CTGCCCCCCCTGCTGACCGACGAGATGATCGCCCAGTACACCAGCGCCCTGCTGGCCGGC
ACCATCACCAGCGGCTGGACCTTCGGCGCCGGCGCCGCCCTGCAGATCCCCTTCGCCATG
CAGATGGCCTACAGATTCAACGGCATCGGCGTGACCCAGAACGTGCTGTACGAGAACCAG
AAGCTGATCGCCAACCAGTTCAACAGCGCCATCGGCAAGATCCAGGACAGCCTGAGCAGC
ACCGCCAGCGCCCTGGGCAAGCTGCAGGACGTGGTGAACCAGAACGCCCAGGCCCTGAAC
ACCCTGGTGAAGCAGCTGAGCAGCAACTTCGGCGCCATCAGCAGCGTGCTGAACGACATC
CTGAGCAGACTGGACAAGGTGGAGGCCGAGGTGCAGATCGACAGACTGATCACCGGCAGA
CTGCAGAGCCTGCAGACCTACGTGACCCAGCAGCTGATCAGAGCCGCCGAGATCAGAGCC
AGCGCCAACCTGGCCGCCACCAAGATGAGCGAGTGCGTGCTGGGCCAGAGCAAGAGAGTG
GACTTCTGCGGCAAGGGCTACCACCTGATGAGCTTCCCCCAGAGCGCCCCCCACGGCGTG
GTGTTCCTGCACGTGACCTACGTGCCCGCCCAGGAGAAGAACTTCACCACCGCCCCCGCC
ATCTGCCACGACGGCAAGGCCCACTTCCCCAGAGAGGGCGTGTTCGTGAGCAACGGCACC
CACTGGTTCGTGACCCAGAGAAACTTCTACGAGCCCCAGATCATCACCACCGACAACACC
TTCGTGAGCGGCAACTGCGACGTGGTGATCGGCATCGTGAACAACACCGTGTACGACCCC
CTGCAGCCCGAGCTGGACAGCTTCAAGGAGGAGCTGGACAAGTACTTCAAGAACCACACC
AGCCCCGACGTGGACCTGGGCGACATCAGCGGCATCAACGCCAGCGTGGTGAACATCCAG
AAGGAGATCGACAGACTGAACGAGGTGGCCAAGAACCTGAACGAGAGCCTGATCGACCTG
CAGGAGCTGGGCAAGTACGAGCAGTACATCAAGTGGCCCTAG
Note:
the above sequence consists of signal peptide-extracellular domain.
SEQ ID NO: 45:
Extracellular nucleic acid sequence of wild S antigen (optimization
of insect cells):
ATGTTCGTGTTCCTGGTGCTGCTGCCCCTGGTGTCCTCCCAGTGCGTGAACCTGACCACC
CGTACCCAGCTGCCCCCCGCTTACACCAACTCCTTCACCCGTGGTGTGTACTACCCCGAC
AAGGTGTTCCGTTCCTCCGTGCTGCACTCCACCCAGGACCTGTTCCTGCCCTTCTTCTCC
AACGTGACCTGGTTCCACGCTATCCACGTGTCCGGTACCAACGGTACCAAGCGTTTCGAC
AACCCCGTGCTGCCCTTCAACGACGGTGTGTACTTCGCTTCCACCGAGAAGTCCAACATC
ATCCGTGGTTGGATCTTCGGTACCACCCTGGACTCCAAGACCCAGTCCCTGCTGATCGTG
AACAACGCTACCAACGTGGTGATCAAGGTGTGCGAGTTCCAGTTCTGCAACGACCCCTTC
CTGGGTGTGTACTACCACAAGAACAACAAGTCCTGGATGGAGTCCGAGTTCCGTGTGTAC
TCCTCCGCTAACAACTGCACCTTCGAGTACGTGTCCCAGCCCTTCCTGATGGACCTGGAG
GGTAAGCAGGGTAACTTCAAGAACCTGCGTGAGTTCGTGTTCAAGAACATCGACGGTTAC
TTCAAGATCTACTCCAAGCACACCCCCATCAACCTGGTGCGTGACCTGCCCCAGGGTTTC
TCCGCTCTGGAGCCCCTGGTGGACCTGCCCATCGGTATCAACATCACCCGTTTCCAGACC
CTGCTGGCTCTGCACCGTTCCTACCTGACCCCCGGTGACTCCTCCTCCGGTTGGACCGCT
GGTGCTGCTGCTTACTACGTGGGTTACCTGCAGCCCCGTACCTTCCTGCTGAAGTACAAC
GAGAACGGTACCATCACCGACGCTGTGGACTGCGCTCTGGACCCCCTGTCCGAGACCAAG
TGCACCCTGAAGTCCTTCACCGTGGAGAAGGGTATCTACCAGACCTCCAACTTCCGTGTG
CAGCCCACCGAGTCCATCGTGCGTTTCCCCAACATCACCAACCTGTGCCCCTTCGGTGAG
GTGTTCAACGCTACCCGTTTCGCTTCCGTGTACGCTTGGAACCGTAAGCGTATCTCCAAC
TGCGTGGCTGACTACTCCGTGCTGTACAACTCCGCTTCCTTCTCCACCTTCAAGTGCTAC
GGTGTGTCCCCCACCAAGCTGAACGACCTGTGCTTCACCAACGTGTACGCTGACTCCTTC
GTGATCCGTGGTGACGAGGTGCGTCAGATCGCTCCCGGTCAGACCGGTAAGATCGCTGAC
TACAACTACAAGCTGCCCGACGACTTCACCGGTTGCGTGATCGCTTGGAACTCCAACAAC
CTGGACTCCAAGGTGGGTGGTAACTACAACTACCTGTACCGTCTGTTCCGTAAGTCCAAC
CTGAAGCCCTTCGAGCGTGACATCTCCACCGAGATCTACCAGGCTGGTTCCACCCCCTGC
AACGGTGTGGAGGGTTTCAACTGCTACTTCCCCCTGCAGTCCTACGGTTTCCAGCCCACC
AACGGTGTGGGTTACCAGCCCTACCGTGTGGTGGTGCTGTCCTTCGAGCTGCTGCACGCT
CCCGCTACCGTGTGCGGTCCCAAGAAGTCCACCAACCTGGTGAAGAACAAGTGCGTGAAC
TTCAACTTCAACGGTCTGACCGGTACCGGTGTGCTGACCGAGTCCAACAAGAAGTTCCTG
CCCTTCCAGCAGTTCGGTCGTGACATCGCTGACACCACCGACGCTGTGCGTGACCCCCAG
ACCCTGGAGATCCTGGACATCACCCCCTGCTCCTTCGGTGGTGTGTCCGTGATCACCCCC
GGTACCAACACCTCCAACCAGGTGGCTGTGCTGTACCAGGACGTGAACTGCACCGAGGTG
CCCGTGGCTATCCACGCTGACCAGCTGACCCCCACCTGGCGTGTGTACTCCACCGGTTCC
AACGTGTTCCAGACCCGTGCTGGTTGCCTGATCGGTGCTGAGCACGTGAACAACTCCTAC
GAGTGCGACATCCCCATCGGTGCTGGTATCTGCGCTTCCTACCAGACCCAGACCAACTCC
CCCCGTCGTGCTCGTTCCGTGGCTTCCCAGTCCATCATCGCTTACACCATGTCCCTGGGT
GCTGAGAACTCCGTGGCTTACTCCAACAACTCCATCGCTATCCCCACCAACTTCACCATC
TCCGTGACCACCGAGATCCTGCCCGTGTCCATGACCAAGACCTCCGTGGACTGCACCATG
TACATCTGCGGTGACTCCACCGAGTGCTCCAACCTGCTGCTGCAGTACGGTTCCTTCTGC
ACCCAGCTGAACCGTGCTCTGACCGGTATCGCTGTGGAGCAGGACAAGAACACCCAGGAG
GTGTTCGCTCAGGTGAAGCAGATCTACAAGACCCCCCCCATCAAGGACTTCGGTGGTTTC
AACTTCTCCCAGATCCTGCCCGACCCCTCCAAGCCCTCCAAGCGTTCCTTCATCGAGGAC
CTGCTGTTCAACAAGGTGACCCTGGCTGACGCTGGTTTCATCAAGCAGTACGGTGACTGC
CTGGGTGACATCGCTGCTCGTGACCTGATCTGCGCTCAGAAGTTCAACGGTCTGACCGTG
CTGCCCCCCCTGCTGACCGACGAGATGATCGCTCAGTACACCTCCGCTCTGCTGGCTGGT
ACCATCACCTCCGGTTGGACCTTCGGTGCTGGTGCTGCTCTGCAGATCCCCTTCGCTATG
CAGATGGCTTACCGTTTCAACGGTATCGGTGTGACCCAGAACGTGCTGTACGAGAACCAG
AAGCTGATCGCTAACCAGTTCAACTCCGCTATCGGTAAGATCCAGGACTCCCTGTCCTCC
ACCGCTTCCGCTCTGGGTAAGCTGCAGGACGTGGTGAACCAGAACGCTCAGGCTCTGAAC
ACCCTGGTGAAGCAGCTGTCCTCCAACTTCGGTGCTATCTCCTCCGTGCTGAACGACATC
CTGTCCCGTCTGGACAAGGTGGAGGCTGAGGTGCAGATCGACCGTCTGATCACCGGTCGT
CTGCAGTCCCTGCAGACCTACGTGACCCAGCAGCTGATCCGTGCTGCTGAGATCCGTGCT
TCCGCTAACCTGGCTGCTACCAAGATGTCCGAGTGCGTGCTGGGTCAGTCCAAGCGTGTG
GACTTCTGCGGTAAGGGTTACCACCTGATGTCCTTCCCCCAGTCCGCTCCCCACGGTGTG
GTGTTCCTGCACGTGACCTACGTGCCCGCTCAGGAGAAGAACTTCACCACCGCTCCCGCT
ATCTGCCACGACGGTAAGGCTCACTTCCCCCGTGAGGGTGTGTTCGTGTCCAACGGTACC
CACTGGTTCGTGACCCAGCGTAACTTCTACGAGCCCCAGATCATCACCACCGACAACACC
TTCGTGTCCGGTAACTGCGACGTGGTGATCGGTATCGTGAACAACACCGTGTACGACCCC
CTGCAGCCCGAGCTGGACTCCTTCAAGGAGGAGCTGGACAAGTACTTCAAGAACCACACC
TCCCCCGACGTGGACCTGGGTGACATCTCCGGTATCAACGCTTCCGTGGTGAACATCCAG
AAGGAGATCGACCGTCTGAACGAGGTGGCTAAGAACCTGAACGAGTCCCTGATCGACCTG
CAGGAGCTGGGTAAGTACGAGCAGTACATCAAGTGGCCCTAG
Note:
the above sequence consists of signal peptide-extracellular domain.
SEQ ID NO: 46:
Full-length amino acid sequence of Beta S antigen:
MFVFLVLLPLVSSQCVNLTTRTQLPPAYTNSFTRGVYYPDKVFRSSVLHSTQDLFLPFFS
NVTWFHAIHVSGTNGTKRFANPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRGLPQGFSALEPLVDLPIGINITRFQT
LHRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVENATRFASVYAWNRKRISNCVA
DYSVLYNSASFSTFKCYGVSPTKLNDLCFTNVYADSFVIRGDEVRQIAPGQTGNIADYNY
KLPDDFTGCVIAWNSNNLDSKVGGNYNYLYRLFRKSNLKPFERDISTEIYQAGSTPCNGV
KGFNCYFPLQSYGFQPTYGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEHVNNSYECD
IPIGAGICASYQTQTNSPRRARSVASQSIIAYTMSLGVENSVAYSNNSIAIPTNFTISVT
TEILPVSMTKTSVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAGFIKQYGDCLGD
IAARDLICAQKFNGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVNQNAQALNTLV
KQLSSNFGAISSVLNDILSRLDKVEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEP
VLKGVKLHYT
Note:
the above sequence consists of signal peptide-extracellular domain-
transmembrane domain-intracellular domain.
SEQ ID NO: 47:
Full-length nucleic acid sequence of Beta S antigen (optimization of
CHO cells):
ATGTTCGTGTTCCTGGTGCTGCTGCCCCTGGTGAGCAGCCAGTGCGTGAACCTGACCACC
AGAACCCAGCTGCCCCCCGCCTACACCAACAGCTTCACCAGAGGCGTGTACTACCCCGAC
AAGGTGTTCAGAAGCAGCGTGCTGCACAGCACCCAGGACCTGTTCCTGCCCTTCTTCAGC
AACGTGACCTGGTTCCACGCCATCCACGTGAGCGGCACCAACGGCACCAAGAGATTCGCC
AACCCCGTGCTGCCCTTCAACGACGGCGTGTACTTCGCCAGCACCGAGAAGAGCAACATC
ATCAGAGGCTGGATCTTCGGCACCACCCTGGACAGCAAGACCCAGAGCCTGCTGATCGTG
AACAACGCCACCAACGTGGTGATCAAGGTGTGCGAGTTCCAGTTCTGCAACGACCCCTTC
CTGGGCGTGTACTACCACAAGAACAACAAGAGCTGGATGGAGAGCGAGTTCAGAGTGTAC
AGCAGCGCCAACAACTGCACCTTCGAGTACGTGAGCCAGCCCTTCCTGATGGACCTGGAG
GGCAAGCAGGGCAACTTCAAGAACCTGAGAGAGTTCGTGTTCAAGAACATCGACGGCTAC
TTCAAGATCTACAGCAAGCACACCCCCATCAACCTGGTGAGAGGCCTGCCCCAGGGCTTC
AGCGCCCTGGAGCCCCTGGTGGACCTGCCCATCGGCATCAACATCACCAGATTCCAGACC
CTGCACAGAAGCTACCTGACCCCCGGCGACAGCAGCAGCGGCTGGACCGCCGGCGCCGCC
GCCTACTACGTGGGCTACCTGCAGCCCAGAACCTTCCTGCTGAAGTACAACGAGAACGGC
ACCATCACCGACGCCGTGGACTGCGCCCTGGACCCCCTGAGCGAGACCAAGTGCACCCTG
AAGAGCTTCACCGTGGAGAAGGGCATCTACCAGACCAGCAACTTCAGAGTGCAGCCCACC
GAGAGCATCGTGAGATTCCCCAACATCACCAACCTGTGCCCCTTCGGCGAGGTGTTCAAC
GCCACCAGATTCGCCAGCGTGTACGCCTGGAACAGAAAGAGAATCAGCAACTGCGTGGCC
GACTACAGCGTGCTGTACAACAGCGCCAGCTTCAGCACCTTCAAGTGCTACGGCGTGAGC
CCCACCAAGCTGAACGACCTGTGCTTCACCAACGTGTACGCCGACAGCTTCGTGATCAGA
GGCGACGAGGTGAGACAGATCGCCCCCGGCCAGACCGGCAACATCGCCGACTACAACTAC
AAGCTGCCCGACGACTTCACCGGCTGCGTGATCGCCTGGAACAGCAACAACCTGGACAGC
AAGGTGGGCGGCAACTACAACTACCTGTACAGACTGTTCAGAAAGAGCAACCTGAAGCCC
TTCGAGAGAGACATCAGCACCGAGATCTACCAGGCCGGCAGCACCCCCTGCAACGGCGTG
AAGGGCTTCAACTGCTACTTCCCCCTGCAGAGCTACGGCTTCCAGCCCACCTACGGCGTG
GGCTACCAGCCCTACAGAGTGGTGGTGCTGAGCTTCGAGCTGCTGCACGCCCCCGCCACC
GTGTGCGGCCCCAAGAAGAGCACCAACCTGGTGAAGAACAAGTGCGTGAACTTCAACTTC
AACGGCCTGACCGGCACCGGCGTGCTGACCGAGAGCAACAAGAAGTTCCTGCCCTTCCAG
CAGTTCGGCAGAGACATCGCCGACACCACCGACGCCGTGAGAGACCCCCAGACCCTGGAG
ATCCTGGACATCACCCCCTGCAGCTTCGGCGGCGTGAGCGTGATCACCCCCGGCACCAAC
ACCAGCAACCAGGTGGCCGTGCTGTACCAGGGCGTGAACTGCACCGAGGTGCCCGTGGCC
ATCCACGCCGACCAGCTGACCCCCACCTGGAGAGTGTACAGCACCGGCAGCAACGTGTTC
CAGACCAGAGCCGGCTGCCTGATCGGCGCCGAGCACGTGAACAACAGCTACGAGTGCGAC
ATCCCCATCGGCGCCGGCATCTGCGCCAGCTACCAGACCCAGACCAACAGCCCCAGAAGA
GCCAGAAGCGTGGCCAGCCAGAGCATCATCGCCTACACCATGAGCCTGGGCGTGGAGAAC
AGCGTGGCCTACAGCAACAACAGCATCGCCATCCCCACCAACTTCACCATCAGCGTGACC
ACCGAGATCCTGCCCGTGAGCATGACCAAGACCAGCGTGGACTGCACCATGTACATCTGC
GGCGACAGCACCGAGTGCAGCAACCTGCTGCTGCAGTACGGCAGCTTCTGCACCCAGCTG
AACAGAGCCCTGACCGGCATCGCCGTGGAGCAGGACAAGAACACCCAGGAGGTGTTCGCC
CAGGTGAAGCAGATCTACAAGACCCCCCCCATCAAGGACTTCGGCGGCTTCAACTTCAGC
CAGATCCTGCCCGACCCCAGCAAGCCCAGCAAGAGAAGCTTCATCGAGGACCTGCTGTTC
AACAAGGTGACCCTGGCCGACGCCGGCTTCATCAAGCAGTACGGCGACTGCCTGGGCGAC
ATCGCCGCCAGAGACCTGATCTGCGCCCAGAAGTTCAACGGCCTGACCGTGCTGCCCCCC
CTGCTGACCGACGAGATGATCGCCCAGTACACCAGCGCCCTGCTGGCCGGCACCATCACC
AGCGGCTGGACCTTCGGCGCCGGCGCCGCCCTGCAGATCCCCTTCGCCATGCAGATGGCC
TACAGATTCAACGGCATCGGCGTGACCCAGAACGTGCTGTACGAGAACCAGAAGCTGATC
GCCAACCAGTTCAACAGCGCCATCGGCAAGATCCAGGACAGCCTGAGCAGCACCGCCAGC
GCCCTGGGCAAGCTGCAGGACGTGGTGAACCAGAACGCCCAGGCCCTGAACACCCTGGTG
AAGCAGCTGAGCAGCAACTTCGGCGCCATCAGCAGCGTGCTGAACGACATCCTGAGCAGA
CTGGACAAGGTGGAGGCCGAGGTGCAGATCGACAGACTGATCACCGGCAGACTGCAGAGC
CTGCAGACCTACGTGACCCAGCAGCTGATCAGAGCCGCCGAGATCAGAGCCAGCGCCAAC
CTGGCCGCCACCAAGATGAGCGAGTGCGTGCTGGGCCAGAGCAAGAGAGTGGACTTCTGC
GGCAAGGGCTACCACCTGATGAGCTTCCCCCAGAGCGCCCCCCACGGCGTGGTGTTCCTG
CACGTGACCTACGTGCCCGCCCAGGAGAAGAACTTCACCACCGCCCCCGCCATCTGCCAC
GACGGCAAGGCCCACTTCCCCAGAGAGGGCGTGTTCGTGAGCAACGGCACCCACTGGTTC
GTGACCCAGAGAAACTTCTACGAGCCCCAGATCATCACCACCGACAACACCTTCGTGAGC
GGCAACTGCGACGTGGTGATCGGCATCGTGAACAACACCGTGTACGACCCCCTGCAGCCC
GAGCTGGACAGCTTCAAGGAGGAGCTGGACAAGTACTTCAAGAACCACACCAGCCCCGAC
GTGGACCTGGGCGACATCAGCGGCATCAACGCCAGCGTGGTGAACATCCAGAAGGAGATC
GACAGACTGAACGAGGTGGCCAAGAACCTGAACGAGAGCCTGATCGACCTGCAGGAGCTG
GGCAAGTACGAGCAGTACATCAAGTGGCCCTGGTACATCTGGCTGGGCTTCATCGCCGGC
CTGATCGCCATCGTGATGGTGACCATCATGCTGTGCTGCATGACCAGCTGCTGCAGCTGC
CTGAAGGGCTGCTGCAGCTGCGGCAGCTGCTGCAAGTTCGACGAGGACGACAGCGAGCCC
GTGCTGAAGGGCGTGAAGCTGCACTACACCTAG
Note:
the above sequence consists of signal peptide-extracellular domain-
transmembrane domain-intracellular domain.
SEQ ID NO: 48:
Full-length nucleic acid sequence of Beta S antigen (optimization of
insect cells):
ATGTTCGTGTTCCTGGTGCTGCTGCCCCTGGTGTCCTCCCAGTGCGTGAACCTGACCACC
CGTACCCAGCTGCCCCCCGCTTACACCAACTCCTTCACCCGTGGTGTGTACTACCCCGAC
AAGGTGTTCCGTTCCTCCGTGCTGCACTCCACCCAGGACCTGTTCCTGCCCTTCTTCTCC
AACGTGACCTGGTTCCACGCTATCCACGTGTCCGGTACCAACGGTACCAAGCGTTTCGCT
AACCCCGTGCTGCCCTTCAACGACGGTGTGTACTTCGCTTCCACCGAGAAGTCCAACATC
ATCCGTGGTTGGATCTTCGGTACCACCCTGGACTCCAAGACCCAGTCCCTGCTGATCGTG
AACAACGCTACCAACGTGGTGATCAAGGTGTGCGAGTTCCAGTTCTGCAACGACCCCTTC
CTGGGTGTGTACTACCACAAGAACAACAAGTCCTGGATGGAGTCCGAGTTCCGTGTGTAC
TCCTCCGCTAACAACTGCACCTTCGAGTACGTGTCCCAGCCCTTCCTGATGGACCTGGAG
GGTAAGCAGGGTAACTTCAAGAACCTGCGTGAGTTCGTGTTCAAGAACATCGACGGTTAC
TTCAAGATCTACTCCAAGCACACCCCCATCAACCTGGTGCGTGGTCTGCCCCAGGGTTTC
TCCGCTCTGGAGCCCCTGGTGGACCTGCCCATCGGTATCAACATCACCCGTTTCCAGACC
CTGCACCGTTCCTACCTGACCCCCGGTGACTCCTCCTCCGGTTGGACCGCTGGTGCTGCT
GCTTACTACGTGGGTTACCTGCAGCCCCGTACCTTCCTGCTGAAGTACAACGAGAACGGT
ACCATCACCGACGCTGTGGACTGCGCTCTGGACCCCCTGTCCGAGACCAAGTGCACCCTG
AAGTCCTTCACCGTGGAGAAGGGTATCTACCAGACCTCCAACTTCCGTGTGCAGCCCACC
GAGTCCATCGTGCGTTTCCCCAACATCACCAACCTGTGCCCCTTCGGTGAGGTGTTCAAC
GCTACCCGTTTCGCTTCCGTGTACGCTTGGAACCGTAAGCGTATCTCCAACTGCGTGGCT
GACTACTCCGTGCTGTACAACTCCGCTTCCTTCTCCACCTTCAAGTGCTACGGTGTGTCC
CCCACCAAGCTGAACGACCTGTGCTTCACCAACGTGTACGCTGACTCCTTCGTGATCCGT
GGTGACGAGGTGCGTCAGATCGCTCCCGGTCAGACCGGTAACATCGCTGACTACAACTAC
AAGCTGCCCGACGACTTCACCGGTTGCGTGATCGCTTGGAACTCCAACAACCTGGACTCC
AAGGTGGGTGGTAACTACAACTACCTGTACCGTCTGTTCCGTAAGTCCAACCTGAAGCCC
TTCGAGCGTGACATCTCCACCGAGATCTACCAGGCTGGTTCCACCCCCTGCAACGGTGTG
AAGGGTTTCAACTGCTACTTCCCCCTGCAGTCCTACGGTTTCCAGCCCACCTACGGTGTG
GGTTACCAGCCCTACCGTGTGGTGGTGCTGTCCTTCGAGCTGCTGCACGCTCCCGCTACC
GTGTGCGGTCCCAAGAAGTCCACCAACCTGGTGAAGAACAAGTGCGTGAACTTCAACTTC
AACGGTCTGACCGGTACCGGTGTGCTGACCGAGTCCAACAAGAAGTTCCTGCCCTTCCAG
CAGTTCGGTCGTGACATCGCTGACACCACCGACGCTGTGCGTGACCCCCAGACCCTGGAG
ATCCTGGACATCACCCCCTGCTCCTTCGGTGGTGTGTCCGTGATCACCCCCGGTACCAAC
ACCTCCAACCAGGTGGCTGTGCTGTACCAGGGTGTGAACTGCACCGAGGTGCCCGTGGCT
ATCCACGCTGACCAGCTGACCCCCACCTGGCGTGTGTACTCCACCGGTTCCAACGTGTTC
CAGACCCGTGCTGGTTGCCTGATCGGTGCTGAGCACGTGAACAACTCCTACGAGTGCGAC
ATCCCCATCGGTGCTGGTATCTGCGCTTCCTACCAGACCCAGACCAACTCCCCCCGTCGT
GCTCGTTCCGTGGCTTCCCAGTCCATCATCGCTTACACCATGTCCCTGGGTGTGGAGAAC
TCCGTGGCTTACTCCAACAACTCCATCGCTATCCCCACCAACTTCACCATCTCCGTGACC
ACCGAGATCCTGCCCGTGTCCATGACCAAGACCTCCGTGGACTGCACCATGTACATCTGC
GGTGACTCCACCGAGTGCTCCAACCTGCTGCTGCAGTACGGTTCCTTCTGCACCCAGCTG
AACCGTGCTCTGACCGGTATCGCTGTGGAGCAGGACAAGAACACCCAGGAGGTGTTCGCT
CAGGTGAAGCAGATCTACAAGACCCCCCCCATCAAGGACTTCGGTGGTTTCAACTTCTCC
CAGATCCTGCCCGACCCCTCCAAGCCCTCCAAGCGTTCCTTCATCGAGGACCTGCTGTTC
AACAAGGTGACCCTGGCTGACGCTGGTTTCATCAAGCAGTACGGTGACTGCCTGGGTGAC
ATCGCTGCTCGTGACCTGATCTGCGCTCAGAAGTTCAACGGTCTGACCGTGCTGCCCCCC
CTGCTGACCGACGAGATGATCGCTCAGTACACCTCCGCTCTGCTGGCTGGTACCATCACC
TCCGGTTGGACCTTCGGTGCTGGTGCTGCTCTGCAGATCCCCTTCGCTATGCAGATGGCT
TACCGTTTCAACGGTATCGGTGTGACCCAGAACGTGCTGTACGAGAACCAGAAGCTGATC
GCTAACCAGTTCAACTCCGCTATCGGTAAGATCCAGGACTCCCTGTCCTCCACCGCTTCC
GCTCTGGGTAAGCTGCAGGACGTGGTGAACCAGAACGCTCAGGCTCTGAACACCCTGGTG
AAGCAGCTGTCCTCCAACTTCGGTGCTATCTCCTCCGTGCTGAACGACATCCTGTCCCGT
CTGGACAAGGTGGAGGCTGAGGTGCAGATCGACCGTCTGATCACCGGTCGTCTGCAGTCC
CTGCAGACCTACGTGACCCAGCAGCTGATCCGTGCTGCTGAGATCCGTGCTTCCGCTAAC
CTGGCTGCTACCAAGATGTCCGAGTGCGTGCTGGGTCAGTCCAAGCGTGTGGACTTCTGC
GGTAAGGGTTACCACCTGATGTCCTTCCCCCAGTCCGCTCCCCACGGTGTGGTGTTCCTG
CACGTGACCTACGTGCCCGCTCAGGAGAAGAACTTCACCACCGCTCCCGCTATCTGCCAC
GACGGTAAGGCTCACTTCCCCCGTGAGGGTGTGTTCGTGTCCAACGGTACCCACTGGTTC
GTGACCCAGCGTAACTTCTACGAGCCCCAGATCATCACCACCGACAACACCTTCGTGTCC
GGTAACTGCGACGTGGTGATCGGTATCGTGAACAACACCGTGTACGACCCCCTGCAGCCC
GAGCTGGACTCCTTCAAGGAGGAGCTGGACAAGTACTTCAAGAACCACACCTCCCCCGAC
GTGGACCTGGGTGACATCTCCGGTATCAACGCTTCCGTGGTGAACATCCAGAAGGAGATC
GACCGTCTGAACGAGGTGGCTAAGAACCTGAACGAGTCCCTGATCGACCTGCAGGAGCTG
GGTAAGTACGAGCAGTACATCAAGTGGCCCTGGTACATCTGGCTGGGTTTCATCGCTGGT
CTGATCGCTATCGTGATGGTGACCATCATGCTGTGCTGCATGACCTCCTGCTGCTCCTGC
CTGAAGGGTTGCTGCTCCTGCGGTTCCTGCTGCAAGTTCGACGAGGACGACTCCGAGCCC
GTGCTGAAGGGTGTGAAGCTGCACTACACCTAG
Note:
the above sequence consists of signal peptide-extracellular domain-
transmembrane domain-intracellular domain.
SEQ ID NO: 49:
Extracellular amino acid sequence of Beta S antigen:
MFVFLVLLPLVSSQCVNLTTRTQLPPAYTNSFTRGVYYPDKVFRSSVLHSTQDLFLPFFS
NVTWFHAIHVSGTNGTKRFANPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRGLPQGFSALEPLVDLPIGINITRFQT
LHRSYLTPGDSSSGWTAGAAAYYVGYLQPRTFLLKYNENGTITDAVDCALDPLSETKCTL
KSFTVEKGIYQTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPGQTGNIADYNY
KLPDDFTGCVIAWNSNNLDSKVGGNYNYLYRLFRKSNLKPFERDISTEIYQAGSTPCNGV
KGFNCYFPLQSYGFQPTYGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEHVNNSYECD
IPIGAGICASYQTQTNSPRRARSVASQSIIAYTMSLGVENSVAYSNNSIAIPTNFTISVT
TEILPVSMTKTSVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAGFIKQYGDCLGD
IAARDLICAQKFNGLTVLPPLLTDEMIAQYTSALLAGTITSGWTFGAGAALQIPFAMQMA
YRENGIGVTQNVLYENQKLIANQFNSAIGKIQDSLSSTASALGKLQDVVNQNAQALNTLV
KQLSSNFGAISSVLNDILSRLDKVEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWP
Note:
the above sequence consists of signal peptide-extracellular domain.
SEQ ID NO: 50:
Extracellular nucleic acid sequence of Beta S antigen (optimization
of CHO cells):
ATGTTCGTGTTCCTGGTGCTGCTGCCCCTGGTGAGCAGCCAGTGCGTGAACCTGACCACC
AGAACCCAGCTGCCCCCCGCCTACACCAACAGCTTCACCAGAGGCGTGTACTACCCCGAC
AAGGTGTTCAGAAGCAGCGTGCTGCACAGCACCCAGGACCTGTTCCTGCCCTTCTTCAGC
AACGTGACCTGGTTCCACGCCATCCACGTGAGCGGCACCAACGGCACCAAGAGATTCGCC
AACCCCGTGCTGCCCTTCAACGACGGCGTGTACTTCGCCAGCACCGAGAAGAGCAACATC
ATCAGAGGCTGGATCTTCGGCACCACCCTGGACAGCAAGACCCAGAGCCTGCTGATCGTG
AACAACGCCACCAACGTGGTGATCAAGGTGTGCGAGTTCCAGTTCTGCAACGACCCCTTC
CTGGGCGTGTACTACCACAAGAACAACAAGAGCTGGATGGAGAGCGAGTTCAGAGTGTAC
AGCAGCGCCAACAACTGCACCTTCGAGTACGTGAGCCAGCCCTTCCTGATGGACCTGGAG
GGCAAGCAGGGCAACTTCAAGAACCTGAGAGAGTTCGTGTTCAAGAACATCGACGGCTAC
TTCAAGATCTACAGCAAGCACACCCCCATCAACCTGGTGAGAGGCCTGCCCCAGGGCTTC
AGCGCCCTGGAGCCCCTGGTGGACCTGCCCATCGGCATCAACATCACCAGATTCCAGACC
CTGCACAGAAGCTACCTGACCCCCGGCGACAGCAGCAGCGGCTGGACCGCCGGCGCCGCC
GCCTACTACGTGGGCTACCTGCAGCCCAGAACCTTCCTGCTGAAGTACAACGAGAACGGC
ACCATCACCGACGCCGTGGACTGCGCCCTGGACCCCCTGAGCGAGACCAAGTGCACCCTG
AAGAGCTTCACCGTGGAGAAGGGCATCTACCAGACCAGCAACTTCAGAGTGCAGCCCACC
GAGAGCATCGTGAGATTCCCCAACATCACCAACCTGTGCCCCTTCGGCGAGGTGTTCAAC
GCCACCAGATTCGCCAGCGTGTACGCCTGGAACAGAAAGAGAATCAGCAACTGCGTGGCC
GACTACAGCGTGCTGTACAACAGCGCCAGCTTCAGCACCTTCAAGTGCTACGGCGTGAGC
CCCACCAAGCTGAACGACCTGTGCTTCACCAACGTGTACGCCGACAGCTTCGTGATCAGA
GGCGACGAGGTGAGACAGATCGCCCCCGGCCAGACCGGCAACATCGCCGACTACAACTAC
AAGCTGCCCGACGACTTCACCGGCTGCGTGATCGCCTGGAACAGCAACAACCTGGACAGC
AAGGTGGGCGGCAACTACAACTACCTGTACAGACTGTTCAGAAAGAGCAACCTGAAGCCC
TTCGAGAGAGACATCAGCACCGAGATCTACCAGGCCGGCAGCACCCCCTGCAACGGCGTG
AAGGGCTTCAACTGCTACTTCCCCCTGCAGAGCTACGGCTTCCAGCCCACCTACGGCGTG
GGCTACCAGCCCTACAGAGTGGTGGTGCTGAGCTTCGAGCTGCTGCACGCCCCCGCCACC
GTGTGCGGCCCCAAGAAGAGCACCAACCTGGTGAAGAACAAGTGCGTGAACTTCAACTTC
AACGGCCTGACCGGCACCGGCGTGCTGACCGAGAGCAACAAGAAGTTCCTGCCCTTCCAG
CAGTTCGGCAGAGACATCGCCGACACCACCGACGCCGTGAGAGACCCCCAGACCCTGGAG
ATCCTGGACATCACCCCCTGCAGCTTCGGCGGCGTGAGCGTGATCACCCCCGGCACCAAC
ACCAGCAACCAGGTGGCCGTGCTGTACCAGGGCGTGAACTGCACCGAGGTGCCCGTGGCC
ATCCACGCCGACCAGCTGACCCCCACCTGGAGAGTGTACAGCACCGGCAGCAACGTGTTC
CAGACCAGAGCCGGCTGCCTGATCGGCGCCGAGCACGTGAACAACAGCTACGAGTGCGAC
ATCCCCATCGGCGCCGGCATCTGCGCCAGCTACCAGACCCAGACCAACAGCCCCAGAAGA
GCCAGAAGCGTGGCCAGCCAGAGCATCATCGCCTACACCATGAGCCTGGGCGTGGAGAAC
AGCGTGGCCTACAGCAACAACAGCATCGCCATCCCCACCAACTTCACCATCAGCGTGACC
ACCGAGATCCTGCCCGTGAGCATGACCAAGACCAGCGTGGACTGCACCATGTACATCTGC
GGCGACAGCACCGAGTGCAGCAACCTGCTGCTGCAGTACGGCAGCTTCTGCACCCAGCTG
AACAGAGCCCTGACCGGCATCGCCGTGGAGCAGGACAAGAACACCCAGGAGGTGTTCGCC
CAGGTGAAGCAGATCTACAAGACCCCCCCCATCAAGGACTTCGGCGGCTTCAACTTCAGC
CAGATCCTGCCCGACCCCAGCAAGCCCAGCAAGAGAAGCTTCATCGAGGACCTGCTGTTC
AACAAGGTGACCCTGGCCGACGCCGGCTTCATCAAGCAGTACGGCGACTGCCTGGGCGAC
ATCGCCGCCAGAGACCTGATCTGCGCCCAGAAGTTCAACGGCCTGACCGTGCTGCCCCCC
CTGCTGACCGACGAGATGATCGCCCAGTACACCAGCGCCCTGCTGGCCGGCACCATCACC
AGCGGCTGGACCTTCGGCGCCGGCGCCGCCCTGCAGATCCCCTTCGCCATGCAGATGGCC
TACAGATTCAACGGCATCGGCGTGACCCAGAACGTGCTGTACGAGAACCAGAAGCTGATC
GCCAACCAGTTCAACAGCGCCATCGGCAAGATCCAGGACAGCCTGAGCAGCACCGCCAGC
GCCCTGGGCAAGCTGCAGGACGTGGTGAACCAGAACGCCCAGGCCCTGAACACCCTGGTG
AAGCAGCTGAGCAGCAACTTCGGCGCCATCAGCAGCGTGCTGAACGACATCCTGAGCAGA
CTGGACAAGGTGGAGGCCGAGGTGCAGATCGACAGACTGATCACCGGCAGACTGCAGAGC
CTGCAGACCTACGTGACCCAGCAGCTGATCAGAGCCGCCGAGATCAGAGCCAGCGCCAAC
CTGGCCGCCACCAAGATGAGCGAGTGCGTGCTGGGCCAGAGCAAGAGAGTGGACTTCTGC
GGCAAGGGCTACCACCTGATGAGCTTCCCCCAGAGCGCCCCCCACGGCGTGGTGTTCCTG
CACGTGACCTACGTGCCCGCCCAGGAGAAGAACTTCACCACCGCCCCCGCCATCTGCCAC
GACGGCAAGGCCCACTTCCCCAGAGAGGGCGTGTTCGTGAGCAACGGCACCCACTGGTTC
GTGACCCAGAGAAACTTCTACGAGCCCCAGATCATCACCACCGACAACACCTTCGTGAGC
GGCAACTGCGACGTGGTGATCGGCATCGTGAACAACACCGTGTACGACCCCCTGCAGCCC
GAGCTGGACAGCTTCAAGGAGGAGCTGGACAAGTACTTCAAGAACCACACCAGCCCCGAC
GTGGACCTGGGCGACATCAGCGGCATCAACGCCAGCGTGGTGAACATCCAGAAGGAGATC
GACAGACTGAACGAGGTGGCCAAGAACCTGAACGAGAGCCTGATCGACCTGCAGGAGCTG
GGCAAGTACGAGCAGTACATCAAGTGGCCCTAG
Note:
the above sequence consists of signal peptide-extracellular domain.
SEQ ID NO: 51:
extracellular nucleic acid sequence of Beta S antigen (optimization
of insect cells):
ATGTTCGTGTTCCTGGTGCTGCTGCCCCTGGTGTCCTCCCAGTGCGTGAACCTGACCACC
CGTACCCAGCTGCCCCCCGCTTACACCAACTCCTTCACCCGTGGTGTGTACTACCCCGAC
AAGGTGTTCCGTTCCTCCGTGCTGCACTCCACCCAGGACCTGTTCCTGCCCTTCTTCTCC
AACGTGACCTGGTTCCACGCTATCCACGTGTCCGGTACCAACGGTACCAAGCGTTTCGCT
AACCCCGTGCTGCCCTTCAACGACGGTGTGTACTTCGCTTCCACCGAGAAGTCCAACATC
ATCCGTGGTTGGATCTTCGGTACCACCCTGGACTCCAAGACCCAGTCCCTGCTGATCGTG
AACAACGCTACCAACGTGGTGATCAAGGTGTGCGAGTTCCAGTTCTGCAACGACCCCTTC
CTGGGTGTGTACTACCACAAGAACAACAAGTCCTGGATGGAGTCCGAGTTCCGTGTGTAC
TCCTCCGCTAACAACTGCACCTTCGAGTACGTGTCCCAGCCCTTCCTGATGGACCTGGAG
GGTAAGCAGGGTAACTTCAAGAACCTGCGTGAGTTCGTGTTCAAGAACATCGACGGTTAC
TTCAAGATCTACTCCAAGCACACCCCCATCAACCTGGTGCGTGGTCTGCCCCAGGGTTTC
TCCGCTCTGGAGCCCCTGGTGGACCTGCCCATCGGTATCAACATCACCCGTTTCCAGACC
CTGCACCGTTCCTACCTGACCCCCGGTGACTCCTCCTCCGGTTGGACCGCTGGTGCTGCT
GCTTACTACGTGGGTTACCTGCAGCCCCGTACCTTCCTGCTGAAGTACAACGAGAACGGT
ACCATCACCGACGCTGTGGACTGCGCTCTGGACCCCCTGTCCGAGACCAAGTGCACCCTG
AAGTCCTTCACCGTGGAGAAGGGTATCTACCAGACCTCCAACTTCCGTGTGCAGCCCACC
GAGTCCATCGTGCGTTTCCCCAACATCACCAACCTGTGCCCCTTCGGTGAGGTGTTCAAC
GCTACCCGTTTCGCTTCCGTGTACGCTTGGAACCGTAAGCGTATCTCCAACTGCGTGGCT
GACTACTCCGTGCTGTACAACTCCGCTTCCTTCTCCACCTTCAAGTGCTACGGTGTGTCC
CCCACCAAGCTGAACGACCTGTGCTTCACCAACGTGTACGCTGACTCCTTCGTGATCCGT
GGTGACGAGGTGCGTCAGATCGCTCCCGGTCAGACCGGTAACATCGCTGACTACAACTAC
AAGCTGCCCGACGACTTCACCGGTTGCGTGATCGCTTGGAACTCCAACAACCTGGACTCC
AAGGTGGGTGGTAACTACAACTACCTGTACCGTCTGTTCCGTAAGTCCAACCTGAAGCCC
TTCGAGCGTGACATCTCCACCGAGATCTACCAGGCTGGTTCCACCCCCTGCAACGGTGTG
AAGGGTTTCAACTGCTACTTCCCCCTGCAGTCCTACGGTTTCCAGCCCACCTACGGTGTG
GGTTACCAGCCCTACCGTGTGGTGGTGCTGTCCTTCGAGCTGCTGCACGCTCCCGCTACC
GTGTGCGGTCCCAAGAAGTCCACCAACCTGGTGAAGAACAAGTGCGTGAACTTCAACTTC
AACGGTCTGACCGGTACCGGTGTGCTGACCGAGTCCAACAAGAAGTTCCTGCCCTTCCAG
CAGTTCGGTCGTGACATCGCTGACACCACCGACGCTGTGCGTGACCCCCAGACCCTGGAG
ATCCTGGACATCACCCCCTGCTCCTTCGGTGGTGTGTCCGTGATCACCCCCGGTACCAAC
ACCTCCAACCAGGTGGCTGTGCTGTACCAGGGTGTGAACTGCACCGAGGTGCCCGTGGCT
ATCCACGCTGACCAGCTGACCCCCACCTGGCGTGTGTACTCCACCGGTTCCAACGTGTTC
CAGACCCGTGCTGGTTGCCTGATCGGTGCTGAGCACGTGAACAACTCCTACGAGTGCGAC
ATCCCCATCGGTGCTGGTATCTGCGCTTCCTACCAGACCCAGACCAACTCCCCCCGTCGT
GCTCGTTCCGTGGCTTCCCAGTCCATCATCGCTTACACCATGTCCCTGGGTGTGGAGAAC
TCCGTGGCTTACTCCAACAACTCCATCGCTATCCCCACCAACTTCACCATCTCCGTGACC
ACCGAGATCCTGCCCGTGTCCATGACCAAGACCTCCGTGGACTGCACCATGTACATCTGC
GGTGACTCCACCGAGTGCTCCAACCTGCTGCTGCAGTACGGTTCCTTCTGCACCCAGCTG
AACCGTGCTCTGACCGGTATCGCTGTGGAGCAGGACAAGAACACCCAGGAGGTGTTCGCT
CAGGTGAAGCAGATCTACAAGACCCCCCCCATCAAGGACTTCGGTGGTTTCAACTTCTCC
CAGATCCTGCCCGACCCCTCCAAGCCCTCCAAGCGTTCCTTCATCGAGGACCTGCTGTTC
AACAAGGTGACCCTGGCTGACGCTGGTTTCATCAAGCAGTACGGTGACTGCCTGGGTGAC
ATCGCTGCTCGTGACCTGATCTGCGCTCAGAAGTTCAACGGTCTGACCGTGCTGCCCCCC
CTGCTGACCGACGAGATGATCGCTCAGTACACCTCCGCTCTGCTGGCTGGTACCATCACC
TCCGGTTGGACCTTCGGTGCTGGTGCTGCTCTGCAGATCCCCTTCGCTATGCAGATGGCT
TACCGTTTCAACGGTATCGGTGTGACCCAGAACGTGCTGTACGAGAACCAGAAGCTGATC
GCTAACCAGTTCAACTCCGCTATCGGTAAGATCCAGGACTCCCTGTCCTCCACCGCTTCC
GCTCTGGGTAAGCTGCAGGACGTGGTGAACCAGAACGCTCAGGCTCTGAACACCCTGGTG
AAGCAGCTGTCCTCCAACTTCGGTGCTATCTCCTCCGTGCTGAACGACATCCTGTCCCGT
CTGGACAAGGTGGAGGCTGAGGTGCAGATCGACCGTCTGATCACCGGTCGTCTGCAGTCC
CTGCAGACCTACGTGACCCAGCAGCTGATCCGTGCTGCTGAGATCCGTGCTTCCGCTAAC
CTGGCTGCTACCAAGATGTCCGAGTGCGTGCTGGGTCAGTCCAAGCGTGTGGACTTCTGC
GGTAAGGGTTACCACCTGATGTCCTTCCCCCAGTCCGCTCCCCACGGTGTGGTGTTCCTG
CACGTGACCTACGTGCCCGCTCAGGAGAAGAACTTCACCACCGCTCCCGCTATCTGCCAC
GACGGTAAGGCTCACTTCCCCCGTGAGGGTGTGTTCGTGTCCAACGGTACCCACTGGTTC
GTGACCCAGCGTAACTTCTACGAGCCCCAGATCATCACCACCGACAACACCTTCGTGTCC
GGTAACTGCGACGTGGTGATCGGTATCGTGAACAACACCGTGTACGACCCCCTGCAGCCC
GAGCTGGACTCCTTCAAGGAGGAGCTGGACAAGTACTTCAAGAACCACACCTCCCCCGAC
GTGGACCTGGGTGACATCTCCGGTATCAACGCTTCCGTGGTGAACATCCAGAAGGAGATC
GACCGTCTGAACGAGGTGGCTAAGAACCTGAACGAGTCCCTGATCGACCTGCAGGAGCTG
GGTAAGTACGAGCAGTACATCAAGTGGCCCTAG
Note:
the above sequence consists of signal peptide-extracellular domain.
SEQ ID NO: 52:
Full-length amino acid sequence of Delta S antigen:
MFVFLVLLPLVSSQCVNLRTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLDVYYHKNNKSWMESGVYSSANNCTFEYVSQPFLMDLEGK
QGNFKNLREFVFKNIDGYFKIYSKHTPINLVRDLPQGFSALEPLVDLPIGINITRFQTLL
ALHRSYLTPGDSSSGWTAGAAAYYVGYLQPRTFLLKYNENGTITDAVDCALDPLSETKCT
LKSFTVEKGIYQTSNFRVQPTESIVRFPNITNLCPFGEVENATRFASVYAWNRKRISNCV
ADYSVLYNSASFSTFKCYGVSPTKLNDLCFTNVYADSFVIRGDEVRQIAPGQTGKIADYN
YKLPDDFTGCVIAWNSNNLDSKVGGNYNYRYRLFRKSNLKPFERDISTEIYQAGSKPCNG
VEGFNCYFPLQSYGFQPTNGVGYQPYRVVVLSFELLHAPATVCGPKKSTNLVKNKCVNFN
FNGLTGTGVLTESNKKFLPFQQFGRDIADTTDAVRDPQTLEILDITPCSFGGVSVITPGT
NTSNQVAVLYQGVNCTEVPVAIHADQLTPTWRVYSTGSNVFQTRAGCLIGAEHVNNSYEC
DIPIGAGICASYQTQTNSRRRARSVASQSIIAYTMSLGAENSVAYSNNSIAIPTNFTISV
TTEILPVSMTKTSVDCTMYICGDSTECSNLLLQYGSFCTQLNRALTGIAVEQDKNTQEVF
AQVKQIYKTPPIKDFGGFNFSQILPDPSKPSKRSFIEDLLFNKVTLADAGFIKQYGDCLG
DIAARDLICAQKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQNVVNQNAQALNTL
VKQLSSNFGAISSVLNDILSRLDKVEAEVQIDRLITGRLQSLQTYVTQQLIRAAEIRASA
NLAATKMSECVLGQSKRVDFCGKGYHLMSFPQSAPHGVVFLHVTYVPAQEKNFTTAPAIC
HDGKAHFPREGVFVSNGTHWFVTQRNFYEPQIITTDNTFVSGNCDVVIGIVNNTVYDPLQ
PELDSFKEELDKYFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSCGSCCKFDEDDSE
PVLKGVKLHYT
Note:
the above sequence consists of signal peptide-extracellular domain-
transmembrane domain-intracellular domain.
SEQ ID NO: 53:
Full-length nucleic acid sequence of Delta S antigen (optimization
of CHO cells):
ATGTTCGTGTTCCTGGTGCTGCTGCCCCTGGTGAGCAGCCAGTGCGTGAACCTGAGAACC
AGAACCCAGCTGCCCCCCGCCTACACCAACAGCTTCACCAGAGGCGTGTACTACCCCGAC
AAGGTGTTCAGAAGCAGCGTGCTGCACAGCACCCAGGACCTGTTCCTGCCCTTCTTCAGC
AACGTGACCTGGTTCCACGCCATCCACGTGAGCGGCACCAACGGCACCAAGAGATTCGAC
AACCCCGTGCTGCCCTTCAACGACGGCGTGTACTTCGCCAGCACCGAGAAGAGCAACATC
ATCAGAGGCTGGATCTTCGGCACCACCCTGGACAGCAAGACCCAGAGCCTGCTGATCGTG
AACAACGCCACCAACGTGGTGATCAAGGTGTGCGAGTTCCAGTTCTGCAACGACCCCTTC
CTGGACGTGTACTACCACAAGAACAACAAGAGCTGGATGGAGAGCGGCGTGTACAGCAGC
GCCAACAACTGCACCTTCGAGTACGTGAGCCAGCCCTTCCTGATGGACCTGGAGGGCAAG
CAGGGCAACTTCAAGAACCTGAGAGAGTTCGTGTTCAAGAACATCGACGGCTACTTCAAG
ATCTACAGCAAGCACACCCCCATCAACCTGGTGAGAGACCTGCCCCAGGGCTTCAGCGCC
CTGGAGCCCCTGGTGGACCTGCCCATCGGCATCAACATCACCAGATTCCAGACCCTGCTG
GCCCTGCACAGAAGCTACCTGACCCCCGGCGACAGCAGCAGCGGCTGGACCGCCGGCGCC
GCCGCCTACTACGTGGGCTACCTGCAGCCCAGAACCTTCCTGCTGAAGTACAACGAGAAC
GGCACCATCACCGACGCCGTGGACTGCGCCCTGGACCCCCTGAGCGAGACCAAGTGCACC
CTGAAGAGCTTCACCGTGGAGAAGGGCATCTACCAGACCAGCAACTTCAGAGTGCAGCCC
ACCGAGAGCATCGTGAGATTCCCCAACATCACCAACCTGTGCCCCTTCGGCGAGGTGTTC
AACGCCACCAGATTCGCCAGCGTGTACGCCTGGAACAGAAAGAGAATCAGCAACTGCGTG
GCCGACTACAGCGTGCTGTACAACAGCGCCAGCTTCAGCACCTTCAAGTGCTACGGCGTG
AGCCCCACCAAGCTGAACGACCTGTGCTTCACCAACGTGTACGCCGACAGCTTCGTGATC
AGAGGCGACGAGGTGAGACAGATCGCCCCCGGCCAGACCGGCAAGATCGCCGACTACAAC
TACAAGCTGCCCGACGACTTCACCGGCTGCGTGATCGCCTGGAACAGCAACAACCTGGAC
AGCAAGGTGGGCGGCAACTACAACTACAGATACAGACTGTTCAGAAAGAGCAACCTGAAG
CCCTTCGAGAGAGACATCAGCACCGAGATCTACCAGGCCGGCAGCAAGCCCTGCAACGGC
GTGGAGGGCTTCAACTGCTACTTCCCCCTGCAGAGCTACGGCTTCCAGCCCACCAACGGC
GTGGGCTACCAGCCCTACAGAGTGGTGGTGCTGAGCTTCGAGCTGCTGCACGCCCCCGCC
ACCGTGTGCGGCCCCAAGAAGAGCACCAACCTGGTGAAGAACAAGTGCGTGAACTTCAAC
TTCAACGGCCTGACCGGCACCGGCGTGCTGACCGAGAGCAACAAGAAGTTCCTGCCCTTC
CAGCAGTTCGGCAGAGACATCGCCGACACCACCGACGCCGTGAGAGACCCCCAGACCCTG
GAGATCCTGGACATCACCCCCTGCAGCTTCGGCGGCGTGAGCGTGATCACCCCCGGCACC
AACACCAGCAACCAGGTGGCCGTGCTGTACCAGGGCGTGAACTGCACCGAGGTGCCCGTG
GCCATCCACGCCGACCAGCTGACCCCCACCTGGAGAGTGTACAGCACCGGCAGCAACGTG
TTCCAGACCAGAGCCGGCTGCCTGATCGGCGCCGAGCACGTGAACAACAGCTACGAGTGC
GACATCCCCATCGGCGCCGGCATCTGCGCCAGCTACCAGACCCAGACCAACAGCAGAAGA
AGAGCCAGAAGCGTGGCCAGCCAGAGCATCATCGCCTACACCATGAGCCTGGGCGCCGAG
AACAGCGTGGCCTACAGCAACAACAGCATCGCCATCCCCACCAACTTCACCATCAGCGTG
ACCACCGAGATCCTGCCCGTGAGCATGACCAAGACCAGCGTGGACTGCACCATGTACATC
TGCGGCGACAGCACCGAGTGCAGCAACCTGCTGCTGCAGTACGGCAGCTTCTGCACCCAG
CTGAACAGAGCCCTGACCGGCATCGCCGTGGAGCAGGACAAGAACACCCAGGAGGTGTTC
GCCCAGGTGAAGCAGATCTACAAGACCCCCCCCATCAAGGACTTCGGCGGCTTCAACTTC
AGCCAGATCCTGCCCGACCCCAGCAAGCCCAGCAAGAGAAGCTTCATCGAGGACCTGCTG
TTCAACAAGGTGACCCTGGCCGACGCCGGCTTCATCAAGCAGTACGGCGACTGCCTGGGC
GACATCGCCGCCAGAGACCTGATCTGCGCCCAGAAGTTCAACGGCCTGACCGTGCTGCCC
CCCCTGCTGACCGACGAGATGATCGCCCAGTACACCAGCGCCCTGCTGGCCGGCACCATC
ACCAGCGGCTGGACCTTCGGCGCCGGCGCCGCCCTGCAGATCCCCTTCGCCATGCAGATG
GCCTACAGATTCAACGGCATCGGCGTGACCCAGAACGTGCTGTACGAGAACCAGAAGCTG
ATCGCCAACCAGTTCAACAGCGCCATCGGCAAGATCCAGGACAGCCTGAGCAGCACCGCC
AGCGCCCTGGGCAAGCTGCAGAACGTGGTGAACCAGAACGCCCAGGCCCTGAACACCCTG
GTGAAGCAGCTGAGCAGCAACTTCGGCGCCATCAGCAGCGTGCTGAACGACATCCTGAGC
AGACTGGACAAGGTGGAGGCCGAGGTGCAGATCGACAGACTGATCACCGGCAGACTGCAG
AGCCTGCAGACCTACGTGACCCAGCAGCTGATCAGAGCCGCCGAGATCAGAGCCAGCGCC
AACCTGGCCGCCACCAAGATGAGCGAGTGCGTGCTGGGCCAGAGCAAGAGAGTGGACTTC
TGCGGCAAGGGCTACCACCTGATGAGCTTCCCCCAGAGCGCCCCCCACGGCGTGGTGTTC
CTGCACGTGACCTACGTGCCCGCCCAGGAGAAGAACTTCACCACCGCCCCCGCCATCTGC
CACGACGGCAAGGCCCACTTCCCCAGAGAGGGCGTGTTCGTGAGCAACGGCACCCACTGG
TTCGTGACCCAGAGAAACTTCTACGAGCCCCAGATCATCACCACCGACAACACCTTCGTG
AGCGGCAACTGCGACGTGGTGATCGGCATCGTGAACAACACCGTGTACGACCCCCTGCAG
CCCGAGCTGGACAGCTTCAAGGAGGAGCTGGACAAGTACTTCAAGAACCACACCAGCCCC
GACGTGGACCTGGGCGACATCAGCGGCATCAACGCCAGCGTGGTGAACATCCAGAAGGAG
ATCGACAGACTGAACGAGGTGGCCAAGAACCTGAACGAGAGCCTGATCGACCTGCAGGAG
CTGGGCAAGTACGAGCAGTACATCAAGTGGCCCTGGTACATCTGGCTGGGCTTCATCGCC
GGCCTGATCGCCATCGTGATGGTGACCATCATGCTGTGCTGCATGACCAGCTGCTGCAGC
TGCCTGAAGGGCTGCTGCAGCTGCGGCAGCTGCTGCAAGTTCGACGAGGACGACAGCGAG
CCCGTGCTGAAGGGCGTGAAGCTGCACTACACCTAG
Note:
the above sequence consists of signal peptide-extracellular domain-
transmembrane domain-intracellular domain.
SEQ ID NO: 54:
Full-length nucleic acid sequence of Delta S antigen (optimization
of insect cells):
ATGTTCGTGTTCCTGGTGCTGCTGCCCCTGGTGTCCTCCCAGTGCGTGAACCTGCGTACC
CGTACCCAGCTGCCCCCCGCTTACACCAACTCCTTCACCCGTGGTGTGTACTACCCCGAC
AAGGTGTTCCGTTCCTCCGTGCTGCACTCCACCCAGGACCTGTTCCTGCCCTTCTTCTCC
AACGTGACCTGGTTCCACGCTATCCACGTGTCCGGTACCAACGGTACCAAGCGTTTCGAC
AACCCCGTGCTGCCCTTCAACGACGGTGTGTACTTCGCTTCCACCGAGAAGTCCAACATC
ATCCGTGGTTGGATCTTCGGTACCACCCTGGACTCCAAGACCCAGTCCCTGCTGATCGTG
AACAACGCTACCAACGTGGTGATCAAGGTGTGCGAGTTCCAGTTCTGCAACGACCCCTTC
CTGGACGTGTACTACCACAAGAACAACAAGTCCTGGATGGAGTCCGGTGTGTACTCCTCC
GCTAACAACTGCACCTTCGAGTACGTGTCCCAGCCCTTCCTGATGGACCTGGAGGGTAAG
CAGGGTAACTTCAAGAACCTGCGTGAGTTCGTGTTCAAGAACATCGACGGTTACTTCAAG
ATCTACTCCAAGCACACCCCCATCAACCTGGTGCGTGACCTGCCCCAGGGTTTCTCCGCT
CTGGAGCCCCTGGTGGACCTGCCCATCGGTATCAACATCACCCGTTTCCAGACCCTGCTG
GCTCTGCACCGTTCCTACCTGACCCCCGGTGACTCCTCCTCCGGTTGGACCGCTGGTGCT
GCTGCTTACTACGTGGGTTACCTGCAGCCCCGTACCTTCCTGCTGAAGTACAACGAGAAC
GGTACCATCACCGACGCTGTGGACTGCGCTCTGGACCCCCTGTCCGAGACCAAGTGCACC
CTGAAGTCCTTCACCGTGGAGAAGGGTATCTACCAGACCTCCAACTTCCGTGTGCAGCCC
ACCGAGTCCATCGTGCGTTTCCCCAACATCACCAACCTGTGCCCCTTCGGTGAGGTGTTC
AACGCTACCCGTTTCGCTTCCGTGTACGCTTGGAACCGTAAGCGTATCTCCAACTGCGTG
GCTGACTACTCCGTGCTGTACAACTCCGCTTCCTTCTCCACCTTCAAGTGCTACGGTGTG
TCCCCCACCAAGCTGAACGACCTGTGCTTCACCAACGTGTACGCTGACTCCTTCGTGATC
CGTGGTGACGAGGTGCGTCAGATCGCTCCCGGTCAGACCGGTAAGATCGCTGACTACAAC
TACAAGCTGCCCGACGACTTCACCGGTTGCGTGATCGCTTGGAACTCCAACAACCTGGAC
TCCAAGGTGGGTGGTAACTACAACTACCGTTACCGTCTGTTCCGTAAGTCCAACCTGAAG
CCCTTCGAGCGTGACATCTCCACCGAGATCTACCAGGCTGGTTCCAAGCCCTGCAACGGT
GTGGAGGGTTTCAACTGCTACTTCCCCCTGCAGTCCTACGGTTTCCAGCCCACCAACGGT
GTGGGTTACCAGCCCTACCGTGTGGTGGTGCTGTCCTTCGAGCTGCTGCACGCTCCCGCT
ACCGTGTGCGGTCCCAAGAAGTCCACCAACCTGGTGAAGAACAAGTGCGTGAACTTCAAC
TTCAACGGTCTGACCGGTACCGGTGTGCTGACCGAGTCCAACAAGAAGTTCCTGCCCTTC
CAGCAGTTCGGTCGTGACATCGCTGACACCACCGACGCTGTGCGTGACCCCCAGACCCTG
GAGATCCTGGACATCACCCCCTGCTCCTTCGGTGGTGTGTCCGTGATCACCCCCGGTACC
AACACCTCCAACCAGGTGGCTGTGCTGTACCAGGGTGTGAACTGCACCGAGGTGCCCGTG
GCTATCCACGCTGACCAGCTGACCCCCACCTGGCGTGTGTACTCCACCGGTTCCAACGTG
TTCCAGACCCGTGCTGGTTGCCTGATCGGTGCTGAGCACGTGAACAACTCCTACGAGTGC
GACATCCCCATCGGTGCTGGTATCTGCGCTTCCTACCAGACCCAGACCAACTCCCGTCGT
CGTGCTCGTTCCGTGGCTTCCCAGTCCATCATCGCTTACACCATGTCCCTGGGTGCTGAG
AACTCCGTGGCTTACTCCAACAACTCCATCGCTATCCCCACCAACTTCACCATCTCCGTG
ACCACCGAGATCCTGCCCGTGTCCATGACCAAGACCTCCGTGGACTGCACCATGTACATC
TGCGGTGACTCCACCGAGTGCTCCAACCTGCTGCTGCAGTACGGTTCCTTCTGCACCCAG
CTGAACCGTGCTCTGACCGGTATCGCTGTGGAGCAGGACAAGAACACCCAGGAGGTGTTC
GCTCAGGTGAAGCAGATCTACAAGACCCCCCCCATCAAGGACTTCGGTGGTTTCAACTTC
TCCCAGATCCTGCCCGACCCCTCCAAGCCCTCCAAGCGTTCCTTCATCGAGGACCTGCTG
TTCAACAAGGTGACCCTGGCTGACGCTGGTTTCATCAAGCAGTACGGTGACTGCCTGGGT
GACATCGCTGCTCGTGACCTGATCTGCGCTCAGAAGTTCAACGGTCTGACCGTGCTGCCC
CCCCTGCTGACCGACGAGATGATCGCTCAGTACACCTCCGCTCTGCTGGCTGGTACCATC
ACCTCCGGTTGGACCTTCGGTGCTGGTGCTGCTCTGCAGATCCCCTTCGCTATGCAGATG
GCTTACCGTTTCAACGGTATCGGTGTGACCCAGAACGTGCTGTACGAGAACCAGAAGCTG
ATCGCTAACCAGTTCAACTCCGCTATCGGTAAGATCCAGGACTCCCTGTCCTCCACCGCT
TCCGCTCTGGGTAAGCTGCAGAACGTGGTGAACCAGAACGCTCAGGCTCTGAACACCCTG
GTGAAGCAGCTGTCCTCCAACTTCGGTGCTATCTCCTCCGTGCTGAACGACATCCTGTCC
CGTCTGGACAAGGTGGAGGCTGAGGTGCAGATCGACCGTCTGATCACCGGTCGTCTGCAG
TCCCTGCAGACCTACGTGACCCAGCAGCTGATCCGTGCTGCTGAGATCCGTGCTTCCGCT
AACCTGGCTGCTACCAAGATGTCCGAGTGCGTGCTGGGTCAGTCCAAGCGTGTGGACTTC
TGCGGTAAGGGTTACCACCTGATGTCCTTCCCCCAGTCCGCTCCCCACGGTGTGGTGTTC
CTGCACGTGACCTACGTGCCCGCTCAGGAGAAGAACTTCACCACCGCTCCCGCTATCTGC
CACGACGGTAAGGCTCACTTCCCCCGTGAGGGTGTGTTCGTGTCCAACGGTACCCACTGG
TTCGTGACCCAGCGTAACTTCTACGAGCCCCAGATCATCACCACCGACAACACCTTCGTG
TCCGGTAACTGCGACGTGGTGATCGGTATCGTGAACAACACCGTGTACGACCCCCTGCAG
CCCGAGCTGGACTCCTTCAAGGAGGAGCTGGACAAGTACTTCAAGAACCACACCTCCCCC
GACGTGGACCTGGGTGACATCTCCGGTATCAACGCTTCCGTGGTGAACATCCAGAAGGAG
ATCGACCGTCTGAACGAGGTGGCTAAGAACCTGAACGAGTCCCTGATCGACCTGCAGGAG
CTGGGTAAGTACGAGCAGTACATCAAGTGGCCCTGGTACATCTGGCTGGGTTTCATCGCT
GGTCTGATCGCTATCGTGATGGTGACCATCATGCTGTGCTGCATGACCTCCTGCTGCTCC
TGCCTGAAGGGTTGCTGCTCCTGCGGTTCCTGCTGCAAGTTCGACGAGGACGACTCCGAG
CCCGTGCTGAAGGGTGTGAAGCTGCACTACACCTAG
Note:
the above sequence consists of signal peptide-extracellular domain-
transmembrane domain-intracellular domain.
SEQ ID NO: 55:
Extracellular amino acid sequence of Delta S antigen:
MFVFLVLLPLVSSQCVNLRTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLDVYYHKNNKSWMESGVYSSANNCTFEYVSQPFLMDLEGK
QGNFKNLREFVFKNIDGYFKIYSKHTPINLVRDLPQGFSALEPLVDLPIGINITRFQTLL
ALHRSYLTPGDSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYAWNRKRISNCV
ADYSVLYNSASFSTFKCYGVSPTKLNDLCFTNVYADSFVIRGDEVRQIAPGQTGKIADYN
YKLPDDFTGCVIAWNSNNLDSKVGGNYNYRYRLFRKSNLKPFERDISTEIYQAGSKPCNG
VEGENCYFPLQSYGFQPTNGVGYQPYRVVVLSFELLHAPATVCGPKKSTNLVKNKCVNFN
FNGLTGTGVLTESNKKFLPFQQFGRDIADTTDAVRDPQTLEILDITPCSFGGVSVITPGT
NTSNQVAVLYQGVNCTEVPVAIHADQLTPTWRVYSTGSNVFQTRAGCLIGAEHVNNSYEC
DIPIGAGICASYQTQTNSRRRARSVASQSIIAYTMSLGAENSVAYSNNSIAIPTNFTISV
TTEILPVSMTKTSVDCTMYICGDSTECSNLLLQYGSFCTQLNRALTGIAVEQDKNTQEVF
AQVKQIYKTPPIKDFGGFNFSQILPDPSKPSKRSFIEDLLFNKVTLADAGFIKQYGDCLG
DIAARDLICAQKFNGLTVLPPLLTDEMIAQYTSALLAGTITSGWTFGAGAALQIPFAMQM
AYRENGIGVTQNVLYENQKLIANQFNSAIGKIQDSLSSTASALGKLQNVVNQNAQALNTL
VKQLSSNFGAISSVLNDILSRLDKVEAEVQIDRLITGRLQSLQTYVTQQLIRAAEIRASA
NLAATKMSECVLGQSKRVDFCGKGYHLMSFPQSAPHGVVFLHVTYVPAQEKNFTTAPAIC
HDGKAHFPREGVFVSNGTHWFVTQRNFYEPQIITTDNTFVSGNCDVVIGIVNNTVYDPLQ
PELDSFKEELDKYFKNHTSPDVDLGDISGINASVVNIQKEIDRLNEVAKNLNESLIDLQE
LGKYEQYIKWP
Note:
the above sequence consists of signal peptide-extracellular domain.
SEQ ID NO: 56:
Extracellular nucleic acid sequence of Delta S antigen (optimization
of CHO cells):
ATGTTCGTGTTCCTGGTGCTGCTGCCCCTGGTGAGCAGCCAGTGCGTGAACCTGAGAACC
AGAACCCAGCTGCCCCCCGCCTACACCAACAGCTTCACCAGAGGCGTGTACTACCCCGAC
AAGGTGTTCAGAAGCAGCGTGCTGCACAGCACCCAGGACCTGTTCCTGCCCTTCTTCAGC
AACGTGACCTGGTTCCACGCCATCCACGTGAGCGGCACCAACGGCACCAAGAGATTCGAC
AACCCCGTGCTGCCCTTCAACGACGGCGTGTACTTCGCCAGCACCGAGAAGAGCAACATC
ATCAGAGGCTGGATCTTCGGCACCACCCTGGACAGCAAGACCCAGAGCCTGCTGATCGTG
AACAACGCCACCAACGTGGTGATCAAGGTGTGCGAGTTCCAGTTCTGCAACGACCCCTTC
CTGGACGTGTACTACCACAAGAACAACAAGAGCTGGATGGAGAGCGGCGTGTACAGCAGC
GCCAACAACTGCACCTTCGAGTACGTGAGCCAGCCCTTCCTGATGGACCTGGAGGGCAAG
CAGGGCAACTTCAAGAACCTGAGAGAGTTCGTGTTCAAGAACATCGACGGCTACTTCAAG
ATCTACAGCAAGCACACCCCCATCAACCTGGTGAGAGACCTGCCCCAGGGCTTCAGCGCC
CTGGAGCCCCTGGTGGACCTGCCCATCGGCATCAACATCACCAGATTCCAGACCCTGCTG
GCCCTGCACAGAAGCTACCTGACCCCCGGCGACAGCAGCAGCGGCTGGACCGCCGGCGCC
GCCGCCTACTACGTGGGCTACCTGCAGCCCAGAACCTTCCTGCTGAAGTACAACGAGAAC
GGCACCATCACCGACGCCGTGGACTGCGCCCTGGACCCCCTGAGCGAGACCAAGTGCACC
CTGAAGAGCTTCACCGTGGAGAAGGGCATCTACCAGACCAGCAACTTCAGAGTGCAGCCC
ACCGAGAGCATCGTGAGATTCCCCAACATCACCAACCTGTGCCCCTTCGGCGAGGTGTTC
AACGCCACCAGATTCGCCAGCGTGTACGCCTGGAACAGAAAGAGAATCAGCAACTGCGTG
GCCGACTACAGCGTGCTGTACAACAGCGCCAGCTTCAGCACCTTCAAGTGCTACGGCGTG
AGCCCCACCAAGCTGAACGACCTGTGCTTCACCAACGTGTACGCCGACAGCTTCGTGATC
AGAGGCGACGAGGTGAGACAGATCGCCCCCGGCCAGACCGGCAAGATCGCCGACTACAAC
TACAAGCTGCCCGACGACTTCACCGGCTGCGTGATCGCCTGGAACAGCAACAACCTGGAC
AGCAAGGTGGGCGGCAACTACAACTACAGATACAGACTGTTCAGAAAGAGCAACCTGAAG
CCCTTCGAGAGAGACATCAGCACCGAGATCTACCAGGCCGGCAGCAAGCCCTGCAACGGC
GTGGAGGGCTTCAACTGCTACTTCCCCCTGCAGAGCTACGGCTTCCAGCCCACCAACGGC
GTGGGCTACCAGCCCTACAGAGTGGTGGTGCTGAGCTTCGAGCTGCTGCACGCCCCCGCC
ACCGTGTGCGGCCCCAAGAAGAGCACCAACCTGGTGAAGAACAAGTGCGTGAACTTCAAC
TTCAACGGCCTGACCGGCACCGGCGTGCTGACCGAGAGCAACAAGAAGTTCCTGCCCTTC
CAGCAGTTCGGCAGAGACATCGCCGACACCACCGACGCCGTGAGAGACCCCCAGACCCTG
GAGATCCTGGACATCACCCCCTGCAGCTTCGGCGGCGTGAGCGTGATCACCCCCGGCACC
AACACCAGCAACCAGGTGGCCGTGCTGTACCAGGGCGTGAACTGCACCGAGGTGCCCGTG
GCCATCCACGCCGACCAGCTGACCCCCACCTGGAGAGTGTACAGCACCGGCAGCAACGTG
TTCCAGACCAGAGCCGGCTGCCTGATCGGCGCCGAGCACGTGAACAACAGCTACGAGTGC
GACATCCCCATCGGCGCCGGCATCTGCGCCAGCTACCAGACCCAGACCAACAGCAGAAGA
AGAGCCAGAAGCGTGGCCAGCCAGAGCATCATCGCCTACACCATGAGCCTGGGCGCCGAG
AACAGCGTGGCCTACAGCAACAACAGCATCGCCATCCCCACCAACTTCACCATCAGCGTG
ACCACCGAGATCCTGCCCGTGAGCATGACCAAGACCAGCGTGGACTGCACCATGTACATC
TGCGGCGACAGCACCGAGTGCAGCAACCTGCTGCTGCAGTACGGCAGCTTCTGCACCCAG
CTGAACAGAGCCCTGACCGGCATCGCCGTGGAGCAGGACAAGAACACCCAGGAGGTGTTC
GCCCAGGTGAAGCAGATCTACAAGACCCCCCCCATCAAGGACTTCGGCGGCTTCAACTTC
AGCCAGATCCTGCCCGACCCCAGCAAGCCCAGCAAGAGAAGCTTCATCGAGGACCTGCTG
TTCAACAAGGTGACCCTGGCCGACGCCGGCTTCATCAAGCAGTACGGCGACTGCCTGGGC
GACATCGCCGCCAGAGACCTGATCTGCGCCCAGAAGTTCAACGGCCTGACCGTGCTGCCC
CCCCTGCTGACCGACGAGATGATCGCCCAGTACACCAGCGCCCTGCTGGCCGGCACCATC
ACCAGCGGCTGGACCTTCGGCGCCGGCGCCGCCCTGCAGATCCCCTTCGCCATGCAGATG
GCCTACAGATTCAACGGCATCGGCGTGACCCAGAACGTGCTGTACGAGAACCAGAAGCTG
ATCGCCAACCAGTTCAACAGCGCCATCGGCAAGATCCAGGACAGCCTGAGCAGCACCGCC
AGCGCCCTGGGCAAGCTGCAGAACGTGGTGAACCAGAACGCCCAGGCCCTGAACACCCTG
GTGAAGCAGCTGAGCAGCAACTTCGGCGCCATCAGCAGCGTGCTGAACGACATCCTGAGC
AGACTGGACAAGGTGGAGGCCGAGGTGCAGATCGACAGACTGATCACCGGCAGACTGCAG
AGCCTGCAGACCTACGTGACCCAGCAGCTGATCAGAGCCGCCGAGATCAGAGCCAGCGCC
AACCTGGCCGCCACCAAGATGAGCGAGTGCGTGCTGGGCCAGAGCAAGAGAGTGGACTTC
TGCGGCAAGGGCTACCACCTGATGAGCTTCCCCCAGAGCGCCCCCCACGGCGTGGTGTTC
CTGCACGTGACCTACGTGCCCGCCCAGGAGAAGAACTTCACCACCGCCCCCGCCATCTGC
CACGACGGCAAGGCCCACTTCCCCAGAGAGGGCGTGTTCGTGAGCAACGGCACCCACTGG
TTCGTGACCCAGAGAAACTTCTACGAGCCCCAGATCATCACCACCGACAACACCTTCGTG
AGCGGCAACTGCGACGTGGTGATCGGCATCGTGAACAACACCGTGTACGACCCCCTGCAG
CCCGAGCTGGACAGCTTCAAGGAGGAGCTGGACAAGTACTTCAAGAACCACACCAGCCCC
GACGTGGACCTGGGCGACATCAGCGGCATCAACGCCAGCGTGGTGAACATCCAGAAGGAG
ATCGACAGACTGAACGAGGTGGCCAAGAACCTGAACGAGAGCCTGATCGACCTGCAGGAG
CTGGGCAAGTACGAGCAGTACATCAAGTGGCCCTAG
Note:
the above sequence consists of signal peptide-extracellular domain.
SEQ ID NO: 57:
Extracellular nucleic acid sequence of Delta S antigen (optimization
of insect cells):
ATGTTCGTGTTCCTGGTGCTGCTGCCCCTGGTGTCCTCCCAGTGCGTGAACCTGCGTACC
CGTACCCAGCTGCCCCCCGCTTACACCAACTCCTTCACCCGTGGTGTGTACTACCCCGAC
AAGGTGTTCCGTTCCTCCGTGCTGCACTCCACCCAGGACCTGTTCCTGCCCTTCTTCTCC
AACGTGACCTGGTTCCACGCTATCCACGTGTCCGGTACCAACGGTACCAAGCGTTTCGAC
AACCCCGTGCTGCCCTTCAACGACGGTGTGTACTTCGCTTCCACCGAGAAGTCCAACATC
ATCCGTGGTTGGATCTTCGGTACCACCCTGGACTCCAAGACCCAGTCCCTGCTGATCGTG
AACAACGCTACCAACGTGGTGATCAAGGTGTGCGAGTTCCAGTTCTGCAACGACCCCTTC
CTGGACGTGTACTACCACAAGAACAACAAGTCCTGGATGGAGTCCGGTGTGTACTCCTCC
GCTAACAACTGCACCTTCGAGTACGTGTCCCAGCCCTTCCTGATGGACCTGGAGGGTAAG
CAGGGTAACTTCAAGAACCTGCGTGAGTTCGTGTTCAAGAACATCGACGGTTACTTCAAG
ATCTACTCCAAGCACACCCCCATCAACCTGGTGCGTGACCTGCCCCAGGGTTTCTCCGCT
CTGGAGCCCCTGGTGGACCTGCCCATCGGTATCAACATCACCCGTTTCCAGACCCTGCTG
GCTCTGCACCGTTCCTACCTGACCCCCGGTGACTCCTCCTCCGGTTGGACCGCTGGTGCT
GCTGCTTACTACGTGGGTTACCTGCAGCCCCGTACCTTCCTGCTGAAGTACAACGAGAAC
GGTACCATCACCGACGCTGTGGACTGCGCTCTGGACCCCCTGTCCGAGACCAAGTGCACC
CTGAAGTCCTTCACCGTGGAGAAGGGTATCTACCAGACCTCCAACTTCCGTGTGCAGCCC
ACCGAGTCCATCGTGCGTTTCCCCAACATCACCAACCTGTGCCCCTTCGGTGAGGTGTTC
AACGCTACCCGTTTCGCTTCCGTGTACGCTTGGAACCGTAAGCGTATCTCCAACTGCGTG
GCTGACTACTCCGTGCTGTACAACTCCGCTTCCTTCTCCACCTTCAAGTGCTACGGTGTG
TCCCCCACCAAGCTGAACGACCTGTGCTTCACCAACGTGTACGCTGACTCCTTCGTGATC
CGTGGTGACGAGGTGCGTCAGATCGCTCCCGGTCAGACCGGTAAGATCGCTGACTACAAC
TACAAGCTGCCCGACGACTTCACCGGTTGCGTGATCGCTTGGAACTCCAACAACCTGGAC
TCCAAGGTGGGTGGTAACTACAACTACCGTTACCGTCTGTTCCGTAAGTCCAACCTGAAG
CCCTTCGAGCGTGACATCTCCACCGAGATCTACCAGGCTGGTTCCAAGCCCTGCAACGGT
GTGGAGGGTTTCAACTGCTACTTCCCCCTGCAGTCCTACGGTTTCCAGCCCACCAACGGT
GTGGGTTACCAGCCCTACCGTGTGGTGGTGCTGTCCTTCGAGCTGCTGCACGCTCCCGCT
ACCGTGTGCGGTCCCAAGAAGTCCACCAACCTGGTGAAGAACAAGTGCGTGAACTTCAAC
TTCAACGGTCTGACCGGTACCGGTGTGCTGACCGAGTCCAACAAGAAGTTCCTGCCCTTC
CAGCAGTTCGGTCGTGACATCGCTGACACCACCGACGCTGTGCGTGACCCCCAGACCCTG
GAGATCCTGGACATCACCCCCTGCTCCTTCGGTGGTGTGTCCGTGATCACCCCCGGTACC
AACACCTCCAACCAGGTGGCTGTGCTGTACCAGGGTGTGAACTGCACCGAGGTGCCCGTG
GCTATCCACGCTGACCAGCTGACCCCCACCTGGCGTGTGTACTCCACCGGTTCCAACGTG
TTCCAGACCCGTGCTGGTTGCCTGATCGGTGCTGAGCACGTGAACAACTCCTACGAGTGC
GACATCCCCATCGGTGCTGGTATCTGCGCTTCCTACCAGACCCAGACCAACTCCCGTCGT
CGTGCTCGTTCCGTGGCTTCCCAGTCCATCATCGCTTACACCATGTCCCTGGGTGCTGAG
AACTCCGTGGCTTACTCCAACAACTCCATCGCTATCCCCACCAACTTCACCATCTCCGTG
ACCACCGAGATCCTGCCCGTGTCCATGACCAAGACCTCCGTGGACTGCACCATGTACATC
TGCGGTGACTCCACCGAGTGCTCCAACCTGCTGCTGCAGTACGGTTCCTTCTGCACCCAG
CTGAACCGTGCTCTGACCGGTATCGCTGTGGAGCAGGACAAGAACACCCAGGAGGTGTTC
GCTCAGGTGAAGCAGATCTACAAGACCCCCCCCATCAAGGACTTCGGTGGTTTCAACTTC
TCCCAGATCCTGCCCGACCCCTCCAAGCCCTCCAAGCGTTCCTTCATCGAGGACCTGCTG
TTCAACAAGGTGACCCTGGCTGACGCTGGTTTCATCAAGCAGTACGGTGACTGCCTGGGT
GACATCGCTGCTCGTGACCTGATCTGCGCTCAGAAGTTCAACGGTCTGACCGTGCTGCCC
CCCCTGCTGACCGACGAGATGATCGCTCAGTACACCTCCGCTCTGCTGGCTGGTACCATC
ACCTCCGGTTGGACCTTCGGTGCTGGTGCTGCTCTGCAGATCCCCTTCGCTATGCAGATG
GCTTACCGTTTCAACGGTATCGGTGTGACCCAGAACGTGCTGTACGAGAACCAGAAGCTG
ATCGCTAACCAGTTCAACTCCGCTATCGGTAAGATCCAGGACTCCCTGTCCTCCACCGCT
TCCGCTCTGGGTAAGCTGCAGAACGTGGTGAACCAGAACGCTCAGGCTCTGAACACCCTG
GTGAAGCAGCTGTCCTCCAACTTCGGTGCTATCTCCTCCGTGCTGAACGACATCCTGTCC
CGTCTGGACAAGGTGGAGGCTGAGGTGCAGATCGACCGTCTGATCACCGGTCGTCTGCAG
TCCCTGCAGACCTACGTGACCCAGCAGCTGATCCGTGCTGCTGAGATCCGTGCTTCCGCT
AACCTGGCTGCTACCAAGATGTCCGAGTGCGTGCTGGGTCAGTCCAAGCGTGTGGACTTC
TGCGGTAAGGGTTACCACCTGATGTCCTTCCCCCAGTCCGCTCCCCACGGTGTGGTGTTC
CTGCACGTGACCTACGTGCCCGCTCAGGAGAAGAACTTCACCACCGCTCCCGCTATCTGC
CACGACGGTAAGGCTCACTTCCCCCGTGAGGGTGTGTTCGTGTCCAACGGTACCCACTGG
TTCGTGACCCAGCGTAACTTCTACGAGCCCCAGATCATCACCACCGACAACACCTTCGTG
TCCGGTAACTGCGACGTGGTGATCGGTATCGTGAACAACACCGTGTACGACCCCCTGCAG
CCCGAGCTGGACTCCTTCAAGGAGGAGCTGGACAAGTACTTCAAGAACCACACCTCCCCC
GACGTGGACCTGGGTGACATCTCCGGTATCAACGCTTCCGTGGTGAACATCCAGAAGGAG
ATCGACCGTCTGAACGAGGTGGCTAAGAACCTGAACGAGTCCCTGATCGACCTGCAGGAG
CTGGGTAAGTACGAGCAGTACATCAAGTGGCCCTAG
Note:
the above sequence consists of signal peptide-extracellular domain.
SEQ ID NO: 58:
Full-length amino acid sequence of Omicron-BA.1 S antigen:
MFVFLVLLPLVSSQCVNLTTRTQLPPAYTNSFTRGVYYPDKVFRSSVLHSTQDLFLPFFS
NVTWFHVISGTNGTKRFDNPVLPFNDGVYFASIEKSNIIRGWIFGTTLDSKTQSLLIVNN
ATNVVIKVCEFQFCNDPFLDHKNNKSWMESEFRVYSSANNCTFEYVSQPFLMDLEGKQGN
FKNLREFVFKNIDGYFKIYSKHTPIIVRDLPQGFSALEPLVDLPIGINITRFQTLLALHR
SYLTPGDSSSGWTAGAAAYYVGYLQPRTFLLKYNENGTITDAVDCALDPLSETKCTLKSF
TVEKGIYQTSNFRVQPTESIVRFPNITNLCPFDEVFNATRFASVYAWNRKRISNCVADYS
VLYNLAPFFTFKCYGVSPTKLNDLCFTNVYADSFVIRGDEVRQIAPGQTGNIADYNYKLP
DDFTGCVIAWNSNKLDSKVSGNYNYLYRLFRKSNLKPFERDISTEIYQAGNKPCNGVAGF
NCYFPLRSYSFRPTYGVGHQPYRVVVLSFELLHAPATVCGPKKSTNLVKNKCVNFNFNGL
KGTGVLTESNKKFLPFQQFGRDIADTTDAVRDPQTLEILDITPCSFGGVSVITPGTNTSN
QVAVLYQGVNCTEVPVAIHADQLTPTWRVYSTGSNVFQTRAGCLIGAEYVNNSYECDIPI
GAGICASYQTQTKSHRRARSVASQSIIAYTMSLGAENSVAYSNNSIAIPTNFTISVTTEI
LPVSMTKTSVDCTMYICGDSTECSNLLLQYGSFCTQLKRALTGIAVEQDKNTQEVFAQVK
QIYKTPPIKYFGGFNFSQILPDPSKPSKRSFIEDLLFNKVTLADAGFIKQYGDCLGDIAA
RDLICAQKFKGLTVLPPLLTDEMIAQYTSALLAGTITSGWTFGAGAALQIPFAMQMAYRE
NGIGVTQNVLYENQKLIANQFNSAIGKIQDSLSSTASALGKLQDVVNHNAQALNTLVKQL
SSKFGAISSVLNDIFSRLDKVEAEVQIDRLITGRLQSLQTYVTQQLIRAAEIRASANLAA
TKMSECVLGQSKRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNNTVYDPLQPELD
SFKEELDKYFKNHTSPDVDLGDISGINASVVNIQKEIDRLNEVAKNLNESLIDLQELGKY
EQYIKWPWYIWLGFIAGLIAIVMVTIMLCCMTSCCSCLKGCCSCGSCCKFDEDDSEPVLK
GVKLHYT
Note:
the above sequence consists of signal peptide-extracellular domain-
transmembrane domain-intracellular domain.
SEQ ID NO: 59:
Full-length nucleic acid sequence of Omicron-BA.1 S antigen
(optimization of CHO cells):
ATGTTCGTGTTCCTGGTGCTGCTGCCCCTGGTGAGCAGCCAGTGCGTGAACCTGACCACC
AGAACCCAGCTGCCCCCCGCCTACACCAACAGCTTCACCAGAGGCGTGTACTACCCCGAC
AAGGTGTTCAGAAGCAGCGTGCTGCACAGCACCCAGGACCTGTTCCTGCCCTTCTTCAGC
AACGTGACCTGGTTCCACGTGATCAGCGGCACCAACGGCACCAAGAGATTCGACAACCCC
GTGCTGCCCTTCAACGACGGCGTGTACTTCGCCAGCATCGAGAAGAGCAACATCATCAGA
GGCTGGATCTTCGGCACCACCCTGGACAGCAAGACCCAGAGCCTGCTGATCGTGAACAAC
GCCACCAACGTGGTGATCAAGGTGTGCGAGTTCCAGTTCTGCAACGACCCCTTCCTGGAC
CACAAGAACAACAAGAGCTGGATGGAGAGCGAGTTCAGAGTGTACAGCAGCGCCAACAAC
TGCACCTTCGAGTACGTGAGCCAGCCCTTCCTGATGGACCTGGAGGGCAAGCAGGGCAAC
TTCAAGAACCTGAGAGAGTTCGTGTTCAAGAACATCGACGGCTACTTCAAGATCTACAGC
AAGCACACCCCCATCATCGTGAGAGACCTGCCCCAGGGCTTCAGCGCCCTGGAGCCCCTG
GTGGACCTGCCCATCGGCATCAACATCACCAGATTCCAGACCCTGCTGGCCCTGCACAGA
AGCTACCTGACCCCCGGCGACAGCAGCAGCGGCTGGACCGCCGGCGCCGCCGCCTACTAC
GTGGGCTACCTGCAGCCCAGAACCTTCCTGCTGAAGTACAACGAGAACGGCACCATCACC
GACGCCGTGGACTGCGCCCTGGACCCCCTGAGCGAGACCAAGTGCACCCTGAAGAGCTTC
ACCGTGGAGAAGGGCATCTACCAGACCAGCAACTTCAGAGTGCAGCCCACCGAGAGCATC
GTGAGATTCCCCAACATCACCAACCTGTGCCCCTTCGACGAGGTGTTCAACGCCACCAGA
TTCGCCAGCGTGTACGCCTGGAACAGAAAGAGAATCAGCAACTGCGTGGCCGACTACAGC
GTGCTGTACAACCTGGCCCCCTTCTTCACCTTCAAGTGCTACGGCGTGAGCCCCACCAAG
CTGAACGACCTGTGCTTCACCAACGTGTACGCCGACAGCTTCGTGATCAGAGGCGACGAG
GTGAGACAGATCGCCCCCGGCCAGACCGGCAACATCGCCGACTACAACTACAAGCTGCCC
GACGACTTCACCGGCTGCGTGATCGCCTGGAACAGCAACAAGCTGGACAGCAAGGTGAGC
GGCAACTACAACTACCTGTACAGACTGTTCAGAAAGAGCAACCTGAAGCCCTTCGAGAGA
GACATCAGCACCGAGATCTACCAGGCCGGCAACAAGCCCTGCAACGGCGTGGCCGGCTTC
AACTGCTACTTCCCCCTGAGAAGCTACAGCTTCAGACCCACCTACGGCGTGGGCCACCAG
CCCTACAGAGTGGTGGTGCTGAGCTTCGAGCTGCTGCACGCCCCCGCCACCGTGTGCGGC
CCCAAGAAGAGCACCAACCTGGTGAAGAACAAGTGCGTGAACTTCAACTTCAACGGCCTG
AAGGGCACCGGCGTGCTGACCGAGAGCAACAAGAAGTTCCTGCCCTTCCAGCAGTTCGGC
AGAGACATCGCCGACACCACCGACGCCGTGAGAGACCCCCAGACCCTGGAGATCCTGGAC
ATCACCCCCTGCAGCTTCGGCGGCGTGAGCGTGATCACCCCCGGCACCAACACCAGCAAC
CAGGTGGCCGTGCTGTACCAGGGCGTGAACTGCACCGAGGTGCCCGTGGCCATCCACGCC
GACCAGCTGACCCCCACCTGGAGAGTGTACAGCACCGGCAGCAACGTGTTCCAGACCAGA
GCCGGCTGCCTGATCGGCGCCGAGTACGTGAACAACAGCTACGAGTGCGACATCCCCATC
GGCGCCGGCATCTGCGCCAGCTACCAGACCCAGACCAAGAGCCACAGAAGAGCCAGAAGC
GTGGCCAGCCAGAGCATCATCGCCTACACCATGAGCCTGGGCGCCGAGAACAGCGTGGCC
TACAGCAACAACAGCATCGCCATCCCCACCAACTTCACCATCAGCGTGACCACCGAGATC
CTGCCCGTGAGCATGACCAAGACCAGCGTGGACTGCACCATGTACATCTGCGGCGACAGC
ACCGAGTGCAGCAACCTGCTGCTGCAGTACGGCAGCTTCTGCACCCAGCTGAAGAGAGCC
CTGACCGGCATCGCCGTGGAGCAGGACAAGAACACCCAGGAGGTGTTCGCCCAGGTGAAG
CAGATCTACAAGACCCCCCCCATCAAGTACTTCGGCGGCTTCAACTTCAGCCAGATCCTG
CCCGACCCCAGCAAGCCCAGCAAGAGAAGCTTCATCGAGGACCTGCTGTTCAACAAGGTG
ACCCTGGCCGACGCCGGCTTCATCAAGCAGTACGGCGACTGCCTGGGCGACATCGCCGCC
AGAGACCTGATCTGCGCCCAGAAGTTCAAGGGCCTGACCGTGCTGCCCCCCCTGCTGACC
GACGAGATGATCGCCCAGTACACCAGCGCCCTGCTGGCCGGCACCATCACCAGCGGCTGG
ACCTTCGGCGCCGGCGCCGCCCTGCAGATCCCCTTCGCCATGCAGATGGCCTACAGATTC
AACGGCATCGGCGTGACCCAGAACGTGCTGTACGAGAACCAGAAGCTGATCGCCAACCAG
TTCAACAGCGCCATCGGCAAGATCCAGGACAGCCTGAGCAGCACCGCCAGCGCCCTGGGC
AAGCTGCAGGACGTGGTGAACCACAACGCCCAGGCCCTGAACACCCTGGTGAAGCAGCTG
AGCAGCAAGTTCGGCGCCATCAGCAGCGTGCTGAACGACATCTTCAGCAGACTGGACAAG
GTGGAGGCCGAGGTGCAGATCGACAGACTGATCACCGGCAGACTGCAGAGCCTGCAGACC
TACGTGACCCAGCAGCTGATCAGAGCCGCCGAGATCAGAGCCAGCGCCAACCTGGCCGCC
ACCAAGATGAGCGAGTGCGTGCTGGGCCAGAGCAAGAGAGTGGACTTCTGCGGCAAGGGC
TACCACCTGATGAGCTTCCCCCAGAGCGCCCCCCACGGCGTGGTGTTCCTGCACGTGACC
TACGTGCCCGCCCAGGAGAAGAACTTCACCACCGCCCCCGCCATCTGCCACGACGGCAAG
GCCCACTTCCCCAGAGAGGGCGTGTTCGTGAGCAACGGCACCCACTGGTTCGTGACCCAG
AGAAACTTCTACGAGCCCCAGATCATCACCACCGACAACACCTTCGTGAGCGGCAACTGC
GACGTGGTGATCGGCATCGTGAACAACACCGTGTACGACCCCCTGCAGCCCGAGCTGGAC
AGCTTCAAGGAGGAGCTGGACAAGTACTTCAAGAACCACACCAGCCCCGACGTGGACCTG
GGCGACATCAGCGGCATCAACGCCAGCGTGGTGAACATCCAGAAGGAGATCGACAGACTG
AACGAGGTGGCCAAGAACCTGAACGAGAGCCTGATCGACCTGCAGGAGCTGGGCAAGTAC
GAGCAGTACATCAAGTGGCCCTGGTACATCTGGCTGGGCTTCATCGCCGGCCTGATCGCC
ATCGTGATGGTGACCATCATGCTGTGCTGCATGACCAGCTGCTGCAGCTGCCTGAAGGGC
TGCTGCAGCTGCGGCAGCTGCTGCAAGTTCGACGAGGACGACAGCGAGCCCGTGCTGAAG
GGCGTGAAGCTGCACTACACCTAG
Note:
the above sequence consists of signal peptide-extracellular domain-
transmembrane domain-intracellular domain.
SEQ ID NO: 60:
Full-length nucleic acid sequence of Omicron-BA.1 S antigen
(optimization of insect cells):
ATGTTCGTGTTCCTGGTGCTGCTGCCCCTGGTGTCCTCCCAGTGCGTGAACCTGACCACC
CGTACCCAGCTGCCCCCCGCTTACACCAACTCCTTCACCCGTGGTGTGTACTACCCCGAC
AAGGTGTTCCGTTCCTCCGTGCTGCACTCCACCCAGGACCTGTTCCTGCCCTTCTTCTCC
AACGTGACCTGGTTCCACGTGATCTCCGGTACCAACGGTACCAAGCGTTTCGACAACCCC
GTGCTGCCCTTCAACGACGGTGTGTACTTCGCTTCCATCGAGAAGTCCAACATCATCCGT
GGTTGGATCTTCGGTACCACCCTGGACTCCAAGACCCAGTCCCTGCTGATCGTGAACAAC
GCTACCAACGTGGTGATCAAGGTGTGCGAGTTCCAGTTCTGCAACGACCCCTTCCTGGAC
CACAAGAACAACAAGTCCTGGATGGAGTCCGAGTTCCGTGTGTACTCCTCCGCTAACAAC
TGCACCTTCGAGTACGTGTCCCAGCCCTTCCTGATGGACCTGGAGGGTAAGCAGGGTAAC
TTCAAGAACCTGCGTGAGTTCGTGTTCAAGAACATCGACGGTTACTTCAAGATCTACTCC
AAGCACACCCCCATCATCGTGCGTGACCTGCCCCAGGGTTTCTCCGCTCTGGAGCCCCTG
GTGGACCTGCCCATCGGTATCAACATCACCCGTTTCCAGACCCTGCTGGCTCTGCACCGT
TCCTACCTGACCCCCGGTGACTCCTCCTCCGGTTGGACCGCTGGTGCTGCTGCTTACTAC
GTGGGTTACCTGCAGCCCCGTACCTTCCTGCTGAAGTACAACGAGAACGGTACCATCACC
GACGCTGTGGACTGCGCTCTGGACCCCCTGTCCGAGACCAAGTGCACCCTGAAGTCCTTC
ACCGTGGAGAAGGGTATCTACCAGACCTCCAACTTCCGTGTGCAGCCCACCGAGTCCATC
GTGCGTTTCCCCAACATCACCAACCTGTGCCCCTTCGACGAGGTGTTCAACGCTACCCGT
TTCGCTTCCGTGTACGCTTGGAACCGTAAGCGTATCTCCAACTGCGTGGCTGACTACTCC
GTGCTGTACAACCTGGCTCCCTTCTTCACCTTCAAGTGCTACGGTGTGTCCCCCACCAAG
CTGAACGACCTGTGCTTCACCAACGTGTACGCTGACTCCTTCGTGATCCGTGGTGACGAG
GTGCGTCAGATCGCTCCCGGTCAGACCGGTAACATCGCTGACTACAACTACAAGCTGCCC
GACGACTTCACCGGTTGCGTGATCGCTTGGAACTCCAACAAGCTGGACTCCAAGGTGTCC
GGTAACTACAACTACCTGTACCGTCTGTTCCGTAAGTCCAACCTGAAGCCCTTCGAGCGT
GACATCTCCACCGAGATCTACCAGGCTGGTAACAAGCCCTGCAACGGTGTGGCTGGTTTC
AACTGCTACTTCCCCCTGCGTTCCTACTCCTTCCGTCCCACCTACGGTGTGGGTCACCAG
CCCTACCGTGTGGTGGTGCTGTCCTTCGAGCTGCTGCACGCTCCCGCTACCGTGTGCGGT
CCCAAGAAGTCCACCAACCTGGTGAAGAACAAGTGCGTGAACTTCAACTTCAACGGTCTG
AAGGGTACCGGTGTGCTGACCGAGTCCAACAAGAAGTTCCTGCCCTTCCAGCAGTTCGGT
CGTGACATCGCTGACACCACCGACGCTGTGCGTGACCCCCAGACCCTGGAGATCCTGGAC
ATCACCCCCTGCTCCTTCGGTGGTGTGTCCGTGATCACCCCCGGTACCAACACCTCCAAC
CAGGTGGCTGTGCTGTACCAGGGTGTGAACTGCACCGAGGTGCCCGTGGCTATCCACGCT
GACCAGCTGACCCCCACCTGGCGTGTGTACTCCACCGGTTCCAACGTGTTCCAGACCCGT
GCTGGTTGCCTGATCGGTGCTGAGTACGTGAACAACTCCTACGAGTGCGACATCCCCATC
GGTGCTGGTATCTGCGCTTCCTACCAGACCCAGACCAAGTCCCACCGTCGTGCTCGTTCC
GTGGCTTCCCAGTCCATCATCGCTTACACCATGTCCCTGGGTGCTGAGAACTCCGTGGCT
TACTCCAACAACTCCATCGCTATCCCCACCAACTTCACCATCTCCGTGACCACCGAGATC
CTGCCCGTGTCCATGACCAAGACCTCCGTGGACTGCACCATGTACATCTGCGGTGACTCC
ACCGAGTGCTCCAACCTGCTGCTGCAGTACGGTTCCTTCTGCACCCAGCTGAAGCGTGCT
CTGACCGGTATCGCTGTGGAGCAGGACAAGAACACCCAGGAGGTGTTCGCTCAGGTGAAG
CAGATCTACAAGACCCCCCCCATCAAGTACTTCGGTGGTTTCAACTTCTCCCAGATCCTG
CCCGACCCCTCCAAGCCCTCCAAGCGTTCCTTCATCGAGGACCTGCTGTTCAACAAGGTG
ACCCTGGCTGACGCTGGTTTCATCAAGCAGTACGGTGACTGCCTGGGTGACATCGCTGCT
CGTGACCTGATCTGCGCTCAGAAGTTCAAGGGTCTGACCGTGCTGCCCCCCCTGCTGACC
GACGAGATGATCGCTCAGTACACCTCCGCTCTGCTGGCTGGTACCATCACCTCCGGTTGG
ACCTTCGGTGCTGGTGCTGCTCTGCAGATCCCCTTCGCTATGCAGATGGCTTACCGTTTC
AACGGTATCGGTGTGACCCAGAACGTGCTGTACGAGAACCAGAAGCTGATCGCTAACCAG
TTCAACTCCGCTATCGGTAAGATCCAGGACTCCCTGTCCTCCACCGCTTCCGCTCTGGGT
AAGCTGCAGGACGTGGTGAACCACAACGCTCAGGCTCTGAACACCCTGGTGAAGCAGCTG
TCCTCCAAGTTCGGTGCTATCTCCTCCGTGCTGAACGACATCTTCTCCCGTCTGGACAAG
GTGGAGGCTGAGGTGCAGATCGACCGTCTGATCACCGGTCGTCTGCAGTCCCTGCAGACC
TACGTGACCCAGCAGCTGATCCGTGCTGCTGAGATCCGTGCTTCCGCTAACCTGGCTGCT
ACCAAGATGTCCGAGTGCGTGCTGGGTCAGTCCAAGCGTGTGGACTTCTGCGGTAAGGGT
TACCACCTGATGTCCTTCCCCCAGTCCGCTCCCCACGGTGTGGTGTTCCTGCACGTGACC
TACGTGCCCGCTCAGGAGAAGAACTTCACCACCGCTCCCGCTATCTGCCACGACGGTAAG
GCTCACTTCCCCCGTGAGGGTGTGTTCGTGTCCAACGGTACCCACTGGTTCGTGACCCAG
CGTAACTTCTACGAGCCCCAGATCATCACCACCGACAACACCTTCGTGTCCGGTAACTGC
GACGTGGTGATCGGTATCGTGAACAACACCGTGTACGACCCCCTGCAGCCCGAGCTGGAC
TCCTTCAAGGAGGAGCTGGACAAGTACTTCAAGAACCACACCTCCCCCGACGTGGACCTG
GGTGACATCTCCGGTATCAACGCTTCCGTGGTGAACATCCAGAAGGAGATCGACCGTCTG
AACGAGGTGGCTAAGAACCTGAACGAGTCCCTGATCGACCTGCAGGAGCTGGGTAAGTAC
GAGCAGTACATCAAGTGGCCCTGGTACATCTGGCTGGGTTTCATCGCTGGTCTGATCGCT
ATCGTGATGGTGACCATCATGCTGTGCTGCATGACCTCCTGCTGCTCCTGCCTGAAGGGT
TGCTGCTCCTGCGGTTCCTGCTGCAAGTTCGACGAGGACGACTCCGAGCCCGTGCTGAAG
GGTGTGAAGCTGCACTACACCTAG
Note:
the above sequence consists of signal peptide-extracellular domain-
transmembrane domain-intracellular domain.
SEQ ID NO: 61:
Extracellular amino acid sequence of Omicron-BA.1 S antigen:
MFVFLVLLPLVSSQCVNLTTRTQLPPAYTNSFTRGVYYPDKVFRSSVLHSTQDLFLPFFS
NVTWFHVISGTNGTKRFDNPVLPFNDGVYFASIEKSNIIRGWIFGTTLDSKTQSLLIVNN
ATNVVIKVCEFQFCNDPFLDHKNNKSWMESEFRVYSSANNCTFEYVSQPFLMDLEGKQGN
FKNLREFVFKNIDGYFKIYSKHTPIIVRDLPQGFSALEPLVDLPIGINITRFQTLLALHR
SYLTPGDSSSGWTAGAAAYYVGYLQPRTFLLKYNENGTITDAVDCALDPLSETKCTLKSF
TVEKGIYQTSNFRVQPTESIVRFPNITNLCPFDEVFNATRFASVYAWNRKRISNCVADYS
VLYNLAPFFTFKCYGVSPTKLNDLCFTNVYADSFVIRGDEVRQIAPGQTGNIADYNYKLP
DDFTGCVIAWNSNKLDSKVSGNYNYLYRLFRKSNLKPFERDISTEIYQAGNKPCNGVAGF
NCYFPLRSYSFRPTYGVGHQPYRVVVLSFELLHAPATVCGPKKSTNLVKNKCVNFNFNGL
KGTGVLTESNKKFLPFQQFGRDIADTTDAVRDPQTLEILDITPCSFGGVSVITPGTNTSN
QVAVLYQGVNCTEVPVAIHADQLTPTWRVYSTGSNVFQTRAGCLIGAEYVNNSYECDIPI
GAGICASYQTQTKSHRRARSVASQSIIAYTMSLGAENSVAYSNNSIAIPTNFTISVTTEI
LPVSMTKTSVDCTMYICGDSTECSNLLLQYGSFCTQLKRALTGIAVEQDKNTQEVFAQVK
QIYKTPPIKYFGGFNFSQILPDPSKPSKRSFIEDLLFNKVTLADAGFIKQYGDCLGDIAA
RDLICAQKFKGLTVLPPLLTDEMIAQYTSALLAGTITSGWTFGAGAALQIPFAMQMAYRE
NGIGVTQNVLYENQKLIANQFNSAIGKIQDSLSSTASALGKLQDVVNHNAQALNTLVKQL
SSKFGAISSVLNDIFSRLDKVEAEVQIDRLITGRLQSLQTYVTQQLIRAAEIRASANLAA
TKMSECVLGQSKRVDFCGKGYHLMSFPQSAPHGVVFLHVTYVPAQEKNFTTAPAICHDGK
AHFPREGVFVSNGTHWFVTQRNFYEPQIITTDNTFVSGNCDVVIGIVNNTVYDPLQPELD
SFKEELDKYFKNHTSPDVDLGDISGINASVVNIQKEIDRLNEVAKNLNESLIDLQELGKY
EQYIKWP
Note:
the above sequence consists of signal peptide-extracellular domain.
SEQ ID NO: 62:
Extracellular nucleic acid sequence of Omicron-BA.1 S antigen
(optimization of CHO cells):
ATGTTCGTGTTCCTGGTGCTGCTGCCCCTGGTGAGCAGCCAGTGCGTGAACCTGACCACC
AGAACCCAGCTGCCCCCCGCCTACACCAACAGCTTCACCAGAGGCGTGTACTACCCCGAC
AAGGTGTTCAGAAGCAGCGTGCTGCACAGCACCCAGGACCTGTTCCTGCCCTTCTTCAGC
AACGTGACCTGGTTCCACGTGATCAGCGGCACCAACGGCACCAAGAGATTCGACAACCCC
GTGCTGCCCTTCAACGACGGCGTGTACTTCGCCAGCATCGAGAAGAGCAACATCATCAGA
GGCTGGATCTTCGGCACCACCCTGGACAGCAAGACCCAGAGCCTGCTGATCGTGAACAAC
GCCACCAACGTGGTGATCAAGGTGTGCGAGTTCCAGTTCTGCAACGACCCCTTCCTGGAC
CACAAGAACAACAAGAGCTGGATGGAGAGCGAGTTCAGAGTGTACAGCAGCGCCAACAAC
TGCACCTTCGAGTACGTGAGCCAGCCCTTCCTGATGGACCTGGAGGGCAAGCAGGGCAAC
TTCAAGAACCTGAGAGAGTTCGTGTTCAAGAACATCGACGGCTACTTCAAGATCTACAGC
AAGCACACCCCCATCATCGTGAGAGACCTGCCCCAGGGCTTCAGCGCCCTGGAGCCCCTG
GTGGACCTGCCCATCGGCATCAACATCACCAGATTCCAGACCCTGCTGGCCCTGCACAGA
AGCTACCTGACCCCCGGCGACAGCAGCAGCGGCTGGACCGCCGGCGCCGCCGCCTACTAC
GTGGGCTACCTGCAGCCCAGAACCTTCCTGCTGAAGTACAACGAGAACGGCACCATCACC
GACGCCGTGGACTGCGCCCTGGACCCCCTGAGCGAGACCAAGTGCACCCTGAAGAGCTTC
ACCGTGGAGAAGGGCATCTACCAGACCAGCAACTTCAGAGTGCAGCCCACCGAGAGCATC
GTGAGATTCCCCAACATCACCAACCTGTGCCCCTTCGACGAGGTGTTCAACGCCACCAGA
TTCGCCAGCGTGTACGCCTGGAACAGAAAGAGAATCAGCAACTGCGTGGCCGACTACAGC
GTGCTGTACAACCTGGCCCCCTTCTTCACCTTCAAGTGCTACGGCGTGAGCCCCACCAAG
CTGAACGACCTGTGCTTCACCAACGTGTACGCCGACAGCTTCGTGATCAGAGGCGACGAG
GTGAGACAGATCGCCCCCGGCCAGACCGGCAACATCGCCGACTACAACTACAAGCTGCCC
GACGACTTCACCGGCTGCGTGATCGCCTGGAACAGCAACAAGCTGGACAGCAAGGTGAGC
GGCAACTACAACTACCTGTACAGACTGTTCAGAAAGAGCAACCTGAAGCCCTTCGAGAGA
GACATCAGCACCGAGATCTACCAGGCCGGCAACAAGCCCTGCAACGGCGTGGCCGGCTTC
AACTGCTACTTCCCCCTGAGAAGCTACAGCTTCAGACCCACCTACGGCGTGGGCCACCAG
CCCTACAGAGTGGTGGTGCTGAGCTTCGAGCTGCTGCACGCCCCCGCCACCGTGTGCGGC
CCCAAGAAGAGCACCAACCTGGTGAAGAACAAGTGCGTGAACTTCAACTTCAACGGCCTG
AAGGGCACCGGCGTGCTGACCGAGAGCAACAAGAAGTTCCTGCCCTTCCAGCAGTTCGGC
AGAGACATCGCCGACACCACCGACGCCGTGAGAGACCCCCAGACCCTGGAGATCCTGGAC
ATCACCCCCTGCAGCTTCGGCGGCGTGAGCGTGATCACCCCCGGCACCAACACCAGCAAC
CAGGTGGCCGTGCTGTACCAGGGCGTGAACTGCACCGAGGTGCCCGTGGCCATCCACGCC
GACCAGCTGACCCCCACCTGGAGAGTGTACAGCACCGGCAGCAACGTGTTCCAGACCAGA
GCCGGCTGCCTGATCGGCGCCGAGTACGTGAACAACAGCTACGAGTGCGACATCCCCATC
GGCGCCGGCATCTGCGCCAGCTACCAGACCCAGACCAAGAGCCACAGAAGAGCCAGAAGC
GTGGCCAGCCAGAGCATCATCGCCTACACCATGAGCCTGGGCGCCGAGAACAGCGTGGCC
TACAGCAACAACAGCATCGCCATCCCCACCAACTTCACCATCAGCGTGACCACCGAGATC
CTGCCCGTGAGCATGACCAAGACCAGCGTGGACTGCACCATGTACATCTGCGGCGACAGC
ACCGAGTGCAGCAACCTGCTGCTGCAGTACGGCAGCTTCTGCACCCAGCTGAAGAGAGCC
CTGACCGGCATCGCCGTGGAGCAGGACAAGAACACCCAGGAGGTGTTCGCCCAGGTGAAG
CAGATCTACAAGACCCCCCCCATCAAGTACTTCGGCGGCTTCAACTTCAGCCAGATCCTG
CCCGACCCCAGCAAGCCCAGCAAGAGAAGCTTCATCGAGGACCTGCTGTTCAACAAGGTG
ACCCTGGCCGACGCCGGCTTCATCAAGCAGTACGGCGACTGCCTGGGCGACATCGCCGCC
AGAGACCTGATCTGCGCCCAGAAGTTCAAGGGCCTGACCGTGCTGCCCCCCCTGCTGACC
GACGAGATGATCGCCCAGTACACCAGCGCCCTGCTGGCCGGCACCATCACCAGCGGCTGG
ACCTTCGGCGCCGGCGCCGCCCTGCAGATCCCCTTCGCCATGCAGATGGCCTACAGATTC
AACGGCATCGGCGTGACCCAGAACGTGCTGTACGAGAACCAGAAGCTGATCGCCAACCAG
TTCAACAGCGCCATCGGCAAGATCCAGGACAGCCTGAGCAGCACCGCCAGCGCCCTGGGC
AAGCTGCAGGACGTGGTGAACCACAACGCCCAGGCCCTGAACACCCTGGTGAAGCAGCTG
AGCAGCAAGTTCGGCGCCATCAGCAGCGTGCTGAACGACATCTTCAGCAGACTGGACAAG
GTGGAGGCCGAGGTGCAGATCGACAGACTGATCACCGGCAGACTGCAGAGCCTGCAGACC
TACGTGACCCAGCAGCTGATCAGAGCCGCCGAGATCAGAGCCAGCGCCAACCTGGCCGCC
ACCAAGATGAGCGAGTGCGTGCTGGGCCAGAGCAAGAGAGTGGACTTCTGCGGCAAGGGC
TACCACCTGATGAGCTTCCCCCAGAGCGCCCCCCACGGCGTGGTGTTCCTGCACGTGACC
TACGTGCCCGCCCAGGAGAAGAACTTCACCACCGCCCCCGCCATCTGCCACGACGGCAAG
GCCCACTTCCCCAGAGAGGGCGTGTTCGTGAGCAACGGCACCCACTGGTTCGTGACCCAG
AGAAACTTCTACGAGCCCCAGATCATCACCACCGACAACACCTTCGTGAGCGGCAACTGC
GACGTGGTGATCGGCATCGTGAACAACACCGTGTACGACCCCCTGCAGCCCGAGCTGGAC
AGCTTCAAGGAGGAGCTGGACAAGTACTTCAAGAACCACACCAGCCCCGACGTGGACCTG
GGCGACATCAGCGGCATCAACGCCAGCGTGGTGAACATCCAGAAGGAGATCGACAGACTG
AACGAGGTGGCCAAGAACCTGAACGAGAGCCTGATCGACCTGCAGGAGCTGGGCAAGTAC
GAGCAGTACATCAAGTGGCCCTAG
Note:
the above sequence consists of signal peptide-extracellular domain.
SEQ ID NO: 63:
Extracellular nucleic acid sequence of Omicron-BA.1 S antigen
(optimization of insect cells):
ATGTTCGTGTTCCTGGTGCTGCTGCCCCTGGTGTCCTCCCAGTGCGTGAACCTGACCACC
CGTACCCAGCTGCCCCCCGCTTACACCAACTCCTTCACCCGTGGTGTGTACTACCCCGAC
AAGGTGTTCCGTTCCTCCGTGCTGCACTCCACCCAGGACCTGTTCCTGCCCTTCTTCTCC
AACGTGACCTGGTTCCACGTGATCTCCGGTACCAACGGTACCAAGCGTTTCGACAACCCC
GTGCTGCCCTTCAACGACGGTGTGTACTTCGCTTCCATCGAGAAGTCCAACATCATCCGT
GGTTGGATCTTCGGTACCACCCTGGACTCCAAGACCCAGTCCCTGCTGATCGTGAACAAC
GCTACCAACGTGGTGATCAAGGTGTGCGAGTTCCAGTTCTGCAACGACCCCTTCCTGGAC
CACAAGAACAACAAGTCCTGGATGGAGTCCGAGTTCCGTGTGTACTCCTCCGCTAACAAC
TGCACCTTCGAGTACGTGTCCCAGCCCTTCCTGATGGACCTGGAGGGTAAGCAGGGTAAC
TTCAAGAACCTGCGTGAGTTCGTGTTCAAGAACATCGACGGTTACTTCAAGATCTACTCC
AAGCACACCCCCATCATCGTGCGTGACCTGCCCCAGGGTTTCTCCGCTCTGGAGCCCCTG
GTGGACCTGCCCATCGGTATCAACATCACCCGTTTCCAGACCCTGCTGGCTCTGCACCGT
TCCTACCTGACCCCCGGTGACTCCTCCTCCGGTTGGACCGCTGGTGCTGCTGCTTACTAC
GTGGGTTACCTGCAGCCCCGTACCTTCCTGCTGAAGTACAACGAGAACGGTACCATCACC
GACGCTGTGGACTGCGCTCTGGACCCCCTGTCCGAGACCAAGTGCACCCTGAAGTCCTTC
ACCGTGGAGAAGGGTATCTACCAGACCTCCAACTTCCGTGTGCAGCCCACCGAGTCCATC
GTGCGTTTCCCCAACATCACCAACCTGTGCCCCTTCGACGAGGTGTTCAACGCTACCCGT
TTCGCTTCCGTGTACGCTTGGAACCGTAAGCGTATCTCCAACTGCGTGGCTGACTACTCC
GTGCTGTACAACCTGGCTCCCTTCTTCACCTTCAAGTGCTACGGTGTGTCCCCCACCAAG
CTGAACGACCTGTGCTTCACCAACGTGTACGCTGACTCCTTCGTGATCCGTGGTGACGAG
GTGCGTCAGATCGCTCCCGGTCAGACCGGTAACATCGCTGACTACAACTACAAGCTGCCC
GACGACTTCACCGGTTGCGTGATCGCTTGGAACTCCAACAAGCTGGACTCCAAGGTGTCC
GGTAACTACAACTACCTGTACCGTCTGTTCCGTAAGTCCAACCTGAAGCCCTTCGAGCGT
GACATCTCCACCGAGATCTACCAGGCTGGTAACAAGCCCTGCAACGGTGTGGCTGGTTTC
AACTGCTACTTCCCCCTGCGTTCCTACTCCTTCCGTCCCACCTACGGTGTGGGTCACCAG
CCCTACCGTGTGGTGGTGCTGTCCTTCGAGCTGCTGCACGCTCCCGCTACCGTGTGCGGT
CCCAAGAAGTCCACCAACCTGGTGAAGAACAAGTGCGTGAACTTCAACTTCAACGGTCTG
AAGGGTACCGGTGTGCTGACCGAGTCCAACAAGAAGTTCCTGCCCTTCCAGCAGTTCGGT
CGTGACATCGCTGACACCACCGACGCTGTGCGTGACCCCCAGACCCTGGAGATCCTGGAC
ATCACCCCCTGCTCCTTCGGTGGTGTGTCCGTGATCACCCCCGGTACCAACACCTCCAAC
CAGGTGGCTGTGCTGTACCAGGGTGTGAACTGCACCGAGGTGCCCGTGGCTATCCACGCT
GACCAGCTGACCCCCACCTGGCGTGTGTACTCCACCGGTTCCAACGTGTTCCAGACCCGT
GCTGGTTGCCTGATCGGTGCTGAGTACGTGAACAACTCCTACGAGTGCGACATCCCCATC
GGTGCTGGTATCTGCGCTTCCTACCAGACCCAGACCAAGTCCCACCGTCGTGCTCGTTCC
GTGGCTTCCCAGTCCATCATCGCTTACACCATGTCCCTGGGTGCTGAGAACTCCGTGGCT
TACTCCAACAACTCCATCGCTATCCCCACCAACTTCACCATCTCCGTGACCACCGAGATC
CTGCCCGTGTCCATGACCAAGACCTCCGTGGACTGCACCATGTACATCTGCGGTGACTCC
ACCGAGTGCTCCAACCTGCTGCTGCAGTACGGTTCCTTCTGCACCCAGCTGAAGCGTGCT
CTGACCGGTATCGCTGTGGAGCAGGACAAGAACACCCAGGAGGTGTTCGCTCAGGTGAAG
CAGATCTACAAGACCCCCCCCATCAAGTACTTCGGTGGTTTCAACTTCTCCCAGATCCTG
CCCGACCCCTCCAAGCCCTCCAAGCGTTCCTTCATCGAGGACCTGCTGTTCAACAAGGTG
ACCCTGGCTGACGCTGGTTTCATCAAGCAGTACGGTGACTGCCTGGGTGACATCGCTGCT
CGTGACCTGATCTGCGCTCAGAAGTTCAAGGGTCTGACCGTGCTGCCCCCCCTGCTGACC
GACGAGATGATCGCTCAGTACACCTCCGCTCTGCTGGCTGGTACCATCACCTCCGGTTGG
ACCTTCGGTGCTGGTGCTGCTCTGCAGATCCCCTTCGCTATGCAGATGGCTTACCGTTTC
AACGGTATCGGTGTGACCCAGAACGTGCTGTACGAGAACCAGAAGCTGATCGCTAACCAG
TTCAACTCCGCTATCGGTAAGATCCAGGACTCCCTGTCCTCCACCGCTTCCGCTCTGGGT
AAGCTGCAGGACGTGGTGAACCACAACGCTCAGGCTCTGAACACCCTGGTGAAGCAGCTG
TCCTCCAAGTTCGGTGCTATCTCCTCCGTGCTGAACGACATCTTCTCCCGTCTGGACAAG
GTGGAGGCTGAGGTGCAGATCGACCGTCTGATCACCGGTCGTCTGCAGTCCCTGCAGACC
TACGTGACCCAGCAGCTGATCCGTGCTGCTGAGATCCGTGCTTCCGCTAACCTGGCTGCT
ACCAAGATGTCCGAGTGCGTGCTGGGTCAGTCCAAGCGTGTGGACTTCTGCGGTAAGGGT
TACCACCTGATGTCCTTCCCCCAGTCCGCTCCCCACGGTGTGGTGTTCCTGCACGTGACC
TACGTGCCCGCTCAGGAGAAGAACTTCACCACCGCTCCCGCTATCTGCCACGACGGTAAG
GCTCACTTCCCCCGTGAGGGTGTGTTCGTGTCCAACGGTACCCACTGGTTCGTGACCCAG
CGTAACTTCTACGAGCCCCAGATCATCACCACCGACAACACCTTCGTGTCCGGTAACTGC
GACGTGGTGATCGGTATCGTGAACAACACCGTGTACGACCCCCTGCAGCCCGAGCTGGAC
TCCTTCAAGGAGGAGCTGGACAAGTACTTCAAGAACCACACCTCCCCCGACGTGGACCTG
GGTGACATCTCCGGTATCAACGCTTCCGTGGTGAACATCCAGAAGGAGATCGACCGTCTG
AACGAGGTGGCTAAGAACCTGAACGAGTCCCTGATCGACCTGCAGGAGCTGGGTAAGTAC
GAGCAGTACATCAAGTGGCCCTAG
Note:
the above sequence consists of signal peptide-extracellular domain.
SEQ ID NO: 64:
Full-length amino acid sequence of Omicron-BA.2 S antigen:
MFVFLVLLPLVSSQCVNLITRTQSYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNA
TNVVIKVCEFQFCNDPFLDVYYHKNNKSWMESEFRVYSSANNCTFEYVSQPFLMDLEGKQ
GNFKNLREFVFKNIDGYFKIYSKHTPINLGRDLPQGFSALEPLVDLPIGINITRFQTLLA
LHRSYLTPGDSSSGWTAGAAAYYVGYLQPRTFLLKYNENGTITDAVDCALDPLSETKCTL
KSFTVEKGIYQTSNFRVQPTESIVRFPNITNLCPFDEVFNATRFASVYAWNRKRISNCVA
DYSVLYNFAPFFAFKCYGVSPTKLNDLCFTNVYADSFVIRGNEVSQIAPGQTGNIADYNY
KLPDDFTGCVIAWNSNKLDSKVGGNYNYLYRLFRKSNLKPFERDISTEIYQAGNKPCNGV
AGFNCYFPLRSYGFRPTYGVGHQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEYVNNSYECD
IPIGAGICASYQTQTKSHRRARSVASQSIIAYTMSLGAENSVAYSNNSIAIPTNFTISVT
TEILPVSMTKTSVDCTMYICGDSTECSNLLLQYGSFCTQLKRALTGIAVEQDKNTQEVFA
QVKQIYKTPPIKYFGGFNFSQILPDPSKPSKRSFIEDLLFNKVTLADAGFIKQYGDCLGD
IAARDLICAQKFNGLTVLPPLLTDEMIAQYTSALLAGTITSGWTFGAGAALQIPFAMQMA
YRENGIGVTQNVLYENQKLIANQFNSAIGKIQDSLSSTASALGKLQDVVNHNAQALNTLV
KQLSSKFGAISSVLNDILSRLDKVEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSCCKFDEDDSEP
VLKGVKLHYT
Note:
the above sequence consists of signal peptide-extracellular domain-
transmembrane domain-intracellular domain.
SEQ ID NO: 65:
Full-length nucleic acid sequence of Omicron-BA.2 S antigen
(optimization of CHO cells):
ATGTTCGTGTTCCTGGTGCTGCTGCCCCTGGTGAGCAGCCAGTGCGTGAACCTGATCACC
AGAACCCAGAGCTACACCAACAGCTTCACCAGAGGCGTGTACTACCCCGACAAGGTGTTC
AGAAGCAGCGTGCTGCACAGCACCCAGGACCTGTTCCTGCCCTTCTTCAGCAACGTGACC
TGGTTCCACGCCATCCACGTGAGCGGCACCAACGGCACCAAGAGATTCGACAACCCCGTG
CTGCCCTTCAACGACGGCGTGTACTTCGCCAGCACCGAGAAGAGCAACATCATCAGAGGC
TGGATCTTCGGCACCACCCTGGACAGCAAGACCCAGAGCCTGCTGATCGTGAACAACGCC
ACCAACGTGGTGATCAAGGTGTGCGAGTTCCAGTTCTGCAACGACCCCTTCCTGGACGTG
TACTACCACAAGAACAACAAGAGCTGGATGGAGAGCGAGTTCAGAGTGTACAGCAGCGCC
AACAACTGCACCTTCGAGTACGTGAGCCAGCCCTTCCTGATGGACCTGGAGGGCAAGCAG
GGCAACTTCAAGAACCTGAGAGAGTTCGTGTTCAAGAACATCGACGGCTACTTCAAGATC
TACAGCAAGCACACCCCCATCAACCTGGGCAGAGACCTGCCCCAGGGCTTCAGCGCCCTG
GAGCCCCTGGTGGACCTGCCCATCGGCATCAACATCACCAGATTCCAGACCCTGCTGGCC
CTGCACAGAAGCTACCTGACCCCCGGCGACAGCAGCAGCGGCTGGACCGCCGGCGCCGCC
GCCTACTACGTGGGCTACCTGCAGCCCAGAACCTTCCTGCTGAAGTACAACGAGAACGGC
ACCATCACCGACGCCGTGGACTGCGCCCTGGACCCCCTGAGCGAGACCAAGTGCACCCTG
AAGAGCTTCACCGTGGAGAAGGGCATCTACCAGACCAGCAACTTCAGAGTGCAGCCCACC
GAGAGCATCGTGAGATTCCCCAACATCACCAACCTGTGCCCCTTCGACGAGGTGTTCAAC
GCCACCAGATTCGCCAGCGTGTACGCCTGGAACAGAAAGAGAATCAGCAACTGCGTGGCC
GACTACAGCGTGCTGTACAACTTCGCCCCCTTCTTCGCCTTCAAGTGCTACGGCGTGAGC
CCCACCAAGCTGAACGACCTGTGCTTCACCAACGTGTACGCCGACAGCTTCGTGATCAGA
GGCAACGAGGTGAGCCAGATCGCCCCCGGCCAGACCGGCAACATCGCCGACTACAACTAC
AAGCTGCCCGACGACTTCACCGGCTGCGTGATCGCCTGGAACAGCAACAAGCTGGACAGC
AAGGTGGGCGGCAACTACAACTACCTGTACAGACTGTTCAGAAAGAGCAACCTGAAGCCC
TTCGAGAGAGACATCAGCACCGAGATCTACCAGGCCGGCAACAAGCCCTGCAACGGCGTG
GCCGGCTTCAACTGCTACTTCCCCCTGAGAAGCTACGGCTTCAGACCCACCTACGGCGTG
GGCCACCAGCCCTACAGAGTGGTGGTGCTGAGCTTCGAGCTGCTGCACGCCCCCGCCACC
GTGTGCGGCCCCAAGAAGAGCACCAACCTGGTGAAGAACAAGTGCGTGAACTTCAACTTC
AACGGCCTGACCGGCACCGGCGTGCTGACCGAGAGCAACAAGAAGTTCCTGCCCTTCCAG
CAGTTCGGCAGAGACATCGCCGACACCACCGACGCCGTGAGAGACCCCCAGACCCTGGAG
ATCCTGGACATCACCCCCTGCAGCTTCGGCGGCGTGAGCGTGATCACCCCCGGCACCAAC
ACCAGCAACCAGGTGGCCGTGCTGTACCAGGGCGTGAACTGCACCGAGGTGCCCGTGGCC
ATCCACGCCGACCAGCTGACCCCCACCTGGAGAGTGTACAGCACCGGCAGCAACGTGTTC
CAGACCAGAGCCGGCTGCCTGATCGGCGCCGAGTACGTGAACAACAGCTACGAGTGCGAC
ATCCCCATCGGCGCCGGCATCTGCGCCAGCTACCAGACCCAGACCAAGAGCCACAGAAGA
GCCAGAAGCGTGGCCAGCCAGAGCATCATCGCCTACACCATGAGCCTGGGCGCCGAGAAC
AGCGTGGCCTACAGCAACAACAGCATCGCCATCCCCACCAACTTCACCATCAGCGTGACC
ACCGAGATCCTGCCCGTGAGCATGACCAAGACCAGCGTGGACTGCACCATGTACATCTGC
GGCGACAGCACCGAGTGCAGCAACCTGCTGCTGCAGTACGGCAGCTTCTGCACCCAGCTG
AAGAGAGCCCTGACCGGCATCGCCGTGGAGCAGGACAAGAACACCCAGGAGGTGTTCGCC
CAGGTGAAGCAGATCTACAAGACCCCCCCCATCAAGTACTTCGGCGGCTTCAACTTCAGC
CAGATCCTGCCCGACCCCAGCAAGCCCAGCAAGAGAAGCTTCATCGAGGACCTGCTGTTC
AACAAGGTGACCCTGGCCGACGCCGGCTTCATCAAGCAGTACGGCGACTGCCTGGGCGAC
ATCGCCGCCAGAGACCTGATCTGCGCCCAGAAGTTCAACGGCCTGACCGTGCTGCCCCCC
CTGCTGACCGACGAGATGATCGCCCAGTACACCAGCGCCCTGCTGGCCGGCACCATCACC
AGCGGCTGGACCTTCGGCGCCGGCGCCGCCCTGCAGATCCCCTTCGCCATGCAGATGGCC
TACAGATTCAACGGCATCGGCGTGACCCAGAACGTGCTGTACGAGAACCAGAAGCTGATC
GCCAACCAGTTCAACAGCGCCATCGGCAAGATCCAGGACAGCCTGAGCAGCACCGCCAGC
GCCCTGGGCAAGCTGCAGGACGTGGTGAACCACAACGCCCAGGCCCTGAACACCCTGGTG
AAGCAGCTGAGCAGCAAGTTCGGCGCCATCAGCAGCGTGCTGAACGACATCCTGAGCAGA
CTGGACAAGGTGGAGGCCGAGGTGCAGATCGACAGACTGATCACCGGCAGACTGCAGAGC
CTGCAGACCTACGTGACCCAGCAGCTGATCAGAGCCGCCGAGATCAGAGCCAGCGCCAAC
CTGGCCGCCACCAAGATGAGCGAGTGCGTGCTGGGCCAGAGCAAGAGAGTGGACTTCTGC
GGCAAGGGCTACCACCTGATGAGCTTCCCCCAGAGCGCCCCCCACGGCGTGGTGTTCCTG
CACGTGACCTACGTGCCCGCCCAGGAGAAGAACTTCACCACCGCCCCCGCCATCTGCCAC
GACGGCAAGGCCCACTTCCCCAGAGAGGGCGTGTTCGTGAGCAACGGCACCCACTGGTTC
GTGACCCAGAGAAACTTCTACGAGCCCCAGATCATCACCACCGACAACACCTTCGTGAGC
GGCAACTGCGACGTGGTGATCGGCATCGTGAACAACACCGTGTACGACCCCCTGCAGCCC
GAGCTGGACAGCTTCAAGGAGGAGCTGGACAAGTACTTCAAGAACCACACCAGCCCCGAC
GTGGACCTGGGCGACATCAGCGGCATCAACGCCAGCGTGGTGAACATCCAGAAGGAGATC
GACAGACTGAACGAGGTGGCCAAGAACCTGAACGAGAGCCTGATCGACCTGCAGGAGCTG
GGCAAGTACGAGCAGTACATCAAGTGGCCCTGGTACATCTGGCTGGGCTTCATCGCCGGC
CTGATCGCCATCGTGATGGTGACCATCATGCTGTGCTGCATGACCAGCTGCTGCAGCTGC
CTGAAGGGCTGCTGCAGCTGCGGCAGCTGCTGCAAGTTCGACGAGGACGACAGCGAGCCC
GTGCTGAAGGGCGTGAAGCTGCACTACACCTAG
Note:
the above sequence consists of signal peptide-extracellular domain-
transmembrane domain-intracellular domain.
SEQ ID NO: 66:
Full-length nucleic acid sequence of Omicron-BA.2 S antigen
(optimization of insect cells):
ATGTTCGTGTTCCTGGTGCTGCTGCCCCTGGTGTCCTCCCAGTGCGTGAACCTGATCACC
CGTACCCAGTCCTACACCAACTCCTTCACCCGTGGTGTGTACTACCCCGACAAGGTGTTC
CGTTCCTCCGTGCTGCACTCCACCCAGGACCTGTTCCTGCCCTTCTTCTCCAACGTGACC
TGGTTCCACGCTATCCACGTGTCCGGTACCAACGGTACCAAGCGTTTCGACAACCCCGTG
CTGCCCTTCAACGACGGTGTGTACTTCGCTTCCACCGAGAAGTCCAACATCATCCGTGGT
TGGATCTTCGGTACCACCCTGGACTCCAAGACCCAGTCCCTGCTGATCGTGAACAACGCT
ACCAACGTGGTGATCAAGGTGTGCGAGTTCCAGTTCTGCAACGACCCCTTCCTGGACGTG
TACTACCACAAGAACAACAAGTCCTGGATGGAGTCCGAGTTCCGTGTGTACTCCTCCGCT
AACAACTGCACCTTCGAGTACGTGTCCCAGCCCTTCCTGATGGACCTGGAGGGTAAGCAG
GGTAACTTCAAGAACCTGCGTGAGTTCGTGTTCAAGAACATCGACGGTTACTTCAAGATC
TACTCCAAGCACACCCCCATCAACCTGGGTCGTGACCTGCCCCAGGGTTTCTCCGCTCTG
GAGCCCCTGGTGGACCTGCCCATCGGTATCAACATCACCCGTTTCCAGACCCTGCTGGCT
CTGCACCGTTCCTACCTGACCCCCGGTGACTCCTCCTCCGGTTGGACCGCTGGTGCTGCT
GCTTACTACGTGGGTTACCTGCAGCCCCGTACCTTCCTGCTGAAGTACAACGAGAACGGT
ACCATCACCGACGCTGTGGACTGCGCTCTGGACCCCCTGTCCGAGACCAAGTGCACCCTG
AAGTCCTTCACCGTGGAGAAGGGTATCTACCAGACCTCCAACTTCCGTGTGCAGCCCACC
GAGTCCATCGTGCGTTTCCCCAACATCACCAACCTGTGCCCCTTCGACGAGGTGTTCAAC
GCTACCCGTTTCGCTTCCGTGTACGCTTGGAACCGTAAGCGTATCTCCAACTGCGTGGCT
GACTACTCCGTGCTGTACAACTTCGCTCCCTTCTTCGCTTTCAAGTGCTACGGTGTGTCC
CCCACCAAGCTGAACGACCTGTGCTTCACCAACGTGTACGCTGACTCCTTCGTGATCCGT
GGTAACGAGGTGTCCCAGATCGCTCCCGGTCAGACCGGTAACATCGCTGACTACAACTAC
AAGCTGCCCGACGACTTCACCGGTTGCGTGATCGCTTGGAACTCCAACAAGCTGGACTCC
AAGGTGGGTGGTAACTACAACTACCTGTACCGTCTGTTCCGTAAGTCCAACCTGAAGCCC
TTCGAGCGTGACATCTCCACCGAGATCTACCAGGCTGGTAACAAGCCCTGCAACGGTGTG
GCTGGTTTCAACTGCTACTTCCCCCTGCGTTCCTACGGTTTCCGTCCCACCTACGGTGTG
GGTCACCAGCCCTACCGTGTGGTGGTGCTGTCCTTCGAGCTGCTGCACGCTCCCGCTACC
GTGTGCGGTCCCAAGAAGTCCACCAACCTGGTGAAGAACAAGTGCGTGAACTTCAACTTC
AACGGTCTGACCGGTACCGGTGTGCTGACCGAGTCCAACAAGAAGTTCCTGCCCTTCCAG
CAGTTCGGTCGTGACATCGCTGACACCACCGACGCTGTGCGTGACCCCCAGACCCTGGAG
ATCCTGGACATCACCCCCTGCTCCTTCGGTGGTGTGTCCGTGATCACCCCCGGTACCAAC
ACCTCCAACCAGGTGGCTGTGCTGTACCAGGGTGTGAACTGCACCGAGGTGCCCGTGGCT
ATCCACGCTGACCAGCTGACCCCCACCTGGCGTGTGTACTCCACCGGTTCCAACGTGTTC
CAGACCCGTGCTGGTTGCCTGATCGGTGCTGAGTACGTGAACAACTCCTACGAGTGCGAC
ATCCCCATCGGTGCTGGTATCTGCGCTTCCTACCAGACCCAGACCAAGTCCCACCGTCGT
GCTCGTTCCGTGGCTTCCCAGTCCATCATCGCTTACACCATGTCCCTGGGTGCTGAGAAC
TCCGTGGCTTACTCCAACAACTCCATCGCTATCCCCACCAACTTCACCATCTCCGTGACC
ACCGAGATCCTGCCCGTGTCCATGACCAAGACCTCCGTGGACTGCACCATGTACATCTGC
GGTGACTCCACCGAGTGCTCCAACCTGCTGCTGCAGTACGGTTCCTTCTGCACCCAGCTG
AAGCGTGCTCTGACCGGTATCGCTGTGGAGCAGGACAAGAACACCCAGGAGGTGTTCGCT
CAGGTGAAGCAGATCTACAAGACCCCCCCCATCAAGTACTTCGGTGGTTTCAACTTCTCC
CAGATCCTGCCCGACCCCTCCAAGCCCTCCAAGCGTTCCTTCATCGAGGACCTGCTGTTC
AACAAGGTGACCCTGGCTGACGCTGGTTTCATCAAGCAGTACGGTGACTGCCTGGGTGAC
ATCGCTGCTCGTGACCTGATCTGCGCTCAGAAGTTCAACGGTCTGACCGTGCTGCCCCCC
CTGCTGACCGACGAGATGATCGCTCAGTACACCTCCGCTCTGCTGGCTGGTACCATCACC
TCCGGTTGGACCTTCGGTGCTGGTGCTGCTCTGCAGATCCCCTTCGCTATGCAGATGGCT
TACCGTTTCAACGGTATCGGTGTGACCCAGAACGTGCTGTACGAGAACCAGAAGCTGATC
GCTAACCAGTTCAACTCCGCTATCGGTAAGATCCAGGACTCCCTGTCCTCCACCGCTTCC
GCTCTGGGTAAGCTGCAGGACGTGGTGAACCACAACGCTCAGGCTCTGAACACCCTGGTG
AAGCAGCTGTCCTCCAAGTTCGGTGCTATCTCCTCCGTGCTGAACGACATCCTGTCCCGT
CTGGACAAGGTGGAGGCTGAGGTGCAGATCGACCGTCTGATCACCGGTCGTCTGCAGTCC
CTGCAGACCTACGTGACCCAGCAGCTGATCCGTGCTGCTGAGATCCGTGCTTCCGCTAAC
CTGGCTGCTACCAAGATGTCCGAGTGCGTGCTGGGTCAGTCCAAGCGTGTGGACTTCTGC
GGTAAGGGTTACCACCTGATGTCCTTCCCCCAGTCCGCTCCCCACGGTGTGGTGTTCCTG
CACGTGACCTACGTGCCCGCTCAGGAGAAGAACTTCACCACCGCTCCCGCTATCTGCCAC
GACGGTAAGGCTCACTTCCCCCGTGAGGGTGTGTTCGTGTCCAACGGTACCCACTGGTTC
GTGACCCAGCGTAACTTCTACGAGCCCCAGATCATCACCACCGACAACACCTTCGTGTCC
GGTAACTGCGACGTGGTGATCGGTATCGTGAACAACACCGTGTACGACCCCCTGCAGCCC
GAGCTGGACTCCTTCAAGGAGGAGCTGGACAAGTACTTCAAGAACCACACCTCCCCCGAC
GTGGACCTGGGTGACATCTCCGGTATCAACGCTTCCGTGGTGAACATCCAGAAGGAGATC
GACCGTCTGAACGAGGTGGCTAAGAACCTGAACGAGTCCCTGATCGACCTGCAGGAGCTG
GGTAAGTACGAGCAGTACATCAAGTGGCCCTGGTACATCTGGCTGGGTTTCATCGCTGGT
CTGATCGCTATCGTGATGGTGACCATCATGCTGTGCTGCATGACCTCCTGCTGCTCCTGC
CTGAAGGGTTGCTGCTCCTGCGGTTCCTGCTGCAAGTTCGACGAGGACGACTCCGAGCCC
GTGCTGAAGGGTGTGAAGCTGCACTACACCTAG
Note:
the above sequence consists of signal peptide-extracellular domain-
transmembrane domain-intracellular domain.
SEQ ID NO: 67:
Extracellular amino acid sequence of Omicron-BA.2 S antigen:
MFVFLVLLPLVSSQCVNLITRTQSYTNSFTRGVYYPDKVFRSSVLHSTQDLFLPFFSNVT
WFHAIHVSGTNGTKRFDNPVLPFNDGVYFASTEKSNIIRGWIFGTTLDSKTQSLLIVNNA
TNVVIKVCEFQFCNDPFLDVYYHKNNKSWMESEFRVYSSANNCTFEYVSQPFLMDLEGKQ
GNFKNLREFVFKNIDGYFKIYSKHTPINLGRDLPQGFSALEPLVDLPIGINITRFQTLLA
LHRSYLTPGDSSSGWTAGAAAYYVGYLQPRTFLLKYNENGTITDAVDCALDPLSETKCTL
KSFTVEKGIYQTSNFRVQPTESIVRFPNITNLCPFDEVFNATRFASVYAWNRKRISNCVA
DYSVLYNFAPFFAFKCYGVSPTKLNDLCFTNVYADSFVIRGNEVSQIAPGQTGNIADYNY
KLPDDFTGCVIAWNSNKLDSKVGGNYNYLYRLFRKSNLKPFERDISTEIYQAGNKPCNGV
AGENCYFPLRSYGFRPTYGVGHQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGAEYVNNSYECD
IPIGAGICASYQTQTKSHRRARSVASQSIIAYTMSLGAENSVAYSNNSIAIPTNFTISVT
TEILPVSMTKTSVDCTMYICGDSTECSNLLLQYGSFCTQLKRALTGIAVEQDKNTQEVFA
QVKQIYKTPPIKYFGGFNFSQILPDPSKPSKRSFIEDLLFNKVTLADAGFIKQYGDCLGD
IAARDLICAQKFNGLTVLPPLLTDEMIAQYTSALLAGTITSGWTFGAGAALQIPFAMQMA
YRENGIGVTQNVLYENQKLIANQFNSAIGKIQDSLSSTASALGKLQDVVNHNAQALNTLV
KQLSSKFGAISSVLNDILSRLDKVEAEVQIDRLITGRLQSLQTYVTQQLIRAAEIRASAN
LAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQP
ELDSFKEELDKYFKNHTSPDVDLGDISGINASVVNIQKEIDRLNEVAKNLNESLIDLQEL
GKYEQYIKWP
Note:
the above sequence consists of signal peptide-extracellular domain.
SEQ ID NO: 68:
Extracellular nucleic acid sequence of Omicron-BA.2 S antigen
(optimization of CHO cells):
ATGTTCGTGTTCCTGGTGCTGCTGCCCCTGGTGAGCAGCCAGTGCGTGAACCTGATCACC
AGAACCCAGAGCTACACCAACAGCTTCACCAGAGGCGTGTACTACCCCGACAAGGTGTTC
AGAAGCAGCGTGCTGCACAGCACCCAGGACCTGTTCCTGCCCTTCTTCAGCAACGTGACC
TGGTTCCACGCCATCCACGTGAGCGGCACCAACGGCACCAAGAGATTCGACAACCCCGTG
CTGCCCTTCAACGACGGCGTGTACTTCGCCAGCACCGAGAAGAGCAACATCATCAGAGGC
TGGATCTTCGGCACCACCCTGGACAGCAAGACCCAGAGCCTGCTGATCGTGAACAACGCC
ACCAACGTGGTGATCAAGGTGTGCGAGTTCCAGTTCTGCAACGACCCCTTCCTGGACGTG
TACTACCACAAGAACAACAAGAGCTGGATGGAGAGCGAGTTCAGAGTGTACAGCAGCGCC
AACAACTGCACCTTCGAGTACGTGAGCCAGCCCTTCCTGATGGACCTGGAGGGCAAGCAG
GGCAACTTCAAGAACCTGAGAGAGTTCGTGTTCAAGAACATCGACGGCTACTTCAAGATC
TACAGCAAGCACACCCCCATCAACCTGGGCAGAGACCTGCCCCAGGGCTTCAGCGCCCTG
GAGCCCCTGGTGGACCTGCCCATCGGCATCAACATCACCAGATTCCAGACCCTGCTGGCC
CTGCACAGAAGCTACCTGACCCCCGGCGACAGCAGCAGCGGCTGGACCGCCGGCGCCGCC
GCCTACTACGTGGGCTACCTGCAGCCCAGAACCTTCCTGCTGAAGTACAACGAGAACGGC
ACCATCACCGACGCCGTGGACTGCGCCCTGGACCCCCTGAGCGAGACCAAGTGCACCCTG
AAGAGCTTCACCGTGGAGAAGGGCATCTACCAGACCAGCAACTTCAGAGTGCAGCCCACC
GAGAGCATCGTGAGATTCCCCAACATCACCAACCTGTGCCCCTTCGACGAGGTGTTCAAC
GCCACCAGATTCGCCAGCGTGTACGCCTGGAACAGAAAGAGAATCAGCAACTGCGTGGCC
GACTACAGCGTGCTGTACAACTTCGCCCCCTTCTTCGCCTTCAAGTGCTACGGCGTGAGC
CCCACCAAGCTGAACGACCTGTGCTTCACCAACGTGTACGCCGACAGCTTCGTGATCAGA
GGCAACGAGGTGAGCCAGATCGCCCCCGGCCAGACCGGCAACATCGCCGACTACAACTAC
AAGCTGCCCGACGACTTCACCGGCTGCGTGATCGCCTGGAACAGCAACAAGCTGGACAGC
AAGGTGGGCGGCAACTACAACTACCTGTACAGACTGTTCAGAAAGAGCAACCTGAAGCCC
TTCGAGAGAGACATCAGCACCGAGATCTACCAGGCCGGCAACAAGCCCTGCAACGGCGTG
GCCGGCTTCAACTGCTACTTCCCCCTGAGAAGCTACGGCTTCAGACCCACCTACGGCGTG
GGCCACCAGCCCTACAGAGTGGTGGTGCTGAGCTTCGAGCTGCTGCACGCCCCCGCCACC
GTGTGCGGCCCCAAGAAGAGCACCAACCTGGTGAAGAACAAGTGCGTGAACTTCAACTTC
AACGGCCTGACCGGCACCGGCGTGCTGACCGAGAGCAACAAGAAGTTCCTGCCCTTCCAG
CAGTTCGGCAGAGACATCGCCGACACCACCGACGCCGTGAGAGACCCCCAGACCCTGGAG
ATCCTGGACATCACCCCCTGCAGCTTCGGCGGCGTGAGCGTGATCACCCCCGGCACCAAC
ACCAGCAACCAGGTGGCCGTGCTGTACCAGGGCGTGAACTGCACCGAGGTGCCCGTGGCC
ATCCACGCCGACCAGCTGACCCCCACCTGGAGAGTGTACAGCACCGGCAGCAACGTGTTC
CAGACCAGAGCCGGCTGCCTGATCGGCGCCGAGTACGTGAACAACAGCTACGAGTGCGAC
ATCCCCATCGGCGCCGGCATCTGCGCCAGCTACCAGACCCAGACCAAGAGCCACAGAAGA
GCCAGAAGCGTGGCCAGCCAGAGCATCATCGCCTACACCATGAGCCTGGGCGCCGAGAAC
AGCGTGGCCTACAGCAACAACAGCATCGCCATCCCCACCAACTTCACCATCAGCGTGACC
ACCGAGATCCTGCCCGTGAGCATGACCAAGACCAGCGTGGACTGCACCATGTACATCTGC
GGCGACAGCACCGAGTGCAGCAACCTGCTGCTGCAGTACGGCAGCTTCTGCACCCAGCTG
AAGAGAGCCCTGACCGGCATCGCCGTGGAGCAGGACAAGAACACCCAGGAGGTGTTCGCC
CAGGTGAAGCAGATCTACAAGACCCCCCCCATCAAGTACTTCGGCGGCTTCAACTTCAGC
CAGATCCTGCCCGACCCCAGCAAGCCCAGCAAGAGAAGCTTCATCGAGGACCTGCTGTTC
AACAAGGTGACCCTGGCCGACGCCGGCTTCATCAAGCAGTACGGCGACTGCCTGGGCGAC
ATCGCCGCCAGAGACCTGATCTGCGCCCAGAAGTTCAACGGCCTGACCGTGCTGCCCCCC
CTGCTGACCGACGAGATGATCGCCCAGTACACCAGCGCCCTGCTGGCCGGCACCATCACC
AGCGGCTGGACCTTCGGCGCCGGCGCCGCCCTGCAGATCCCCTTCGCCATGCAGATGGCC
TACAGATTCAACGGCATCGGCGTGACCCAGAACGTGCTGTACGAGAACCAGAAGCTGATC
GCCAACCAGTTCAACAGCGCCATCGGCAAGATCCAGGACAGCCTGAGCAGCACCGCCAGC
GCCCTGGGCAAGCTGCAGGACGTGGTGAACCACAACGCCCAGGCCCTGAACACCCTGGTG
AAGCAGCTGAGCAGCAAGTTCGGCGCCATCAGCAGCGTGCTGAACGACATCCTGAGCAGA
CTGGACAAGGTGGAGGCCGAGGTGCAGATCGACAGACTGATCACCGGCAGACTGCAGAGC
CTGCAGACCTACGTGACCCAGCAGCTGATCAGAGCCGCCGAGATCAGAGCCAGCGCCAAC
CTGGCCGCCACCAAGATGAGCGAGTGCGTGCTGGGCCAGAGCAAGAGAGTGGACTTCTGC
GGCAAGGGCTACCACCTGATGAGCTTCCCCCAGAGCGCCCCCCACGGCGTGGTGTTCCTG
CACGTGACCTACGTGCCCGCCCAGGAGAAGAACTTCACCACCGCCCCCGCCATCTGCCAC
GACGGCAAGGCCCACTTCCCCAGAGAGGGCGTGTTCGTGAGCAACGGCACCCACTGGTTC
GTGACCCAGAGAAACTTCTACGAGCCCCAGATCATCACCACCGACAACACCTTCGTGAGC
GGCAACTGCGACGTGGTGATCGGCATCGTGAACAACACCGTGTACGACCCCCTGCAGCCC
GAGCTGGACAGCTTCAAGGAGGAGCTGGACAAGTACTTCAAGAACCACACCAGCCCCGAC
GTGGACCTGGGCGACATCAGCGGCATCAACGCCAGCGTGGTGAACATCCAGAAGGAGATC
GACAGACTGAACGAGGTGGCCAAGAACCTGAACGAGAGCCTGATCGACCTGCAGGAGCTG
GGCAAGTACGAGCAGTACATCAAGTGGCCCTAG
Note:
the above sequence consists of signal peptide-extracellular domain.
SEQ ID NO: 69:
Extracellular nucleic acid sequence of Omicron-BA.2 S antigen
(optimization of insect cells):
ATGTTCGTGTTCCTGGTGCTGCTGCCCCTGGTGTCCTCCCAGTGCGTGAACCTGATCACC
CGTACCCAGTCCTACACCAACTCCTTCACCCGTGGTGTGTACTACCCCGACAAGGTGTTC
CGTTCCTCCGTGCTGCACTCCACCCAGGACCTGTTCCTGCCCTTCTTCTCCAACGTGACC
TGGTTCCACGCTATCCACGTGTCCGGTACCAACGGTACCAAGCGTTTCGACAACCCCGTG
CTGCCCTTCAACGACGGTGTGTACTTCGCTTCCACCGAGAAGTCCAACATCATCCGTGGT
TGGATCTTCGGTACCACCCTGGACTCCAAGACCCAGTCCCTGCTGATCGTGAACAACGCT
ACCAACGTGGTGATCAAGGTGTGCGAGTTCCAGTTCTGCAACGACCCCTTCCTGGACGTG
TACTACCACAAGAACAACAAGTCCTGGATGGAGTCCGAGTTCCGTGTGTACTCCTCCGCT
AACAACTGCACCTTCGAGTACGTGTCCCAGCCCTTCCTGATGGACCTGGAGGGTAAGCAG
GGTAACTTCAAGAACCTGCGTGAGTTCGTGTTCAAGAACATCGACGGTTACTTCAAGATC
TACTCCAAGCACACCCCCATCAACCTGGGTCGTGACCTGCCCCAGGGTTTCTCCGCTCTG
GAGCCCCTGGTGGACCTGCCCATCGGTATCAACATCACCCGTTTCCAGACCCTGCTGGCT
CTGCACCGTTCCTACCTGACCCCCGGTGACTCCTCCTCCGGTTGGACCGCTGGTGCTGCT
GCTTACTACGTGGGTTACCTGCAGCCCCGTACCTTCCTGCTGAAGTACAACGAGAACGGT
ACCATCACCGACGCTGTGGACTGCGCTCTGGACCCCCTGTCCGAGACCAAGTGCACCCTG
AAGTCCTTCACCGTGGAGAAGGGTATCTACCAGACCTCCAACTTCCGTGTGCAGCCCACC
GAGTCCATCGTGCGTTTCCCCAACATCACCAACCTGTGCCCCTTCGACGAGGTGTTCAAC
GCTACCCGTTTCGCTTCCGTGTACGCTTGGAACCGTAAGCGTATCTCCAACTGCGTGGCT
GACTACTCCGTGCTGTACAACTTCGCTCCCTTCTTCGCTTTCAAGTGCTACGGTGTGTCC
CCCACCAAGCTGAACGACCTGTGCTTCACCAACGTGTACGCTGACTCCTTCGTGATCCGT
GGTAACGAGGTGTCCCAGATCGCTCCCGGTCAGACCGGTAACATCGCTGACTACAACTAC
AAGCTGCCCGACGACTTCACCGGTTGCGTGATCGCTTGGAACTCCAACAAGCTGGACTCC
AAGGTGGGTGGTAACTACAACTACCTGTACCGTCTGTTCCGTAAGTCCAACCTGAAGCCC
TTCGAGCGTGACATCTCCACCGAGATCTACCAGGCTGGTAACAAGCCCTGCAACGGTGTG
GCTGGTTTCAACTGCTACTTCCCCCTGCGTTCCTACGGTTTCCGTCCCACCTACGGTGTG
GGTCACCAGCCCTACCGTGTGGTGGTGCTGTCCTTCGAGCTGCTGCACGCTCCCGCTACC
GTGTGCGGTCCCAAGAAGTCCACCAACCTGGTGAAGAACAAGTGCGTGAACTTCAACTTC
AACGGTCTGACCGGTACCGGTGTGCTGACCGAGTCCAACAAGAAGTTCCTGCCCTTCCAG
CAGTTCGGTCGTGACATCGCTGACACCACCGACGCTGTGCGTGACCCCCAGACCCTGGAG
ATCCTGGACATCACCCCCTGCTCCTTCGGTGGTGTGTCCGTGATCACCCCCGGTACCAAC
ACCTCCAACCAGGTGGCTGTGCTGTACCAGGGTGTGAACTGCACCGAGGTGCCCGTGGCT
ATCCACGCTGACCAGCTGACCCCCACCTGGCGTGTGTACTCCACCGGTTCCAACGTGTTC
CAGACCCGTGCTGGTTGCCTGATCGGTGCTGAGTACGTGAACAACTCCTACGAGTGCGAC
ATCCCCATCGGTGCTGGTATCTGCGCTTCCTACCAGACCCAGACCAAGTCCCACCGTCGT
GCTCGTTCCGTGGCTTCCCAGTCCATCATCGCTTACACCATGTCCCTGGGTGCTGAGAAC
TCCGTGGCTTACTCCAACAACTCCATCGCTATCCCCACCAACTTCACCATCTCCGTGACC
ACCGAGATCCTGCCCGTGTCCATGACCAAGACCTCCGTGGACTGCACCATGTACATCTGC
GGTGACTCCACCGAGTGCTCCAACCTGCTGCTGCAGTACGGTTCCTTCTGCACCCAGCTG
AAGCGTGCTCTGACCGGTATCGCTGTGGAGCAGGACAAGAACACCCAGGAGGTGTTCGCT
CAGGTGAAGCAGATCTACAAGACCCCCCCCATCAAGTACTTCGGTGGTTTCAACTTCTCC
CAGATCCTGCCCGACCCCTCCAAGCCCTCCAAGCGTTCCTTCATCGAGGACCTGCTGTTC
AACAAGGTGACCCTGGCTGACGCTGGTTTCATCAAGCAGTACGGTGACTGCCTGGGTGAC
ATCGCTGCTCGTGACCTGATCTGCGCTCAGAAGTTCAACGGTCTGACCGTGCTGCCCCCC
CTGCTGACCGACGAGATGATCGCTCAGTACACCTCCGCTCTGCTGGCTGGTACCATCACC
TCCGGTTGGACCTTCGGTGCTGGTGCTGCTCTGCAGATCCCCTTCGCTATGCAGATGGCT
TACCGTTTCAACGGTATCGGTGTGACCCAGAACGTGCTGTACGAGAACCAGAAGCTGATC
GCTAACCAGTTCAACTCCGCTATCGGTAAGATCCAGGACTCCCTGTCCTCCACCGCTTCC
GCTCTGGGTAAGCTGCAGGACGTGGTGAACCACAACGCTCAGGCTCTGAACACCCTGGTG
AAGCAGCTGTCCTCCAAGTTCGGTGCTATCTCCTCCGTGCTGAACGACATCCTGTCCCGT
CTGGACAAGGTGGAGGCTGAGGTGCAGATCGACCGTCTGATCACCGGTCGTCTGCAGTCC
CTGCAGACCTACGTGACCCAGCAGCTGATCCGTGCTGCTGAGATCCGTGCTTCCGCTAAC
CTGGCTGCTACCAAGATGTCCGAGTGCGTGCTGGGTCAGTCCAAGCGTGTGGACTTCTGC
GGTAAGGGTTACCACCTGATGTCCTTCCCCCAGTCCGCTCCCCACGGTGTGGTGTTCCTG
CACGTGACCTACGTGCCCGCTCAGGAGAAGAACTTCACCACCGCTCCCGCTATCTGCCAC
GACGGTAAGGCTCACTTCCCCCGTGAGGGTGTGTTCGTGTCCAACGGTACCCACTGGTTC
GTGACCCAGCGTAACTTCTACGAGCCCCAGATCATCACCACCGACAACACCTTCGTGTCC
GGTAACTGCGACGTGGTGATCGGTATCGTGAACAACACCGTGTACGACCCCCTGCAGCCC
GAGCTGGACTCCTTCAAGGAGGAGCTGGACAAGTACTTCAAGAACCACACCTCCCCCGAC
GTGGACCTGGGTGACATCTCCGGTATCAACGCTTCCGTGGTGAACATCCAGAAGGAGATC
GACCGTCTGAACGAGGTGGCTAAGAACCTGAACGAGTCCCTGATCGACCTGCAGGAGCTG
GGTAAGTACGAGCAGTACATCAAGTGGCCCTAG
Note:
the above sequence consists of signal peptide-extracellular domain.
SEQ ID NO: 70:
Full-length amino acid sequence of Omicron-BA.4/5 S antigen:
MFVFLVLLPLVSSQCVNLITRTQSYTNSFTRGVYYPDKVFRSSVLHSTQDLFLPFFSNVT
WFHAISGTNGTKRFDNPVLPFNDGVYFASTEKSNIIRGWIFGTTLDSKTQSLLIVNNATN
VVIKVCEFQFCNDPFLDVYYHKNNKSWMESEFRVYSSANNCTFEYVSQPFLMDLEGKQGN
FKNLREFVFKNIDGYFKIYSKHTPINLGRDLPQGFSALEPLVDLPIGINITRFQTLLALH
RSYLTPGDSSSGWTAGAAAYYVGYLQPRTFLLKYNENGTITDAVDCALDPLSETKCTLKS
FTVEKGIYQTSNFRVQPTESIVRFPNITNLCPFDEVFNATRFASVYAWNRKRISNCVADY
SVLYNFAPFFAFKCYGVSPTKLNDLCFTNVYADSFVIRGNEVSQIAPGQTGNIADYNYKL
PDDFTGCVIAWNSNKLDSKVGGNYNYRYRLFRKSNLKPFERDISTEIYQAGNKPCNGVAG
VNCYFPLQSYGFRPTYGVGHQPYRVVVLSFELLHAPATVCGPKKSTNLVKNKCVNFNFNG
LTGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEYVNNSYECDIP
IGAGICASYQTQTKSHRRARSVASQSIIAYTMSLGAENSVAYSNNSIAIPTNFTISVTTE
ILPVSMTKTSVDCTMYICGDSTECSNLLLQYGSFCTQLKRALTGIAVEQDKNTQEVFAQV
KQIYKTPPIKYFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQIPFAMQMAYR
ENGIGVTQNVLYENQKLIANQFNSAIGKIQDSLSSTASALGKLQDVVNHNAQALNTLVKQ
LSSKFGAISSVLNDILSRLDKVEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK
YEQYIKWPWYIWLGFIAGLIAIVMVTIMLCCMTSCCSCLKGCCSCGSCCKFDEDDSEPVL
KGVKLHYT
Note:
the above sequence consists of signal peptide-extracellular domain-
transmembrane domain-intracellular domain.
SEQ ID NO: 71:
Full-length nucleic acid sequence of Omicron-BA.4/5 S antigen
(optimization of CHO cells):
ATGTTCGTGTTCCTGGTGCTGCTGCCCCTGGTGAGCAGCCAGTGCGTGAACCTGATCACC
AGAACCCAGAGCTACACCAACAGCTTCACCAGAGGCGTGTACTACCCCGACAAGGTGTTC
AGAAGCAGCGTGCTGCACAGCACCCAGGACCTGTTCCTGCCCTTCTTCAGCAACGTGACC
TGGTTCCACGCCATCAGCGGCACCAACGGCACCAAGAGATTCGACAACCCCGTGCTGCCC
TTCAACGACGGCGTGTACTTCGCCAGCACCGAGAAGAGCAACATCATCAGAGGCTGGATC
TTCGGCACCACCCTGGACAGCAAGACCCAGAGCCTGCTGATCGTGAACAACGCCACCAAC
GTGGTGATCAAGGTGTGCGAGTTCCAGTTCTGCAACGACCCCTTCCTGGACGTGTACTAC
CACAAGAACAACAAGAGCTGGATGGAGAGCGAGTTCAGAGTGTACAGCAGCGCCAACAAC
TGCACCTTCGAGTACGTGAGCCAGCCCTTCCTGATGGACCTGGAGGGCAAGCAGGGCAAC
TTCAAGAACCTGAGAGAGTTCGTGTTCAAGAACATCGACGGCTACTTCAAGATCTACAGC
AAGCACACCCCCATCAACCTGGGCAGAGACCTGCCCCAGGGCTTCAGCGCCCTGGAGCCC
CTGGTGGACCTGCCCATCGGCATCAACATCACCAGATTCCAGACCCTGCTGGCCCTGCAC
AGAAGCTACCTGACCCCCGGCGACAGCAGCAGCGGCTGGACCGCCGGCGCCGCCGCCTAC
TACGTGGGCTACCTGCAGCCCAGAACCTTCCTGCTGAAGTACAACGAGAACGGCACCATC
ACCGACGCCGTGGACTGCGCCCTGGACCCCCTGAGCGAGACCAAGTGCACCCTGAAGAGC
TTCACCGTGGAGAAGGGCATCTACCAGACCAGCAACTTCAGAGTGCAGCCCACCGAGAGC
ATCGTGAGATTCCCCAACATCACCAACCTGTGCCCCTTCGACGAGGTGTTCAACGCCACC
AGATTCGCCAGCGTGTACGCCTGGAACAGAAAGAGAATCAGCAACTGCGTGGCCGACTAC
AGCGTGCTGTACAACTTCGCCCCCTTCTTCGCCTTCAAGTGCTACGGCGTGAGCCCCACC
AAGCTGAACGACCTGTGCTTCACCAACGTGTACGCCGACAGCTTCGTGATCAGAGGCAAC
GAGGTGAGCCAGATCGCCCCCGGCCAGACCGGCAACATCGCCGACTACAACTACAAGCTG
CCCGACGACTTCACCGGCTGCGTGATCGCCTGGAACAGCAACAAGCTGGACAGCAAGGTG
GGCGGCAACTACAACTACAGATACAGACTGTTCAGAAAGAGCAACCTGAAGCCCTTCGAG
AGAGACATCAGCACCGAGATCTACCAGGCCGGCAACAAGCCCTGCAACGGCGTGGCCGGC
GTGAACTGCTACTTCCCCCTGCAGAGCTACGGCTTCAGACCCACCTACGGCGTGGGCCAC
CAGCCCTACAGAGTGGTGGTGCTGAGCTTCGAGCTGCTGCACGCCCCCGCCACCGTGTGC
GGCCCCAAGAAGAGCACCAACCTGGTGAAGAACAAGTGCGTGAACTTCAACTTCAACGGC
CTGACCGGCACCGGCGTGCTGACCGAGAGCAACAAGAAGTTCCTGCCCTTCCAGCAGTTC
GGCAGAGACATCGCCGACACCACCGACGCCGTGAGAGACCCCCAGACCCTGGAGATCCTG
GACATCACCCCCTGCAGCTTCGGCGGCGTGAGCGTGATCACCCCCGGCACCAACACCAGC
AACCAGGTGGCCGTGCTGTACCAGGGCGTGAACTGCACCGAGGTGCCCGTGGCCATCCAC
GCCGACCAGCTGACCCCCACCTGGAGAGTGTACAGCACCGGCAGCAACGTGTTCCAGACC
AGAGCCGGCTGCCTGATCGGCGCCGAGTACGTGAACAACAGCTACGAGTGCGACATCCCC
ATCGGCGCCGGCATCTGCGCCAGCTACCAGACCCAGACCAAGAGCCACAGAAGAGCCAGA
AGCGTGGCCAGCCAGAGCATCATCGCCTACACCATGAGCCTGGGCGCCGAGAACAGCGTG
GCCTACAGCAACAACAGCATCGCCATCCCCACCAACTTCACCATCAGCGTGACCACCGAG
ATCCTGCCCGTGAGCATGACCAAGACCAGCGTGGACTGCACCATGTACATCTGCGGCGAC
AGCACCGAGTGCAGCAACCTGCTGCTGCAGTACGGCAGCTTCTGCACCCAGCTGAAGAGA
GCCCTGACCGGCATCGCCGTGGAGCAGGACAAGAACACCCAGGAGGTGTTCGCCCAGGTG
AAGCAGATCTACAAGACCCCCCCCATCAAGTACTTCGGCGGCTTCAACTTCAGCCAGATC
CTGCCCGACCCCAGCAAGCCCAGCAAGAGAAGCTTCATCGAGGACCTGCTGTTCAACAAG
GTGACCCTGGCCGACGCCGGCTTCATCAAGCAGTACGGCGACTGCCTGGGCGACATCGCC
GCCAGAGACCTGATCTGCGCCCAGAAGTTCAACGGCCTGACCGTGCTGCCCCCCCTGCTG
ACCGACGAGATGATCGCCCAGTACACCAGCGCCCTGCTGGCCGGCACCATCACCAGCGGC
TGGACCTTCGGCGCCGGCGCCGCCCTGCAGATCCCCTTCGCCATGCAGATGGCCTACAGA
TTCAACGGCATCGGCGTGACCCAGAACGTGCTGTACGAGAACCAGAAGCTGATCGCCAAC
CAGTTCAACAGCGCCATCGGCAAGATCCAGGACAGCCTGAGCAGCACCGCCAGCGCCCTG
GGCAAGCTGCAGGACGTGGTGAACCACAACGCCCAGGCCCTGAACACCCTGGTGAAGCAG
CTGAGCAGCAAGTTCGGCGCCATCAGCAGCGTGCTGAACGACATCCTGAGCAGACTGGAC
AAGGTGGAGGCCGAGGTGCAGATCGACAGACTGATCACCGGCAGACTGCAGAGCCTGCAG
ACCTACGTGACCCAGCAGCTGATCAGAGCCGCCGAGATCAGAGCCAGCGCCAACCTGGCC
GCCACCAAGATGAGCGAGTGCGTGCTGGGCCAGAGCAAGAGAGTGGACTTCTGCGGCAAG
GGCTACCACCTGATGAGCTTCCCCCAGAGCGCCCCCCACGGCGTGGTGTTCCTGCACGTG
ACCTACGTGCCCGCCCAGGAGAAGAACTTCACCACCGCCCCCGCCATCTGCCACGACGGC
AAGGCCCACTTCCCCAGAGAGGGCGTGTTCGTGAGCAACGGCACCCACTGGTTCGTGACC
CAGAGAAACTTCTACGAGCCCCAGATCATCACCACCGACAACACCTTCGTGAGCGGCAAC
TGCGACGTGGTGATCGGCATCGTGAACAACACCGTGTACGACCCCCTGCAGCCCGAGCTG
GACAGCTTCAAGGAGGAGCTGGACAAGTACTTCAAGAACCACACCAGCCCCGACGTGGAC
CTGGGCGACATCAGCGGCATCAACGCCAGCGTGGTGAACATCCAGAAGGAGATCGACAGA
CTGAACGAGGTGGCCAAGAACCTGAACGAGAGCCTGATCGACCTGCAGGAGCTGGGCAAG
TACGAGCAGTACATCAAGTGGCCCTGGTACATCTGGCTGGGCTTCATCGCCGGCCTGATC
GCCATCGTGATGGTGACCATCATGCTGTGCTGCATGACCAGCTGCTGCAGCTGCCTGAAG
GGCTGCTGCAGCTGCGGCAGCTGCTGCAAGTTCGACGAGGACGACAGCGAGCCCGTGCTG
AAGGGCGTGAAGCTGCACTACACCTAG
Note:
the above sequence consists of signal peptide-extracellular domain-
transmembrane domain-intracellular domain.
SEQ ID NO: 72:
Full-length nucleic acid sequence of Omicron-BA.4/5 S antigen
(optimization of insect cells):
ATGTTCGTGTTCCTGGTGCTGCTGCCCCTGGTGTCCTCCCAGTGCGTGAACCTGATCACC
CGTACCCAGTCCTACACCAACTCCTTCACCCGTGGTGTGTACTACCCCGACAAGGTGTTC
CGTTCCTCCGTGCTGCACTCCACCCAGGACCTGTTCCTGCCCTTCTTCTCCAACGTGACC
TGGTTCCACGCTATCTCCGGTACCAACGGTACCAAGCGTTTCGACAACCCCGTGCTGCCC
TTCAACGACGGTGTGTACTTCGCTTCCACCGAGAAGTCCAACATCATCCGTGGTTGGATC
TTCGGTACCACCCTGGACTCCAAGACCCAGTCCCTGCTGATCGTGAACAACGCTACCAAC
GTGGTGATCAAGGTGTGCGAGTTCCAGTTCTGCAACGACCCCTTCCTGGACGTGTACTAC
CACAAGAACAACAAGTCCTGGATGGAGTCCGAGTTCCGTGTGTACTCCTCCGCTAACAAC
TGCACCTTCGAGTACGTGTCCCAGCCCTTCCTGATGGACCTGGAGGGTAAGCAGGGTAAC
TTCAAGAACCTGCGTGAGTTCGTGTTCAAGAACATCGACGGTTACTTCAAGATCTACTCC
AAGCACACCCCCATCAACCTGGGTCGTGACCTGCCCCAGGGTTTCTCCGCTCTGGAGCCC
CTGGTGGACCTGCCCATCGGTATCAACATCACCCGTTTCCAGACCCTGCTGGCTCTGCAC
CGTTCCTACCTGACCCCCGGTGACTCCTCCTCCGGTTGGACCGCTGGTGCTGCTGCTTAC
TACGTGGGTTACCTGCAGCCCCGTACCTTCCTGCTGAAGTACAACGAGAACGGTACCATC
ACCGACGCTGTGGACTGCGCTCTGGACCCCCTGTCCGAGACCAAGTGCACCCTGAAGTCC
TTCACCGTGGAGAAGGGTATCTACCAGACCTCCAACTTCCGTGTGCAGCCCACCGAGTCC
ATCGTGCGTTTCCCCAACATCACCAACCTGTGCCCCTTCGACGAGGTGTTCAACGCTACC
CGTTTCGCTTCCGTGTACGCTTGGAACCGTAAGCGTATCTCCAACTGCGTGGCTGACTAC
TCCGTGCTGTACAACTTCGCTCCCTTCTTCGCTTTCAAGTGCTACGGTGTGTCCCCCACC
AAGCTGAACGACCTGTGCTTCACCAACGTGTACGCTGACTCCTTCGTGATCCGTGGTAAC
GAGGTGTCCCAGATCGCTCCCGGTCAGACCGGTAACATCGCTGACTACAACTACAAGCTG
CCCGACGACTTCACCGGTTGCGTGATCGCTTGGAACTCCAACAAGCTGGACTCCAAGGTG
GGTGGTAACTACAACTACCGTTACCGTCTGTTCCGTAAGTCCAACCTGAAGCCCTTCGAG
CGTGACATCTCCACCGAGATCTACCAGGCTGGTAACAAGCCCTGCAACGGTGTGGCTGGT
GTGAACTGCTACTTCCCCCTGCAGTCCTACGGTTTCCGTCCCACCTACGGTGTGGGTCAC
CAGCCCTACCGTGTGGTGGTGCTGTCCTTCGAGCTGCTGCACGCTCCCGCTACCGTGTGC
GGTCCCAAGAAGTCCACCAACCTGGTGAAGAACAAGTGCGTGAACTTCAACTTCAACGGT
CTGACCGGTACCGGTGTGCTGACCGAGTCCAACAAGAAGTTCCTGCCCTTCCAGCAGTTC
GGTCGTGACATCGCTGACACCACCGACGCTGTGCGTGACCCCCAGACCCTGGAGATCCTG
GACATCACCCCCTGCTCCTTCGGTGGTGTGTCCGTGATCACCCCCGGTACCAACACCTCC
AACCAGGTGGCTGTGCTGTACCAGGGTGTGAACTGCACCGAGGTGCCCGTGGCTATCCAC
GCTGACCAGCTGACCCCCACCTGGCGTGTGTACTCCACCGGTTCCAACGTGTTCCAGACC
CGTGCTGGTTGCCTGATCGGTGCTGAGTACGTGAACAACTCCTACGAGTGCGACATCCCC
ATCGGTGCTGGTATCTGCGCTTCCTACCAGACCCAGACCAAGTCCCACCGTCGTGCTCGT
TCCGTGGCTTCCCAGTCCATCATCGCTTACACCATGTCCCTGGGTGCTGAGAACTCCGTG
GCTTACTCCAACAACTCCATCGCTATCCCCACCAACTTCACCATCTCCGTGACCACCGAG
ATCCTGCCCGTGTCCATGACCAAGACCTCCGTGGACTGCACCATGTACATCTGCGGTGAC
TCCACCGAGTGCTCCAACCTGCTGCTGCAGTACGGTTCCTTCTGCACCCAGCTGAAGCGT
GCTCTGACCGGTATCGCTGTGGAGCAGGACAAGAACACCCAGGAGGTGTTCGCTCAGGTG
AAGCAGATCTACAAGACCCCCCCCATCAAGTACTTCGGTGGTTTCAACTTCTCCCAGATC
CTGCCCGACCCCTCCAAGCCCTCCAAGCGTTCCTTCATCGAGGACCTGCTGTTCAACAAG
GTGACCCTGGCTGACGCTGGTTTCATCAAGCAGTACGGTGACTGCCTGGGTGACATCGCT
GCTCGTGACCTGATCTGCGCTCAGAAGTTCAACGGTCTGACCGTGCTGCCCCCCCTGCTG
ACCGACGAGATGATCGCTCAGTACACCTCCGCTCTGCTGGCTGGTACCATCACCTCCGGT
TGGACCTTCGGTGCTGGTGCTGCTCTGCAGATCCCCTTCGCTATGCAGATGGCTTACCGT
TTCAACGGTATCGGTGTGACCCAGAACGTGCTGTACGAGAACCAGAAGCTGATCGCTAAC
CAGTTCAACTCCGCTATCGGTAAGATCCAGGACTCCCTGTCCTCCACCGCTTCCGCTCTG
GGTAAGCTGCAGGACGTGGTGAACCACAACGCTCAGGCTCTGAACACCCTGGTGAAGCAG
CTGTCCTCCAAGTTCGGTGCTATCTCCTCCGTGCTGAACGACATCCTGTCCCGTCTGGAC
AAGGTGGAGGCTGAGGTGCAGATCGACCGTCTGATCACCGGTCGTCTGCAGTCCCTGCAG
ACCTACGTGACCCAGCAGCTGATCCGTGCTGCTGAGATCCGTGCTTCCGCTAACCTGGCT
GCTACCAAGATGTCCGAGTGCGTGCTGGGTCAGTCCAAGCGTGTGGACTTCTGCGGTAAG
GGTTACCACCTGATGTCCTTCCCCCAGTCCGCTCCCCACGGTGTGGTGTTCCTGCACGTG
ACCTACGTGCCCGCTCAGGAGAAGAACTTCACCACCGCTCCCGCTATCTGCCACGACGGT
AAGGCTCACTTCCCCCGTGAGGGTGTGTTCGTGTCCAACGGTACCCACTGGTTCGTGACC
CAGCGTAACTTCTACGAGCCCCAGATCATCACCACCGACAACACCTTCGTGTCCGGTAAC
TGCGACGTGGTGATCGGTATCGTGAACAACACCGTGTACGACCCCCTGCAGCCCGAGCTG
GACTCCTTCAAGGAGGAGCTGGACAAGTACTTCAAGAACCACACCTCCCCCGACGTGGAC
CTGGGTGACATCTCCGGTATCAACGCTTCCGTGGTGAACATCCAGAAGGAGATCGACCGT
CTGAACGAGGTGGCTAAGAACCTGAACGAGTCCCTGATCGACCTGCAGGAGCTGGGTAAG
TACGAGCAGTACATCAAGTGGCCCTGGTACATCTGGCTGGGTTTCATCGCTGGTCTGATC
GCTATCGTGATGGTGACCATCATGCTGTGCTGCATGACCTCCTGCTGCTCCTGCCTGAAG
GGTTGCTGCTCCTGCGGTTCCTGCTGCAAGTTCGACGAGGACGACTCCGAGCCCGTGCTG
AAGGGTGTGAAGCTGCACTACACCTAG
Note:
the above sequence consists of signal peptide-extracellular domain-
transmembrane domain-intracellular domain.
SEQ ID NO: 73:
Extracellular amino acid sequence of Omicron-BA.4 5 S antigen:
MFVFLVLLPLVSSQCVNLITRTQSYTNSFTRGVYYPDKVFRSSVLHSTQDLFLPFFSNVT
WFHAISGTNGTKRFDNPVLPFNDGVYFASTEKSNIIRGWIFGTTLDSKTQSLLIVNNATN
VVIKVCEFQFCNDPFLDVYYHKNNKSWMESEFRVYSSANNCTFEYVSQPFLMDLEGKQGN
FKNLREFVFKNIDGYFKIYSKHTPINLGRDLPQGFSALEPLVDLPIGINITRFQTLLALH
RSYLTPGDSSSGWTAGAAAYYVGYLQPRTFLLKYNENGTITDAVDCALDPLSETKCTLKS
FTVEKGIYQTSNFRVQPTESIVRFPNITNLCPFDEVFNATRFASVYAWNRKRISNCVADY
SVLYNFAPFFAFKCYGVSPTKLNDLCFTNVYADSFVIRGNEVSQIAPGQTGNIADYNYKL
PDDFTGCVIAWNSNKLDSKVGGNYNYRYRLFRKSNLKPFERDISTEIYQAGNKPCNGVAG
VNCYFPLQSYGFRPTYGVGHQPYRVVVLSFELLHAPATVCGPKKSTNLVKNKCVNFNFNG
LTGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEYVNNSYECDIP
IGAGICASYQTQTKSHRRARSVASQSIIAYTMSLGAENSVAYSNNSIAIPTNFTISVTTE
ILPVSMTKTSVDCTMYICGDSTECSNLLLQYGSFCTQLKRALTGIAVEQDKNTQEVFAQV
KQIYKTPPIKYFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQIPFAMQMAYR
ENGIGVTQNVLYENQKLIANQFNSAIGKIQDSLSSTASALGKLQDVVNHNAQALNTLVKQ
LSSKFGAISSVLNDILSRLDKVEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK
YEQYIKWP
Note:
the above sequence consists of signal peptide-extracellular domain.
SEQ ID NO: 74:
Extracellular nucleic acid sequence of Omicron-BA.4/5 S antigen
(optimization of CHO cells):
ATGTTCGTGTTCCTGGTGCTGCTGCCCCTGGTGAGCAGCCAGTGCGTGAACCTGATCACC
AGAACCCAGAGCTACACCAACAGCTTCACCAGAGGCGTGTACTACCCCGACAAGGTGTTC
AGAAGCAGCGTGCTGCACAGCACCCAGGACCTGTTCCTGCCCTTCTTCAGCAACGTGACC
TGGTTCCACGCCATCAGCGGCACCAACGGCACCAAGAGATTCGACAACCCCGTGCTGCCC
TTCAACGACGGCGTGTACTTCGCCAGCACCGAGAAGAGCAACATCATCAGAGGCTGGATC
TTCGGCACCACCCTGGACAGCAAGACCCAGAGCCTGCTGATCGTGAACAACGCCACCAAC
GTGGTGATCAAGGTGTGCGAGTTCCAGTTCTGCAACGACCCCTTCCTGGACGTGTACTAC
CACAAGAACAACAAGAGCTGGATGGAGAGCGAGTTCAGAGTGTACAGCAGCGCCAACAAC
TGCACCTTCGAGTACGTGAGCCAGCCCTTCCTGATGGACCTGGAGGGCAAGCAGGGCAAC
TTCAAGAACCTGAGAGAGTTCGTGTTCAAGAACATCGACGGCTACTTCAAGATCTACAGC
AAGCACACCCCCATCAACCTGGGCAGAGACCTGCCCCAGGGCTTCAGCGCCCTGGAGCCC
CTGGTGGACCTGCCCATCGGCATCAACATCACCAGATTCCAGACCCTGCTGGCCCTGCAC
AGAAGCTACCTGACCCCCGGCGACAGCAGCAGCGGCTGGACCGCCGGCGCCGCCGCCTAC
TACGTGGGCTACCTGCAGCCCAGAACCTTCCTGCTGAAGTACAACGAGAACGGCACCATC
ACCGACGCCGTGGACTGCGCCCTGGACCCCCTGAGCGAGACCAAGTGCACCCTGAAGAGC
TTCACCGTGGAGAAGGGCATCTACCAGACCAGCAACTTCAGAGTGCAGCCCACCGAGAGC
ATCGTGAGATTCCCCAACATCACCAACCTGTGCCCCTTCGACGAGGTGTTCAACGCCACC
AGATTCGCCAGCGTGTACGCCTGGAACAGAAAGAGAATCAGCAACTGCGTGGCCGACTAC
AGCGTGCTGTACAACTTCGCCCCCTTCTTCGCCTTCAAGTGCTACGGCGTGAGCCCCACC
AAGCTGAACGACCTGTGCTTCACCAACGTGTACGCCGACAGCTTCGTGATCAGAGGCAAC
GAGGTGAGCCAGATCGCCCCCGGCCAGACCGGCAACATCGCCGACTACAACTACAAGCTG
CCCGACGACTTCACCGGCTGCGTGATCGCCTGGAACAGCAACAAGCTGGACAGCAAGGTG
GGCGGCAACTACAACTACAGATACAGACTGTTCAGAAAGAGCAACCTGAAGCCCTTCGAG
AGAGACATCAGCACCGAGATCTACCAGGCCGGCAACAAGCCCTGCAACGGCGTGGCCGGC
GTGAACTGCTACTTCCCCCTGCAGAGCTACGGCTTCAGACCCACCTACGGCGTGGGCCAC
CAGCCCTACAGAGTGGTGGTGCTGAGCTTCGAGCTGCTGCACGCCCCCGCCACCGTGTGC
GGCCCCAAGAAGAGCACCAACCTGGTGAAGAACAAGTGCGTGAACTTCAACTTCAACGGC
CTGACCGGCACCGGCGTGCTGACCGAGAGCAACAAGAAGTTCCTGCCCTTCCAGCAGTTC
GGCAGAGACATCGCCGACACCACCGACGCCGTGAGAGACCCCCAGACCCTGGAGATCCTG
GACATCACCCCCTGCAGCTTCGGCGGCGTGAGCGTGATCACCCCCGGCACCAACACCAGC
AACCAGGTGGCCGTGCTGTACCAGGGCGTGAACTGCACCGAGGTGCCCGTGGCCATCCAC
GCCGACCAGCTGACCCCCACCTGGAGAGTGTACAGCACCGGCAGCAACGTGTTCCAGACC
AGAGCCGGCTGCCTGATCGGCGCCGAGTACGTGAACAACAGCTACGAGTGCGACATCCCC
ATCGGCGCCGGCATCTGCGCCAGCTACCAGACCCAGACCAAGAGCCACAGAAGAGCCAGA
AGCGTGGCCAGCCAGAGCATCATCGCCTACACCATGAGCCTGGGCGCCGAGAACAGCGTG
GCCTACAGCAACAACAGCATCGCCATCCCCACCAACTTCACCATCAGCGTGACCACCGAG
ATCCTGCCCGTGAGCATGACCAAGACCAGCGTGGACTGCACCATGTACATCTGCGGCGAC
AGCACCGAGTGCAGCAACCTGCTGCTGCAGTACGGCAGCTTCTGCACCCAGCTGAAGAGA
GCCCTGACCGGCATCGCCGTGGAGCAGGACAAGAACACCCAGGAGGTGTTCGCCCAGGTG
AAGCAGATCTACAAGACCCCCCCCATCAAGTACTTCGGCGGCTTCAACTTCAGCCAGATC
CTGCCCGACCCCAGCAAGCCCAGCAAGAGAAGCTTCATCGAGGACCTGCTGTTCAACAAG
GTGACCCTGGCCGACGCCGGCTTCATCAAGCAGTACGGCGACTGCCTGGGCGACATCGCC
GCCAGAGACCTGATCTGCGCCCAGAAGTTCAACGGCCTGACCGTGCTGCCCCCCCTGCTG
ACCGACGAGATGATCGCCCAGTACACCAGCGCCCTGCTGGCCGGCACCATCACCAGCGGC
TGGACCTTCGGCGCCGGCGCCGCCCTGCAGATCCCCTTCGCCATGCAGATGGCCTACAGA
TTCAACGGCATCGGCGTGACCCAGAACGTGCTGTACGAGAACCAGAAGCTGATCGCCAAC
CAGTTCAACAGCGCCATCGGCAAGATCCAGGACAGCCTGAGCAGCACCGCCAGCGCCCTG
GGCAAGCTGCAGGACGTGGTGAACCACAACGCCCAGGCCCTGAACACCCTGGTGAAGCAG
CTGAGCAGCAAGTTCGGCGCCATCAGCAGCGTGCTGAACGACATCCTGAGCAGACTGGAC
AAGGTGGAGGCCGAGGTGCAGATCGACAGACTGATCACCGGCAGACTGCAGAGCCTGCAG
ACCTACGTGACCCAGCAGCTGATCAGAGCCGCCGAGATCAGAGCCAGCGCCAACCTGGCC
GCCACCAAGATGAGCGAGTGCGTGCTGGGCCAGAGCAAGAGAGTGGACTTCTGCGGCAAG
GGCTACCACCTGATGAGCTTCCCCCAGAGCGCCCCCCACGGCGTGGTGTTCCTGCACGTG
ACCTACGTGCCCGCCCAGGAGAAGAACTTCACCACCGCCCCCGCCATCTGCCACGACGGC
AAGGCCCACTTCCCCAGAGAGGGCGTGTTCGTGAGCAACGGCACCCACTGGTTCGTGACC
CAGAGAAACTTCTACGAGCCCCAGATCATCACCACCGACAACACCTTCGTGAGCGGCAAC
TGCGACGTGGTGATCGGCATCGTGAACAACACCGTGTACGACCCCCTGCAGCCCGAGCTG
GACAGCTTCAAGGAGGAGCTGGACAAGTACTTCAAGAACCACACCAGCCCCGACGTGGAC
CTGGGCGACATCAGCGGCATCAACGCCAGCGTGGTGAACATCCAGAAGGAGATCGACAGA
CTGAACGAGGTGGCCAAGAACCTGAACGAGAGCCTGATCGACCTGCAGGAGCTGGGCAAG
TACGAGCAGTACATCAAGTGGCCCTAG
Note:
the above sequence consists of signal peptide-extracellular domain.
SEQ ID NO: 75:
Extracellular nucleic acid sequence of Omicron-BA.4/5 S antigen
(optimization of insect cells):
ATGTTCGTGTTCCTGGTGCTGCTGCCCCTGGTGTCCTCCCAGTGCGTGAACCTGATCACC
CGTACCCAGTCCTACACCAACTCCTTCACCCGTGGTGTGTACTACCCCGACAAGGTGTTC
CGTTCCTCCGTGCTGCACTCCACCCAGGACCTGTTCCTGCCCTTCTTCTCCAACGTGACC
TGGTTCCACGCTATCTCCGGTACCAACGGTACCAAGCGTTTCGACAACCCCGTGCTGCCC
TTCAACGACGGTGTGTACTTCGCTTCCACCGAGAAGTCCAACATCATCCGTGGTTGGATC
TTCGGTACCACCCTGGACTCCAAGACCCAGTCCCTGCTGATCGTGAACAACGCTACCAAC
GTGGTGATCAAGGTGTGCGAGTTCCAGTTCTGCAACGACCCCTTCCTGGACGTGTACTAC
CACAAGAACAACAAGTCCTGGATGGAGTCCGAGTTCCGTGTGTACTCCTCCGCTAACAAC
TGCACCTTCGAGTACGTGTCCCAGCCCTTCCTGATGGACCTGGAGGGTAAGCAGGGTAAC
TTCAAGAACCTGCGTGAGTTCGTGTTCAAGAACATCGACGGTTACTTCAAGATCTACTCC
AAGCACACCCCCATCAACCTGGGTCGTGACCTGCCCCAGGGTTTCTCCGCTCTGGAGCCC
CTGGTGGACCTGCCCATCGGTATCAACATCACCCGTTTCCAGACCCTGCTGGCTCTGCAC
CGTTCCTACCTGACCCCCGGTGACTCCTCCTCCGGTTGGACCGCTGGTGCTGCTGCTTAC
TACGTGGGTTACCTGCAGCCCCGTACCTTCCTGCTGAAGTACAACGAGAACGGTACCATC
ACCGACGCTGTGGACTGCGCTCTGGACCCCCTGTCCGAGACCAAGTGCACCCTGAAGTCC
TTCACCGTGGAGAAGGGTATCTACCAGACCTCCAACTTCCGTGTGCAGCCCACCGAGTCC
ATCGTGCGTTTCCCCAACATCACCAACCTGTGCCCCTTCGACGAGGTGTTCAACGCTACC
CGTTTCGCTTCCGTGTACGCTTGGAACCGTAAGCGTATCTCCAACTGCGTGGCTGACTAC
TCCGTGCTGTACAACTTCGCTCCCTTCTTCGCTTTCAAGTGCTACGGTGTGTCCCCCACC
AAGCTGAACGACCTGTGCTTCACCAACGTGTACGCTGACTCCTTCGTGATCCGTGGTAAC
GAGGTGTCCCAGATCGCTCCCGGTCAGACCGGTAACATCGCTGACTACAACTACAAGCTG
CCCGACGACTTCACCGGTTGCGTGATCGCTTGGAACTCCAACAAGCTGGACTCCAAGGTG
GGTGGTAACTACAACTACCGTTACCGTCTGTTCCGTAAGTCCAACCTGAAGCCCTTCGAG
CGTGACATCTCCACCGAGATCTACCAGGCTGGTAACAAGCCCTGCAACGGTGTGGCTGGT
GTGAACTGCTACTTCCCCCTGCAGTCCTACGGTTTCCGTCCCACCTACGGTGTGGGTCAC
CAGCCCTACCGTGTGGTGGTGCTGTCCTTCGAGCTGCTGCACGCTCCCGCTACCGTGTGC
GGTCCCAAGAAGTCCACCAACCTGGTGAAGAACAAGTGCGTGAACTTCAACTTCAACGGT
CTGACCGGTACCGGTGTGCTGACCGAGTCCAACAAGAAGTTCCTGCCCTTCCAGCAGTTC
GGTCGTGACATCGCTGACACCACCGACGCTGTGCGTGACCCCCAGACCCTGGAGATCCTG
GACATCACCCCCTGCTCCTTCGGTGGTGTGTCCGTGATCACCCCCGGTACCAACACCTCC
AACCAGGTGGCTGTGCTGTACCAGGGTGTGAACTGCACCGAGGTGCCCGTGGCTATCCAC
GCTGACCAGCTGACCCCCACCTGGCGTGTGTACTCCACCGGTTCCAACGTGTTCCAGACC
CGTGCTGGTTGCCTGATCGGTGCTGAGTACGTGAACAACTCCTACGAGTGCGACATCCCC
ATCGGTGCTGGTATCTGCGCTTCCTACCAGACCCAGACCAAGTCCCACCGTCGTGCTCGT
TCCGTGGCTTCCCAGTCCATCATCGCTTACACCATGTCCCTGGGTGCTGAGAACTCCGTG
GCTTACTCCAACAACTCCATCGCTATCCCCACCAACTTCACCATCTCCGTGACCACCGAG
ATCCTGCCCGTGTCCATGACCAAGACCTCCGTGGACTGCACCATGTACATCTGCGGTGAC
TCCACCGAGTGCTCCAACCTGCTGCTGCAGTACGGTTCCTTCTGCACCCAGCTGAAGCGT
GCTCTGACCGGTATCGCTGTGGAGCAGGACAAGAACACCCAGGAGGTGTTCGCTCAGGTG
AAGCAGATCTACAAGACCCCCCCCATCAAGTACTTCGGTGGTTTCAACTTCTCCCAGATC
CTGCCCGACCCCTCCAAGCCCTCCAAGCGTTCCTTCATCGAGGACCTGCTGTTCAACAAG
GTGACCCTGGCTGACGCTGGTTTCATCAAGCAGTACGGTGACTGCCTGGGTGACATCGCT
GCTCGTGACCTGATCTGCGCTCAGAAGTTCAACGGTCTGACCGTGCTGCCCCCCCTGCTG
ACCGACGAGATGATCGCTCAGTACACCTCCGCTCTGCTGGCTGGTACCATCACCTCCGGT
TGGACCTTCGGTGCTGGTGCTGCTCTGCAGATCCCCTTCGCTATGCAGATGGCTTACCGT
TTCAACGGTATCGGTGTGACCCAGAACGTGCTGTACGAGAACCAGAAGCTGATCGCTAAC
CAGTTCAACTCCGCTATCGGTAAGATCCAGGACTCCCTGTCCTCCACCGCTTCCGCTCTG
GGTAAGCTGCAGGACGTGGTGAACCACAACGCTCAGGCTCTGAACACCCTGGTGAAGCAG
CTGTCCTCCAAGTTCGGTGCTATCTCCTCCGTGCTGAACGACATCCTGTCCCGTCTGGAC
AAGGTGGAGGCTGAGGTGCAGATCGACCGTCTGATCACCGGTCGTCTGCAGTCCCTGCAG
ACCTACGTGACCCAGCAGCTGATCCGTGCTGCTGAGATCCGTGCTTCCGCTAACCTGGCT
GCTACCAAGATGTCCGAGTGCGTGCTGGGTCAGTCCAAGCGTGTGGACTTCTGCGGTAAG
GGTTACCACCTGATGTCCTTCCCCCAGTCCGCTCCCCACGGTGTGGTGTTCCTGCACGTG
ACCTACGTGCCCGCTCAGGAGAAGAACTTCACCACCGCTCCCGCTATCTGCCACGACGGT
AAGGCTCACTTCCCCCGTGAGGGTGTGTTCGTGTCCAACGGTACCCACTGGTTCGTGACC
CAGCGTAACTTCTACGAGCCCCAGATCATCACCACCGACAACACCTTCGTGTCCGGTAAC
TGCGACGTGGTGATCGGTATCGTGAACAACACCGTGTACGACCCCCTGCAGCCCGAGCTG
GACTCCTTCAAGGAGGAGCTGGACAAGTACTTCAAGAACCACACCTCCCCCGACGTGGAC
CTGGGTGACATCTCCGGTATCAACGCTTCCGTGGTGAACATCCAGAAGGAGATCGACCGT
CTGAACGAGGTGGCTAAGAACCTGAACGAGTCCCTGATCGACCTGCAGGAGCTGGGTAAG
TACGAGCAGTACATCAAGTGGCCCTAG
Note:
the above sequence consists of signal peptide-extracellular domain.
SEQ ID NO: 76:
Nucleotide sequence of S protein of BQ.1.1 variant strain
ATGTTCGTGTTCCTGGTGCTGCTGCCCCTGGTGAGCAGCCAATGCGTGAACCTGATCACA
AGAACACAGAGCTACACCAACAGCTTCACAAGAGGCGTGTACTACCCCGACAAGGTGTTC
AGAAGCAGCGTGCTGCACAGCACCCAAGACCTGTTTCTCCCCTTCTTCAGCAACGTGACC
TGGTTCCACGCCATCAGCGGCACCAACGGCACCAAGAGATTCGACAACCCCGTGCTGCCC
TTCAACGACGGCGTGTACTTCGCTAGCACCGAGAAGAGCAACATCATCAGAGGCTGGATC
TTCGGCACCACCCTGGACAGCAAGACACAGAGCCTGCTGATCGTGAATAACGCCACCAAC
GTGGTGATCAAGGTGTGCGAGTTTCAGTTCTGCAACGACCCCTTCCTGGACGTGTACTAC
CACAAGAACAACAAGAGCTGGATGGAGAGCGAGTTCAGAGTGTACAGCAGCGCTAACAAC
TGCACCTTCGAGTACGTGAGCCAACCCTTCCTGATGGACCTGGAGGGCAAGCAAGGCAAC
TTCAAGAACCTGAGAGAGTTCGTGTTCAAGAACATCGACGGCTACTTCAAGATCTACAGC
AAGCACACCCCCATCAACCTGGGCAGAGACCTGCCCCAAGGCTTCAGCGCCCTGGAGCCC
CTGGTGGACCTGCCCATCGGCATCAACATCACAAGATTTCAGACCCTGCTGGCCCTGCAC
AGAAGCTACCTCACCCCTGGGGACAGCAGCAGCGGCTGGACCGCTGGCGCCGCTGCCTAC
TACGTGGGCTACCTGCAGCCTAGAACCTTCCTGCTGAAGTACAACGAGAACGGCACCATT
ACAGACGCCGTGGACTGCGCCCTGGACCCCCTGAGCGAGACCAAGTGCACCCTGAAGAGC
TTCACCGTGGAGAAGGGCATCTATCAGACAAGCAACTTCAGAGTGCAGCCCACCGAGAGC
ATCGTGAGATTCCCCAACATCACCAACCTGTGCCCCTTCGACGAGGTGTTCAACGCCACC
ACCTTCGCTAGCGTGTACGCCTGGAATAGAAAGAGAATCAGCAACTGCGTGGCCGACTAC
AGCGTGCTGTACAACTTCGCCCCCTTCTTCGCCTTCAAATGCTATGGCGTGAGCCCCACC
AAGCTGAACGACCTGTGCTTCACCAACGTGTACGCCGACAGCTTCGTGATCAGAGGCAAC
GAGGTGAGCCAAATCGCCCCTGGGCAGACCGGCAACATCGCCGATTACAACTACAAACTG
CCCGACGACTTCACCGGCTGCGTGATCGCCTGGAACAGCAACAAGCTGGACAGCACCGTG
GGCGGCAACTACAACTACCGGTACAGACTGTTCAGAAAGAGCAAGCTGAAGCCCTTCGAG
AGAGACATCAGCACCGAGATCTACCAAGCCGGCAACAAGCCCTGCAACGGCGTGGCCGGC
GTGAACTGCTACTTCCCCCTGCAGAGCTACGGCTTCAGACCCACCTACGGCGTGGGCCAT
CAGCCCTACAGAGTGGTCGTGCTGAGCTTCGAGCTGCTGCACGCCCCCGCCACCGTGTGC
GGCCCCAAGAAGAGCACCAACCTGGTGAAGAACAAGTGCGTGAACTTCAACTTCAACGGC
CTGACCGGCACCGGCGTGCTGACCGAGAGCAACAAGAAGTTCCTGCCCTTTCAGCAGTTC
GGCAGAGACATCGCCGACACCACCGACGCCGTGCGGGACCCTCAGACCCTGGAGATCCTG
GACATCACCCCCTGCAGCTTTGGCGGCGTGAGCGTGATCACCCCCGGCACCAACACAAGC
AACCAAGTGGCCGTGCTGTACCAAGGCGTCAATTGCACCGAGGTGCCCGTGGCCATCCAC
GCCGATCAGCTGACCCCCACCTGGAGAGTCTACAGCACCGGCAGCAACGTGTTTCAGACA
AGAGCCGGCTGCCTGATCGGCGCCGAGTACGTGAACAACAGCTACGAGTGCGACATCCCC
ATCGGCGCCGGCATCTGCGCTAGCTATCAGACACAGACCAAGAGCCACGGCAGCGCTAGC
AGCGTGGCTAGCCAAAGCATCATCGCCTACACCATGAGCCTGGGCGCCGAGAACAGCGTG
GCCTACAGCAACAACAGCATCGCCATCCCCACCAACTTCACCATCAGCGTGACCACCGAG
ATCCTGCCCGTCAGCATGACCAAGACAAGCGTGGACTGCACCATGTACATCTGCGGCGAC
AGCACCGAGTGCAGCAACCTGCTCCTGCAGTACGGCAGCTTCTGCACACAGCTGAAGAGA
GCCCTGACCGGCATCGCCGTGGAGCAAGACAAGAACACCCAAGAGGTGTTCGCCCAAGTG
AAGCAGATCTACAAGACCCCCCCCATCAAGTACTTCGGCGGCTTCAACTTCAGCCAAATC
CTGCCCGATCCTAGCAAGCCTAGCAAGAGAAGCTTCATCGAGGACCTGCTGTTCAACAAG
GTGACCCTGGCCGACGCCGGCTTCATCAAGCAGTACGGCGACTGCCTGGGCGATATCGCC
GCTAGAGACCTGATCTGCGCTCAGAAGTTCAATGGCCTGACCGTGCTGCCCCCCCTGCTG
ACCGACGAGATGATCGCTCAGTACACAAGCGCCCTGCTGGCCGGCACCATCACAAGCGGC
TGGACATTCGGCGCCGGCGCTGCTCTGCAGATCCCCTTCGCCATGCAGATGGCCTACAGA
TTCAACGGCATCGGCGTGACACAGAACGTGCTGTACGAGAATCAGAAGCTGATCGCCAAT
CAGTTCAACAGCGCCATCGGCAAGATCCAAGACAGCCTGAGCAGCACCGCTAGCGCCCTG
GGCAAGCTGCAAGACGTGGTGAACCACAACGCCCAAGCCCTGAACACCCTGGTGAAGCAG
CTGAGCAGCAAGTTCGGCGCCATCAGCAGCGTGCTGAACGATATCCTGAGCAGACTGGAC
CCCCCCGAGGCCGAGGTGCAGATCGACAGACTGATTACCGGCAGACTGCAGAGCCTGCAG
ACCTACGTGACACAGCAGCTGATCAGAGCCGCCGAGATCAGAGCTAGCGCCAACCTGGCC
GCCACCAAGATGAGCGAGTGCGTGCTGGGGCAGAGCAAGAGAGTGGACTTCTGCGGCAAG
GGCTACCACCTGATGAGCTTCCCTCAGAGCGCCCCCCACGGCGTGGTGTTCCTGCACGTG
ACCTACGTGCCCGCCCAAGAGAAGAACTTCACCACCGCTCCCGCTATCTGCCACGACGGC
AAGGCCCACTTCCCTAGAGAGGGCGTGTTCGTGAGCAACGGCACCCACTGGTTCGTGACA
CAGAGAAACTTCTACGAGCCTCAGATCATCACCACCGACAACACCTTCGTGAGCGGCAAC
TGCGACGTGGTGATCGGCATCGTGAACAACACCGTGTACGACCCCCTGCAACCCGAGCTG
GACAGCTTCAAGGAGGAGCTGGACAAGTACTTTAAGAACCACACAAGCCCCGACGTGGAC
CTGGGCGACATTAGCGGCATCAACGCTAGCGTGGTGAACATTCAGAAGGAGATTGACAGA
CTGAACGAGGTGGCCAAGAACCTGAACGAGAGCCTGATCGACCTGCAAGAGCTGGGCAAG
TACGAGCAGTACATCAAGTGGCCCTGGTACATCTGGCTGGGCTTCATCGCCGGCCTGATC
GCCATCGTGATGGTGACCATCATGCTGTGCTGCATGACAAGCTGTTGCAGCTGCCTCAAG
GGCTGCTGCAGCTGCGGCAGCTGCTGCAAGTTCGACGAGGACGACAGCGAGCCCGTGCTG
AAGGGCGTGAAGCTGCACTACACCTGA
SEQ ID NO: 77:
Amino acid sequence of S protein of BQ.1.1 variant strain
MFVFLVLLPLVSSQCVNLITRTQSYTNSFTRGVYYPDKVFRSSVLHSTQDLFLPFFSNVT
WFHAISGTNGTKRFDNPVLPFNDGVYFASTEKSNIIRGWIFGTTLDSKTQSLLIVNNATN
VVIKVCEFQFCNDPFLDVYYHKNNKSWMESEFRVYSSANNCTFEYVSQPFLMDLEGKQGN
FKNLREFVFKNIDGYFKIYSKHTPINLGRDLPQGFSALEPLVDLPIGINITRFQTLLALH
RSYLTPGDSSSGWTAGAAAYYVGYLQPRTFLLKYNENGTITDAVDCALDPLSETKCTLKS
FTVEKGIYQTSNFRVQPTESIVRFPNITNLCPFDEVFNATTFASVYAWNRKRISNCVADY
SVLYNFAPFFAFKCYGVSPTKLNDLCFTNVYADSFVIRGNEVSQIAPGQTGNIADYNYKL
PDDFTGCVIAWNSNKLDSTVGGNYNYRYRLFRKSKLKPFERDISTEIYQAGNKPCNGVAG
VNCYFPLQSYGFRPTYGVGHQPYRVVVLSFELLHAPATVCGPKKSTNLVKNKCVNFNFNG
LTGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEYVNNSYECDIP
IGAGICASYQTQTKSHGSASSVASQSIIAYTMSLGAENSVAYSNNSIAIPTNFTISVTTE
ILPVSMTKTSVDCTMYICGDSTECSNLLLQYGSFCTQLKRALTGIAVEQDKNTQEVFAQV
KQIYKTPPIKYFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNHNAQALNTLVKQ
LSSKFGAISSVLNDILSRLDPPEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK
YEQYIKWPWYIWLGFIAGLIAIVMVTIMLCCMTSCCSCLKGCCSCGSCCKFDEDDSEPVL
KGVKLHYT

Beneficial effect: the present invention provides a broad-spectrum protein for resisting infection with SARS-COV-2 or a mutant thereof and a vaccine thereof, and specifically provides a recombinant protein vaccine for resisting infection with SARS-COV-2 or the mutant thereof, a adenovirus vector vaccine which takes the human type 5 replication-deficient adenovirus as vector; and the nucleotide sequences encoding the two vaccines are selected from at least of SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:19, SEQ ID NO:21, SEQ ID NO: 23, SEQ ID NO:24, SEQ ID NO:26, SEQ ID NO:28, SEQ ID NO: 30, SEQ ID NO: 32, SEQ ID NO: 34, SEQ ID NO:36, SEQ ID NO:38, SEQ ID NO:41, SEQ ID NO:42, SEQ ID NO:44, SEQ ID NO:45, SEQ ID NO:47, SEQ ID NO:48, SEQ ID NO:50, SEQ ID NO:51, SEQ ID NO: 53, SEQ ID NO:54, SEQ ID NO:56, SEQ ID NO: 57, SEQ ID NO: 59, SEQ ID NO: 60, SEQ ID NO: 62, SEQ ID NO:63, SEQ ID NO:65, SEQ ID NO:66, SEQ ID NO:68, SEQ ID NO:69, SEQ ID NO:71, SEQ ID NO:72, SEQ ID NO:74, SEQ ID NO:75, or SEQ ID NO:76. Meanwhile, in the present invention, the two vaccines are combined, where the recombinant protein vaccine is mainly prepared based on the RBD sequence of SARS-COV-2, the optimized sequence composed of RBD and HR of the SARS-COV-2 mutant, and the full-length S protein sequence of CHO cell optimized expression; the adenovirus vaccine is constructed by optimizing the full-length sequence of S antigen of SARS-COV-2 or its mutant, so as to prepare a pharmaceutical composition or combined drug for preventing and/or treating infection with SARS-COV-2 or its mutant, namely bivalent vaccine. The present invention can induce antibodies and other immune reactions in the body through intramuscular injection, nasal spray administration or combined administration of the two vaccines and block the binding of the S protein of SARS-COV-2 with the ACE2 receptor of the host cell, thereby helping the host resist coronavirus infection, and especially having better prevention and treatment effect on the mutant virus.

### BRIEF DESCRIPTION OF THE DRAWINGS

FIG. 1 shows a schematic diagram of construction of a full-length gene of recombinant Ad5_{Beta/Delta} virus and an expression level diagram of spike induced by a recombinant adenovirus in Experiment 1.1.

FIG. 2 shows a testing diagram of anti-RBD specific binding antibody levels of recombinant adenovirus vaccines Ad5_{WT}, Ad5_{Beta}, Ad5_{Delta}, Ad5_{Omicron} and Ad5_{Beta/Delta} in the blood of immunized mice in Experiment 1.3.

FIG. 3 shows a testing result diagram of neutralizing antibodies of recombinant adenovirus vaccines Ad5_{WT}, Ad5_{Beta}, Ad5_{Delta}, Ad5_{BA.1}, Ad5_{Beta/Delta} and Ad5_{BA.4/5} against pseudovirus in Experiment 1.4.

FIG. 4 shows a testing result diagram of anti-RBD specific IgG and IgA antibodies of recombinant adenovirus vaccine Ad5 Beta Delta in bronchoalveolar lavage fluid of immunized mice in Experiment 1.5.

FIG. 5 shows a testing result diagram of adenovirus vaccine Ad5 Beta Delta on tissue resident T cells in Experiment 1.6.

FIG. 6 shows a testing result diagram of recombinant adenovirus vaccine Ad5 Beta Delta on antigen-specific T cells in lung tissue in Experiment 1.7.

FIG. 7 shows a testing result diagram of reactions of a germinal center of recombinant adenovirus vaccine Ad5 Beta Delta in Experiment 1.8.

FIG. 8 shows a design diagram of pFastBac1-GP67-Trx-His-EK-S-RBD (Omicron_BA.4/.5)-HR in Embodiment 2.1.

FIG. 9 shows a 1% agarose gel electrophoresis map of cloning PCR products at GP67-Trx-His-EK-S-RBD (Omicron_BA.4/.5)-HR fragments 1 #-5 # in Embodiment 2.1.

FIG. 10 shows a 1% agarose gel electrophoresis map of PCR identification of recombinant bacmid in Embodiment 2.1.

FIG. 11 shows a flow diagram of packaging and amplification of recombinant baculovirus in Embodiment 2.1.

FIG. 12 shows a WB verification result diagram of target protein during baculovirus expansion in Embodiment 2.1.

FIG. 13 shows an electrophoresis and WB verification diagram of target protein of CHO cell expression in Embodiment 2.2.

FIG. 14 shows a pseudovirus neutralization assay after immunization with a single protein vaccine in Experiment 2.2.

FIG. 15 shows a testing result diagram of neutralizing antibodies induced by bivalent RBD protein vaccine in Experiment 2.3.

FIG. 16 shows a pseudovirus neutralization assay of S protein vaccines of WT, Beta and Delta strains in Experiment 2.4.

FIG. 17 shows a pseudovirus neutralization assay of S protein vaccines of different Omicron variant strains in Experiment 2.4.

FIG. 18 shows a flow chart of immunization preparation of a nasal spray prepared by mixing adenovirus Ad5_{BA.4/5} with RDB-HR_{Delta}, and a testing result diagram of expression levels of recombinant Ad5_{BA.4/5} virus-induced spike.

FIG. 19 shows a time cycle chart of intranasal immunization for an animal in Experiment 3.2.

FIG. 20 shows a testing result diagram of anti-RBD specific antibodies in serum and bronchoalveolar lavage fluid of immunized mice in Experiment 3.3.

FIG. 21 shows a testing result diagram of pseudovirus neutralization assay in Experiment 3.4.

FIG. 22 shows a pseudovirus neutralization assay of immunization preparation of a nasal spray prepared by mixing Ad5_{WT}+RDB-WT.

FIG. 23 shows a pseudovirus neutralization assay of immunization preparation of a nasal spray prepared by mixing Ad5_{BA.4/5}+S-BA.4/5.

FIG. 24 shows a mouse challenge test (Ad5_{BA.4/5}+S-BA.4/5) in Experiment 3.5.

FIG. 25 shows a testing result diagram of tissue resident T cells in bronchoalveolar lavage fluid in Experiment 3.6.

FIG. 26 shows a testing diagram of antigen-specific T cells in lung tissue in Experiment 3.7.

FIG. 27 shows detection of reactions of a germinal center in Experiment 3.8.

### DETAILED DESCRIPTION

### Abbreviations of Terms

Monophosphoryl lipid A (MPL), squalene oil-in-water emulsion (MF59), recombinant cholera toxin (rCTB), astragalus polysaccharide (APS), phosphatidylethanolamine (PE), phosphatidylcholine (PC), cholesterol (Chol), dioleoyl phosphatidylethanolamine (DOPE), (2,3-dioleoxypropyl) trimethyl ammonium chloride (DOTAP), N-[1-(2,3-dioleoyl chloride) propyl]-N,N,N-trimethylamine chloride (DOTMA), cationic cholesterol (DC-Chol), dimethyl-2, 3-dioleoxypropyl-2-(2-arginine formyl amino) ethyl ammonium trimethyldodecylammonium bromide (DOSPA), dodecyltrimethylammonium bromide (DTAB), tetradecyltrimethylammonium bromide (TTAB), hexadecyltrimethylammonium bromide (CTAB), dimethyldioctadecylammonium bromide (DDAB), or CpG ODN (nucleotide sequence containing non-methylated cytosine-phosphate-guanine as core sequence, synthetic CpG).

In the present invention, a recombinant protein vaccine and an adenovirus vaccine for resisting infection with SARS-COV-2 are designed mainly based on the amino acid sequence at sites 320-545 in the S protein of SARS-COV-2 or its mutant, heptapeptide repeat region 1 (HR1) and heptapeptide repeat region 2 (HR2). Meanwhile, new sequences are constructed based on RBD sequences, HR1 and HR2 sequences of different SARS-COV-2 mutants, and finally, drugs for resisting infection with SARS-COV-2 are constructed based on full-length nucleotides of the S protein of SARS-COV-2 or its mutant virus, extracellular domains or transmembrane domains or intracellular domains. In order to improve the stability and expression level of the S protein, the amino acid sequence of the S protein of SARS-COV-2 or its mutant is further artificially mutated when the drug for resisting infection with SARS-COV-2 is designed.

The present invention further provides a recombinant protein vaccine and an adenovirus vaccine for resisting infection with SARS-COV-2 or its mutants or a pharmaceutical composition or a combined drug of the two vaccines. This is mainly aimed at the S protein of SARS-COV-2 virus or its mutants. Particularly, the ACE2 receptor binding domain of the S protein is blocked to induce immune reactions such as antibodies in the body, thereby blocking the binding of the S protein of SARS-COV-2 or its mutant to the host cell ACE2 receptor, helping the host resist coronavirus infection and especially having better prevention and treatment effect against mutant viruses such as Alpha, Beta, Gamma, Delta, Omicron and Omicron variant strains.

The present invention also proves that a pharmaceutical composition taking a recombinant protein vaccine and an inactivated influenza virus vaccine for resisting infection with SARS-COV-2 or its mutants as active ingredients or a pharmaceutical composition taking an adenovirus vaccine and an influenza recombinant protein vaccine for resisting infection with SARS-COV-2 or its mutants as active ingredients has a better prevention and treatment effect on respiratory virus infection or diseases therefrom.

The following will explain the solution of the present invention with reference to embodiments. Those skilled in the art will understand that the following embodiments are used only to illustrate the present invention and should not be regarded as limiting the scope of the present invention. If the specific technologies or conditions are not specified in the embodiments, it shall be carried out according to the technologies or conditions described in the literature in the art or according to the product specification. Reagents or devices used, where the manufacturer is not indicated, are conventional products that can be purchased from the market.

### Part I: Immune Protection Induced by Individual Adenoviruses

### Embodiment 1.1 Construction and Preparation of Recombinant Adenovirus Vaccine

### 1. Optimization and Synthesis of S Protein Gene (Wild Strains and Variant Strains) Sequences

Wuhan virus strain (GenBank accession number: YP_009724390.1) are used as a template to obtain the gene sequence of wild spike(S), and the signal peptide of the S protein is retained. On this basis, in order to improve the expression level of the protein, codons are optimized, and then the optimized sequence is synthesized.

On the basis of the spike(S) sequence of the Wuhan virus strain (GenBank accession number: YP_009724390.1), according to the mutation sites of SARS-COV-2 Beta, Delta, Omicron and other variant strains provided on the website of https://covariants.org/, the nucleotide sequence of S protein of each variant strain is obtained, and the signal peptide of each S protein mutant is retained. The gene sequence of Beta-Delta chimeric S protein is based on the mutation site of S protein in Beta variant strain and the mutation site of S protein RBD in Delta variant strain. On this basis, in order to ensure the stability of the protein and improve its expression level, a protein structure is mutated. Firstly, lysine (L) at site 986 is replaced with proline (P), and valine (V) at site 987 is replaced with proline (P) to improve the stability and expression level of the S protein. Secondly, the codons are optimized. Finally, the S protein genes of each variant strain are synthesized according to the mutation and optimized sequence.

The S protein genomes of SARS-COV-2 and its variant strain synthesized according to the above method comprise WT (wild type), Beta, Delta, Beta/Delta chimeric type, Omicron BA.1 and Omicron BA.4/5, and their nucleotide sequences are as follows: SEQ ID NO:24, SEQ ID NO: 26, SEQ ID NO:28, SEQ ID NO:30, SEQ ID NO:32 and SEQ ID NO:38.

### 2. Packaging of Recombinant Adenovirus SARS-COV-2 Vaccine

In a gene synthesis process, S genesare cloned into a pDC316 vector by a recombinant cloning strategy, and the shuttle plasmids (pDC316-S) are obtained. The above-mentioned pDC316-S containing S genes of wild strains and variant strains is co-transfected into HEK293 cells with skeleton plasmids pBHGlox_E1,3Cre of AdMax adenovirus system, respectively; and the recombinant adenovirus is packaged by the following steps:
1) HEK293A cells with 8×10⁵ cells/well are inoculated into a six-well plate, and a high-glucose DMEM+10% FBS culture medium is overnight cultured in a cell incubator with 5% CO₂ at 37° C.
2) Next day, the above solution is replaced with high-glucose DMEM+2% FBS, and the skeleton plasmids (pBHGlox_E1, 3Cre) and the shuttle plasmids are co-transfected into HEK293A cells with lipofectamine3000. Specifically, 4 μg skeleton plasmids and 2 μg shuttle plasmids are taken for each transfection well and diluted with 125 μL Opti-MEM medium; 12 μL P3000 reagent is added into the diluted plasmids; another 1.5 ml EP tube is taken; 7.5 μL lipofectamine3000 is diluted with 125 μL Opti-MEM medium; the diluted plasmids and diluted lipofectamine3000 are mixed at the ratio of 1:1, incubated at room temperature for 10-15 minutes and then added into the cells; the cells continued to be cultured. After the overgrowth of the cells, they are passed on to a 25 cm² cell culture bottle, and the virus generation signs of the cells are observed every day; after the overgrowth of the cells at the bottom of the bottle, they are passed on to a 75 cm 2 cell culture bottle until the cells appeared obvious plaque, and virus is collected when most of the cells are diseased and shed from the bottom.
3) The cell culture with virus is collected and centrifuged at 1200 rpm for 3 minutes; a supernatant containing virus is sucked; a cell pellet is re-suspended with the supernatant containing virus at 1/10 culture volume, repeatedly frozen and thawed for three times in a refrigerator at −80° C. and a water bath at 37° C., and centrifuged at 3000 rpm for 20 minutes; and the supernatant containing virus is collected and mixed with the above supernatant containing virus, and the obtained product is the virus strain of adenovirus vaccine.
4) 50 μL vaccine candidate virus strain solution is taken and mixed with 2 μL protease K, the mixture is dissociated at 50° C. for 30 min to release the virus genome, and this is used as a PCR amplification S gene sequence. After the PCR product electrophoresis gel is recovered, it is sequenced and identified. The conditions for PCR amplification are as follows:
degeneration: 95° C., 10 min; degeneration: 95° C., 10 s; annealing: 64° C., 30 s; elongation: 72° C., 2 min; elongation: 72° C., 5 min; number of cycles, 40; primers for PCR amplification are as follows:

pDC516-F1:
(SEQ ID NO: 78)
ACACGTCAATGGGAAGTGAAA
pDC516-R1:
(SEQ ID NO: 79)
GCTAGACGATCCAGACATGAT

### 3. Amplification of Recombinant Adenovirus SARS-COV-2 Vaccine

The correctly identified recombinant adenovirus vaccine strains are amplified step by step in 293 cells by the following specific steps: 80%-90% 293 cells overgrown are added according to MOI=10, a virus culture is collected after most of cell viruses become round, and a main virus seed stock and a working virus seed stock are prepared by the above repeated freezing and thawing method. The recombinant adenovirus vaccine is amplified in a cell factory or bioreactor, and the virus culture is collected after most of the cells are diseased. The cell and virus are amplified in the bioreactor by the following steps: 3-5 g/L Cytodex1 microvector is added into the bioreactor and sterilized; a cell culture fluid is added into the bioreactor; when operating conditions are kept at 37° C., pH 7.0, DO 50% and 50 rpm, HEK293 cells amplified in cell factory are dissociated and collected and inoculated into the bioreactor, where the inoculated cells have a density of 1.0-5.0×10⁵ cells/ml, and the cell culture fluid is replenished to 5 L; when the culture conditions of the cells in the bioreactor are at 37° C., 30-50 rpm, and pH 7.15-7.25, and DO 30%-50%, samples are taken every day to test glucose concentration, cell density and cell morphology on the microvector; when the cell density in the bioreactor reaches 1.0-5.0×10⁶ cells/ml, the recombinant adenovirus vaccine strains are inoculated in the bioreactor with MOI of 5-30; after inoculation, samples are taken every day to test the glucose concentration, virus titer in a culture supernatant and a cell pellet, and the morphology of cells on the microvector is observed; when most of the cells are shed from the microvector, the culture is terminated, a virus lysate is added into the bioreactor with the final concentration of 0.05%-1% Tween 20, and lysed at 37° C. for 2-4 hours, and then the virus solution is collected.

### 4. Purification of Recombinant Adenovirus SARS-COV-2 Vaccine

Collected viruses are purified through cesium chloride ultracentrifugation or ion-exchange column chromatography by the following specific process steps:

### (1) Purification of Adenovirus Vaccine Through Cesium Chloride Ultracentrifugation

1200 g collected culture with virus is centrifuged for 10 minutes; a culture supernatant containing virus is sucked; a cell pellet is re-suspended with the supernatant containing virus at 1/10 culture volume and is repeatedly frozen and thawed for three times in a refrigerator at −80° C. and a water bath at 37° C., centrifuged for 10-20 minutes at 3000 rpm; and then the supernatant is sucked. The culture supernatant containing virus is concentrated 10 times with a 100K-300K ultrafiltration membrane; 1.4 g/ml cesium chloride solution (53 g cesium chloride+87 ml of 10 mM Tris-HCl, PH 7.9) and 1.2 g/ml cesium chloride solution (26.8 g cesium chloride+92 ml of 10 mM Tris-HCl, PH 7.9) are prepared; 8 ml of 1.4 g/ml cesium chloride solution and 6 ml of 1.2 g/ml cesium chloride solution are slowly added into an overspeed tube sequentially; finally, 20 ml supernatant containing virus is added at the top of discontinuous gradient and centrifuged at 100000×g at 4° C. for 90 minutes after balanced; and after centrifugation, a blue virus band is aspirated by a syringe, and cesium chloride is removed through dialysis and stored at −80° C.

### (2) Purification of Adenovirus Through Ion Exchange Chromatography

A virus culture is collected, and lysed with 0.05%-1% Tween 20 at 37° C. for 2-4 hours; the lysed culture is filtrated and clarified with 1.2 μm and 0.45 um bag filters; the sample is concentrated 10 times with a tangential flow membrane with a molecular weight of 100 kD-300 kD, and then difiltrated with buffer (10 times the concentrated sample volume) (50 mM Tris-HCl, 2 mM MgCl₂, 0 mM-500 mM NaCl, PH 8.0); the filtrated sample is collected and mixed with nuclease; the mixture has a final concentration of 10 U/ml-50 U/ml and dissociated at 37° C. for 1-3 hours; and the sample is subjected to anion exchange chromatography with Q Sepharose XL, Source 30Q or Source 15Q and other fillers by the following specific steps: balancing the buffer at a flow rate of 20 ml/min to balance five column volumes; after balancing, loading the sample at a flow rate of 10 ml/min; after sample loading, balancing the buffer to a conductivity level; eluting the sample through linear gradient under the elution conditions of 100% low-salt buffer to 100% high-salt buffer, the elution column volume of 10V and the flow rate of 10 ml/min; collecting each elution peak; after elution, regenerating 5-10 column volumes for the column with 2M NaCl buffer solution at a flow rate of 20 ml/min; collecting the virus peaks, and then conducting buffer replacement for the eluted virus samples through dialysis or tangential flow filtration (buffer 10 mM Tris, 10 mM Na-PO4, 150 mM NaCl, 2 mM MgCl₂, 2% sucrose, 0.15% glycerol and 0.02% Tween 80, PH7.6). The purified adenovirus is filled directly and stored in a dark place at −20° C.

Adenoviruses or vaccines prepared by Embodiment 1 are defined as Ad5_{WT} (SEQ ID NO: 24) [symbol] Ad5_{Beta} (SEQ ID NO:26) [symbol] Ad5_{Delta} (SEQ ID NO:28) [symbol] Ad5_{BA.1} (SEQ ID NO: 32) [symbol] Ad5_{BA.4/5} (SEQ ID NO:38) [symbol] Ad5_{Beta/Delta} (SEQ ID NO:30) recombinant adenoviruses, respectively.

The effect of the recombinant adenovirus SARS-COV-2 vaccine is proved by the following experimental examples.

### Experiment 1.1 Identification of Recombinant Adenovirus Vaccine

We expressed full-length spike glycoprotein of SARS-COV-2 original strain, Beta strain, Delta strain, Omicron BA.1 and Omicron BA.4/5 respectively by human replication-deficient Ad5 adenovirus vectors. Meanwhile, we designed and constructed adenovirus Ad5-Beta/Delta (FIG. 1) of two mutations L452R and T478K of Delta strain added in the RBD domain based on the full-length spike glycoprotein of Beta strain. The expression level of Spike in 293T cells infected with recombinant Ad5 Beta Delta virus for 48 hours was detected by Western blot. As shown in FIG. 1, 293T cells infected with adenovirus Ad5 Beta Delta could induce high level of Spike expression, while such cells infected with empty adenovirus control Ad5 Empty could not do this, indicating that our recombinant adenovirus protein vaccine was successfully prepared.

### Experiment 1.2 Animal Preparation and Mouse Immunization and Sample Collection

Female BALB/c mice at 6-8 weeks of age were purchased from Charles River, and the mice were housed in a pathogen-free environment at the State Key Laboratory of Biotherapy, Sichuan University. Mice were divided into seven groups: (1) PBS group; (2) Ad5_{Empty}; (3) Ad5_{WT}; (4) Ad5_{Beta}; (5) Ad5_{Delta}; (6) Ad5_{BA.1}; and (7) Ad5_{Beta/Delta} group. BALB/c mice were subjected to intranasal immunization respectively at week 0, 4, and 8 at a dose of 5×10⁹ viral particles VP/piece. Blood samples were collected through orbital veins at week 3 and 7; mice were killed at week 11; blood, bronchoalveolar lavage fluid and lung and mediastinal lymph node tissues were collected; and the tissues were prepared into single-cell suspensions and subjected to flow cytometry. The blood was centrifuged for 10 minutes at 4° C. and 6000 rpm. Serum samples were stored at −20° C. prior to use for subsequent detection of binding and neutralizing antibodies in serums.

### Experiment 1.3 Detection of Anti-RBD Specific Antibody

RBD specific antibody in serum was detected by ELISA. We coated 96-well NUNC-MaxiSorp plates (Thermo Fisher Scientific, USA) with recombinant RBD proteins (0.1 μg/well) at 4° C. for 12 hours. Such plates were washed three times with 1×PBST (1×PBS+0.1% Tween-20) and blocked with 1% bovine serum albumin (BSA) at 37° C. for 1 hour. The culture plates and serum samples with 2× dilution gradient were incubated at room temperature for 1 hour, and then washed three times by 1×PBST. The culture plates were added with 1:10000 diluted horseradish peroxidase (HRP)-goat anti-mouse IgG antibody, incubated at room temperature for 1 hour, and washed five times. The culture plates were added with 3,3′, 5,5′-tetramethylbenzidine (TMB) and incubated in a dark place for 10 minutes. The reaction was stopped with 1 M H₂SO₄ (100 μl/well) and the absorbance was measured at a position of 450 nm.

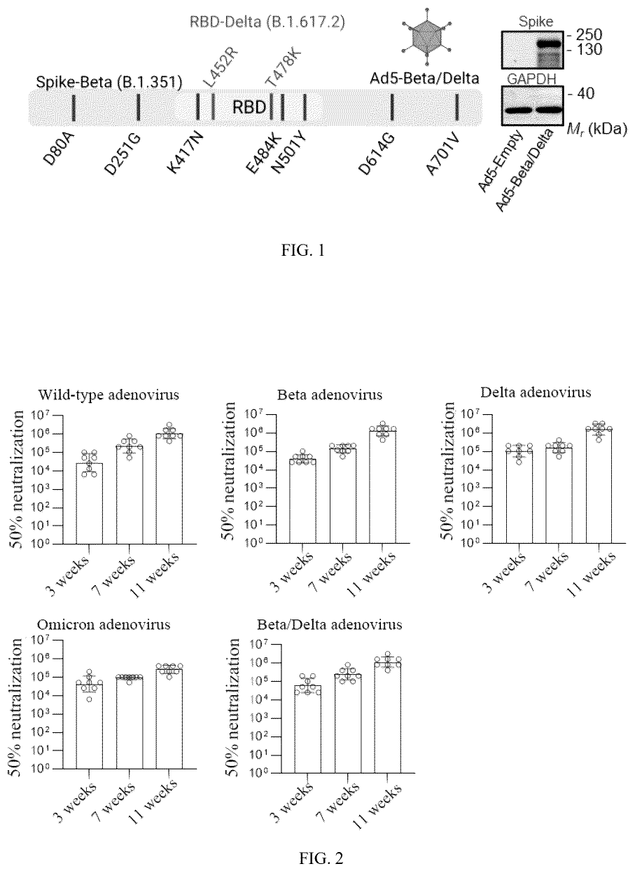

As shown in FIG. 2, recombinant adenovirus vaccines Ad5_{WT}, Ad5_{Beta}, Ad5_{Delta}, Ad5_{BA.1}, and Ad5_{Beta/Delta} all induced the high levels of anti-RBD specific binding antibodies in the blood at week 3, 7, and 11 after vaccination, and thus the levels of the neutralizing antibodies in the serums were further detected to evaluate broad-spectrum neutralizing immunoreaction induced by various recombinant adenovirus vaccines.

### Experiment 1.4 Pseudovirus Neutralization Assay

In addition to BA.3 and BA.4/5 pseudoviruses purchased from Vazyme Biotech Co., Ltd. (China), we purchased other SARS-COV-2 variant pseudoviruses of SARS-COV-2 pseudovirus (GFP-Luciferase) from Genomeditech (Shanghai) Co., Ltd. Serum samples collected after the mice were killed at week 11 were inactivated at 60° C. for 30 min and then were diluted in a three-fold gradient with DMEM dual culture medium containing serum and antibiotics. Diluted serums were incubated with luciferase pseudoviruses (wild type, B.1.1.7, B.1.351, P.1, B.1.617, C.37, BA.1, BA.2, BA.2.12.1, BA.3, and BA.4/5) for 1 hour at 37° C. 1.2×10+293T/ACE2 cells were added to each well to express reporter genes. After 48 hours, the supernatant of the infected cells was removed, and 100 μl lysis reagent containing luciferase substrate was added to each well. Finally, a multi-mode microplate reader (PerkinElmer, USA) was used for detection.

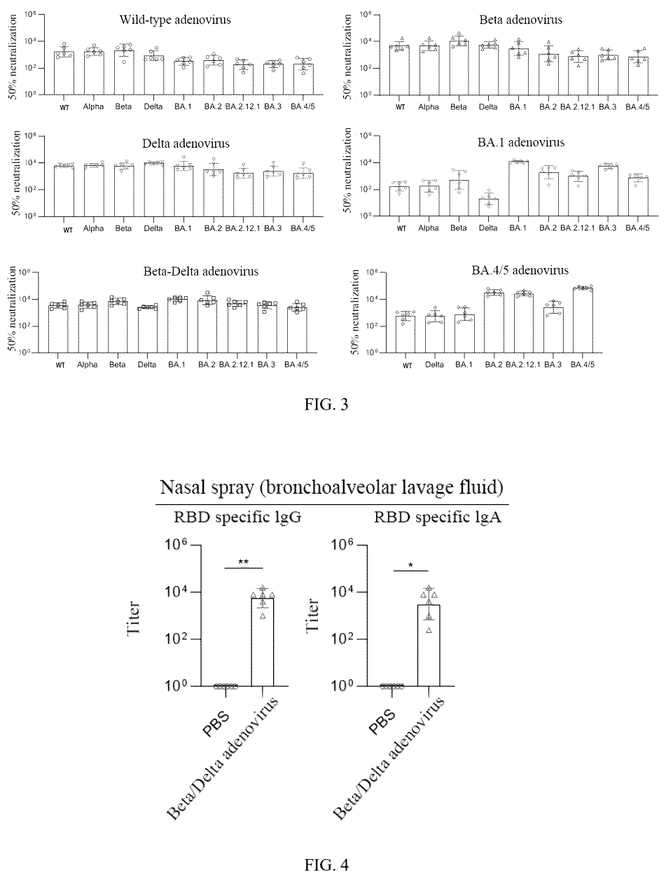

As shown in FIG. 3, Ad5_{WT}, Ad5_{Beta}, and Ad5_{Delta} induced high levels of neutralizing antibodies against their own viruses, but such vaccines induced low levels of neutralizing antibodies against Omicron subvariant due to the strong immune escape capability of Omicron strain. Ad5-Omicron vaccine could induce high levels of neutralizing antibodies against the Omicron subvariant, but was unable to induce high levels of neutralizing antibodies against WT, Alpha, Beta, Delta and others. Compared with other adenovirus vaccines, Ad5_{Beta/Delta} could induce high levels of broad-spectrum neutralizing antibodies against a wide range of viruses including WT, Alpha, Beta, Delta, and Omicron BA.1, BA.2, BA.2.12.1, BA.3 and BA.4/5.

### Experiment 1.5 Detection of Anti-RBD Specific IgG and IgA Antibodies in Bronchoalveolar Lavage Fluid

RBD specific antibody IgG and IgA in bronchoalveolar lavage fluid were detected by ELISA. We coated 96-well NUNC-MaxiSorp plates (Thermo Fisher Scientific, USA) with recombinant RBD proteins (0.1 μg/well) at 4° C. for 12 hours. Such plates were washed three times with 1×PBST (1×PBS+0.1% Tween-20) and blocked with 1% bovine serum albumin (BSA) at 37° C. for 1 hour. The culture plates and bronchoalveolar lavage fluid samples with 2× dilution gradient were incubated at room temperature for 1 hour, and then washed three times by 1×PBST. The culture plates were added with 1:10000 diluted horseradish peroxidase (HRP)-goat anti-mouse IgG or IgA (diluted at 1:5000) antibody, incubated at room temperature for 1 hour, and washed five times. The culture plates were added with 3,3′, 5,5′-tetramethylbenzidine (TMB) and incubated in a dark place for 10 minutes. The reaction was stopped with 1 M H₂SO₄ (100 μl/well) and the absorbance was measured at a position of 450 nm.

As shown in FIG. 4, the recombinant adenovirus vaccine Ad5_{Beta/Delta} induced high levels of anti-RBD-specific conjugated antibodies IgG and IgA in the bronchoalveolar lavage fluid at week 11 after vaccination, indicating that our recombinant adenovirus vaccine can induce strong antibody protection in the mucosal portion of the respiratory tract.

### Experiment 1.6 Detection of Tissue Resident T Cells in Bronchoalveolar Lavage Fluid

The number of tissue resident T cells (TRM) in the bronchoalveolar lavage fluid was detected using flow cytometry. 1 ml collected bronchoalveolar lavage fluid was centrifuged (400×g, 5 minutes), and the supernatant was removed to obtain the cell pellet. After the cells were re-suspended with 100 μl PBS buffer, PerCP/Cyanine5.5-conjugated anti-mouse CD3 (BioLegend, 100718), Brilliant Violet 421-conjugated anti-mouse CD4 (BioLegend, 100412), Brilliant Violet 510-conjugated anti-mouse CD8, PE-conjugated anti-mouse CD44, FITC-conjugated anti-mouse CD69, and APC-conjugated anti-mouse CD103 were added and incubated at 4° C. for 30 minutes. The cells were re-selected after being washed once with PBS, and detected by flow cytometry.

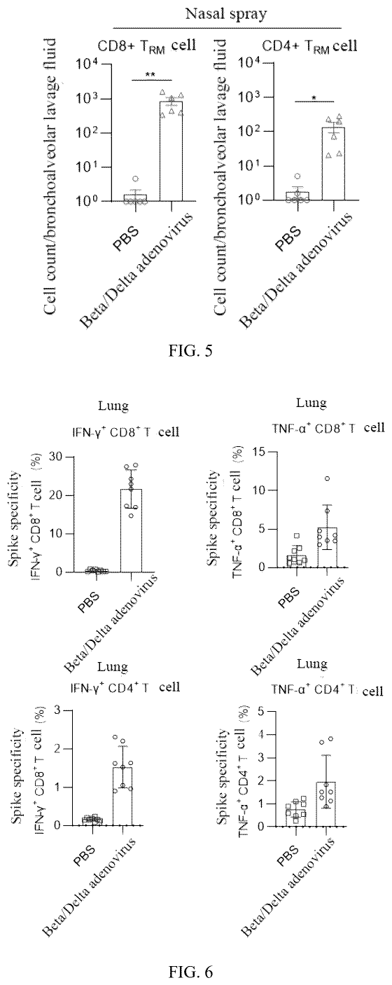

As shown in FIG. 5, when such mice subjected to intranasal immunization with Ad5Beta Delta were compared with PBS-treated mice, adenovirus vaccine could recruit a large number of tissue resident CD4⁺ and CD8⁺ T cells in lung tissue, resulting in cellular immunoprotection.

### Experiment 1.7 Detection of Antigen-Specific T Cells in Lung Tissue

Antigen-specific T cells in lung tissue were detected through flow cytometry. After the collected lung tissue was cut to a size of 1 mm³, the prepared collagenase digestive fluid was added and incubated at 37° C. for 1 hour, and a tissue digestive fluid was filtrated through a 70 uM sieve to obtain a single-cell suspension. Single cells of the lung tissue were stimulated and cultured in dual 1640 culture medium containing Spike peptide library for 12 hours, and subjected to intracellular cytokine staining (ICS). The 1640 culture medium was added with 10% fetal bovine serum, 100 μg/ml streptomycin, 100 U/ml penicillin, 1 mM pyruvic acid (all purchased from Gibco), 50 μM β-mercaptoethanol and 20 U/ml IL-2 (all purchased from Sigma-Aldrich). To block the secretion of intracellular cytokines, brefeldin A (BFA, BD Biosciences) was incubated for 6 hours prior to staining. The cells were washed in a 1×PBS coolant, and stained with PerCP/Cyanine5.5-conjugated anti-mouse CD3 (BioLegend, 100718), APC-conjugated anti-mouse CD4 (BioLegend, 100412), FITC-conjugated anti-mouse CD8, and PE-conjugated anti-mouse CD44 antibodies at 4° C. for 30 minutes. Then, the cells were immobilized and perforated, so that they could be stained with PE-Cy7-conjugated anti-mouse IFN-γ and Brilliant Violet 510-conjugated anti-mouse TNF-α (all flow antibodies from BioLegend) after left for 2 hours at room temperature. The cells were washed with 1×PBS and then detected by flow cytometry.

T cell response is a major determinant of clinical outcome and plays an important role in the prevention of SARS-COV-2. As shown in FIG. 6, the cells subjected to intranasal immunization with Ad5_{Beta/Delta} vaccine induce strong antigen-specific cellular immunity, including T cells that secrete IFN-γ and TNF-α.

### Experiment 1.8 Detection of Reactions of a Germinal Center

After a single-cell suspension from mediastinal lymphocyte was prepared, we used PerCP/Cyanine5.5-conjugated anti-mouse CD3, PE-Cy7-conjugated anti-mouse CD45R/B220, Brilliant Violet 421-conjugated anti-mouse GL-7, APC-conjugated anti-mouse CD95, Brilliant Violet 421-conjugated anti-mouse CD19, APC-conjugated anti-mouse CD4, PE-conjugated anti-mouse CXCR5, and Brilliant Violet 510-conjugated PD-1 antibodies to stain Tfh cells and GC B cells (flow cytometry antibodies were derived from BioLegend). The cells were stained in the dark at 4° C. After 30 minutes, the cells were washed with 1×PBS and then detected by a flow cytometer.

A durable anamnestic reaction provides rapid and effective protective immunity in case of reinfection. The increase of Tfh and GCB cells indicates that the vaccine can provide long-lasting protective immunity to the body. As shown in FIG. 7, the cells subjected to intranasal immunization with Ad5 Beta Delta vaccine could induce the significant increase of Tfh and GCB cells in mediastinal lymph nodes, demonstrating that our recombinant adenovirus vaccine can improve long-lasting immunoprotection.

### Part II: Immunoprotection Induced by Individual Proteins

### Embodiment 2.1 Preparation of Recombinant Protein Using Insect Baculovirus Expression System (Taking S-RBD-HR_{BA.4/5} as an Example)

### 1. Construction Design of S-RBD (Omicron_BA.4/.5)-HR

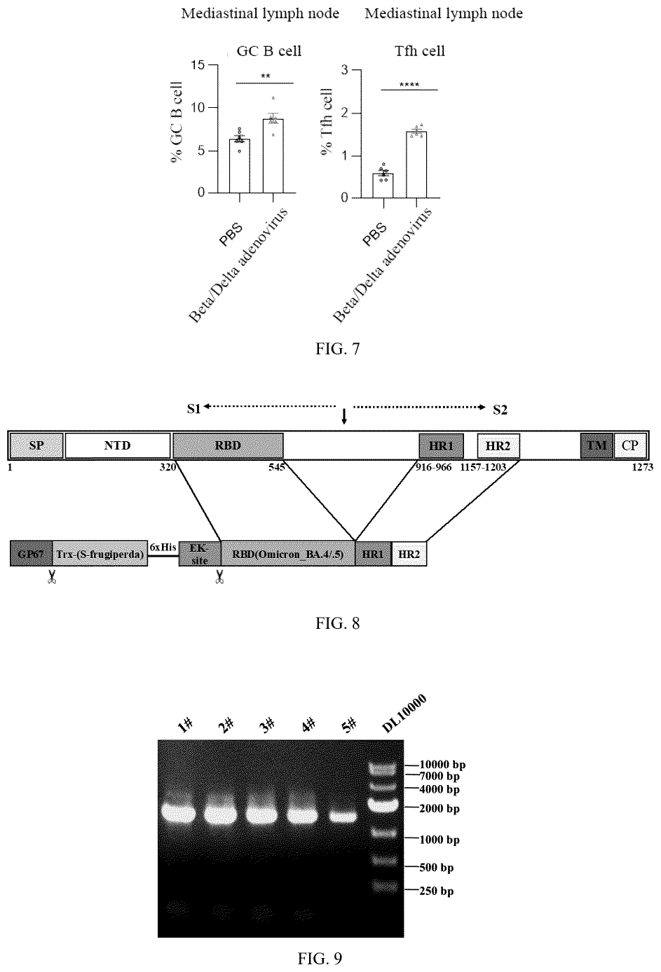

The S protein of SARS-COV-2 is a membrane-localized protein. To ensure the simulation of its secretion process, we added a signal peptide sequence of GP67 at the N-terminus of the protein when constructing the SARS-COV-2 S-RBD (Omicron_BA.4/.5)-HR protein expression to assist the secretion expression of the protein. This signal peptide will be spontaneously removed by insect cells in a protein secretion process. In addition, we added the thioredoxin (Trx) tag of Spodoptera frugiperda, S. frugiperda behind the GP67 signal peptide to assist the folding of the S-RBD (Omicron_BA.4/.5)-HR, and added a 6×his tag to assist the subsequent purification and an EK restriction enzyme cutting site to remove the Trx and 6×his tags. The protein expression can remove all non-S-RBD (Omicron_BA,4/5)-HR redundant amino acids by EK restriction enzyme. A protein amino acid design sequence is shown in SEQ ID NO: 6. An expression construction design pattern is shown in FIG. 8.

### 2. Identification of Recombinant Plasmid Construction

The designed coding fragments are cloned into pFastBac1 vector plasmids and identified through bacterial PCR. Identification results through bacterial PCR show that GP67-Trx-His-EK-S-RBD (Omicron_BA.4/.5)-HR fragments are successfully expanded in all five selected clones, as shown in FIG. 9.

### 3. Identification of Recombinant Bacmid

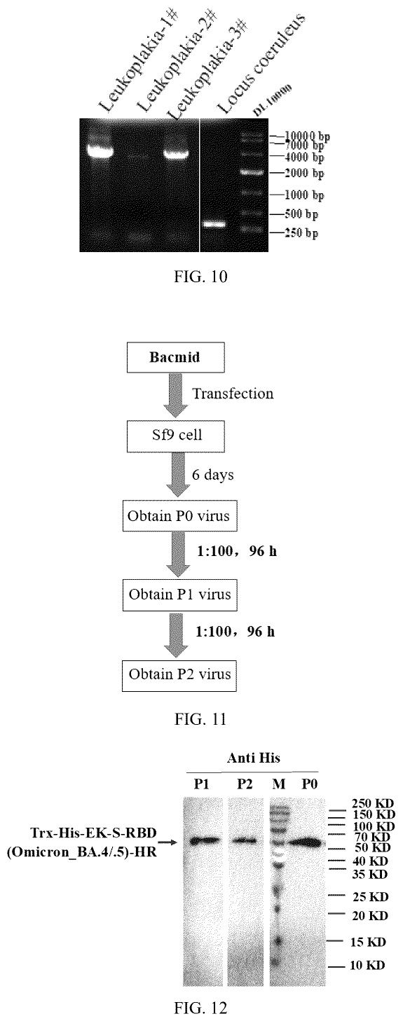

Correct pFastBac1-GP67-Trx-His-EK-S-RBD (Omicron_BA.4/.5)-HR recombinant clones are selected to extract the recombinant plasmids, and DH10b competent cells are transformed and identified through colony PCR. Colony PCR products are detected through 1% agarose gel electrophoresis, and the identification results are shown in FIG. 10. Leukoplakia is recombinant bacmid clone and locus coeruleus is non-recombinant bacmid clone.

### 4. Packaging of Recombinant Baculovirus

Recombinant bacmid is transfected into sf9 insect cells. After 5-6 days, recombinant baculovirus P0 is harvested and inoculated into fresh sf9 insect cells at a ratio of 1:100. After 4 days, recombinant baculovirus P1 is harvested and inoculated into fresh sf9 insect cells at the ratio of 1:100. After 4 days, recombinant baculovirus P2 is harvested. A flow diagram of packaging and amplification of recombinant baculovirus is shown in FIG. 11.

### 5. Verification of Target Protein Expression

Baculovirus amplification is accompanied by target protein expression. Before the removal of tags, the target protein contains His tag, so we verify a recombinant protein expression through anti-His WB experiment. Verification results show that an obvious band is observed between 40 KD and 55 KD Marker bands, and its size is consistent with the size of Trx-His-EK-S-RBD (Omicron_BA.4/.5)-HR protein, indicating that the baculovirus amplification and the target protein expression are successful. Detection results are shown in FIG. 12.

Recombinant protein vaccines RBD-WT, RBD-HR_{Delta}, RBD-HR_{BA.4/5}, and RBD-HR_{BA.1 Delta/Beta} are respectively constructed by the construction method of the recombinant protein based on the amino acid sequence of S protein RBD of SARS-COV-2 or the amino acid sequence of S protein RBD-HR of SARS-COV-2 mutant: SEQ ID NO: 1, SEQ ID NO:3, SEQ ID NO: 6 and SEQ ID NO:18, all of which are used for animal immunity and other subsequent studies.

### Embodiment 2.2 Preparation of Recombinant Protein Using CHO Cell Expression System (Taking S-BA.4/5 as an Example)

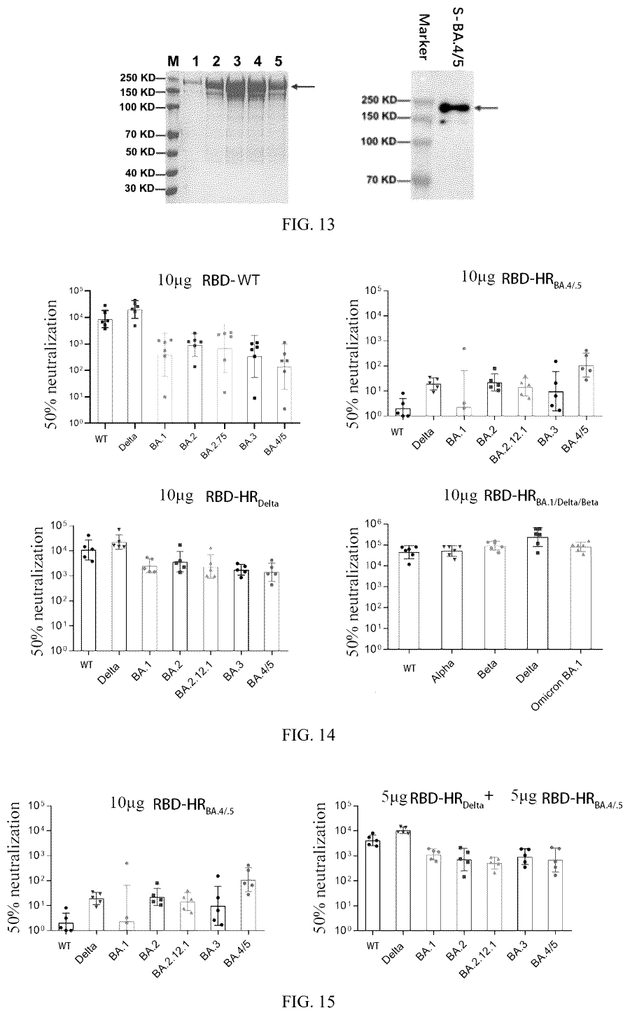

Recombinant protein vaccines produced by CHO cells are mainly targeted at full-length S proteins, and these fragments are genetically synthesized according to codon preference, and polyhistidine is used as a purification tag (6His), and the complete nucleotide sequence is shown in SEQ ID NO:71. Subsequently, the high expression vector pTT5 is constructed to express a precursor protein with an amino acid sequence as shown in SEQ ID NO: 70. After vector construction, CHO cells are transfected, and a harvested solution is taken for SDS-PAGE and Western-blot tests, as shown in FIG. 13. This proves that the target protein has been successfully expressed.

Recombinant proteins and vaccines S-WT, S-Beta, S-Delta, S-BA.1, S-BA.2, and S-BA.4/5 are respectively constructed by the construction method of the recombinant protein based on the full-length amino acid sequence of S protein of SARS-COV-2: SEQ ID NO:40, SEQ ID NO:46, SEQ ID NO:52, SEQ ID NO:58, SEQ ID NO: 64, and SEQ ID NO:70, all of which are used for animal immunity and other subsequent studies.

The effect of the recombinant protein vaccine is proved by the following experimental examples.

### Experiment 2.1 Animal Preparation and Mouse Immunization and Sample Collection

Female BALB/c mice at 6-8 weeks of age were purchased from Charles River, and the mice were housed in a pathogen-free environment at the State Key Laboratory of Biotherapy, Sichuan University.

BALB/c mice were subjected to intranasal immunization at day 0, 14 and 28. Blood samples were collected through orbital veins at day 42; mice were killed at week 11; blood, bronchoalveolar lavage fluid and lung and mediastinal lymph node tissues were collected; and the tissues were prepared into single-cell suspensions subjected to flow cytometry. The blood was centrifuged for 10 minutes at 4° C. and 6000 rpm. Serum samples were stored at −20° C. prior to use for subsequent detection of binding and neutralizing antibodies in serums.

Experiment 2.2 Pseudovirus Neutralization Assay after Immunization with a Single Protein Vaccine

We used pseudoviruses to detect neutralizing antibodies in serums of immunized mice, where BA.3 and BA.4/5 pseudoviruses were purchased from Vazyme Co., Ltd. (China); and wild-type B.1.1.7, B.1.351, P.1, B.1.617, C.37, BA.1, BA.2, BA.2.12.1 and other pseudoviruses were purchased from Genomeditech (Shanghai) Co., Ltd. Serum samples collected after the mice were killed at week 11 were inactivated at 60° C. for 30 min and then were diluted in a three-fold gradient with DMEM dual culture medium containing serum and antibiotics. Diluted serums were incubated with luciferase pseudoviruses (wild type, B.1.1.7, B.1.351, P.1, B.1.617, C.37, BA.1, BA.2, BA.2.12.1, BA.3, and BA.4/5) for 1 hour at 37° C. 1.2×10^{4 293}T/ACE2 cells were added to each well to express reporter genes. After 48 hours, the supernatant of the infected cells was removed, and 100 μl lysis reagent containing luciferase substrate was added to each well. Finally, a multi-mode microplate reader (PerkinElmer, USA) was used for detection.

As shown in FIG. 14, wild-type RBD-WT, RBD-HR_{Delta}, and RBD-HRB_{A.1/Delta/Beta} proteins induced higher levels of neutralizing antibodies against WT, Alpha, Beta, Delta, and Omicron viruses, where individual RBD-HR_{Delta} protein and chimeric RBD-HR_{BA.1/Delta/Beta} proteins containing Beta and Delta mutants showed strong immunoprotection against WT, Alpha, Beta, Delta, Omicron and other viruses; individual RBD-WT protein showed strong immunoprotection only against WT and Delta mutant strains and weak immunoprotection against Omicron; and individual RBD-HR_{BA.4/5} protein showed no strong immunoprotection against WT, Delta and Omicron. The RBD-HR_{BA.1/Delta/Beta} protein showed strong immunoprotection against various types of SARS-COV-2.

### Experiment 2.3 Neutralizing Antibodies Induced by Bivalent RBD Protein

In the above experiments, we found that individual RBD-HRBA.4/.5 protein had no obvious immunoprotection against WT, Delta, Omicron and other viruses. After mice were immunized with a bivalent combination vaccine (5 micrograms of RBD-HR_{Delta}+5 micrograms of RBD-HR_{BA.4/5} protein SEQ ID NO:3+SEQ ID NO:6), a high-titer neutralizing antibody was induced and had significantly stronger immunoprotection than the individual RBD-HRBA.4/.5 protein, indicating the effectiveness of the bivalent vaccine, as shown in FIG. 15.

### Experiment 2.4 Pseudovirus Neutralization Assay for Different S Proteins

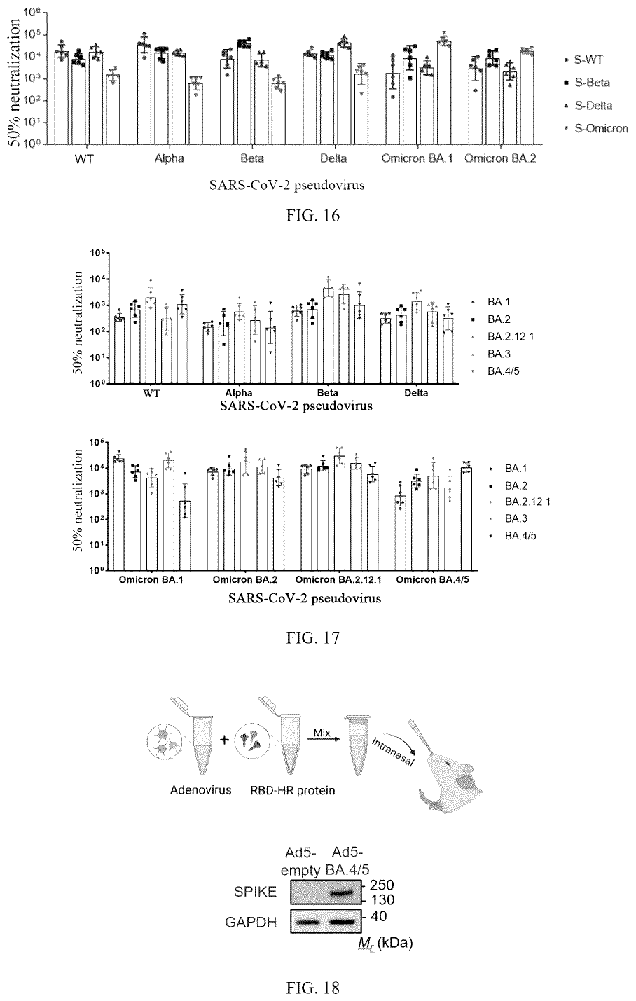

In order to verify the immunoprotection induced by S proteins (full-length trimers) of different virus strains, we immunized mice on day 0, 14, and 28 with prepared S proteins, where S proteins of BA.2.12.1 and BA.3 were commercially available S proteins. S-WT, S-Beta, S-Delta, S-BA.1, S-BA.2 and S-BA.4/5 were all recombinant S proteins prepared with reference to Embodiment 2.2. In an immunization protocol, 10 micrograms of protein+MF59 adjuvant are provided. Pseudovirus neutralizing antibodies were determined by removing the antiserum on day 42, as shown in FIG. 16 and FIG. 17.

As shown in FIG. 16, S proteins of WT, Beta and Delta strains have strong immunoprotection against the WT, Beta and Delta strains, but have weak immunoprotection against Omicron strains. S proteins (S-BA.1 and S-Ba.2) of Omicron strains have significantly stronger immunoprotection against Omicron strains than other strains.

As can be seen from FIG. 17, full-length S proteins of B.1, BA.2, BA.2.12.1, BA.3, and BA.4/5 have significantly stronger immunoprotection against Omicron strains than WT, Alpha, Beta and Delta strains, showing certain specificity.

### Part III: Stronger Immunoprotection Induced by Adenovirus Combined with Trimeric Recombinant Protein

### Experiment 3.1 Preparation of Mixed Preparation of Adenovirus Vaccine and Recombinant Protein Vaccine

We expressed the full-length spike glycoprotein of SARS-COV-2BA.4/5 strain by human replication-deficient Ad5 adenovirus vectors. The expression level of Spike in 293T cells infected with recombinant Ad5_{BA.4/5} virus for 48 hours was detected by Western blot. The detailed steps are shown in Embodiment 1. As shown in FIG. 18, we mixed different doses of adenovirus Ad5_{BA.4/5} with RBD-HR_{Delta} produced in Embodiment 2.1 to prepare the preparation for intranasal immunization. The adenovirus Ad5_{BA.4/5} was prepared based on SEQ ID NO:38. The recombinant protein RBD-HR_{Delta} was prepared based on SEQ ID NO:3.

Similarly, adenovirus Ad5_{WT} was prepared based on SEQ ID NO:24, S-BA.4/5 protein was prepared based on SEQ ID NO:71, and RBD-WT protein was prepared based on SEQ ID NO:1. We mixed the adenovirus Ad5_{WT} with the RBD-WT protein produced in Embodiment 2 respectively to prepare a preparation. The adenovirus Ad5_{BA.4/5} was mixed with the S-BA.4/5 protein produced in Embodiment 2.2 to prepare a preparation for intranasal immunization.

### Experiment 3.2Animal Preparation and Mouse Immunization and Sample Collection

Female BALB/c mice at 6-8 weeks of age were purchased from Charles River, and the mice were housed in a pathogen-free environment at the State Key Laboratory of Biotherapy, Sichuan University. The mice were divided into 6 groups: (1) RBD-HR_{Delta} (10 μg/piece) control group, (2) Ad5_{empty} (5×10⁹ VP/piece)+RBD-HR_{Delta} (10 μg/piece), (3) Ad5_{BA.4/5} (2.5×10⁹ VP/piece) low-dose group, (4) Ad5_{BA.4/5} (2.5×10⁹ VP/piece) low dose+RBD-HR_{Delta} (10 μg) group, (5) Ad5_{BA.4/5} (5×10⁹ VP/piece) high-dose group; and (6) Ad5_{BA.4 5/}(5×10⁹ VP/piece) high-dose+RBD-HR_{Delta} (10 μg) group.

In addition, the experimental grouping of Ad5_{WT}+RBD-WT combined drug was as follows: (a) PBS (5 pieces), (b) RBD-WT vaccine group (10 μg/piece, 5 pieces), (c) adenovirus Ad5_{WT} (5×10⁹ VP/piece), and (d) Ad5_{WT} (5×10⁹ VP/piece)+RBD-WT group (10 μg/piece) (5 pieces).

The experimental grouping of Ad5_{BA.4/5}+S-BA.4/5 combined drug was as follows: (a) PBS (5 pieces), (b)S-BA.4/5 vaccine group (10 μg/piece, 5 pieces), (c) Ad5_{BA.4/5} group (5×10⁹VP/piece, 5 pieces), (d) Ad5_{BA4/5} (5×10⁹ VP/piece) high dose+S-BA.4/5 (10 μg/piece) group (5 pieces).

BALB/c mice were subjected to intranasal immunization at weeks 0, 4 and 8. Blood samples were collected through orbital veins at week 3 and 7; mice were killed at week 11; blood, bronchoalveolar lavage fluid and lung and mediastinal lymph node tissues were collected; and the tissues were prepared into single-cell suspensions and subjected to flow cytometry. The blood was centrifuged for 10 minutes at 4° C. and 6000 rpm. Serum samples were stored at −20° C. prior to use for subsequent detection of binding and neutralizing antibodies in serums. The immunization protocol is shown in FIG. 19.

### Experiment 3.3 Detection of Anti-RBD Specific Antibodies in Serum and Bronchoalveolar Lavage Fluid

RBD specific antibody in serum was detected by ELISA. We coated 96-well NUNC-MaxiSorp plates (Thermo Fisher Scientific, USA) with recombinant RBD proteins (0.1 μg/well) at 4° C. for 12 hours. Such plates were washed three times with 1×PBST (1×PBS+0.1% Tween-20) and blocked with 1% bovine serum albumin (BSA) at 37° C. for 1 hour. The culture plates and serum samples or bronchoalveolar lavage fluid samples with 2× dilution gradient were incubated at room temperature for 1 hour, and then washed three times by 1×PBST. The culture plates were added with 1:10000 diluted horseradish peroxidase (HRP)-goat anti-mouse IgG antibody, incubated at room temperature for 1 hour, and washed five times. The culture plates were added with 3,3′, 5,5′-tetramethylbenzidine (TMB) and incubated in a dark place for 10 minutes. The reaction was stopped with 1 M H₂SO₄ (100 μl/well) and the absorbance was measured at a position of 450 nm.

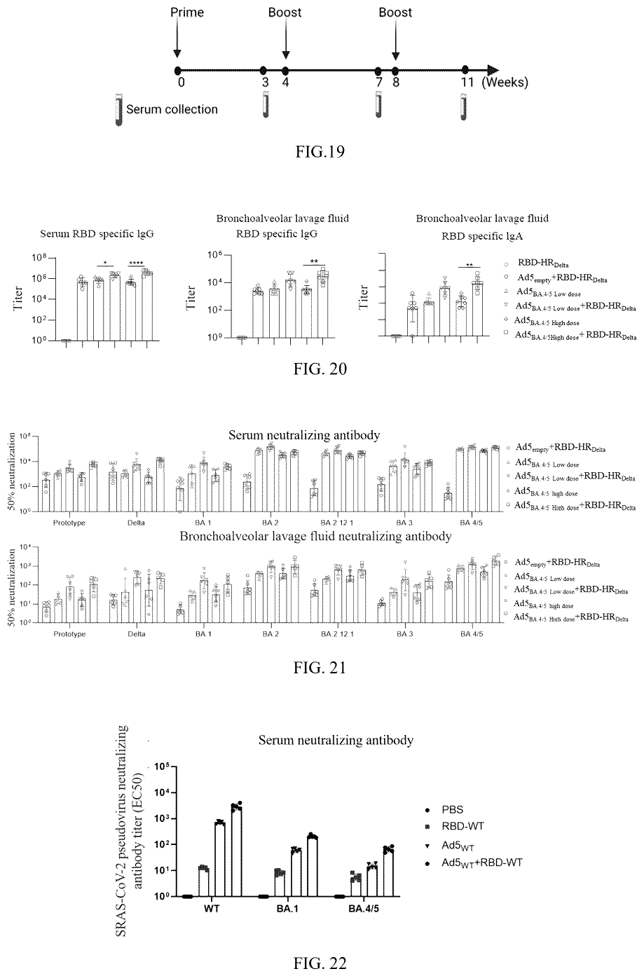

As shown in FIG. 20, after immunization, a preparation of adenovirus Ad5_{BA.4/5} combined with recombinant RBD-HR_{Delta} induces stronger anti-RBD specific binding antibodies in the serum and bronchoalveolar lavage fluid than that of Ad5_{BA.4/5}, indicating that a preparation of adenovirus combined with recombinant subunit can produce stronger humoral immunoprotection. In addition, intranasal immunization with RBD-HR_{Delta} cannot induce strong blood-conjugated antibodies, but RBD-HR_{Delta} being conjugated with Ad5 Empty can significantly increase its immunogenicity. This demonstrates that the adenovirus can be used as an adjuvant of recombinant subunit to enhance the immunogenicity of protein antigens.

### Experiment 3.4 Pseudovirus Neutralization Assay

In addition to BA.3 and BA.4/5 pseudoviruses purchased from Vazyme Biotech Co., Ltd. (China), we purchased other SARS-COV-2 variant pseudoviruses of SARS-COV-2 pseudovirus (GFP-Luciferase) from Genomeditech (Shanghai) Co., Ltd. Serum samples collected after the mice were killed at week 11 were inactivated at 60° C. for 30 min and then were diluted in a three-fold gradient with DMEM dual culture medium containing serum and antibiotics. Diluted serums were incubated with luciferase pseudoviruses (wild type, B.1.617, BA.1, BA.2, BA.2.12.1, BA.3, and BA.4/5) for 1 hour at 37° C. 1.2×10+293T/ACE2 cells were added to each well to express reporter genes. After 48 hours, the supernatant of the infected cells was removed, and 100 μl lysis reagent containing luciferase substrate was added to each well. Finally, a multi-mode microplate reader (PerkinElmer, USA) was used for detection.

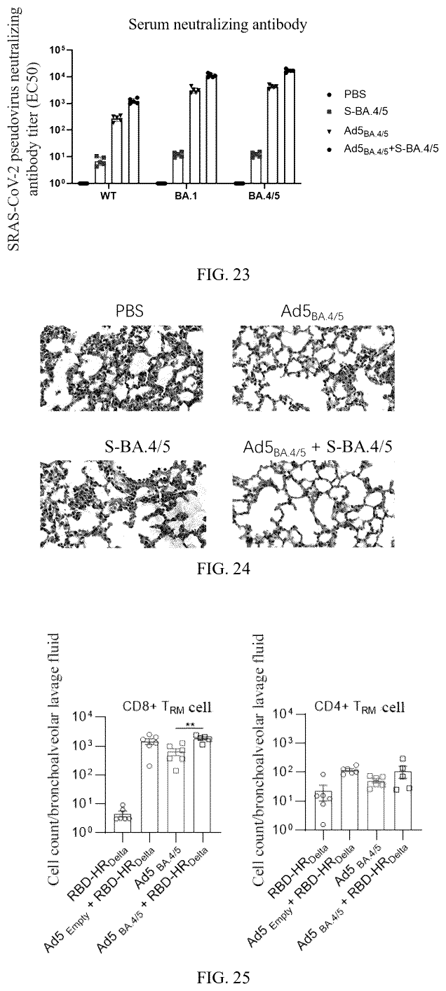

As shown in FIG. 21, a preparation of adenovirus Ad5_{BA.4/5} combined with recombinant RBD-HR_{Delta} induced stronger neutralizing antibodies in serum and bronchoalveolar lavage fluid than that of adenovirus Ad5_{BA.4/5}, indicating that a preparation of adenovirus combined with recombinant subunit can produce stronger blood and local mucosal neutralizing protection ability to prevent virus infection. As shown in FIG. 22, Ad5_{WT}+RDB-WT produced serum neutralizing antibodies against viruses such as WT, BA. 1 and BA.4/5. As shown in FIG. 23, Ad5_{BA.4/5}+S-BA.4/5 produced higher-level serum neutralizing antibodies against WT, BA.1, BA.4/5 and other viruses, and an amount of antibodies was significantly higher than that of Ad5_{WT}+RBD-WT, suggesting that Omicron's S protein adenovirus vaccine combined with S protein can produce stronger immunoprotection against Omicron mutant strains.

Experiment 3.5 Challenge Test of Mice Infected with SARS-COV-2 Virus

Female BALB/c mice at 6-8 weeks of age were purchased from Charles River and tested at the Animal Biosafety Level 4 (ABSL-4) Facility at the National Primate Research Center for Advanced BioSafety in Kunming, Yunnan Province, China. 20 mice were divided into the following groups for challenge test of live SARS-COV-2: (a) PBS (5 pieces), (b) adenovirus Ad5_{BA.4/5} group (5×10⁹VP/piece, 5 pieces), (c)S-BA.4/5 protein vaccine group (10 μg/piece, 5 pieces) and (d) AD5_{BA.4/5} (5×10⁹ VP/piece) high dose+S-BA.4/5 (10 μg) group (5 pieces).

Mice were subjected to intranasal immunization at week 0, 4, and 8, and then treated with SARS-COV-2 for nasal challenge (0.5 ml, 10⁶pfu/ml) 21 days after the final immunization. Mouse lung tissues were collected 4 days after virus attack. As shown in FIG. 24, after the mice were treated with immune serums induced by Ad5_{BA.4/5} (5×10⁹ VP/piece) high dose+S-BA.4/5 (10 μg) combined vaccine, viral loads in turbinate, trachea and lung tissues were not detected, lung histology was normal, alveolar structure was intact, and no obvious inflammation was present. In the PBS control group, minor pathological changes were observed, including multifocal consolidation area, mild alveolar septum thickening and alveolar congestion. In addition, small patches of inflammation consisting of macrophages, neutrophils, and lymphocytes were occasionally observed near small blood vessels. The effect of adenovirus or protein in the nasal drop is significantly weaker than that of the combined drug of adenovirus and protein.

### Experiment 3.6 Detection of Tissue Resident T Cells in Bronchoalveolar Lavage Fluid

The number of tissue resident T cells (TRM) in the bronchoalveolar lavage fluid was detected using flow cytometry. 1 ml collected bronchoalveolar lavage fluid was centrifuged (400×g, 5 minutes), and the supernatant was removed to obtain the cell pellet. After the cells were re-suspended with 100 μl PBS buffer, PerCP/Cyanine5.5-conjugated anti-mouse CD3 (BioLegend, 100718), Brilliant Violet 421-conjugated anti-mouse CD4 (BioLegend, 100412), Brilliant Violet 510-conjugated anti-mouse CD8, PE-conjugated anti-mouse CD44, FITC-conjugated anti-mouse CD69, and APC-conjugated anti-mouse CD103 were incubated at 4° C. for 30 minutes. The cells were re-selected after being washed once with PBS, and detected by flow cytometry.

As shown in FIG. 25, a preparation of high-dose adenovirus Ad5_{BA.4/5} combined with recombinant RBD-HR_{Delta} recruited more lung tissue resident TRM cells than that of adenovirus Ad5_{BA.4/5}. In addition, intranasal immunization with RBD-HR_{Delta} could not induce enough lung tissue TRM cells. However, intranasal immunization with RBD-HR_{Delta} combined with Ad5 Empty could significantly improve its immunogenicity and induce more lung tissue TRM cells, indicating that the preparation of adenovirus combined with recombinant subunit can produce stronger local mucosal cell immune response and prevent viral infection.

### Experiment 3.7 Detection of Antigen-Specific T Cells in Lung Tissue

Antigen-specific T cells in lung tissue were detected through flow cytometry. After the collected lung tissue was cut to a size of 1 mm³, the prepared collagenase digestive fluid was added and incubated at 37° C. for 1 hour, and a tissue digestive fluid was filtrated through a 70 uM sieve to obtain a single-cell suspension. Single cells of the lung tissue were stimulated and cultured in dual 1640 culture medium containing Spike peptide library for 12 hours, and subjected to intracellular cytokine staining (ICS). The 1640 culture medium was added with 10% fetal bovine serum, 100 μg/ml streptomycin, 100 U/ml penicillin, 1 mM pyruvic acid (all purchased from Gibco), 50 μM β-mercaptoethanol and 20 U/ml IL-2 (all purchased from Sigma-Aldrich). To block the secretion of intracellular cytokines, brefeldin A (BFA, BD Biosciences) was incubated for 6 hours prior to staining. The cells were washed in a 1×PBS coolant, and stained with PerCP/Cyanine5.5-conjugated anti-mouse CD3 (BioLegend, 100718), APC-conjugated anti-mouse CD4 (BioLegend, 100412), FITC-conjugated anti-mouse CD8, and PE-conjugated anti-mouse CD44 antibodies at 4° C. for 30 minutes. Then, the cells were immobilized and perforated, so that they could be stained with PE-Cy7-conjugated anti-mouse IFN-γ and Brilliant Violet 510-conjugated anti-mouse TNF-α (all flow antibodies from BioLegend) after left for 2 hours at room temperature. The cells were washed with 1×PBS and then detected by flow cytometry.

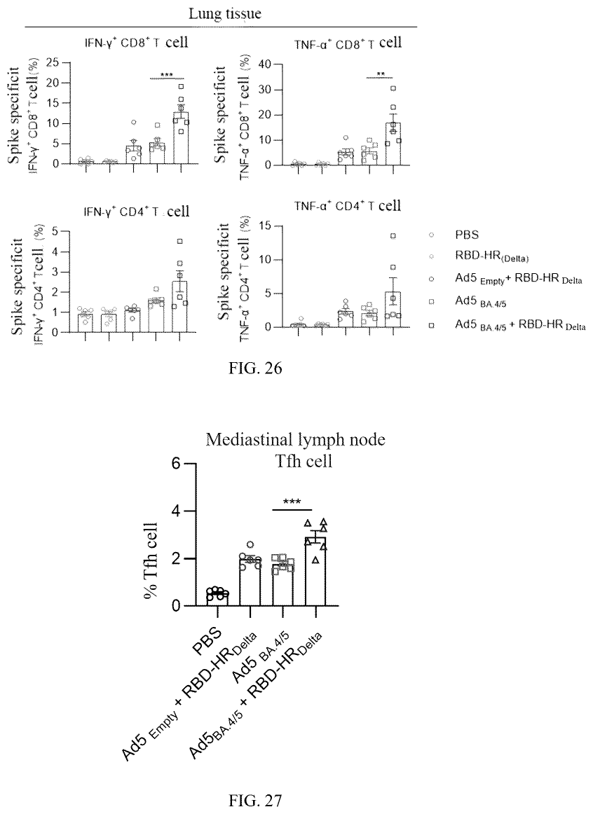

As shown in FIG. 26, a preparation of high-dose adenovirus Ad5_{BA.4/5} combined with recombinant RBD-HR_{Delta} recruited more antigen-specific T cells than that of adenovirus Ad5_{BA.4/5}. In addition, intranasal immunization with RBD-HR_{Delta} could not induce enough lung tissue antigen-specific T cells. However, intranasal immunization with RBD-HR_{Delta} combined with Ad5_{Empty} could significantly improve its immunogenicity and induce more lung tissue antigen specificity, indicating that the preparation of adenovirus combined with recombinant subunit can produce stronger local mucosal cell immune response and prevent viral infection.

### Experiment 3.8 Detection of Reactions of a Germinal Center

After a single-cell suspension from mediastinal lymphocyte was prepared, we used PerCP/Cyanine5.5-conjugated anti-mouse CD3, PE-Cy7-conjugated anti-mouse CD45R/B220, Brilliant Violet 421-conjugated anti-mouse GL-7, APC-conjugated anti-mouse CD95, Brilliant Violet 421-conjugated anti-mouse CD19, APC-conjugated anti-mouse CD4, PE-conjugated anti-mouse CXCR5, and Brilliant Violet 510-conjugated PD-1 antibodies to stain Tfh cells and GC B cells (flow cytometry antibodies were derived from BioLegend). The cells were stained in the dark at 4° C. After 30 minutes, the cells were washed with 1×PBS and then detected by a flow cytometer.

A durable anamnestic reaction provides rapid and effective protective immunity in case of reinfection. The increase of Tfh and GCB cells indicates that the vaccine can provide long-lasting protective immunity to the body. As shown in FIG. 27, a preparation of high-dose adenovirus Ad5_{BA.4/5} combined with recombinant RBD-HR_{Delta} can induce Tfh cells in mediastinal lymph nodes to increase significantly, demonstrating that adenovirus vaccine combined protein vaccine can improve long-lasting immunoprotection.

1. A pharmaceutical composition for preventing and/or treating infection with SARS-COV-2 or a mutant thereof is characterized by being a compound preparation containing active ingredients of a recombinant protein vaccine and an adenovirus vaccine for resisting infection with SARS-COV-2 or the mutant thereof.

2. A combined drug for preventing and/or treating infection with SARS-COV-2 or a mutant thereof, said combined drug comprising a recombinant protein vaccine and an adenovirus vaccine for resisting infection with SARS-CoV-2 or the mutant thereof, wherein the recombinant protein vaccine and the adenovirus vaccine are administered separately or simultaneously.

3. The pharmaceutical composition according to claim 1, which is an intramuscular injection, a nasal drop, a spray, a nasal spray or an inhalation.

4. The pharmaceutical composition according to claim 1, wherein at least one of the recombinant protein vaccine and/or the adenovirus vaccine contain(s) at least one of a protein and/or a protein precursor for resisting infection with SARS-COV-2 or the mutant thereof.

5. The pharmaceutical composition according to claim 4, wherein the protein and the protein precursor contain a full-length S protein or a protein formed by at least one RBD sequence and/or at least one HR sequence in the S protein of SARS-COV-2 or the mutant thereof; and
the RBD sequence is shown in SEQ ID NO:1; or the RBD sequence is a variant that has homology and same or similar biological activity with SEQ ID NO:1 and is obtained by substitution and/or deletion and/or insertion of at least one amino acid in the SEQ ID NO:1 sequence.

6. The pharmaceutical composition according to claim 5, wherein the protein formed by the at least one RBD sequence and/or the at least one HR sequence in the S protein is capable of spontaneously forming a trimer.

7. The pharmaceutical composition according to claim 5, wherein the variant is selected from at least one of RBD sequences of Alpha, Beta, Gamma, Delta and Omicron.

8. The pharmaceutical composition according to claim 5, wherein at least one of a signal peptide and a protein tag is bonded to the protein for resisting infection with SARS-COV-2 or the mutant thereof.

9. The pharmaceutical composition according to claim 8, wherein the protein for resisting infection with SARS-COV-2 or the mutant thereof is further bonded to a protease recognition zone for removing the protein tag.

10. The pharmaceutical composition according to claim 5, wherein the amino acid sequence of the protein and/or the protein precursor are/is selected from at least one of SEQ ID NO:1, SEQ ID NO:2, SEQ ID NO:3, SEQ ID NO: 4, SEQ ID NO:5, SEQ ID NO:6, SEQ ID NO:7, SEQ ID NO:8, SEQ ID NO:9, SEQ ID NO: 18, SEQ ID NO:20, SEQ ID NO:22, SEQ ID NO:40, SEQ ID NO:43, SEQ ID NO:46, SEQ ID NO: 49, SEQ ID NO:52, SEQ ID NO:55, SEQ ID NO:58, SEQ ID NO:61, SEQ ID NO:64, SEQ ID NO:67, SEQ ID NO:70, and SEQ ID NO:73.

11. The pharmaceutical composition according to claim 10, wherein a nucleotide sequence for encoding the amino acid sequence is shown in at least one member selected from the group consisting of SEQ ID NO:10, SEQ ID NO:11, SEQ ID NO:12, SEQ ID NO:13, SEQ ID NO:14, SEQ ID NO:15, SEQ ID NO: 16, SEQ ID NO:17, SEQ ID NO:19, SEQ ID NO:21, SEQ ID NO:23, SEQ ID NO:41, SEQ ID NO: 42, SEQ ID NO:44, SEQ ID NO:45, SEQ ID NO:47, SEQ ID NO:48, SEQ ID NO:50, SEQ ID NO:51, SEQ ID NO:53, SEQ ID NO:54, SEQ ID NO:56, SEQ ID NO:57, SEQ ID NO: 59, SEQ ID NO:60, SEQ ID NO:62, SEQ ID NO:63, SEQ ID NO:65, SEQ ID NO:66, SEQ ID NO: 68, SEQ ID NO:69, SEQ ID NO:71, SEQ ID NO:72, SEQ ID NO:74, and SEQ ID NO: 75.

12. The pharmaceutical composition according to claim 1, wherein the recombinant protein vaccine and/or the adenovirus vaccine contain/contains a nucleic acid for resisting infection with SARS-CoV-2 or the mutant thereof.

13. The pharmaceutical composition according to claim 12, wherein the nucleotide sequence of the nucleic acid is shown in at least one member selected from the group consisting of SEQ ID NO:24, SEQ ID NO:26, SEQ ID NO: 28, SEQ ID NO:30, SEQ ID NO:32, SEQ ID NO:34, SEQ ID NO:36, SEQ ID NO:38, SEQ ID NO:41, SEQ ID NO:42, SEQ ID NO:44, SEQ ID NO:45, SEQ ID NO:47, SEQ ID NO: 48, SEQ ID NO:50, SEQ ID NO:51, SEQ ID NO:53, SEQ ID NO:54, SEQ ID NO:56, SEQ ID NO: 57, SEQ ID NO:59, SEQ ID NO:60, SEQ ID NO:62, SEQ ID NO:63, SEQ ID NO:65, SEQ ID NO:66, SEQ ID NO:68, SEQ ID NO:69, SEQ ID NO:71, SEQ ID NO:72, SEQ ID NO: 74, SEQ ID NO:75, and SEQ ID NO:76.

14. The pharmaceutical composition according to claim 13, wherein the nucleotide sequence is obtained based on at least one encoded amino acid sequence selected from the group consisting of SEQ ID NO:25, SEQ ID NO: 27, SEQ ID NO:29, SEQ ID NO:31, SEQ ID NO:33, SEQ ID NO:35, SEQ ID NO:37, SEQ ID NO:39, SEQ ID NO:40, SEQ ID NO:43, SEQ ID NO:46, SEQ ID NO:49, SEQ ID NO: 52, SEQ ID NO:55, SEQ ID NO:58, SEQ ID NO:61, SEQ ID NO:64, SEQ ID NO:67, SEQ ID NO: 70, SEQ ID NO:73, and SEQ ID NO:77 through optimization of codons or cells; and further, the cells are mammalian cells CHO or insect cells.

15. A recombinant vector or an adenovirus vector comprising a polynucleotide sequence in the recombinant protein vaccine or the adenovirus vaccine in the pharmaceutical composition according to claim 1, wherein the polynucleotide sequence is at least one member selected from the group consisting of SEQ ID NO:10, SEQ ID NO:11, SEQ ID NO:12, SEQ ID NO: 13, SEQ ID NO:14, SEQ ID NO:15, SEQ ID NO:16, SEQ ID NO:17, SEQ ID NO:19, SEQ ID NO:21, SEQ ID NO:23, SEQ ID NO:24, SEQ ID NO:26, SEQ ID NO:28, SEQ ID NO: 30, SEQ ID NO:32, SEQ ID NO:34, SEQ ID NO:36, SEQ ID NO:38, SEQ ID NO:41, SEQ ID NO: 42, SEQ ID NO:44, SEQ ID NO:45, SEQ ID NO:47, SEQ ID NO:48, SEQ ID NO:50, SEQ ID NO:51, SEQ ID NO:53, SEQ ID NO:54, SEQ ID NO:56, SEQ ID NO:57, SEQ ID NO: 59, SEQ ID NO:60, SEQ ID NO:62, SEQ ID NO:63, SEQ ID NO:65, SEQ ID NO:66, SEQ ID NO: 68, SEQ ID NO:69, SEQ ID NO:71, SEQ ID NO:72, SEQ ID NO:74, SEQ ID NO:75, and SEQ ID NO:76.

16. The recombinant vector or adenovirus vector according to claim 15, wherein the recombinant vector is at least one member selected from the group consisting of an insect baculovirus expression vector, a mammalian cell expression vector, an Escherichia coli expression vector and a yeast expression vector.

17. The recombinant vector or adenovirus vector according to claim 15, wherein the adenovirus vector is a human replication-deficient recombinant adenovirus vector.

18. A host cell comprising the recombinant vector or adenovirus vector according to claim 15.

19. The host cell according to claim 18, which is at least one member selected from the group consisting of insect cells, mammalian cells, Escherichia coli and yeasts.

20. The pharmaceutical composition according to claim 1, wherein the recombinant protein vaccine and/or the adenovirus vaccine further comprise/comprises a pharmaceutically acceptable excipient or a complementary ingredient.

21. The pharmaceutical composition according to claim 20, wherein the complementary ingredient is at least one immunologic adjuvant selected from the group consisting of a squalene oil-in-water emulsion, aluminum salt, calcium salt, plant saponin, plant polysaccharide, monophosphoryl lipid A, muramyl dipeptide, muramyl tripeptide, bacterial toxin, GM-CSF cytokines, lipid and cationic liposome materials; further, the at least one immunologic adjuvant satisfies at least one the following requirements: the squalene oil-in-water emulsion is MF59, the aluminum salt is at least one of aluminum hydroxide and alum, the calcium salt is tricalcium phosphate, the plant saponin is QS-21 or ISCOM, the plant polysaccharide is astragalus polysaccharide, the bacterial toxin is at least one of recombinant cholera toxin and diphtheria toxin, the lipid is at least one of phosphatidylethanolamine, phosphatidylcholine, cholesterol and dioleoyl phosphatidylethanolamine, and the cationic liposome material is at least one of (2,3-dioleoxypropyl) trimethyl ammonium chloride, N-[1-(2,3-dioleoyl chloride) propyl]-N,N,N-trimethylamine chloride, cationic cholesterol, dimethyl-2, 3-dioleoxypropyl-2-(2-arginine formyl amino) ethyl ammonium trimethyldodecylammonium bromide, dodecyltrimethylammonium bromide, tetradecyltrimethylammonium bromide, hexadecyltrimethylammonium bromide, dimethyldioctadecylammonium bromide, and CpG ODN.

22. The pharmaceutical composition according to claim 20, wherein the vaccine preparations of the recombinant protein vaccine and the adenovirus vaccine are an intramuscular injection, an injection, a nasal drop, a spray, a nasal spray or an inhalation.

23. A method for preventing and/or treating infection with SARS-CoV-2 or a mutant thereof, said method comprising administering the pharmaceutical composition according to claim 1.

24. A method for preventing and/or treating infection or pathopoiesis with a SARS-COV-2 variant strain, said method comprising administering the pharmaceutical composition according to claim 1, wherein the SARS-COV-2 variant strain is a member selected from the group consisting of at least one of Alpha, Beta, Gamma, Delta and Omicron.

25. A method for treating and/or preventing respiratory virus infection or pathopoiesis, said method comprising administering the pharmaceutical composition according to claim 1.

26. The pharmaceutical composition according to claim 1, comprising the recombinant protein vaccine and an inactivated influenza virus vaccine.

27. The pharmaceutical composition according to claim 1, comprising the adenovirus vaccine and an influenza virus recombinant protein vaccine as active ingredients.

28. A method for treating and/or preventing respiratory virus infection or pathopoiesis, said method comprising administering the pharmaceutical composition according to claim 26.


SEQUENCE LISTING (79 sequences)

SEQ ID NO: 1 - protein, 226 aa, synthetic construct
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNG

SEQ ID NO: 2 - protein, 492 aa, synthetic construct
MLLVNQSHQGFNKEHTSKMVSAIVLYVLLAAAAHSAFAADSIHIKDSDDLKNRLAEAGDKLVVIDFMATWCGPCKMIGPKLDEMANEMSDSIVVLKVDVDECEDIATEYNINSMPTFVFVKNSKKIEEFSGANVDKLRNTIIKLKLAGSGSGHMHHHHHHSSGDDDDKVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSTPCNGVKGFNCYFPLQSYGFQPTYGVGYQPYRVVVLSFELLHAPATVCGPKKSTNLVKNKSVNFNFNGLYENQKLIANQFNSAIGKIQDSLSSTASALGKLQDVVNQNAQALNTLVKQLKNHTSPDVDLGDISGINASVVNIQKEIDRLNEVAKNLNESLIDLQEL

SEQ ID NO: 3 - protein, 492 aa, synthetic construct
MLLVNQSHQGFNKEHTSKMVSAIVLYVLLAAAAHSAFAADSIHIKDSDDLKNRLAEAGDKLVVIDFMATWCGPCKMIGPKLDEMANEMSDSIVVLKVDVDECEDIATEYNINSMPTFVFVKNSKKIEEFSGANVDKLRNTIIKLKLAGSGSGHMHHHHHHSSGDDDDKVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKSVNFNFNGLYENQKLIANQFNSAIGKIQDSLSSTASALGKLQDVVNQNAQALNTLVKQLKNHTSPDVDLGDISGINASVVNIQKEIDRLNEVAKNLNESLIDLQEL

SEQ ID NO: 4 - protein, 492 aa, synthetic construct
MLLVNQSHQGFNKEHTSKMVSAIVLYVLLAAAAHSAFAADSIHIKDSDDLKNRLAEAGDKLVVIDFMATWCGPCKMIGPKLDEMANEMSDSIVVLKVDVDECEDIATEYNINSMPTFVFVKNSKKIEEFSGANVDKLRNTIIKLKLAGSGSGHMHHHHHHSSGDDDDKVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKSVNFNFNGLYENQKLIANQFNSAIGKIQDSLSSTASALGKLQDVVNQNAQALNTLVKQLKNHTSPDVDLGDISGINASVVNIQKEIDRLNEVAKNLNESLIDLQEL

SEQ ID NO: 5 - protein, 492 aa, synthetic construct
MLLVNQSHQGFNKEHTSKMVSAIVLYVLLAAAAHSAFAADSIHIKDSDDLKNRLAEAGDKLVVIDFMATWCGPCKMIGPKLDEMANEMSDSIVVLKVDVDECEDIATEYNINSMPTFVFVKNSKKIEEFSGANVDKLRNTIIKLKLAGSGSGHMHHHHHHSSGDDDDKVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKSVNFNFNGLYENQKLIANQFNSAIGKIQDSLSSTASALGKLQDVVNQNAQALNTLVKQLKNHTSPDVDLGDISGINASVVNIQKEIDRLNEVAKNLNESLIDLQEL

SEQ ID NO: 6 - protein, 492 aa, synthetic construct
MLLVNQSHQGFNKEHTSKMVSAIVLYVLLAAAAHSAFAADSIHIKDSDDLKNRLAEAGDKLVVIDFMATWCGPCKMIGPKLDEMANEMSDSIVVLKVDVDECEDIATEYNINSMPTFVFVKNSKKIEEFSGANVDKLRNTIIKLKLAGSGSGHMHHHHHHSSGDDDDKVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKSVNFNFNGLYENQKLIANQFNSAIGKIQDSLSSTASALGKLQDVVNQNAQALNTLVKQLKNHTSPDVDLGDISGINASVVNIQKEIDRLNEVAKNLNESLIDLQEL

SEQ ID NO: 7 - protein, 492 aa, synthetic construct
MLLVNQSHQGFNKEHTSKMVSAIVLYVLLAAAAHSAFAADSIHIKDSDDLKNRLAEAGDKLVVIDFMATWCGPCKMIGPKLDEMANEMSDSIVVLKVDVDECEDIATEYNINSMPTFVFVKNSKKIEEFSGANVDKLRNTIIKLKLAGSGSGHMHHHHHHSSGDDDDKVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKSVNFNFNGLYENQKLIANQFNSAIGKIQDSLSSTASALGKLQDVVNQNAQALNTLVKQLKNHTSPDVDLGDISGINASVVNIQKEIDRLNEVAKNLNESLIDLQEL

SEQ ID NO: 8 - protein, 492 aa, synthetic construct
MLLVNQSHQGFNKEHTSKMVSAIVLYVLLAAAAHSAFAADSIHIKDSDDLKNRLAEAGDKLVVIDFMATWCGPCKMIGPKLDEMANEMSDSIVVLKVDVDECEDIATEYNINSMPTFVFVKNSKKIEEFSGANVDKLRNTIIKLKLAGSGSGHMHHHHHHSSGDDDDKVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKSVNFNFNGLYENQKLIANQFNSAIGKIQDSLSSTASALGKLQDVVNQNAQALNTLVKQLKNHTSPDVDLGDISGINASVVNIQKEIDRLNEVAKNLNESLIDLQEL

SEQ ID NO: 9 - protein, 492 aa, synthetic construct
MLLVNQSHQGFNKEHTSKMVSAIVLYVLLAAAAHSAFAADSIHIKDSDDLKNRLAEAGDKLVVIDFMATWCGPCKMIGPKLDEMANEMSDSIVVLKVDVDECEDIATEYNINSMPTFVFVKNSKKIEEFSGANVDKLRNTIIKLKLAGSGSGHMHHHHHHSSGDDDDKVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKSVNFNFNGLYENQKLIANQFNSAIGKIQDSLSSTASALGKLQDVVNQNAQALNTLVKQLKNHTSPDVDLGDISGINASVVNIQKEIDRLNEVAKNLNESLIDLQEL

SEQ ID NO: 10 - DNA, 1479 bp, synthetic construct
  source 1..1479: mol_type = other DNA
atgctactagtaaatcagtcacaccaaggcttcaataaggaacacacaagcaagatggtgagcgctatcgtgctgtacgtcctgctggccgctgctgctcacagcgctttcgctgctgactccatccacatcaaggacagcgacgacctgaagaaccgtctggccgaggccggtgacaagctggtcgtcatcgacttcatggccacttggtgcggtccttgcaagatgatcggccctaagctggacgagatggctaacgagatgtccgacagcatcgtggtcctgaaggtggacgtcgacgagtgcgaggacatcgccaccgaatacaacatcaacagcatgcccaccttcgtgttcgtgaagaacagcaagaagatcgaggaattttccggcgctaacgtcgacaagctgcgtaacaccatcatcaagctgaagctggccggctccggctccggccacatgcatcaccaccaccaccattcctccggtgacgacgacgacaaggtgcagccaaccgaatctatcgtcagattcccaaacatcactaacctgtgccctttcggagaggtgttcaacgctaccaggttcgccagcgtctacgcttggaaccgcaagcgtatcagcaactgcgtcgccgactactctgtgctgtacaactccgctagcttctctactttcaagtgctacggcgtgtcacctaccaagctgaacgacctgtgcttcactaacgtctacgccgactccttcgtgatccgcggagacgaagtccgtcagatcgctcctggacagaccggaaatatcgctgactacaactacaagctgccagacgacttcactggctgcgtgatcgcttggaactcaaacaacctggactccaaggtcggtggcaactacaactaccggtacaggctgttcagaaagtcaaacctgaagcctttcgagcgcgacatctcaaccgaaatctaccaggctggttccactccctgcaacggtgtgaagggcttcaactgctacttccccctgcagtcctacggtttccagccaacctatggagtcggttaccagccttaccgtgtggtcgtgctgagcttcgaactgctccacgctcctgctactgtgtgcggtcccaagaagtctactaacctggtcaaaaacaaaagcgtcaacttcaacttcaacggtctgtacgagaaccagaagctgatcgctaaccagttcaacagcgccatcggcaagatccaggactcactgtcatccactgcctccgctctgggaaagctgcaggacgtcgtgaaccagaacgcccaggctctgaacaccctggtcaagcagctgaagaaccacacctctcctgacgtcgacctgggcgacatcagcggaatcaacgcttctgtcgtgaacatccagaaggagatcgaccgcctgaacgaagtggccaagaacctgaacgaatccctgattgacctccaagaactctaa

SEQ ID NO: 11 - DNA, 1479 bp, synthetic construct
  source 1..1479: mol_type = other DNA
atgctactagtaaatcagtcacaccaaggcttcaataaggaacacacaagcaagatggtgagcgctatcgtgctgtacgtcctgctggccgctgctgctcacagcgctttcgctgctgactccatccacatcaaggacagcgacgacctgaagaaccgtctggccgaggccggtgacaagctggtcgtcatcgacttcatggccacttggtgcggtccttgcaagatgatcggccctaagctggacgagatggctaacgagatgtccgacagcatcgtggtcctgaaggtggacgtcgacgagtgcgaggacatcgccaccgaatacaacatcaacagcatgcccaccttcgtgttcgtgaagaacagcaagaagatcgaggaattttccggcgctaacgtcgacaagctgcgtaacaccatcatcaagctgaagctggccggctccggctccggccacatgcatcaccaccaccaccattcctccggtgacgacgacgacaaggtgcagccaaccgaatctatcgtcagattcccaaacatcactaacctgtgccctttcggagaggtgttcaacgctaccaggttcgccagcgtctacgcttggaaccgcaagcgtatcagcaactgcgtcgccgactactctgtgctgtacaactccgctagcttctctactttcaagtgctacggcgtgtcacctaccaagctgaacgacctgtgcttcactaacgtctacgccgactccttcgtgatccgcggagacgaagtccgtcagatcgctcctggacagaccggaaagatcgctgactacaactacaagctgccagacgacttcactggctgcgtgatcgcttggaactcaaacaacctggactccaaggtcggtggcaactacaactaccggtacaggctgttcagaaagtcaaacctgaagcctttcgagcgcgacatctcaaccgaaatctaccaggctggttccaagccctgcaacggtgtggagggcttcaactgctacttccccctgcagtcctacggtttccagccaaccaacggagtcggttaccagccttaccgtgtggtcgtgctgagcttcgaactgctccacgctcctgctactgtgtgcggtcccaagaagtctactaacctggtcaaaaacaaaagcgtcaacttcaacttcaacggtctgtacgagaaccagaagctgatcgctaaccagttcaacagcgccatcggcaagatccaggactcactgtcatccactgcctccgctctgggaaagctgcaggacgtcgtgaaccagaacgcccaggctctgaacaccctggtcaagcagctgaagaaccacacctctcctgacgtcgacctgggcgacatcagcggaatcaacgcttctgtcgtgaacatccagaaggagatcgaccgcctgaacgaagtggccaagaacctgaacgaatccctgattgacctccaagaactctaa

SEQ ID NO: 12 - DNA, 1479 bp, synthetic construct
  source 1..1479: mol_type = other DNA
atgctactagtaaatcagtcacaccaaggcttcaataaggaacacacaagcaagatggtgagcgctatcgtgctgtacgtcctgctggccgctgctgctcacagcgctttcgctgctgactccatccacatcaaggacagcgacgacctgaagaaccgtctggccgaggccggtgacaagctggtcgtcatcgacttcatggccacttggtgcggtccttgcaagatgatcggccctaagctggacgagatggctaacgagatgtccgacagcatcgtggtcctgaaggtggacgtcgacgagtgcgaggacatcgccaccgaatacaacatcaacagcatgcccaccttcgtgttcgtgaagaacagcaagaagatcgaggaattttccggcgctaacgtcgacaagctgcgtaacaccatcatcaagctgaagctggccggctccggctccggccacatgcatcaccaccaccaccattcctccggtgacgacgacgacaaggtgcagccaaccgaatctatcgtcagattcccaaacatcactaacctgtgccctttcgatgaggtgttcaacgctaccaggttcgccagcgtctacgcttggaaccgcaagcgtatcagcaactgcgtcgccgactactctgtgctgtacaacctcgctcctttcttcactttcaagtgctacggcgtgtcacctaccaagctgaacgacctgtgcttcactaacgtctacgccgactccttcgtgatccgcggagacgaagtccgtcagatcgctcctggacagaccggaaatatcgctgactacaactacaagctgccagacgacttcactggctgcgtgatcgcttggaactcaaacaagctggactccaaggtcagtggcaactacaactacctgtacaggctgttcagaaagtcaaacctgaagcctttcgagcgcgacatctcaaccgaaatctaccaggctggtaataagccctgcaacggtgtggctggcttcaactgctacttccccctgagatcctactctttccgaccaacctacggagtcggtcatcagccttaccgtgtggtcgtgctgagcttcgaactgctccacgctcctgctactgtgtgcggtcccaagaagtctactaacctggtcaaaaacaaaagcgtcaacttcaacttcaacggtctgtacgagaaccagaagctgatcgctaaccagttcaacagcgccatcggcaagatccaggactcactgtcatccactgcctccgctctgggaaagctgcaggacgtcgtgaaccagaacgcccaggctctgaacaccctggtcaagcagctgaagaaccacacctctcctgacgtcgacctgggcgacatcagcggaatcaacgcttctgtcgtgaacatccagaaggagatcgaccgcctgaacgaagtggccaagaacctgaacgaatccctgattgacctccaagaactctaa

SEQ ID NO: 13 - DNA, 1479 bp, synthetic construct
  source 1..1479: mol_type = other DNA
atgctgctggtgaaccagagccaccagggtttcaacaaggaacacactagcaagatggtgtctgctatcgtcctctacgtgctgctcgccgccgctgctcactcagctttcgccgctgactctatccacatcaaggactccgacgacctcaagaaccgtttggccgaagccggcgacaagctggtcgttatcgacttcatggctacctggtgtggcccttgcaagatgatcggacctaagctggacgaaatggctaacgagatgagcgacagcatcgtcgttctgaaggtggacgtcgacgaatgtgaggacatcgccactgagtacaacatcaacagcatgcctaccttcgttttcgtgaagaactctaagaagatcgaggagttctctggtgccaacgtggacaagctccgtaacaccatcatcaagctcaagctggccggcagcggtagcggtcacatgcaccaccaccaccaccattcaagcggcgacgacgacgacaaggtgcagccaaccgaatcaatcgtccgcttcccaaacatcactaacctgtgcccattcgacgaggtgttcaacgccacccgtttcgcctctgtctacgcttggaaccgcaagcgcatctccaactgtgtcgccgactacagcgtgctgtacaacttcgctcctttcttcgcattcaagtgttacggagtgtcccctaccaagctcaacgatttgtgcttcacaaacgtgtacgctgacagcttcgtgatccgtggtaacgaggtttctcaaatcgcccctggtcagacaggtaacatcgctgactacaactacaagctcccagacgacttcactggctgtgttatcgcttggaactcaaacaagctggactctaaggtgggtggtaactacaactacctctaccgtctgttccgcaagagcaacctcaagccattcgaacgtgacatcagcacagagatctaccaagccggtaacaagccctgcaacggcgttgccggattcaactgctacttccccctccgttcctacggattccgcccaacctacggcgtgggtcaccagccataccgcgtggttgtgctgagcttcgaactgctgcacgcccccgctaccgtgtgtggccctaagaagagcactaacctcgttaagaacaagtccgtgaacttcaacttcaacggtctctacgaaaaccagaagctgatcgctaaccagttcaactccgccatcggcaagatccaggactccctgtcctccacagcttccgctctgggcaagctgcaggacgttgtgaaccaaaacgctcaggccctgaacacactggtgaagcaactgaagaaccacaccagccccgacgttgacctgggtgacatcagcggtatcaacgctagcgtggtgaacatccagaaggagatcgacaggctgaacgaagtggctaagaacttgaacgagagcctcatcgacctgcaggaactgtaa

SEQ ID NO: 14 - DNA, 1479 bp, synthetic construct
  source 1..1479: mol_type = other DNA
atgctgctggtgaaccagagccaccagggtttcaacaaggaacacactagcaagatggtgtctgctatcgtcctctacgtgctgctcgccgccgctgctcactcagctttcgccgctgactctatccacatcaaggactccgacgacctcaagaaccgtttggccgaagccggcgacaagctggtcgttatcgacttcatggctacctggtgtggcccttgcaagatgatcggacctaagctggacgaaatggctaacgagatgagcgacagcatcgtcgttctgaaggtggacgtcgacgaatgtgaggacatcgccactgagtacaacatcaacagcatgcctaccttcgttttcgtgaagaactctaagaagatcgaggagttctctggtgccaacgtggacaagctccgtaacaccatcatcaagctcaagctggccggcagcggtagcggtcacatgcaccaccaccaccaccattcaagcggcgacgacgacgacaaggtgcagccaaccgaatcaatcgtccgcttcccaaacatcactaacctgtgcccattcgacgaggtgttcaacgccacccgtttcgcctctgtctacgcttggaaccgcaagcgcatctccaactgtgtcgccgactacagcgtgctgtacaacttcgctcctttcttcgcattcaagtgttacggagtgtcccctaccaagctcaacgatttgtgcttcacaaacgtgtacgctgacagcttcgtgatccgtggtaacgaggtttctcaaatcgcccctggtcagacaggtaacatcgctgactacaactacaagctcccagacgacttcactggctgtgttatcgcttggaactcaaacaagctggactctaaggtgggtggtaactacaactaccggtaccgtctgttccgcaagagcaacctcaagccattcgaacgtgacatcagcacagagatctaccaagccggtaacaagccctgcaacggcgttgccggagtcaactgctacttccccctccagtcctacggattccgcccaacctacggcgtgggtcaccagccataccgcgtggttgtgctgagcttcgaactgctgcacgcccccgctaccgtgtgtggccctaagaagagcactaacctcgttaagaacaagtccgtgaacttcaacttcaacggtctctacgaaaaccagaagctgatcgctaaccagttcaactccgccatcggcaagatccaggactccctgtcctccacagcttccgctctgggcaagctgcaggacgttgtgaaccaaaacgctcaggccctgaacacactggtgaagcaactgaagaaccacaccagccccgacgttgacctgggtgacatcagcggtatcaacgctagcgtggtgaacatccagaaggagatcgacaggctgaacgaagtggctaagaacttgaacgagagcctcatcgacctgcaggaactgtaa

SEQ ID NO: 15 - DNA, 1479 bp, synthetic construct
  source 1..1479: mol_type = other DNA
atgctgctggtgaaccagagccaccagggtttcaacaaggaacacactagcaagatggtgtctgctatcgtcctctacgtgctgctcgccgccgctgctcactcagctttcgccgctgactctatccacatcaaggactccgacgacctcaagaaccgtttggccgaagccggcgacaagctggtcgttatcgacttcatggctacctggtgtggcccttgcaagatgatcggacctaagctggacgaaatggctaacgagatgagcgacagcatcgtcgttctgaaggtggacgtcgacgaatgtgaggacatcgccactgagtacaacatcaacagcatgcctaccttcgttttcgtgaagaactctaagaagatcgaggagttctctggtgccaacgtggacaagctccgtaacaccatcatcaagctcaagctggccggcagcggtagcggtcacatgcaccaccaccaccaccattcaagcggcgacgacgacgacaaggtgcagccaaccgaatcaatcgtccgcttcccaaacatcactaacctgtgcccattcgacgaggtgttcaacgccacccgtttcgcctctgtctacgcttggaaccgcaagcgcatctccaactgtgtcgccgactacagcgtgctgtacaacttcgctcctttcttcgcattcaagtgttacggagtgtcccctaccaagctcaacgatttgtgcttcacaaacgtgtacgctgacagcttcgtgatccgtggtaacgaggtttctcaaatcgcccctggtcagacaggtaacatcgctgactacaactacaagctcccagacgacttcactggctgtgttatcgcttggaactcaaacaagctggactctaaggtgggtggtaactacaactaccagtaccgtctgttccgcaagagcaacctcaagccattcgaacgtgacatcagcacagagatctaccaagccggtaacaagccctgcaacggcgttgccggattcaactgctacttccccctccgttcctacggattccgcccaacctacggcgtgggtcaccagccataccgcgtggttgtgctgagcttcgaactgctgcacgcccccgctaccgtgtgtggccctaagaagagcactaacctcgttaagaacaagtccgtgaacttcaacttcaacggtctctacgaaaaccagaagctgatcgctaaccagttcaactccgccatcggcaagatccaggactccctgtcctccacagcttccgctctgggcaagctgcaggacgttgtgaaccaaaacgctcaggccctgaacacactggtgaagcaactgaagaaccacaccagccccgacgttgacctgggtgacatcagcggtatcaacgctagcgtggtgaacatccagaaggagatcgacaggctgaacgaagtggctaagaacttgaacgagagcctcatcgacctgcaggaactgtaa

SEQ ID NO: 16 - DNA, 1479 bp, synthetic construct; identical to SEQ ID NO: 14
  source 1..1479: mol_type = other DNA

SEQ ID NO: 17 - DNA, 1479 bp, synthetic construct
  source 1..1479: mol_type = other DNA
atgctgctggtgaaccagagccaccagggtttcaacaaggaacacactagcaagatggtgtctgctatcgtcctctacgtgctgctcgccgccgctgctcactcagctttcgccgctgactctatccacatcaaggactccgacgacctcaagaaccgtttggccgaagccggcgacaagctggtcgttatcgacttcatggctacctggtgtggcccttgcaagatgatcggacctaagctggacgaaatggctaacgagatgagcgacagcatcgtcgttctgaaggtggacgtcgacgaatgtgaggacatcgccactgagtacaacatcaacagcatgcctaccttcgttttcgtgaagaactctaagaagatcgaggagttctctggtgccaacgtggacaagctccgtaacaccatcatcaagctcaagctggccggcagcggtagcggtcacatgcaccaccaccaccaccattcaagcggcgacgacgacgacaaggtgcagccaaccgaatcaatcgtccgcttcccaaacatcactaacctgtgcccattcgacgaggtgttcaacgccacccgtttcgcctctgtctacgcttggaaccgcaagcgcatctccaactgtgtcgccgactacagcgtgctgtacaacttcgctcctttcttcgcattcaagtgttacggagtgtcccctaccaagctcaacgatttgtgcttcacaaacgtgtacgctgacagcttcgtgatccgtggtaacgaggtttctcaaatcgcccctggtcagacaggtaacatcgctgactacaactacaagctcccagacgacttcactggctgtgttatcgcttggaactcaaacaagctggactctacggtgggtggtaactacaactaccggtaccgtctgttccgcaagagcaagctcaagccattcgaacgtgacatcagcacagagatctaccaagccggtaacaagccctgcaacggcgttgccggagtcaactgctacttccccctccagtcctacggattccgcccaacctacggcgtgggtcaccagccataccgcgtggttgtgctgagcttcgaactgctgcacgcccccgctaccgtgtgtggccctaagaagagcactaacctcgttaagaacaagtccgtgaacttcaacttcaacggtctctacgaaaaccagaagctgatcgctaaccagttcaactccgccatcggcaagatccaggactccctgtcctccacagcttccgctctgggcaagctgcaggacgttgtgaaccaaaacgctcaggccctgaacacactggtgaagcaactgaagaaccacaccagccccgacgttgacctgggtgacatcagcggtatcaacgctagcgtggtgaacatccagaaggagatcgacaggctgaacgaagtggctaagaacttgaacgagagcctcatcgacctgcaggaactgtaa

SEQ ID NO: 18 - protein, 801 aa, synthetic construct
MDAMKRGLCCVLLLCGAVFVSPVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKSVNFNFNGLYENQKLIANQFNSAIGKIQDSLSSTASALGKLQDVVNQNAQALNTLVKQLVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKSVNFNFNGKNHTSPDVDLGDISGINASVVNIQKEIDRLNEVAKNLNESLIDLQELGKYVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKSVNFNFNG

SEQ ID NO: 19 - DNA, 2406 bp, synthetic construct
  source 1..2406: mol_type = other DNA
atggacgccatgaagagaggcctgtgctgcgtgctgctcctgtgcggcgccgtgttcgtgagccccgtgcaacccaccgaaagcattgtgcggtttcccaatatcaccaacctctgtcctttcgacgaggtgttcaacgccacaagatttgctagcgtgtacgcctggaaccggaagagaatctccaattgtgtcgccgactatagcgtcctctataacctggcccccttcttcacattcaagtgctacggcgtgtcccccacaaagctgaacgacctgtgctttaccaatgtgtacgccgacagcttcgtcatcagaggggacgaggtccggcagatcgctcctgggcagaccggcaatatcgccgactacaactataagctccctgacgacttcaccgggtgcgtcattgcttggaactccaacaagctggattccaaagtgagcgggaactacaactatctctacagactgttcagaaagagcaacctgaaacctttcgaaagagacatctccacagagatctaccaagccggcaacaagccctgcaacggcgtggccggctttaattgttacttccccctcagaagctacagcttcagacccacctacggcgtgggccatcagccctatagagtcgtggtgctgtccttcgaactgctgcacgcccccgccaccgtgtgtggccccaagaaaagcacaaatctggtcaagaataagagcgtcaattttaatttcaacggcctgtacgagaatcagaagctgatcgccaatcagttcaacagcgccatcggcaagatccaagacagcctgagcagcaccgctagcgccctgggcaagctgcaagacgtggtgaatcagaacgcccaagccctgaacaccctggtgaagcagctggtgcagcccaccgaatccattgtcagattccccaacatcaccaacctgtgcccctttggcgaagtgtttaacgccacccggttcgctagcgtgtatgcctggaaccggaagagaattagcaactgcgtggccgattacagcgtgctctataacagcgctagcttcagcacctttaagtgctatggcgtgagccctaccaagctgaatgacctctgcttcaccaacgtgtatgctgatagcttcgtgatcagaggcgacgaagtgagacagatcgcccctgggcagaccggcaagattgccgactacaattataagctgcctgatgatttcaccggctgcgtgatcgcctggaacagcaacaacctggattccaaggtcgggggcaattacaattacagatacagactcttcagaaagtccaacctgaagcccttcgagagagacatcagcaccgagatctatcaagccgggagcaagccctgtaacggcgtggagggcttcaattgctactttcccctgcagtcctatgggtttcagcccaccaacggggtgggctatcagccctatagagtcgtcgtcctcagcttcgaactgctgcacgctcctgctacagtgtgcgggcccaagaagagcaccaatctcgtcaagaataaaagcgtcaacttcaacttcaacggcaagaaccacacaagccccgacgtggacctgggcgacatcagcggcatcaacgctagcgtggtgaacattcagaaggagatcgacagactgaacgaggtggccaagaacctgaacgagagcctgatcgacctgcaagagctgggcaagtacgtgcagcccaccgagagcatcgtgagattccccaatatcacaaatctctgcccttttggcgaggtgtttaacgctacaagattcgctagcgtctacgcctggaacagaaaacggatcagcaattgtgtggccgactatagcgtgctgtacaattccgctagcttcagcacattcaaatgctacggggtgtcccccaccaagctgaacgatctctgcttcaccaacgtctacgccgacagcttcgtgattagaggcgacgaggtcagacaaatcgcccctgggcagaccggcaacatcgctgactataattacaagctgcctgacgatttcaccgggtgtgtgatcgcctggaactccaacaatctggactccaaggtggggggcaactataactacctgtacagactctttagaaagagcaatctcaagcccttcgaacgggatattagcaccgaaatttaccaagccggctccacaccctgtaacggcgtgaaggggttcaactgctatttccccctgcagagctacggctttcagcccacctacggcgtggggtatcagccttaccgggtggtcgtgctcagcttcgagctgctgcatgctcctgccaccgtctgtgggcctaagaagagcaccaacctggtgaagaacaaatccgtgaacttcaactttaacggctga

SEQ ID NO: 20 - protein, 801 aa, synthetic construct
MDAMKRGLCCVLLLCGAVFVSPVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKSVNFNFNGLYENQKLIANQFNSAIGKIQDSLSSTASALGKLQDVVNQNAQALNTLVKQLVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKSVNFNFNGKNHTSPDVDLGDISGINASVVNIQKEIDRLNEVAKNLNESLIDLQELGKYVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNFNFNG

SEQ ID NO: 21 - DNA, 2406 bp, synthetic construct
  source 1..2406: mol_type = other DNA
atggacgccatgaagagaggcctgtgctgcgtgctgctcctgtgcggcgccgtgttcgtgagccccgtgcaacccaccgagagcattgtgagattccccaatatcaccaacctctgccccttcgacgaagtgtttaacgccacaagattcgctagcgtctacgcttggaatagaaagcggatcagcaactgcgtcgctgattactccgtgctgtacaacttcgcccccttcttcgctttcaagtgttacggggtgagccctaccaagctgaacgacctctgtttcaccaatgtgtacgccgatagcttcgtgatccggggcaacgaagtgagccaaatcgcccccggccaaaccggcaatatcgctgactacaactacaagctccctgatgacttcaccggctgtgtgatcgcttggaacagcaacaagctggacagcaaagtgggcgggaattataactacagataccggctcttccggaagagcaacctcaagccttttgagcgggacatctccaccgaaatctaccaagccggcaataaaccctgcaatggcgtggccggcgtgaactgctacttccctctgcaaagctacggctttagacccacctacggcgtgggccatcagccctacagagtggtcgtgctcagctttgagctgctccacgctcctgctacagtctgcggccctaaaaagagcacaaacctcgtcaagaacaaaagcgtgaacttcaactttaacggcctgtatgagaatcagaagctgatcgccaatcagttcaacagcgccatcggcaagatccaagacagcctgagcagcaccgctagcgccctgggcaagctgcaagacgtggtgaatcagaacgcccaagccctgaacaccctggtgaagcagctggtgcagcctaccgagtccatcgtgcggttccccaacatcacaaacctgtgtccctttggggaagtctttaacgccacccggttcgctagcgtgtatgcctggaatagaaagagaattagcaattgcgtggctgactatagcgtgctgtacaacagcgcctccttctccacatttaagtgctacggcgtgagccccacaaagctgaatgatctctgcttcaccaacgtgtacgccgacagcttcgtcattagaggcgatgaagtgagacagatcgctcctgggcagaccggcaaaattgctgactacaactataagctgcccgacgatttcaccggctgcgtgattgcctggaacagcaataacctggacagcaaggtcggcggcaactataactacagatacagactgttccggaagtccaacctgaagcccttcgagagagacatctccaccgagatctatcaagccggcagcaagccctgcaacggcgtggagggctttaactgctatttccccctgcagagctacggctttcagcctacaaacggcgtggggtatcagccctacagagtcgtggtcctgagctttgaactgctccatgctcccgctaccgtgtgtggccccaagaagagcaccaacctcgtgaaaaacaagtccgtgaatttcaacttcaacgggaagaaccacacaagccccgacgtggacctgggcgacatcagcggcatcaacgctagcgtggtgaacattcagaaggagatcgacagactgaacgaggtggccaagaacctgaacgagagcctgatcgacctgcaagagctgggcaagtacgtgcagcccacagagagcatcgtgcggttccccaacatcaccaatctgtgtcccttcggcgaggtcttcaatgccacccggttcgctagcgtgtatgcttggaacagaaaacggatcagcaattgcgtggccgactacagcgtgctgtataatagcgctagcttcagcaccttcaaatgttatggcgtctcccccaccaagctgaatgacctgtgtttcacaaacgtgtatgccgactccttcgtgatcagaggcgacgaggtcagacagattgcccctgggcagaccggcaagatcgccgactacaactacaaactgcccgatgacttcaccggctgcgtgatcgcctggaattccaacaatctggactccaaagtgggcgggaactacaactacctgtaccggctgtttagaaaaagcaacctgaaacctttcgagcgggatattagcacagaaatctatcaagccgggagcaccccctgcaatggggtggagggcttcaattgctacttccccctgcaatcctatgggtttcagcccaccaacggcgtgggctaccaaccctatagagtggtggtcctcagctttgagctgctgcacgcccccgccaccgtgtgcgggcccaaaaagagcaccaacctggtcaagaacaagtccgtcaactttaactttaacggctga

SEQ ID NO: 22 - protein, 867 aa, synthetic construct
MDAMKRGLCCVLLLCGAVFVSPVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKSVNFNFNGTQNVLYENQKLIANQFNSAIGKIQDSLSSTASALGKLQDVVNQNAQALNTLVKQLSSNFGAISSVLNDILSRLDKVEVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKSVNFNFNGVYDPLQPELDSFKEELDKYFKNHTSPDVDLGDISGINASVVNIQKEIDRLNEVAKNLNESLIDLQELGKYGKYEQYIKWPWYIWLGFIAGVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKSVNFNFNG

SEQ ID NO: 23 - DNA, 2604 bp, synthetic construct
  source 1..2604: mol_type = other DNA
atggacgccatgaagagaggcctgtgctgcgtgctgctcctgtgcggcgccgtgttcgtgagccccgtgcaacctaccgagagcattgtccggttccccaatattaccaacctctgccccttcgacgaggtctttaacgctacaagattcgcctccgtgtacgcctggaaccggaagagaatcagcaattgcgtcgccgactactccgtgctctacaactttgcccccttcttcgctttcaagtgctacggcgtgagccccacaaagctcaacgacctgtgcttcaccaacgtgtacgccgacagctttgtcatcagaggcaacgaggtgagccaaattgcccctggccaaaccggcaacatcgccgattacaactacaagctccccgacgacttcaccggctgtgtgattgcttggaacagcaataagctcgacagcaaggtggggggcaactacaattaccggtaccggctgttccggaagtccaatctgaagcctttcgagagagacatcagcaccgagatctaccaagccgggaacaaaccctgcaatggcgtggccggcgtgaactgctacttccctctgcagagctacgggtttcggcccacctacggcgtgggccaccaaccctacagagtggtggtcctgagcttcgagctgctgcacgcccctgctacagtgtgtggccccaagaaaagcaccaatctcgtgaagaacaagagcgtcaacttcaattttaacggcacacagaacgtgctgtacgagaatcagaagctgatcgccaatcagttcaacagcgccatcggcaagatccaagacagcctgagcagcaccgctagcgccctgggcaagctgcaagacgtggtgaatcagaacgcccaagccctgaacaccctggtgaagcagctgagcagcaacttcggcgccatcagcagcgtgctgaacgacatcctgagcagactggacaaggtggaggtgcagcctacagagtccatcgtcagattccccaacatcaccaacctgtgccccttcggcgaggtgtttaatgccacacggttcgctagcgtgtacgcctggaatcggaagcggatctccaactgcgtcgctgactattccgtgctctataactccgctagcttcagcacatttaagtgctatggggtgtcccccaccaagctgaatgacctgtgcttcacaaacgtctacgccgactcctttgtcatccggggcgacgaagtccggcagatcgcccctgggcagaccggcaaaatcgctgactacaactacaaactgcccgatgacttcaccggctgtgtgatcgcctggaactccaacaatctggactccaaggtcggcggcaattacaattacagatacagactgttcagaaaatccaacctgaaacccttcgagcgggacatttccacagagatctatcaagccggcagcaaaccttgcaacggcgtggaggggttcaactgctattttcccctgcagagctacgggtttcagcccaccaacggcgtcggctaccaaccctatcgggtggtcgtgctgagcttcgaactcctgcacgctcccgccacagtctgcgggcctaaaaagagcaccaacctggtgaagaataaatccgtcaatttcaacttcaacggcgtgtacgaccccctgcagcccgagctggacagcttcaaggaggagctggacaagtacttcaagaaccacacaagccccgacgtggacctgggcgacatcagcggcatcaacgctagcgtggtgaacattcagaaggagatcgacagactgaacgaggtggccaagaacctgaacgagagcctgatcgacctgcaagagctgggcaagtacggcaagtacgagcagtacatcaagtggccctggtacatctggctgggcttcatcgccggcgtgcagcccaccgagagcatcgtccggtttcccaacattaccaacctgtgtcccttcggcgaggtcttcaatgccacacggttcgctagcgtctatgcctggaaccggaagagaattagcaactgcgtggccgactatagcgtcctgtacaacagcgctagcttctccaccttcaagtgttatggcgtgtcccccaccaagctgaatgacctctgttttaccaatgtctatgccgacagcttcgtgatcagaggcgatgaagtgagacagatcgcccctgggcagaccgggaagatcgccgactacaactataagctccccgatgacttcaccggctgcgtcattgcttggaactccaataatctggacagcaaggtggggggcaattataactacctgtatcggctgttcagaaagagcaacctgaagccttttgagagagatatcagcaccgaaatttatcaagccgggagcaccccctgcaacggggtggaaggcttcaactgttacttccccctgcagagctacggctttcagcctaccaacggggtggggtatcagccttatagagtcgtggtcctgtccttcgagctgctgcacgcccccgccacagtgtgcgggcccaagaagagcaccaatctggtgaaaaacaaaagcgtcaacttcaatttcaacggctga

SEQ ID NO: 24 - DNA, 3822 bp, synthetic construct
  source 1..3822: mol_type = other DNA
atgttcgtgttcctggtgctgctgcccctggtgagcagccagtgtgtgaacctgacaacaaggacccagctgcctcccgcctacaccaacagcttcacaaggggcgtgtactaccctgacaaggtgttcaggagcagcgtgctgcactccacacaggacctgttcctgcccttcttctccaacgtgacatggttccacgccatccacgtgtccggcaccaacggcacaaagagattcgacaaccccgtgctgcctttcaacgacggcgtgtacttcgcctccacagagaagtccaacatcatcaggggctggatcttcggcacaacactggacagcaagacacagagcctgctgatcgtgaacaacgccacaaacgtggtcattaaggtgtgtgagttccagttctgcaacgaccccttcctgggcgtgtactatcacaagaacaacaagagctggatggagagcgagttcagggtgtactccagcgccaacaactgtacattcgagtacgtgtcccagcctttcctgatggacctggagggcaagcagggcaacttcaagaacctgagggagttcgtgttcaagaacatcgacggctacttcaagatctactccaagcacacccccatcaacctggtgagagacctgcctcagggcttctccgccctggagcctctggtggacctgcccatcggcatcaacatcacaagattccagacactgctggccctgcacagaagctacctgacccccggcgattccagcagcggctggaccgctggcgccgctgcttactacgtgggctacctgcagcccaggacattcctgctgaagtacaacgagaacggcacaatcaccgatgccgtggattgcgccctggatcctctgagcgagaccaagtgtacactgaagtccttcacagtggagaagggcatctaccagacctccaacttcagagtgcagcccacagagagcatcgtgagattccctaacatcacaaacctgtgccctttcggcgaggtgttcaacgccacaaggttcgccagcgtgtacgcctggaacagaaagaggatcagcaactgcgtggccgattacagcgtgctgtacaactccgcctccttctccacattcaagtgctacggcgtgtcccccaccaagctgaacgatctgtgtttcaccaacgtgtacgccgactccttcgtgatcagaggcgacgaggtgaggcagatcgcccccggacagaccggcaagatcgccgattacaactacaagctgcccgatgacttcaccggctgtgtgatcgcctggaactccaacaacctggattccaaggtgggcggcaactacaactacctgtacagactgttcagaaagtccaacctgaagcccttcgagagagatatctccaccgagatctaccaggccggctccacaccttgtaacggcgtggagggcttcaactgctacttccccctgcagagctacggcttccagcctaccaacggcgtgggctaccagccttacagagtggtggtgctgtccttcgagctgctgcacgcccccgccacagtgtgtggcccaaagaagtccaccaacctggtgaagaacaagtgtgtgaacttcaacttcaacggcctgaccggcacaggcgtgctgaccgagagcaacaagaagttcctgcctttccagcagttcggcagagacatcgccgataccacagacgccgtgagagaccctcagaccctggagatcctggatatcaccccttgcagcttcggcggcgtgtccgtgatcacccctggcacaaacaccagcaaccaggtggccgtgctgtaccaggacgtgaactgtacagaggtgcccgtggccatccacgccgaccagctgacccccacatggagagtgtactccaccggctccaacgtgttccagacaagggccggctgcctgatcggcgccgagcatgtgaacaacagctacgagtgtgacatccctatcggcgccggcatctgcgccagctaccagacccagaccaacagccccaggagggccagatccgtggccagccagagcatcatcgcctacaccatgtccctgggcgccgagaacagcgtggcctactccaacaacagcatcgccatccccaccaacttcacaatctccgtgaccaccgagatcctgcccgtgagcatgaccaagacatccgtggattgtaccatgtacatctgcggcgacagcacagagtgctccaacctgctgctgcagtacggctccttctgtacccagctgaacagggccctgacaggcatcgccgtggagcaggacaagaacacccaggaggtgttcgcccaggtgaagcagatctacaagacacctcccatcaaggacttcggcggcttcaacttctcccagatcctgcccgacccttccaagccctccaagagatccttcatcgaggacctgctgttcaacaaggtgaccctggccgatgccggcttcatcaagcagtacggcgactgcctgggcgacatcgccgccagagacctgatctgcgcccagaagttcaacggcctcaccgtgctgccccctctgctgaccgatgagatgatcgcccagtacaccagcgccctgctggccggaacaatcaccagcggctggacattcggcgccggcgctgctctgcagatccctttcgccatgcagatggcctacaggttcaacggcatcggcgtgacccagaacgtgctgtacgagaaccagaagctgatcgccaaccagttcaacagcgccatcggcaagatccaggattccctgagcagcacagccagcgccctgggcaagctgcaggatgtggtgaaccagaacgcccaggccctgaacacactggtgaagcagctgagcagcaacttcggcgccatcagcagcgtgctcaacgacatcctgagcagactggataaggtggaggccgaggtgcagatcgatagactgatcaccggcaggctgcagtccctgcagacatacgtgacccagcagctgatcagagccgccgagatcagggccagcgccaacctggccgccaccaagatgtccgagtgtgtgctgggccagagcaagagggtggacttctgtggcaagggctaccacctgatgtccttccctcagtccgccccccacggcgtggtgttcctgcacgtgacatacgtgcctgcccaggagaagaacttcacaacagcccctgccatctgtcacgacggcaaggcccacttccccagggagggcgtgttcgtgagcaacggcacccactggttcgtgacccagaggaacttctacgagcctcagatcatcacaaccgataacacattcgtgagcggcaactgtgatgtggtcattggcatcgtgaacaataccgtgtacgatcccctgcagcctgagctggactccttcaaggaggagctggataagtacttcaagaaccacacctcccccgacgtggatctgggcgacattagcggcatcaacgcctccgtggtgaacatccagaaggagatcgacaggctgaacgaggtggccaagaacctgaacgagtccctgatcgatctgcaggagctgggcaagtacgagcagtacatcaagtggccctggtacatctggctgggcttcatcgccggcctgatcgccatcgtgatggtgacaatcatgctgtgttgcatgacatcctgttgttcctgtctgaagggctgttgtagctgtggctcctgttgcaagttcgacgaggatgactccgagcccgtgctgaagggcgtgaagctgcactacacctga

SEQ ID NO: 25 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 26 - DNA, 3813 bp, synthetic construct
  source 1..3813: mol_type = other DNA
atgttcgtgttcctggtgctgctgcccctggtgagcagccaatgcgtgaacctgaccacaagaacacagctgccccccgcctacaccaacagcttcacaagaggcgtgtattatcccgacaaggtgttcagaagcagcgtgctgcacagcacccaagacctgtttctgccttttttcagcaacgtgacctggttccacgccatccacgtgagcggcaccaacggcaccaagagattcgccaaccccgtgctgcccttcaacgacggcgtgtacttcgctagcaccgagaagagcaacatcatcagaggctggatcttcggcaccaccctggatagcaagacacagagcctgctgatcgtgaacaacgccaccaacgtggtgatcaaggtgtgcgagtttcagttctgcaacgaccccttcctgggcgtgtattaccacaagaacaacaagagctggatggagagcgagttcagagtctacagcagcgccaacaactgcaccttcgagtacgtgagccaacccttcctgatggacctggagggcaagcaaggcaatttcaagaacctgagagagttcgtgttcaagaacatcgacggctacttcaagatctacagcaagcacacccccatcaacctggtgagaggcctgccccaaggcttcagcgccctggagcccctggtggacctgcccatcggcatcaacatcacaagatttcagacactgcaccggtcctatctcacccctggcgacagcagcagcgggtggaccgctggcgccgccgcttactacgtgggctacctgcagcctagaaccttcctgctgaagtacaacgagaacggcaccattaccgacgccgtggactgcgccctggaccccctgagcgagaccaagtgcaccctgaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcctggaaccggaaaagaatcagcaactgcgtggccgactacagcgtgctgtacaacagcgctagcttcagcaccttcaagtgctacggcgtgagccccaccaagctgaacgacctgtgcttcaccaacgtgtacgccgacagcttcgtgatcagaggcgacgaggtgagacagatcgcccccgggcagaccggcaacatcgccgactacaactacaagctgcccgacgacttcaccggctgcgtgatcgcctggaacagcaataacctggacagcaaggtgggcggcaactacaactacctgtacagactgttcagaaagagcaacctgaagcccttcgagagagacatcagcaccgagatctaccaagccggcagcaccccctgcaacggcgtgaagggcttcaactgctacttccccctgcagagctacggctttcagcccacctacggcgtgggctatcagccctacagagtggtcgtgctgagcttcgagctgctgcacgcccctgccaccgtgtgtggccccaagaagagcaccaacctggtgaagaacaagtgcgtgaacttcaacttcaacgggctgaccgggaccggcgtgctgaccgagagcaacaagaagtttctgccctttcaacagttcggcagagacatcgccgacaccaccgacgccgtccgggaccctcagaccctggagatcctggacatcaccccctgctccttcggcggcgtgagcgtgatcacccccggcaccaacacaagcaaccaagtggccgtgctgtaccaaggcgtgaactgcaccgaggtgcccgtggccatccacgccgatcagctgacccccacctggagagtgtacagcaccggcagcaacgtgtttcagacaagagccggctgcctgatcggcgccgagcacgtgaacaacagctacgagtgcgacatccccatcggcgccggcatctgcgctagctatcagacacagaccaacagccctagaagagctagaagcgtggctagccaaagcatcatcgcctacaccatgagcctgggcgtggagaacagcgtggcctacagcaacaacagcatcgccatccccaccaacttcaccatcagcgtgaccaccgaaatcctgcccgtgagcatgaccaagacaagcgtggactgcaccatgtacatctgcggcgacagcaccgagtgcagcaacctgctcctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaattctgcccgaccctagcaagcctagcaagagaagcttcatcgaggacctgctgttcaacaaggtgaccctggccgacgccggcttcatcaagcagtacggcgactgcctgggcgatatcgctgctagagacctgatctgcgctcagaagttcaacggcctgaccgtgctcccccccctgctgaccgacgagatgatcgctcagtacacaagcgctctgctcgccgggaccatcacatccgggtggacattcggggccggcgctgccctgcagatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcagaacgcccaagccctgaacaccctggtgaagcagctgagcagcaacttcggcgccatcagctccgtgctgaacgacatcctgagcagactggacccccccgaggccgaggtgcagattgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgcccccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcattgtgaacaataccgtgtacgaccccctgcagcccgagctggacagcttcaaggaggagctggacaagtacttcaagaaccacacaagccccgacgtggacctgggcgacattagcggcatcaacgctagcgtggtgaacattcagaaggagatcgacagactgaacgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaagggctgctgcagctgcgggagctgctgcaagttcgacgaggacgacagcgagcccgtgctgaagggcgtgaagctgcactacacctga

SEQ ID NO: 27 - protein, 1270 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 28 - DNA, 3816 bp, synthetic construct
  source 1..3816: mol_type = other DNA
atgttcgtgttcctggtgctgctgcccctggtgagcagccaatgcgtgaacctgagaacaagaacacagctgccccccgcctacaccaacagcttcacaagaggggtctactaccccgacaaggtgttcagaagcagcgtgctgcacagcacccaagacctgttcctgcccttcttctccaacgtgacctggttccacgccatccacgtgagcggcaccaacggcaccaagagattcgacaaccccgtgctgcccttcaacgacggcgtgtacttcgctagcaccgagaagagcaacatcatcagaggctggatcttcggcaccaccctggacagcaagacacagagcctgctgattgtgaacaacgctaccaacgtggtgatcaaggtgtgcgagtttcagttctgcaacgaccccttcctgggggtgtactaccacaaaaacaacaagagctggatggagagcggcgtgtacagcagcgccaacaactgcaccttcgagtacgtgagccaacccttcctgatggacctggagggcaagcaaggcaacttcaagaacctcagagagttcgtgttcaagaacatcgacggctacttcaagatctacagcaagcacacccccatcaacctggtgagagacctgccccaaggcttcagcgccctggagcccctggtggacctgcccatcggcatcaacatcacaagatttcagaccctgctggccctgcacagaagctacctgacacccggcgacagcagcagcgggtggacagccggcgctgccgcttactacgtgggctacctgcagcctagaaccttcctgctgaagtacaacgagaacggcaccattaccgacgccgtcgactgcgccctggaccccctgagcgagaccaagtgcaccctgaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcctggaacagaaagagaatcagcaactgcgtggccgactacagcgtgctgtacaacagcgctagcttcagcaccttcaaatgctacggggtcagccccaccaagctgaacgacctgtgcttcaccaacgtgtacgccgacagcttcgtgatcagaggcgacgaggtgagacagatcgcccccgggcagaccggcaagatcgccgactacaactacaagctgcccgacgacttcaccggctgcgtgatcgcctggaactccaacaacctggatagcaaggtgggcggcaactacaactatagatacagactgttcagaaagagcaacctgaagcccttcgagagagacatcagcaccgagatctaccaagccggcagcaagccctgcaacggcgtggagggcttcaactgctacttccccctgcagagctacggctttcagcccaccaacggcgtgggctatcagccctacagagtggtcgtgctgagcttcgagctgctgcacgcccctgccaccgtgtgcggccccaagaagagcaccaacctggtgaagaacaagtgcgtgaacttcaacttcaacggcctgaccggcaccggcgtgctgaccgagagcaacaagaagttcctcccttttcagcagttcggcagagacatcgccgacaccaccgatgccgtgagagaccctcagaccctggagatcctggacatcaccccctgcagctttggcggcgtgagcgtgatcacccccggcaccaacacaagcaaccaagtggccgtgctgtaccaaggcgtgaactgcaccgaggtgcccgtggccatccacgccgatcagctgacccccacctggagagtgtacagcaccggcagcaacgtgtttcagacaagagccggctgcctgatcggcgccgagcacgtgaacaacagctacgagtgcgacatccccatcggcgccggcatctgcgctagctatcagacacagaccaacagcagacggagagctagaagcgtggctagccaaagcatcatcgcctacaccatgagcctgggcgccgagaacagcgtggcctacagcaacaacagcatcgccatccccaccaacttcaccatcagcgtgaccaccgagatcctgcccgtctccatgaccaagacaagcgtggactgcaccatgtacatctgcggcgacagcaccgagtgcagcaacctgctcctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagagaagcttcatcgaggacctgctgttcaacaaggtgaccctggccgacgccggcttcatcaagcagtacggcgactgcctgggcgacatcgccgctagagacctgatctgcgctcagaagttcaatggcctgaccgtcctgccccccctgctgaccgacgagatgatcgctcagtacacaagcgccctgctggccggcacaattacaagcggctggacattcggcgctggcgccgccctgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcagaacgtggtgaatcagaacgcccaagccctgaacaccctggtgaagcagctgagcagcaacttcggcgccatcagctccgtgctgaacgatatcctgagcagactggacccccccgaggccgaggtgcagatcgaccggctgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgcccccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcattgtgaacaatacagtctacgaccctctgcagcccgagctggacagcttcaaggaggagctggacaagtacttcaagaatcacacaagccccgacgtggacctgggggacatcagcggcatcaacgctagcgtggtgaacattcagaaggagatcgacagactgaacgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgctgcagctgcctgaagggctgctgctcctgcggcagctgctgcaagttcgacgaggacgacagcgagcccgtgctgaagggcgtgaagctgcactacacctga

SEQ ID NO: 29 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 30 - DNA, 3813 bp, synthetic construct
  source 1..3813: mol_type = other DNA
atgttcgtgttcctggtgctgctgcccctggtgagcagccaatgcgtgaacctgaccacaagaacacagctgccccccgcctacaccaacagcttcacaagaggcgtgtattatcccgacaaggtgttcagaagcagcgtgctgcacagcacccaagacctgtttctgcccttcttcagcaacgtgacctggttccacgccatccacgtgagcggcaccaacggcaccaagagattcgccaaccccgtgctgcccttcaacgacggcgtgtacttcgctagcaccgagaagagcaacatcatcagaggctggatcttcggcaccaccctggatagcaagacacagagcctgctgatcgtgaacaacgccaccaacgtggtgatcaaggtgtgcgagtttcagttctgcaacgaccccttcctgggcgtgtattaccacaagaacaacaagagctggatggagagcgagttcagagtctacagcagcgccaacaactgcaccttcgagtacgtgagccaacccttcctgatggacctggagggcaagcaaggcaatttcaagaacctgagagagttcgtgttcaagaacatcgacggctacttcaagatctacagcaagcacacccccatcaacctggtgagaggcctgccccaaggcttcagcgccctggagcccctggtggacctgcccatcggcatcaacatcacaagatttcagaccctgcaccggagctatctgacccctggcgactcctcctccgggtggaccgctggcgccgccgcttactacgtgggctacctgcagcctagaaccttcctgctgaagtacaacgagaacggcaccattacagacgccgtggattgcgccctggaccccctgagcgagaccaagtgcaccctgaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcctggaacagaaaaagaatcagcaactgcgtggccgactacagcgtgctgtacaacagcgctagcttcagcaccttcaagtgctacggcgtgagccccaccaagctgaacgacctgtgcttcaccaacgtgtacgccgacagcttcgtgatcagaggcgacgaggtgagacagatcgcccccgggcagaccggcaacatcgccgactacaactataagctccccgacgacttcaccggctgcgtgatcgcttggaactccaacaacctggacagcaaggtgggcggcaactacaactacagatatagactgttcagaaagagcaacctgaagcccttcgagagagacatcagcaccgagatctaccaagccggcagcaagccctgcaacggcgtgaagggcttcaactgctacttccccctgcagagctacggctttcagcccacctacggcgtgggctatcagccctacagagtggtcgtgctgagcttcgagctgctgcacgcccccgccaccgtctgcggccccaagaagagcaccaacctggtgaagaacaagtgcgtgaacttcaacttcaacgggctgaccggcaccggcgtgctgaccgagagcaacaagaagttcctgccctttcagcagttcggcagagacatcgccgacaccaccgacgccgtgagagaccctcagaccctggagatcctggacatcacaccctgcagcttcggcggcgtgagcgtgatcacccccggcaccaacacaagcaaccaagtggccgtgctgtaccaaggcgtgaactgcaccgaggtgcccgtggccatccacgccgatcagctgacccccacctggagagtctatagcaccggcagcaacgtgtttcagacaagagccggctgcctgatcggcgccgagcacgtgaacaacagctacgagtgcgacatccccatcggcgccggcatctgcgctagctatcagacacagaccaacagccctagaagagctagaagcgtggctagccaaagcatcatcgcctacaccatgagcctgggcgtggagaacagcgtggcctacagcaacaacagcatcgccatccccaccaacttcaccatcagcgtgaccaccgagatcctccctgtgagcatgaccaagacaagcgtggactgcaccatgtacatctgcggcgacagcaccgagtgcagcaacctgctcctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagagaagcttcatcgaggacctgctgttcaacaaggtgaccctggccgacgccggcttcatcaagcagtacggcgactgcctgggcgacatcgccgctagagacctgatctgcgctcagaagttcaacggcctgaccgtgctgccccccctgctgaccgacgagatgatcgctcaatacacaagcgccctcctggccgggacaatcacatccggctggaccttcggcgccggggctgccctgcagatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcagaacgcccaagccctgaacaccctggtgaagcagctgagcagcaacttcggcgccatcagcagcgtgctgaacgacatcctgagcagactggacccccccgaggccgaggtgcagatcgatagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatcgtgaacaataccgtctacgaccccctgcaacccgagctggacagcttcaaggaggagctggacaagtacttcaagaaccacacaagccccgacgtggacctgggcgatatcagcggcatcaacgctagcgtggtgaacattcagaaggaaatcgatcggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgctgcagctgcctgaagggctgctgtagctgtggcagctgctgcaagttcgacgaggacgacagcgagcccgtgctgaagggcgtgaagctgcactacacctga

SEQ ID NO: 31 - protein, 1270 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYRYRLFRKSNLKPFERDISTEIYQAGSKPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 32 - DNA, 3813 bp, synthetic construct
  source 1..3813: mol_type = other DNA
atgttcgtgttcctggtgctgctgcccctggtgagcagccaatgcgtgaacctgaccacaagaacacagctgccccccgcctacaccaacagcttcacaagaggcgtgtactaccccgacaaggtgttcagaagcagcgtcctccacagcacccaagacctgttcctgcccttcttcagcaacgtgacctggttccacgtgatcagcggcaccaacggcaccaagagattcgacaaccccgtgctgcccttcaacgacggcgtgtacttcgctagcatcgagaagagcaacatcatcagaggctggatcttcggcaccaccctggacagcaagacacagagcctgctgatcgtcaacaacgccaccaacgtggtgatcaaggtgtgcgagtttcagttctgcaacgaccccttcctggaccacaagaacaacaagagctggatggagagcgagttcagagtgtatagcagcgctaacaactgcaccttcgagtacgtgagccaacccttcctgatggacctggagggcaagcaaggcaacttcaaaaacctgagagagttcgtgttcaagaacatcgacggctacttcaagatctacagcaagcacacccccatcatcgtgagagagcccgaggacctgccccaaggcttcagcgccctggagcccctggtggacctgcccatcggcatcaacatcacaagatttcagaccctgctggccctgcacagatcctatctgacccctggggacagcagcagcggctggaccgctggcgctgccgcctactacgtgggctacctgcagcctagaaccttcctgctgaagtacaacgagaacggcacaatcaccgatgccgtcgactgcgccctggaccccctgagcgagaccaagtgcaccctgaagagcttcaccgtggagaagggcatctatcagacaagcaacttcagagtgcagcccaccgagagcatcgtgagattccccaacatcaccaacctgtgccccttcgacgaggtgttcaacgccacaagattcgctagcgtgtacgcctggaatagaaagagaatcagcaactgcgtggccgactacagcgtgctgtacaacctggcccccttcttcaccttcaaatgctacggcgtgagccccaccaagctgaacgacctgtgcttcaccaacgtgtacgccgacagcttcgtgatcagaggcgacgaggtgagacagatcgcccccgggcagaccggcaacatcgccgactacaactacaagctgcccgacgacttcaccggctgcgtgatcgcctggaacagcaacaagctcgacagcaaggtgagcggcaactacaactacctgtacagactgttcagaaagagcaacctgaagcccttcgagagagacatcagcaccgagatctaccaagccggcaacaagccctgcaacggcgtggccggcttcaactgctacttccccctgagaagctacagcttcagacccacctacggcgtgggccatcagccctacagagtggtcgtgctgagcttcgagctgctgcacgcccccgccaccgtgtgcggccccaagaagagcaccaacctggtgaagaacaagtgcgtgaacttcaacttcaacggcctgaagggcaccggcgtgctgaccgagagcaacaagaagtttctgccctttcagcagttcggcagagacatcgccgacaccaccgacgccgtgagagaccctcagaccctggagatcctggacatcaccccctgctccttcggcggcgtgagcgtgatcacccccggcaccaacacaagcaaccaagtggccgtgctgtaccaaggcgtgaactgcaccgaggtgcccgtggccatccacgccgatcagctgacccccacctggagagtgtacagcaccggcagcaacgtgtttcagacaagagccggctgcctgatcggcgccgagtacgtgaacaacagctacgagtgcgacatccccatcggcgccggcatctgcgctagctatcagacacagaccaagagccacagaagagctagaagcgtggctagccaaagcatcatcgcctacaccatgagcctgggcgccgagaacagcgtggcctacagcaacaacagcatcgccatccccaccaacttcaccatcagcgtgaccaccgaaattctgcccgtgagcatgaccaagacaagcgtggactgcaccatgtacatctgcggcgacagcaccgagtgcagcaacctgctcctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagagaagcttcatcgaggacctgctgttcaacaaggtgaccctggccgacgccggcttcatcaagcagtacggcgactgcctgggcgatattgccgctagagacctgatctgcgctcagaagttcaagggcctgaccgtgctgccccccctgctgaccgacgagatgatcgctcagtatacaagcgccctcctcgctgggaccatcacaagcggctggacattcggggccggcgctgccctgcagatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatcagcagcgtgctgaacgacatcttcagcagactggacccccccgaggccgaggtgcagatcgaccggctcatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatttgccacgacggcaaggcccacttccctagagagggcgtgttcgtgagcaacggcacccactggttcgtgacacagagaaacttctacgagcctcagatcatcaccaccgacaacaccttcgtcagcggcaactgtgacgtggtgatcggcatcgtgaacaataccgtgtacgaccccctgcagcccgaactggacagcttcaaggaggagctggacaagtacttcaagaaccacacaagccccgacgtggacctgggcgacattagcggcatcaacgctagcgtggtgaacattcagaaggagattgacagactgaacgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgctgtagctgcctgaaaggctgctgcagctgcggcagctgctgcaagttcgacgaggacgacagcgagcccgtgctgaagggcgtgaagctgcactacacctga

SEQ ID NO: 33 - protein, 1270 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 34 - DNA, 3813 bp, synthetic construct
  source 1..3813: mol_type = other DNA
atgttcgtgttcctggtgctgctgcccctggtgagcagccaatgcgtgaacctgatcacaagaacacagagctacaccaacagcttcacaagaggcgtgtactaccccgacaaggtgttcagaagcagcgtcctccacagcacccaagacctgttcctgcctttctttagcaacgtgacctggttccacgccatccacgtgagcggcaccaacggcaccaagagattcgacaaccccgtgctgcccttcaacgacggcgtgtacttcgctagcaccgagaagagcaacatcatcagaggctggatcttcggcaccaccctggacagcaagacacagagcctgctgatcgtcaacaacgccaccaacgtggtgatcaaggtgtgcgagtttcagttctgcaacgaccccttcctggacgtgtactaccacaagaacaacaagagctggatggagagcgagttcagagtgtacagcagcgccaacaactgcaccttcgagtacgtgagccaacccttcctgatggacctggagggcaagcaaggcaacttcaaaaacctgagagagttcgtgttcaagaacatcgacggctacttcaagatctacagcaagcacacccccatcaacctgggcagagacctgccccaaggcttcagcgccctggagcccctggtggacctgcccatcggcatcaacatcacaagatttcagaccctgctggccctgcacagatcctacctcacacctggcgacagcagctccggctggaccgctggcgctgccgcttactacgtgggctacctgcagcctagaaccttcctgctgaagtacaacgagaacggcacaatcaccgatgccgtcgactgcgccctggaccccctgagcgagaccaagtgcaccctgaagagcttcaccgtggagaagggcatctatcagacaagcaacttcagagtgcagcccaccgagagcatcgtgagattccccaacatcaccaacctgtgccccttcgacgaggtgttcaacgccacaagattcgctagcgtgtacgcctggaacagaaaaagaatcagcaactgcgtggccgactacagcgtgctgtacaacttcgcccccttcttcgccttcaaatgctacggcgtgagccccaccaagctgaacgacctgtgcttcaccaacgtgtacgccgacagcttcgtgatcagaggcaacgaggtgagccaaatcgcccccgggcagaccggcaacatcgccgactacaactacaagctgcccgacgacttcaccggctgcgtgatcgcctggaactccaacaagctcgacagcaaggtgggcggcaactacaactacctgtacagactgttcagaaagagcaacctgaagcccttcgagagagacatcagcaccgagatctaccaagccggcaacaagccctgcaacggcgtggccggcttcaactgctacttccccctgagaagctacggcttcagacccacctacggcgtgggccatcagccctacagagtggtcgtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctcccctttcagcagttcggcagagacatcgccgacaccaccgacgccgtgagagaccctcagaccctggagatcctggacatcaccccctgcagcttcggcggcgtgagcgtgatcacccccggcaccaacacaagcaaccaagtggccgtgctgtaccaaggcgtgaactgcaccgaggtgcccgtggccatccacgccgatcagctgacccccacctggagagtgtactccaccggcagcaacgtgtttcagacaagagccggctgcctgatcggcgccgagtacgtgaacaacagctacgagtgcgacatccccatcggcgccggcatctgcgctagctatcagacacagaccaagagccacagaagagctagaagcgtggctagccaaagcatcatcgcctacaccatgagcctgggcgccgagaacagcgtggcctacagcaacaacagcatcgccatccccaccaacttcaccatcagcgtgaccaccgagatcctgcctgtgagcatgaccaagacaagcgtggactgcaccatgtacatctgcggcgacagcaccgagtgcagcaacctgctcctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctcggcgacatcgccgctagagacctgatctgcgctcagaagttcaatggcctgaccgtgctgccccccctgctgaccgacgagatgatcgctcagtatacctccgccctgctcgccgggaccatcacaagcggctggaccttcggcgccggcgccgccctccaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatcagcagcgtgctgaacgatatcctgagcagactggacccccccgaggccgaggtgcagatcgatagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcacaaccgcccccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcgggatcgtgaacaacaccgtgtacgatcccctgcagcccgagctggacagcttcaaggaggagctggacaagtacttcaagaaccacacaagccccgacgtggacctgggcgacattagcggcatcaacgctagcgtggtgaacattcagaaggagatcgacagactgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgctgtagctgcctgaagggctgctgcagctgcggctcctgctgcaagttcgacgaggacgacagcgagcccgtgctgaagggcgtgaagctgcactacacctga

SEQ ID NO: 35 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 36 - DNA, 3813 bp, synthetic construct
  source 1..3813: mol_type = other DNA
atgttcgtgttcctggtgctgctgcccctggtgagcagccaatgcgtgaacctgatcacaagaacacagagctacaccaacagcttcacaagaggcgtgtactaccccgacaaggtgttcagaagcagcgtcctccacagcacccaagacctgttcctgcctttctttagcaacgtgacctggttccacgccatccacgtgagcggcaccaacggcaccaagagattcgacaaccccgtgctgcccttcaacgacggcgtgtacttcgctagcaccgagaagagcaacatcatcagaggctggatcttcggcaccaccctggacagcaagacacagagcctgctgatcgtcaacaacgccaccaacgtggtgatcaaggtgtgcgagtttcagttctgcaacgaccccttcctggacgtgtactaccacaagaacaacaagagctggatggagagcgagttcagagtgtacagcagcgccaacaactgcaccttcgagtacgtgagccaacccttcctgatggacctggagggcaagcaaggcaacttcaaaaacctgagagagttcgtgttcaagaacatcgacggctacttcaagatctacagcaagcacacccccatcaacctgggcagagacctgccccaaggcttcagcgccctggagcccctggtggacctgcccatcggcatcaacatcacaagatttcagaccctgctggccctgcacagatcctacctcacacctggcgacagcagctccggctggaccgctggcgctgccgcttactacgtgggctacctgcagcctagaaccttcctgctgaagtacaacgagaacggcacaatcaccgatgccgtcgactgcgccctggaccccctgagcgagaccaagtgcaccctgaagagcttcaccgtggagaagggcatctatcagacaagcaacttcagagtgcagcccaccgagagcatcgtgagattccccaacatcaccaacctgtgccccttcgacgaggtgttcaacgccacaagattcgctagcgtgtacgcctggaacagaaaaagaatcagcaactgcgtggccgactacagcgtgctgtacaacttcgcccccttcttcgccttcaaatgctacggcgtgagccccaccaagctgaacgacctgtgcttcaccaacgtgtacgccgacagcttcgtgatcagaggcaacgaggtgagccaaatcgcccccgggcagaccggcaacatcgccgactacaactacaagctgcccgacgacttcaccggctgcgtgatcgcctggaactccaacaagctcgacagcaaggtgggcggcaactacaactatcagtacagactgttcagaaagagcaacctgaagcccttcgagagagacatcagcaccgagatctaccaagccggcaacaagccctgcaacggcgtggccggcttcaactgctacttccccctgagaagctacggcttcagacccacctacggcgtgggccatcagccctacagagtggtcgtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctcccctttcagcagttcggcagagacatcgccgacaccaccgacgccgtgagagaccctcagaccctggagatcctggacatcaccccctgcagcttcggcggcgtgagcgtgatcacccccggcaccaacacaagcaaccaagtggccgtgctgtaccaaggcgtgaactgcaccgaggtgcccgtggccatccacgccgatcagctgacccccacctggagagtgtactccaccggcagcaacgtgtttcagacaagagccggctgcctgatcggcgccgagtacgtgaacaacagctacgagtgcgacatccccatcggcgccggcatctgcgctagctatcagacacagaccaagagccacagaagagctagaagcgtggctagccaaagcatcatcgcctacaccatgagcctgggcgccgagaacctggtggcctacagcaacaacagcatcgccatccccaccaacttcaccatcagcgtgaccaccgagatcctgcctgtgagcatgaccaagacaagcgtggactgcaccatgtacatctgcggcgacagcaccgagtgcagcaacctgctcctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctcggcgacatcgccgctagagacctgatctgcgctcagaagttcaatggcctgaccgtgctgccccccctgctgaccgacgagatgatcgctcagtatacctccgccctgctcgccgggaccatcacaagcggctggaccttcggcgccggcgccgccctccaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatcagcagcgtgctgaacgatatcctgagcagactggacccccccgaggccgaggtgcagatcgatagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcacaaccgcccccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcgggatcgtgaacaacaccgtgtacgatcccctgcagcccgagctggacagcttcaaggaggagctggacaagtacttcaagaaccacacaagccccgacgtggacctgggcgacattagcggcatcaacgctagcgtggtgaacattcagaaggagatcgacagactgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgctgtagctgcctgaagggctgctgcagctgcggctcctgctgcaagttcgacgaggacgacagcgagcccgtgctgaagggcgtgaagctgcactacacctga

SEQ ID NO: 37 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENLVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 38 - DNA, 3807 bp, synthetic construct
  source 1..3807: mol_type = other DNA
atgttcgtgttcctggtgctgctgcccctggtgagcagccaatgcgtgaacctgatcacaagaacacagagctacaccaacagcttcacaagaggcgtgtactaccccgacaaggtgttcagaagcagcgtcctgcattccacccaagacctgtttctccccttcttcagcaacgtgacctggttccacgccatcagcggcaccaacggcaccaagagattcgacaaccccgtgctgcccttcaacgacggcgtgtacttcgctagcaccgagaagagcaacatcatcagaggctggatcttcggcaccaccctggacagcaagacacagagcctgctgatcgtgaacaacgccacaaacgtggtgatcaaggtgtgcgagtttcagttctgcaacgaccccttcctggacgtgtactaccacaagaacaacaagagctggatggagagcgagttccgggtgtacagcagcgccaacaactgcaccttcgagtacgtgagccaacccttcctgatggacctggagggcaagcaaggcaacttcaagaacctgagagagttcgtgttcaagaacatcgacggctacttcaagatctacagcaagcacacccccatcaacctgggcagagacctgccccaaggcttcagcgccctggagcccctggtggacctgcccatcggcatcaacatcacaagatttcagacactgctcgccctgcaccggagctacctcacccctggcgacagcagctccggctggaccgctggggctgccgcctactacgtgggctacctgcagcctagaaccttcctgctgaagtacaacgagaacggcaccatcaccgacgctgtggactgtgccctggaccccctgagcgagaccaagtgcaccctgaagagcttcaccgtggagaagggcatctatcagacaagcaacttcagagtgcagcccaccgagagcatcgtgagattccccaacatcaccaacctgtgccccttcgacgaggtgttcaacgccacaagattcgctagcgtgtacgcctggaacagaaagagaatcagcaactgcgtggccgactacagcgtgctgtacaacttcgcccccttcttcgccttcaaatgctacggcgtcagccccaccaagctgaacgacctgtgcttcaccaacgtgtacgccgacagcttcgtgatcagaggcaacgaggtgagccaaatcgcccccgggcagaccggcaacatcgccgactataactacaaactgcccgacgacttcaccggctgcgtgatcgcctggaactccaacaagctggacagcaaagtgggcggcaactacaactacagatacagactgttcagaaagagcaacctgaagcccttcgagagagacatcagcaccgagatctaccaagccggcaacaagccctgcaacggcgtggccggcgtgaactgctacttccccctgcagagctacggcttcagacccacctacggcgtgggccatcagccctacagagtggtcgtgctgagcttcgagctgctgcacgcccctgccacagtgtgcggccccaagaagagcaccaacctggtgaagaacaagtgcgtgaacttcaacttcaacggcctgaccggcaccggcgtgctgaccgagagcaacaagaagttcctgccctttcagcagttcggcagagacatcgccgacaccaccgatgctgtgagagaccctcagaccctggagatcctggacatcaccccttgcagcttcggcggcgtgagcgtgatcacccccggcaccaacacaagcaaccaagtggccgtgctgtaccaaggcgtcaactgcacagaggtgcccgtggccatccacgccgatcagctgacccccacctggagagtgtactccaccggcagcaacgtgtttcagacaagagccggctgcctgatcggcgccgagtacgtgaacaacagctacgagtgcgacatccccatcggcgccggcatctgcgctagctatcagacacagaccaagagccacagaagagctagaagcgtggctagccaaagcatcatcgcctacaccatgagcctgggcgccgagaacagcgtggcctacagcaacaacagcatcgccatccccaccaacttcaccatcagcgtgaccaccgagatcctgcctgtgagcatgaccaagacaagcgtggactgcaccatgtacatctgcggcgacagcaccgagtgcagcaacctgctcctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctcggcgacatcgctgctagagacctgatctgcgctcagaagttcaacggcctcacagtgctgccccccctgctgaccgacgagatgatcgctcagtacacctccgctctgctggccggcacaattacatccggctggaccttcggcgccggcgccgccctgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatctcctccgtcctgaacgacatcctgagcagactggacccccccgaggccgaggtgcagattgacagactgattaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatcgtcaacaacaccgtgtacgaccccctgcaacccgagctggacagcttcaaggaggagctggacaagtactttaagaaccacacaagccccgacgtggacctgggggacatctccggcatcaacgctagcgtggtgaacattcagaaggaaattgacagactgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgctgcagctgtctgaagggctgctgctcctgcggcagctgctgcaagttcgacgaggacgacagcgagcccgtgctgaagggcgtgaagctgcactacacctga

SEQ ID NO: 39 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 40 - protein, 1273 aa, synthetic construct; identical to SEQ ID NO: 25

SEQ ID NO: 41 - DNA, 3822 bp, synthetic construct
  source 1..3822: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctag

SEQ ID NO: 42 - DNA, 3822 bp, synthetic construct
  source 1..3822: mol_type = other DNA
atgttcgtgttcctggtgctgctgcccctggtgtcctcccagtgcgtgaacctgaccacccgtacccagctgccccccgcttacaccaactccttcacccgtggtgtgtactaccccgacaaggtgttccgttcctccgtgctgcactccacccaggacctgttcctgcccttcttctccaacgtgacctggttccacgctatccacgtgtccggtaccaacggtaccaagcgtttcgacaaccccgtgctgcccttcaacgacggtgtgtacttcgcttccaccgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctgggtgtgtactaccacaagaacaacaagtcctggatggagtccgagttccgtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcaacctggtgcgtgacctgccccagggtttctccgctctggagcccctggtggacctgcccatcggtatcaacatcacccgtttccagaccctgctggctctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcggtgaggtgttcaacgctacccgtttcgcttccgtgtacgcttggaaccgtaagcgtatctccaactgcgtggctgactactccgtgctgtacaactccgcttccttctccaccttcaagtgctacggtgtgtcccccaccaagctgaacgacctgtgcttcaccaacgtgtacgctgactccttcgtgatccgtggtgacgaggtgcgtcagatcgctcccggtcagaccggtaagatcgctgactacaactacaagctgcccgacgacttcaccggttgcgtgatcgcttggaactccaacaacctggactccaaggtgggtggtaactacaactacctgtaccgtctgttccgtaagtccaacctgaagcccttcgagcgtgacatctccaccgagatctaccaggctggttccaccccctgcaacggtgtggagggtttcaactgctacttccccctgcagtcctacggtttccagcccaccaacggtgtgggttaccagccctaccgtgtggtggtgctgtccttcgagctgctgcacgctcccgctaccgtgtgcggtcccaagaagtccaccaacctggtgaagaacaagtgcgtgaacttcaacttcaacggtctgaccggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccaggacgtgaactgcaccgaggtgcccgtggctatccacgctgaccagctgacccccacctggcgtgtgtactccaccggttccaacgtgttccagacccgtgctggttgcctgatcggtgctgagcacgtgaacaactcctacgagtgcgacatccccatcggtgctggtatctgcgcttcctaccagacccagaccaactccccccgtcgtgctcgttccgtggcttcccagtccatcatcgcttacaccatgtccctgggtgctgagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaaccgtgctctgaccggtatcgctgtggagcaggacaagaacacccaggaggtgttcgctcaggtgaagcagatctacaagaccccccccatcaaggacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaacggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcaggacgtggtgaaccagaacgctcaggctctgaacaccctggtgaagcagctgtcctccaacttcggtgctatctcctccgtgctgaacgacatcctgtcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctggtacatctggctgggtttcatcgctggtctgatcgctatcgtgatggtgaccatcatgctgtgctgcatgacctcctgctgctcctgcctgaagggttgctgctcctgcggttcctgctgcaagttcgacgaggacgactccgagcccgtgctgaagggtgtgaagctgcactacacctag

SEQ ID NO: 43 - protein, 1213 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 44 - DNA, 3642 bp, synthetic construct
  source 1..3642: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctag

SEQ ID NO: 45 - DNA, 3642 bp, synthetic construct
  source 1..3642: mol_type = other DNA
atgttcgtgttcctggtgctgctgcccctggtgtcctcccagtgcgtgaacctgaccacccgtacccagctgccccccgcttacaccaactccttcacccgtggtgtgtactaccccgacaaggtgttccgttcctccgtgctgcactccacccaggacctgttcctgcccttcttctccaacgtgacctggttccacgctatccacgtgtccggtaccaacggtaccaagcgtttcgacaaccccgtgctgcccttcaacgacggtgtgtacttcgcttccaccgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctgggtgtgtactaccacaagaacaacaagtcctggatggagtccgagttccgtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcaacctggtgcgtgacctgccccagggtttctccgctctggagcccctggtggacctgcccatcggtatcaacatcacccgtttccagaccctgctggctctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcggtgaggtgttcaacgctacccgtttcgcttccgtgtacgcttggaaccgtaagcgtatctccaactgcgtggctgactactccgtgctgtacaactccgcttccttctccaccttcaagtgctacggtgtgtcccccaccaagctgaacgacctgtgcttcaccaacgtgtacgctgactccttcgtgatccgtggtgacgaggtgcgtcagatcgctcccggtcagaccggtaagatcgctgactacaactacaagctgcccgacgacttcaccggttgcgtgatcgcttggaactccaacaacctggactccaaggtgggtggtaactacaactacctgtaccgtctgttccgtaagtccaacctgaagcccttcgagcgtgacatctccaccgagatctaccaggctggttccaccccctgcaacggtgtggagggtttcaactgctacttccccctgcagtcctacggtttccagcccaccaacggtgtgggttaccagccctaccgtgtggtggtgctgtccttcgagctgctgcacgctcccgctaccgtgtgcggtcccaagaagtccaccaacctggtgaagaacaagtgcgtgaacttcaacttcaacggtctgaccggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccaggacgtgaactgcaccgaggtgcccgtggctatccacgctgaccagctgacccccacctggcgtgtgtactccaccggttccaacgtgttccagacccgtgctggttgcctgatcggtgctgagcacgtgaacaactcctacgagtgcgacatccccatcggtgctggtatctgcgcttcctaccagacccagaccaactccccccgtcgtgctcgttccgtggcttcccagtccatcatcgcttacaccatgtccctgggtgctgagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaaccgtgctctgaccggtatcgctgtggagcaggacaagaacacccaggaggtgttcgctcaggtgaagcagatctacaagaccccccccatcaaggacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaacggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcaggacgtggtgaaccagaacgctcaggctctgaacaccctggtgaagcagctgtcctccaacttcggtgctatctcctccgtgctgaacgacatcctgtcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctag

SEQ ID NO: 46 - protein, 1270 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 47 - DNA, 3813 bp, synthetic construct
  source 1..3813: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgccaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagaggcctgccccagggcttcagcgccctggagcccctggtggacctgcccatcggcatcaacatcaccagattccagaccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaacatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtgaagggcttcaactgctacttccccctgcagagctacggcttccagcccacctacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgtggagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctag

SEQ ID NO: 48 - DNA, 3813 bp, synthetic construct
  source 1..3813: mol_type = other DNA
atgttcgtgttcctggtgctgctgcccctggtgtcctcccagtgcgtgaacctgaccacccgtacccagctgccccccgcttacaccaactccttcacccgtggtgtgtactaccccgacaaggtgttccgttcctccgtgctgcactccacccaggacctgttcctgcccttcttctccaacgtgacctggttccacgctatccacgtgtccggtaccaacggtaccaagcgtttcgctaaccccgtgctgcccttcaacgacggtgtgtacttcgcttccaccgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctgggtgtgtactaccacaagaacaacaagtcctggatggagtccgagttccgtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcaacctggtgcgtggtctgccccagggtttctccgctctggagcccctggtggacctgcccatcggtatcaacatcacccgtttccagaccctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcggtgaggtgttcaacgctacccgtttcgcttccgtgtacgcttggaaccgtaagcgtatctccaactgcgtggctgactactccgtgctgtacaactccgcttccttctccaccttcaagtgctacggtgtgtcccccaccaagctgaacgacctgtgcttcaccaacgtgtacgctgactccttcgtgatccgtggtgacgaggtgcgtcagatcgctcccggtcagaccggtaacatcgctgactacaactacaagctgcccgacgacttcaccggttgcgtgatcgcttggaactccaacaacctggactccaaggtgggtggtaactacaactacctgtaccgtctgttccgtaagtccaacctgaagcccttcgagcgtgacatctccaccgagatctaccaggctggttccaccccctgcaacggtgtgaagggtttcaactgctacttccccctgcagtcctacggtttccagcccacctacggtgtgggttaccagccctaccgtgtggtggtgctgtccttcgagctgctgcacgctcccgctaccgtgtgcggtcccaagaagtccaccaacctggtgaagaacaagtgcgtgaacttcaacttcaacggtctgaccggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccagggtgtgaactgcaccgaggtgcccgtggctatccacgctgaccagctgacccccacctggcgtgtgtactccaccggttccaacgtgttccagacccgtgctggttgcctgatcggtgctgagcacgtgaacaactcctacgagtgcgacatccccatcggtgctggtatctgcgcttcctaccagacccagaccaactccccccgtcgtgctcgttccgtggcttcccagtccatcatcgcttacaccatgtccctgggtgtggagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaaccgtgctctgaccggtatcgctgtggagcaggacaagaacacccaggaggtgttcgctcaggtgaagcagatctacaagaccccccccatcaaggacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaacggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcaggacgtggtgaaccagaacgctcaggctctgaacaccctggtgaagcagctgtcctccaacttcggtgctatctcctccgtgctgaacgacatcctgtcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctggtacatctggctgggtttcatcgctggtctgatcgctatcgtgatggtgaccatcatgctgtgctgcatgacctcctgctgctcctgcctgaagggttgctgctcctgcggttcctgctgcaagttcgacgaggacgactccgagcccgtgctgaagggtgtgaagctgcactacacctag

SEQ ID NO: 49 - protein, 1210 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 50 - DNA, 3633 bp, synthetic construct
  source 1..3633: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgccaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagaggcctgccccagggcttcagcgccctggagcccctggtggacctgcccatcggcatcaacatcaccagattccagaccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaacatcgccgactacaactacaagctgcccgacgacttcaccggctgcgtgatcgcctggaacagcaacaacctggacagcaaggtgggcggcaactacaactacctgtacagactgttcagaaagagcaacctgaagcccttcgagagagacatcagcaccgagatctaccaggccggcagcaccccctgcaacggcgtgaagggcttcaactgctacttccccctgcagagctacggcttccagcccacctacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagccccagaagagccagaagcgtggccagccagagcatcatcgcctacaccatgagcctgggcgtggagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctag

SEQ ID NO: 51 - DNA, 3633 bp, synthetic construct
  source 1..3633: mol_type = other DNA
atgttcgtgttcctggtgctgctgcccctggtgtcctcccagtgcgtgaacctgaccacccgtacccagctgccccccgcttacaccaactccttcacccgtggtgtgtactaccccgacaaggtgttccgttcctccgtgctgcactccacccaggacctgttcctgcccttcttctccaacgtgacctggttccacgctatccacgtgtccggtaccaacggtaccaagcgtttcgctaaccccgtgctgcccttcaacgacggtgtgtacttcgcttccaccgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctgggtgtgtactaccacaagaacaacaagtcctggatggagtccgagttccgtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcaacctggtgcgtggtctgccccagggtttctccgctctggagcccctggtggacctgcccatcggtatcaacatcacccgtttccagaccctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcggtgaggtgttcaacgctacccgtttcgcttccgtgtacgcttggaaccgtaagcgtatctccaactgcgtggctgactactccgtgctgtacaactccgcttccttctccaccttcaagtgctacggtgtgtcccccaccaagctgaacgacctgtgcttcaccaacgtgtacgctgactccttcgtgatccgtggtgacgaggtgcgtcagatcgctcccggtcagaccggtaacatcgctgactacaactacaagctgcccgacgacttcaccggttgcgtgatcgcttggaactccaacaacctggactccaaggtgggtggtaactacaactacctgtaccgtctgttccgtaagtccaacctgaagcccttcgagcgtgacatctccaccgagatctaccaggctggttccaccccctgcaacggtgtgaagggtttcaactgctacttccccctgcagtcctacggtttccagcccacctacggtgtgggttaccagccctaccgtgtggtggtgctgtccttcgagctgctgcacgctcccgctaccgtgtgcggtcccaagaagtccaccaacctggtgaagaacaagtgcgtgaacttcaacttcaacggtctgaccggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccagggtgtgaactgcaccgaggtgcccgtggctatccacgctgaccagctgacccccacctggcgtgtgtactccaccggttccaacgtgttccagacccgtgctggttgcctgatcggtgctgagcacgtgaacaactcctacgagtgcgacatccccatcggtgctggtatctgcgcttcctaccagacccagaccaactccccccgtcgtgctcgttccgtggcttcccagtccatcatcgcttacaccatgtccctgggtgtggagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaaccgtgctctgaccggtatcgctgtggagcaggacaagaacacccaggaggtgttcgctcaggtgaagcagatctacaagaccccccccatcaaggacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaacggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcaggacgtggtgaaccagaacgctcaggctctgaacaccctggtgaagcagctgtcctccaacttcggtgctatctcctccgtgctgaacgacatcctgtcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctag

SEQ ID NO: 52 - protein, 1271 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 53 - DNA, 3816 bp, synthetic construct
  source 1..3816: mol_type = other DNA
atgttcgtgttcctggtgctgctgcccctggtgagcagccagtgcgtgaacctgagaaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcggcgtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacagatacagactgttcagaaagagcaacctgaagcccttcgagagagacatcagcaccgagatctaccaggccggcagcaagccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagcagaagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcagaacgtggtgaaccagaacgcccaggccctgaacaccctggtgaagcagctgagcagcaacttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctag

SEQ ID NO: 54 - DNA, 3816 bp, synthetic construct
  source 1..3816: mol_type = other DNA
atgttcgtgttcctggtgctgctgcccctggtgtcctcccagtgcgtgaacctgcgtacccgtacccagctgccccccgcttacaccaactccttcacccgtggtgtgtactaccccgacaaggtgttccgttcctccgtgctgcactccacccaggacctgttcctgcccttcttctccaacgtgacctggttccacgctatccacgtgtccggtaccaacggtaccaagcgtttcgacaaccccgtgctgcccttcaacgacggtgtgtacttcgcttccaccgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctggacgtgtactaccacaagaacaacaagtcctggatggagtccggtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcaacctggtgcgtgacctgccccagggtttctccgctctggagcccctggtggacctgcccatcggtatcaacatcacccgtttccagaccctgctggctctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcggtgaggtgttcaacgctacccgtttcgcttccgtgtacgcttggaaccgtaagcgtatctccaactgcgtggctgactactccgtgctgtacaactccgcttccttctccaccttcaagtgctacggtgtgtcccccaccaagctgaacgacctgtgcttcaccaacgtgtacgctgactccttcgtgatccgtggtgacgaggtgcgtcagatcgctcccggtcagaccggtaagatcgctgactacaactacaagctgcccgacgacttcaccggttgcgtgatcgcttggaactccaacaacctggactccaaggtgggtggtaactacaactaccgttaccgtctgttccgtaagtccaacctgaagcccttcgagcgtgacatctccaccgagatctaccaggctggttccaagccctgcaacggtgtggagggtttcaactgctacttccccctgcagtcctacggtttccagcccaccaacggtgtgggttaccagccctaccgtgtggtggtgctgtccttcgagctgctgcacgctcccgctaccgtgtgcggtcccaagaagtccaccaacctggtgaagaacaagtgcgtgaacttcaacttcaacggtctgaccggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccagggtgtgaactgcaccgaggtgcccgtggctatccacgctgaccagctgacccccacctggcgtgtgtactccaccggttccaacgtgttccagacccgtgctggttgcctgatcggtgctgagcacgtgaacaactcctacgagtgcgacatccccatcggtgctggtatctgcgcttcctaccagacccagaccaactcccgtcgtcgtgctcgttccgtggcttcccagtccatcatcgcttacaccatgtccctgggtgctgagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaaccgtgctctgaccggtatcgctgtggagcaggacaagaacacccaggaggtgttcgctcaggtgaagcagatctacaagaccccccccatcaaggacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaacggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcagaacgtggtgaaccagaacgctcaggctctgaacaccctggtgaagcagctgtcctccaacttcggtgctatctcctccgtgctgaacgacatcctgtcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctggtacatctggctgggtttcatcgctggtctgatcgctatcgtgatggtgaccatcatgctgtgctgcatgacctcctgctgctcctgcctgaagggttgctgctcctgcggttcctgctgcaagttcgacgaggacgactccgagcccgtgctgaagggtgtgaagctgcactacacctag

SEQ ID NO: 55 - protein, 1211 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 56 - DNA, 3636 bp, synthetic construct
  source 1..3636: mol_type = other DNA
atgttcgtgttcctggtgctgctgcccctggtgagcagccagtgcgtgaacctgagaaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcggcgtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctggtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcggcgaggtgttcaacgccaccagattcgccagcgtgtacgcctggaacagaaagagaatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatcagaggcgacgaggtgagacagatcgcccccggccagaccggcaagatcgccgactacaactacaagctgcccgacgacttcaccggctgcgtgatcgcctggaacagcaacaacctggacagcaaggtgggcggcaactacaactacagatacagactgttcagaaagagcaacctgaagcccttcgagagagacatcagcaccgagatctaccaggccggcagcaagccctgcaacggcgtggagggcttcaactgctacttccccctgcagagctacggcttccagcccaccaacggcgtgggctaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagcacgtgaacaacagctacgagtgcgacatccccatcggcgccggcatctgcgccagctaccagacccagaccaacagcagaagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaacagagccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcagaacgtggtgaaccagaacgcccaggccctgaacaccctggtgaagcagctgagcagcaacttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctag

SEQ ID NO: 57 - DNA, 3636 bp, synthetic construct
  source 1..3636: mol_type = other DNA
atgttcgtgttcctggtgctgctgcccctggtgtcctcccagtgcgtgaacctgcgtacccgtacccagctgccccccgcttacaccaactccttcacccgtggtgtgtactaccccgacaaggtgttccgttcctccgtgctgcactccacccaggacctgttcctgcccttcttctccaacgtgacctggttccacgctatccacgtgtccggtaccaacggtaccaagcgtttcgacaaccccgtgctgcccttcaacgacggtgtgtacttcgcttccaccgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctggacgtgtactaccacaagaacaacaagtcctggatggagtccggtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcaacctggtgcgtgacctgccccagggtttctccgctctggagcccctggtggacctgcccatcggtatcaacatcacccgtttccagaccctgctggctctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcggtgaggtgttcaacgctacccgtttcgcttccgtgtacgcttggaaccgtaagcgtatctccaactgcgtggctgactactccgtgctgtacaactccgcttccttctccaccttcaagtgctacggtgtgtcccccaccaagctgaacgacctgtgcttcaccaacgtgtacgctgactccttcgtgatccgtggtgacgaggtgcgtcagatcgctcccggtcagaccggtaagatcgctgactacaactacaagctgcccgacgacttcaccggttgcgtgatcgcttggaactccaacaacctggactccaaggtgggtggtaactacaactaccgttaccgtctgttccgtaagtccaacctgaagcccttcgagcgtgacatctccaccgagatctaccaggctggttccaagccctgcaacggtgtggagggtttcaactgctacttccccctgcagtcctacggtttccagcccaccaacggtgtgggttaccagccctaccgtgtggtggtgctgtccttcgagctgctgcacgctcccgctaccgtgtgcggtcccaagaagtccaccaacctggtgaagaacaagtgcgtgaacttcaacttcaacggtctgaccggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccagggtgtgaactgcaccgaggtgcccgtggctatccacgctgaccagctgacccccacctggcgtgtgtactccaccggttccaacgtgttccagacccgtgctggttgcctgatcggtgctgagcacgtgaacaactcctacgagtgcgacatccccatcggtgctggtatctgcgcttcctaccagacccagaccaactcccgtcgtcgtgctcgttccgtggcttcccagtccatcatcgcttacaccatgtccctgggtgctgagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaaccgtgctctgaccggtatcgctgtggagcaggacaagaacacccaggaggtgttcgctcaggtgaagcagatctacaagaccccccccatcaaggacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaacggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcagaacgtggtgaaccagaacgctcaggctctgaacaccctggtgaagcagctgtcctccaacttcggtgctatctcctccgtgctgaacgacatcctgtcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctag

SEQ ID NO: 58 - protein, 1267 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 59 - DNA, 3804 bp, synthetic construct
  source 1..3804: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgtgatcagcggcaccaacggcaccaagagattcgacaaccccgtgctgcccttcaacgacggcgtgtacttcgccagcatcgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcatcgtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcgacgaggtgttcaacgccaccagattcgccagcgtgtacgcctggaacagaaagagaatcagcaactgcgtggccgactacagcgtgctgtacaacctggcccccttcttcaccttcaagtgctacggcgtgagccccaccaagctgaacgacctgtgcttcaccaacgtgtacgccgacagcttcgtgatcagaggcgacgaggtgagacagatcgcccccggccagaccggcaacatcgccgactacaactacaagctgcccgacgacttcaccggctgcgtgatcgcctggaacagcaacaagctggacagcaaggtgagcggcaactacaactacctgtacagactgttcagaaagagcaacctgaagcccttcgagagagacatcagcaccgagatctaccaggccggcaacaagccctgcaacggcgtggccggcttcaactgctacttccccctgagaagctacagcttcagacccacctacggcgtgggccaccagccctacagagtggtggtgctgagcttcgagctgctgcacgcccccgccaccgtgtgcggccccaagaagagcaccaacctggtgaagaacaagtgcgtgaacttcaacttcaacggcctgaagggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagtacgtgaacaacagctacgagtgcgacatccccatcggcgccggcatctgcgccagctaccagacccagaccaagagccacagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagagccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaagggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcttcagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctag

SEQ ID NO: 60 - DNA, 3804 bp, synthetic construct
  source 1..3804: mol_type = other DNA
atgttcgtgttcctggtgctgctgcccctggtgtcctcccagtgcgtgaacctgaccacccgtacccagctgccccccgcttacaccaactccttcacccgtggtgtgtactaccccgacaaggtgttccgttcctccgtgctgcactccacccaggacctgttcctgcccttcttctccaacgtgacctggttccacgtgatctccggtaccaacggtaccaagcgtttcgacaaccccgtgctgcccttcaacgacggtgtgtacttcgcttccatcgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctggaccacaagaacaacaagtcctggatggagtccgagttccgtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcatcgtgcgtgacctgccccagggtttctccgctctggagcccctggtggacctgcccatcggtatcaacatcacccgtttccagaccctgctggctctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcgacgaggtgttcaacgctacccgtttcgcttccgtgtacgcttggaaccgtaagcgtatctccaactgcgtggctgactactccgtgctgtacaacctggctcccttcttcaccttcaagtgctacggtgtgtcccccaccaagctgaacgacctgtgcttcaccaacgtgtacgctgactccttcgtgatccgtggtgacgaggtgcgtcagatcgctcccggtcagaccggtaacatcgctgactacaactacaagctgcccgacgacttcaccggttgcgtgatcgcttggaactccaacaagctggactccaaggtgtccggtaactacaactacctgtaccgtctgttccgtaagtccaacctgaagcccttcgagcgtgacatctccaccgagatctaccaggctggtaacaagccctgcaacggtgtggctggtttcaactgctacttccccctgcgttcctactccttccgtcccacctacggtgtgggtcaccagccctaccgtgtggtggtgctgtccttcgagctgctgcacgctcccgctaccgtgtgcggtcccaagaagtccaccaacctggtgaagaacaagtgcgtgaacttcaacttcaacggtctgaagggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccagggtgtgaactgcaccgaggtgcccgtggctatccacgctgaccagctgacccccacctggcgtgtgtactccaccggttccaacgtgttccagacccgtgctggttgcctgatcggtgctgagtacgtgaacaactcctacgagtgcgacatccccatcggtgctggtatctgcgcttcctaccagacccagaccaagtcccaccgtcgtgctcgttccgtggcttcccagtccatcatcgcttacaccatgtccctgggtgctgagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaagcgtgctctgaccggtatcgctgtggagcaggacaagaacacccaggaggtgttcgctcaggtgaagcagatctacaagaccccccccatcaagtacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaagggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcaggacgtggtgaaccacaacgctcaggctctgaacaccctggtgaagcagctgtcctccaagttcggtgctatctcctccgtgctgaacgacatcttctcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctggtacatctggctgggtttcatcgctggtctgatcgctatcgtgatggtgaccatcatgctgtgctgcatgacctcctgctgctcctgcctgaagggttgctgctcctgcggttcctgctgcaagttcgacgaggacgactccgagcccgtgctgaagggtgtgaagctgcactacacctag

SEQ ID NO: 61 - protein, 1207 aa, synthetic construct
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 62 - DNA, 3624 bp, synthetic construct
  source 1..3624: mol_type = other DNA
atgttcgtgttcctggtgctgctgcccctggtgagcagccagtgcgtgaacctgaccaccagaacccagctgccccccgcctacaccaacagcttcaccagaggcgtgtactaccccgacaaggtgttcagaagcagcgtgctgcacagcacccaggacctgttcctgcccttcttcagcaacgtgacctggttccacgtgatcagcggcaccaacggcaccaagagattcgacaaccccgtgctgcccttcaacgacggcgtgtacttcgccagcatcgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctggaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcatcgtgagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcgacgaggtgttcaacgccaccagattcgccagcgtgtacgcctggaacagaaagagaatcagcaactgcgtggccgactacagcgtgctgtacaacctggcccccttcttcaccttcaagtgctacggcgtgagccccaccaagctgaacgacctgtgcttcaccaacgtgtacgccgacagcttcgtgatcagaggcgacgaggtgagacagatcgcccccggccagaccggcaacatcgccgactacaactacaagctgcccgacgacttcaccggctgcgtgatcgcctggaacagcaacaagctggacagcaaggtgagcggcaactacaactacctgtacagactgttcagaaagagcaacctgaagcccttcgagagagacatcagcaccgagatctaccaggccggcaacaagccctgcaacggcgtggccggcttcaactgctacttccccctgagaagctacagcttcagacccacctacggcgtgggccaccagccctacagagtggtggtgctgagcttcgagctgctgcacgcccccgccaccgtgtgcggccccaagaagagcaccaacctggtgaagaacaagtgcgtgaacttcaacttcaacggcctgaagggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagtacgtgaacaacagctacgagtgcgacatccccatcggcgccggcatctgcgccagctaccagacccagaccaagagccacagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagagccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaagggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcttcagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctag

SEQ ID NO: 63 - DNA, 3624 bp, synthetic construct
  source 1..3624: mol_type = other DNA
atgttcgtgttcctggtgctgctgcccctggtgtcctcccagtgcgtgaacctgaccacccgtacccagctgccccccgcttacaccaactccttcacccgtggtgtgtactaccccgacaaggtgttccgttcctccgtgctgcactccacccaggacctgttcctgcccttcttctccaacgtgacctggttccacgtgatctccggtaccaacggtaccaagcgtttcgacaaccccgtgctgcccttcaacgacggtgtgtacttcgcttccatcgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctggaccacaagaacaacaagtcctggatggagtccgagttccgtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcatcgtgcgtgacctgccccagggtttctccgctctggagcccctggtggacctgcccatcggtatcaacatcacccgtttccagaccctgctggctctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcgacgaggtgttcaacgctacccgtttcgcttccgtgtacgcttggaaccgtaagcgtatctccaactgcgtggctgactactccgtgctgtacaacctggctcccttcttcaccttcaagtgctacggtgtgtcccccaccaagctgaacgacctgtgcttcaccaacgtgtacgctgactccttcgtgatccgtggtgacgaggtgcgtcagatcgctcccggtcagaccggtaacatcgctgactacaactacaagctgcccgacgacttcaccggttgcgtgatcgcttggaactccaacaagctggactccaaggtgtccggtaactacaactacctgtaccgtctgttccgtaagtccaacctgaagcccttcgagcgtgacatctccaccgagatctaccaggctggtaacaagccctgcaacggtgtggctggtttcaactgctacttccccctgcgttcctactccttccgtcccacctacggtgtgggtcaccagccctaccgtgtggtggtgctgtccttcgagctgctgcacgctcccgctaccgtgtgcggtcccaagaagtccaccaacctggtgaagaacaagtgcgtgaacttcaacttcaacggtctgaagggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccagggtgtgaactgcaccgaggtgcccgtggctatccacgctgaccagctgacccccacctggcgtgtgtactccaccggttccaacgtgttccagacccgtgctggttgcctgatcggtgctgagtacgtgaacaactcctacgagtgcgacatccccatcggtgctggtatctgcgcttcctaccagacccagaccaagtcccaccgtcgtgctcgttccgtggcttcccagtccatcatcgcttacaccatgtccctgggtgctgagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaagcgtgctctgaccggtatcgctgtggagcaggacaagaacacccaggaggtgttcgctcaggtgaagcagatctacaagaccccccccatcaagtacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaagggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcaggacgtggtgaaccacaacgctcaggctctgaacaccctggtgaagcagctgtcctccaagttcggtgctatctcctccgtgctgaacgacatcttctcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctag

SEQ ID NO: 64 - protein, 1270 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 65 - DNA, 3813 bp, synthetic construct
  source 1..3813: mol_type = other DNA
atgttcgtgttcctggtgctgctgcccctggtgagcagccagtgcgtgaacctgatcaccagaacccagagctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctgggcagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcgacgaggtgttcaacgccaccagattcgccagcgtgtacgcctggaacagaaagagaatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcagaggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacagactgttcagaaagagcaacctgaagcccttcgagagagacatcagcaccgagatctaccaggccggcaacaagccctgcaacggcgtggccggcttcaactgctacttccccctgagaagctacggcttcagacccacctacggcgtgggccaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagtacgtgaacaacagctacgagtgcgacatccccatcggcgccggcatctgcgccagctaccagacccagaccaagagccacagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagagccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctag

SEQ ID NO: 66 - DNA, 3813 bp, synthetic construct
  source 1..3813: mol_type = other DNA
atgttcgtgttcctggtgctgctgcccctggtgtcctcccagtgcgtgaacctgatcacccgtacccagtcctacaccaactccttcacccgtggtgtgtactaccccgacaaggtgttccgttcctccgtgctgcactccacccaggacctgttcctgcccttcttctccaacgtgacctggttccacgctatccacgtgtccggtaccaacggtaccaagcgtttcgacaaccccgtgctgcccttcaacgacggtgtgtacttcgcttccaccgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctggacgtgtactaccacaagaacaacaagtcctggatggagtccgagttccgtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcaacctgggtcgtgacctgccccagggtttctccgctctggagcccctggtggacctgcccatcggtatcaacatcacccgtttccagaccctgctggctctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcgacgaggtgttcaacgctacccgtttcgcttccgtgtacgcttggaaccgtaagcgtatctccaactgcgtggctgactactccgtgctgtacaacttcgctcccttcttcgctttcaagtgctacggtgtgtcccccaccaagctgaacgacctgtgcttcaccaacgtgtacgctgactccttcgtgatccgtggtaacgaggtgtcccagatcgctcccggtcagaccggtaacatcgctgactacaactacaagctgcccgacgacttcaccggttgcgtgatcgcttggaactccaacaagctggactccaaggtgggtggtaactacaactacctgtaccgtctgttccgtaagtccaacctgaagcccttcgagcgtgacatctccaccgagatctaccaggctggtaacaagccctgcaacggtgtggctggtttcaactgctacttccccctgcgttcctacggtttccgtcccacctacggtgtgggtcaccagccctaccgtgtggtggtgctgtccttcgagctgctgcacgctcccgctaccgtgtgcggtcccaagaagtccaccaacctggtgaagaacaagtgcgtgaacttcaacttcaacggtctgaccggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccagggtgtgaactgcaccgaggtgcccgtggctatccacgctgaccagctgacccccacctggcgtgtgtactccaccggttccaacgtgttccagacccgtgctggttgcctgatcggtgctgagtacgtgaacaactcctacgagtgcgacatccccatcggtgctggtatctgcgcttcctaccagacccagaccaagtcccaccgtcgtgctcgttccgtggcttcccagtccatcatcgcttacaccatgtccctgggtgctgagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaagcgtgctctgaccggtatcgctgtggagcaggacaagaacacccaggaggtgttcgctcaggtgaagcagatctacaagaccccccccatcaagtacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaacggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcaggacgtggtgaaccacaacgctcaggctctgaacaccctggtgaagcagctgtcctccaagttcggtgctatctcctccgtgctgaacgacatcctgtcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctggtacatctggctgggtttcatcgctggtctgatcgctatcgtgatggtgaccatcatgctgtgctgcatgacctcctgctgctcctgcctgaagggttgctgctcctgcggttcctgctgcaagttcgacgaggacgactccgagcccgtgctgaagggtgtgaagctgcactacacctag

SEQ ID NO: 67 - protein, 1210 aa, synthetic construct
MFVFLVLLPLVSSQCVNLITRTQSYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYLYRLFRKSNLKPFERDISTEIYQAGNKPCNGVAGFNCYFPLRSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 68 - DNA, 3633 bp, synthetic construct
  source 1..3633: mol_type = other DNA
atgttcgtgttcctggtgctgctgcccctggtgagcagccagtgcgtgaacctgatcaccagaacccagagctacaccaacagcttcaccagaggcgtgtactaccccgacaaggtgttcagaagcagcgtgctgcacagcacccaggacctgttcctgcccttcttcagcaacgtgacctggttccacgccatccacgtgagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctgggcagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcgacgaggtgttcaacgccaccagattcgccagcgtgtacgcctggaacagaaagagaatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcagaggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacctgtacagactgttcagaaagagcaacctgaagcccttcgagagagacatcagcaccgagatctaccaggccggcaacaagccctgcaacggcgtggccggcttcaactgctacttccccctgagaagctacggcttcagacccacctacggcgtgggccaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagtacgtgaacaacagctacgagtgcgacatccccatcggcgccggcatctgcgccagctaccagacccagaccaagagccacagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagagccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctag

SEQ ID NO: 69 - DNA, 3633 bp, synthetic construct
  source 1..3633: mol_type = other DNA
atgttcgtgttcctggtgctgctgcccctggtgtcctcccagtgcgtgaacctgatcacccgtacccagtcctacaccaactccttcacccgtggtgtgtactaccccgacaaggtgttccgttcctccgtgctgcactccacccaggacctgttcctgcccttcttctccaacgtgacctggttccacgctatccacgtgtccggtaccaacggtaccaagcgtttcgacaaccccgtgctgcccttcaacgacggtgtgtacttcgcttccaccgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctggacgtgtactaccacaagaacaacaagtcctggatggagtccgagttccgtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcaacctgggtcgtgacctgccccagggtttctccgctctggagcccctggtggacctgcccatcggtatcaacatcacccgtttccagaccctgctggctctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcgacgaggtgttcaacgctacccgtttcgcttccgtgtacgcttggaaccgtaagcgtatctccaactgcgtggctgactactccgtgctgtacaacttcgctcccttcttcgctttcaagtgctacggtgtgtcccccaccaagctgaacgacctgtgcttcaccaacgtgtacgctgactccttcgtgatccgtggtaacgaggtgtcccagatcgctcccggtcagaccggtaacatcgctgactacaactacaagctgcccgacgacttcaccggttgcgtgatcgcttggaactccaacaagctggactccaaggtgggtggtaactacaactacctgtaccgtctgttccgtaagtccaacctgaagcccttcgagcgtgacatctccaccgagatctaccaggctggtaacaagccctgcaacggtgtggctggtttcaactgctacttccccctgcgttcctacggtttccgtcccacctacggtgtgggtcaccagccctaccgtgtggtggtgctgtccttcgagctgctgcacgctcccgctaccgtgtgcggtcccaagaagtccaccaacctggtgaagaacaagtgcgtgaacttcaacttcaacggtctgaccggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccagggtgtgaactgcaccgaggtgcccgtggctatccacgctgaccagctgacccccacctggcgtgtgtactccaccggttccaacgtgttccagacccgtgctggttgcctgatcggtgctgagtacgtgaacaactcctacgagtgcgacatccccatcggtgctggtatctgcgcttcctaccagacccagaccaagtcccaccgtcgtgctcgttccgtggcttcccagtccatcatcgcttacaccatgtccctgggtgctgagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaagcgtgctctgaccggtatcgctgtggagcaggacaagaacacccaggaggtgttcgctcaggtgaagcagatctacaagaccccccccatcaagtacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaacggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcaggacgtggtgaaccacaacgctcaggctctgaacaccctggtgaagcagctgtcctccaagttcggtgctatctcctccgtgctgaacgacatcctgtcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctag

SEQ ID NO: 70 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 71 - DNA, 3807 bp, synthetic construct
  source 1..3807: mol_type = other DNA
atgttcgtgttcctggtgctgctgcccctggtgagcagccagtgcgtgaacctgatcaccagaacccagagctacaccaacagcttcaccagaggcgtgtactaccccgacaaggtgttcagaagcagcgtgctgcacagcacccaggacctgttcctgcccttcttcagcaacgtgacctggttccacgccatcagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctgggcagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcgacgaggtgttcaacgccaccagattcgccagcgtgtacgcctggaacagaaagagaatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcagaggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacagatacagactgttcagaaagagcaacctgaagcccttcgagagagacatcagcaccgagatctaccaggccggcaacaagccctgcaacggcgtggccggcgtgaactgctacttccccctgcagagctacggcttcagacccacctacggcgtgggccaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagtacgtgaacaacagctacgagtgcgacatccccatcggcgccggcatctgcgccagctaccagacccagaccaagagccacagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagagccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctag

SEQ ID NO: 72 - DNA, 3807 bp, synthetic construct
  source 1..3807: mol_type = other DNA
atgttcgtgttcctggtgctgctgcccctggtgtcctcccagtgcgtgaacctgatcacccgtacccagtcctacaccaactccttcacccgtggtgtgtactaccccgacaaggtgttccgttcctccgtgctgcactccacccaggacctgttcctgcccttcttctccaacgtgacctggttccacgctatctccggtaccaacggtaccaagcgtttcgacaaccccgtgctgcccttcaacgacggtgtgtacttcgcttccaccgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctggacgtgtactaccacaagaacaacaagtcctggatggagtccgagttccgtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcaacctgggtcgtgacctgccccagggtttctccgctctggagcccctggtggacctgcccatcggtatcaacatcacccgtttccagaccctgctggctctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcgacgaggtgttcaacgctacccgtttcgcttccgtgtacgcttggaaccgtaagcgtatctccaactgcgtggctgactactccgtgctgtacaacttcgctcccttcttcgctttcaagtgctacggtgtgtcccccaccaagctgaacgacctgtgcttcaccaacgtgtacgctgactccttcgtgatccgtggtaacgaggtgtcccagatcgctcccggtcagaccggtaacatcgctgactacaactacaagctgcccgacgacttcaccggttgcgtgatcgcttggaactccaacaagctggactccaaggtgggtggtaactacaactaccgttaccgtctgttccgtaagtccaacctgaagcccttcgagcgtgacatctccaccgagatctaccaggctggtaacaagccctgcaacggtgtggctggtgtgaactgctacttccccctgcagtcctacggtttccgtcccacctacggtgtgggtcaccagccctaccgtgtggtggtgctgtccttcgagctgctgcacgctcccgctaccgtgtgcggtcccaagaagtccaccaacctggtgaagaacaagtgcgtgaacttcaacttcaacggtctgaccggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccagggtgtgaactgcaccgaggtgcccgtggctatccacgctgaccagctgacccccacctggcgtgtgtactccaccggttccaacgtgttccagacccgtgctggttgcctgatcggtgctgagtacgtgaacaactcctacgagtgcgacatccccatcggtgctggtatctgcgcttcctaccagacccagaccaagtcccaccgtcgtgctcgttccgtggcttcccagtccatcatcgcttacaccatgtccctgggtgctgagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaagcgtgctctgaccggtatcgctgtggagcaggacaagaacacccaggaggtgttcgctcaggtgaagcagatctacaagaccccccccatcaagtacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaacggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcaggacgtggtgaaccacaacgctcaggctctgaacaccctggtgaagcagctgtcctccaagttcggtgctatctcctccgtgctgaacgacatcctgtcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctggtacatctggctgggtttcatcgctggtctgatcgctatcgtgatggtgaccatcatgctgtgctgcatgacctcctgctgctcctgcctgaagggttgctgctcctgcggttcctgctgcaagttcgacgaggacgactccgagcccgtgctgaagggtgtgaagctgcactacacctag

SEQ ID NO: 73 - protein, 1208 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 74 - DNA, 3627 bp, synthetic construct
  source 1..3627: mol_type = other DNA
atgttcgtgttcctggtgctgctgcccctggtgagcagccagtgcgtgaacctgatcaccagaacccagagctacaccaacagcttcaccagaggcgtgtactaccccgacaaggtgttcagaagcagcgtgctgcacagcacccaggacctgttcctgcccttcttcagcaacgtgacctggttccacgccatcagcggcaccaacggcaccaagagattcgacaaccccgtgctgcccttcaacgacggcgtgtacttcgccagcaccgagaagagcaacatcatcagaggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcgagttcagagtgtacagcagcgccaacaactgcaccttcgagtacgtgagccagcccttcctgatggacctggagggcaagcagggcaacttcaagaacctgagagagttcgtgttcaagaacatcgacggctacttcaagatctacagcaagcacacccccatcaacctgggcagagacctgccccagggcttcagcgccctggagcccctggtggacctgcccatcggcatcaacatcaccagattccagaccctgctggccctgcacagaagctacctgacccccggcgacagcagcagcggctggaccgccggcgccgccgcctactacgtgggctacctgcagcccagaaccttcctgctgaagtacaacgagaacggcaccatcaccgacgccgtggactgcgccctggaccccctgagcgagaccaagtgcaccctgaagagcttcaccgtggagaagggcatctaccagaccagcaacttcagagtgcagcccaccgagagcatcgtgagattccccaacatcaccaacctgtgccccttcgacgaggtgttcaacgccaccagattcgccagcgtgtacgcctggaacagaaagagaatcagcaactgcgtggccgactacagcgtgctgtacaacttcgcccccttcttcgccttcaagtgctacggcgtgagccccaccaagctgaacgacctgtgcttcaccaacgtgtacgccgacagcttcgtgatcagaggcaacgaggtgagccagatcgcccccggccagaccggcaacatcgccgactacaactacaagctgcccgacgacttcaccggctgcgtgatcgcctggaacagcaacaagctggacagcaaggtgggcggcaactacaactacagatacagactgttcagaaagagcaacctgaagcccttcgagagagacatcagcaccgagatctaccaggccggcaacaagccctgcaacggcgtggccggcgtgaactgctacttccccctgcagagctacggcttcagacccacctacggcgtgggccaccagccctacagagtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggcagagacatcgccgacaccaccgacgccgtgagagacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggagagtgtacagcaccggcagcaacgtgttccagaccagagccggctgcctgatcggcgccgagtacgtgaacaacagctacgagtgcgacatccccatcggcgccggcatctgcgccagctaccagacccagaccaagagccacagaagagccagaagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaagagagccctgaccggcatcgccgtggagcaggacaagaacacccaggaggtgttcgcccaggtgaagcagatctacaagaccccccccatcaagtacttcggcggcttcaacttcagccagatcctgcccgaccccagcaagcccagcaagagaagcttcatcgaggacctgctgttcaacaaggtgaccctggccgacgccggcttcatcaagcagtacggcgactgcctgggcgacatcgccgccagagacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctacagattcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccacaacgcccaggccctgaacaccctggtgaagcagctgagcagcaagttcggcgccatcagcagcgtgctgaacgacatcctgagcagactggacaaggtggaggccgaggtgcagatcgacagactgatcaccggcagactgcagagcctgcagacctacgtgacccagcagctgatcagagccgccgagatcagagccagcgccaacctggccgccaccaagatgagcgagtgcgtgctgggccagagcaagagagtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttccccagagagggcgtgttcgtgagcaacggcacccactggttcgtgacccagagaaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgacagactgaacgaggtggccaagaacctgaacgagagcctgatcgacctgcaggagctgggcaagtacgagcagtacatcaagtggccctag

SEQ ID NO: 75 - DNA, 3627 bp, synthetic construct
  source 1..3627: mol_type = other DNA
atgttcgtgttcctggtgctgctgcccctggtgtcctcccagtgcgtgaacctgatcacccgtacccagtcctacaccaactccttcacccgtggtgtgtactaccccgacaaggtgttccgttcctccgtgctgcactccacccaggacctgttcctgcccttcttctccaacgtgacctggttccacgctatctccggtaccaacggtaccaagcgtttcgacaaccccgtgctgcccttcaacgacggtgtgtacttcgcttccaccgagaagtccaacatcatccgtggttggatcttcggtaccaccctggactccaagacccagtccctgctgatcgtgaacaacgctaccaacgtggtgatcaaggtgtgcgagttccagttctgcaacgaccccttcctggacgtgtactaccacaagaacaacaagtcctggatggagtccgagttccgtgtgtactcctccgctaacaactgcaccttcgagtacgtgtcccagcccttcctgatggacctggagggtaagcagggtaacttcaagaacctgcgtgagttcgtgttcaagaacatcgacggttacttcaagatctactccaagcacacccccatcaacctgggtcgtgacctgccccagggtttctccgctctggagcccctggtggacctgcccatcggtatcaacatcacccgtttccagaccctgctggctctgcaccgttcctacctgacccccggtgactcctcctccggttggaccgctggtgctgctgcttactacgtgggttacctgcagccccgtaccttcctgctgaagtacaacgagaacggtaccatcaccgacgctgtggactgcgctctggaccccctgtccgagaccaagtgcaccctgaagtccttcaccgtggagaagggtatctaccagacctccaacttccgtgtgcagcccaccgagtccatcgtgcgtttccccaacatcaccaacctgtgccccttcgacgaggtgttcaacgctacccgtttcgcttccgtgtacgcttggaaccgtaagcgtatctccaactgcgtggctgactactccgtgctgtacaacttcgctcccttcttcgctttcaagtgctacggtgtgtcccccaccaagctgaacgacctgtgcttcaccaacgtgtacgctgactccttcgtgatccgtggtaacgaggtgtcccagatcgctcccggtcagaccggtaacatcgctgactacaactacaagctgcccgacgacttcaccggttgcgtgatcgcttggaactccaacaagctggactccaaggtgggtggtaactacaactaccgttaccgtctgttccgtaagtccaacctgaagcccttcgagcgtgacatctccaccgagatctaccaggctggtaacaagccctgcaacggtgtggctggtgtgaactgctacttccccctgcagtcctacggtttccgtcccacctacggtgtgggtcaccagccctaccgtgtggtggtgctgtccttcgagctgctgcacgctcccgctaccgtgtgcggtcccaagaagtccaccaacctggtgaagaacaagtgcgtgaacttcaacttcaacggtctgaccggtaccggtgtgctgaccgagtccaacaagaagttcctgcccttccagcagttcggtcgtgacatcgctgacaccaccgacgctgtgcgtgacccccagaccctggagatcctggacatcaccccctgctccttcggtggtgtgtccgtgatcacccccggtaccaacacctccaaccaggtggctgtgctgtaccagggtgtgaactgcaccgaggtgcccgtggctatccacgctgaccagctgacccccacctggcgtgtgtactccaccggttccaacgtgttccagacccgtgctggttgcctgatcggtgctgagtacgtgaacaactcctacgagtgcgacatccccatcggtgctggtatctgcgcttcctaccagacccagaccaagtcccaccgtcgtgctcgttccgtggcttcccagtccatcatcgcttacaccatgtccctgggtgctgagaactccgtggcttactccaacaactccatcgctatccccaccaacttcaccatctccgtgaccaccgagatcctgcccgtgtccatgaccaagacctccgtggactgcaccatgtacatctgcggtgactccaccgagtgctccaacctgctgctgcagtacggttccttctgcacccagctgaagcgtgctctgaccggtatcgctgtggagcaggacaagaacacccaggaggtgttcgctcaggtgaagcagatctacaagaccccccccatcaagtacttcggtggtttcaacttctcccagatcctgcccgacccctccaagccctccaagcgttccttcatcgaggacctgctgttcaacaaggtgaccctggctgacgctggtttcatcaagcagtacggtgactgcctgggtgacatcgctgctcgtgacctgatctgcgctcagaagttcaacggtctgaccgtgctgccccccctgctgaccgacgagatgatcgctcagtacacctccgctctgctggctggtaccatcacctccggttggaccttcggtgctggtgctgctctgcagatccccttcgctatgcagatggcttaccgtttcaacggtatcggtgtgacccagaacgtgctgtacgagaaccagaagctgatcgctaaccagttcaactccgctatcggtaagatccaggactccctgtcctccaccgcttccgctctgggtaagctgcaggacgtggtgaaccacaacgctcaggctctgaacaccctggtgaagcagctgtcctccaagttcggtgctatctcctccgtgctgaacgacatcctgtcccgtctggacaaggtggaggctgaggtgcagatcgaccgtctgatcaccggtcgtctgcagtccctgcagacctacgtgacccagcagctgatccgtgctgctgagatccgtgcttccgctaacctggctgctaccaagatgtccgagtgcgtgctgggtcagtccaagcgtgtggacttctgcggtaagggttaccacctgatgtccttcccccagtccgctccccacggtgtggtgttcctgcacgtgacctacgtgcccgctcaggagaagaacttcaccaccgctcccgctatctgccacgacggtaaggctcacttcccccgtgagggtgtgttcgtgtccaacggtacccactggttcgtgacccagcgtaacttctacgagccccagatcatcaccaccgacaacaccttcgtgtccggtaactgcgacgtggtgatcggtatcgtgaacaacaccgtgtacgaccccctgcagcccgagctggactccttcaaggaggagctggacaagtacttcaagaaccacacctcccccgacgtggacctgggtgacatctccggtatcaacgcttccgtggtgaacatccagaaggagatcgaccgtctgaacgaggtggctaagaacctgaacgagtccctgatcgacctgcaggagctgggtaagtacgagcagtacatcaagtggccctag

SEQ ID NO: 76 - DNA, 3807 bp, synthetic construct
  source 1..3807: mol_type = other DNA
atgttcgtgttcctggtgctgctgcccctggtgagcagccaatgcgtgaacctgatcacaagaacacagagctacaccaacagcttcacaagaggcgtgtactaccccgacaaggtgttcagaagcagcgtgctgcacagcacccaagacctgtttctccccttcttcagcaacgtgacctggttccacgccatcagcggcaccaacggcaccaagagattcgacaaccccgtgctgcccttcaacgacggcgtgtacttcgctagcaccgagaagagcaacatcatcagaggctggatcttcggcaccaccctggacagcaagacacagagcctgctgatcgtgaataacgccaccaacgtggtgatcaaggtgtgcgagtttcagttctgcaacgaccccttcctggacgtgtactaccacaagaacaacaagagctggatggagagcgagttcagagtgtacagcagcgctaacaactgcaccttcgagtacgtgagccaacccttcctgatggacctggagggcaagcaaggcaacttcaagaacctgagagagttcgtgttcaagaacatcgacggctacttcaagatctacagcaagcacacccccatcaacctgggcagagacctgccccaaggcttcagcgccctggagcccctggtggacctgcccatcggcatcaacatcacaagatttcagaccctgctggccctgcacagaagctacctcacccctggggacagcagcagcggctggaccgctggcgccgctgcctactacgtgggctacctgcagcctagaaccttcctgctgaagtacaacgagaacggcaccattacagacgccgtggactgcgccctggaccccctgagcgagaccaagtgcaccctgaagagcttcaccgtggagaagggcatctatcagacaagcaacttcagagtgcagcccaccgagagcatcgtgagattccccaacatcaccaacctgtgccccttcgacgaggtgttcaacgccaccaccttcgctagcgtgtacgcctggaatagaaagagaatcagcaactgcgtggccgactacagcgtgctgtacaacttcgcccccttcttcgccttcaaatgctatggcgtgagccccaccaagctgaacgacctgtgcttcaccaacgtgtacgccgacagcttcgtgatcagaggcaacgaggtgagccaaatcgcccctgggcagaccggcaacatcgccgattacaactacaaactgcccgacgacttcaccggctgcgtgatcgcctggaacagcaacaagctggacagcaccgtgggcggcaactacaactaccggtacagactgttcagaaagagcaagctgaagcccttcgagagagacatcagcaccgagatctaccaagccggcaacaagccctgcaacggcgtggccggcgtgaactgctacttccccctgcagagctacggcttcagacccacctacggcgtgggccatcagccctacagagtggtcgtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgccctttcagcagttcggcagagacatcgccgacaccaccgacgccgtgcgggaccctcagaccctggagatcctggacatcaccccctgcagctttggcggcgtgagcgtgatcacccccggcaccaacacaagcaaccaagtggccgtgctgtaccaaggcgtcaattgcaccgaggtgcccgtggccatccacgccgatcagctgacccccacctggagagtctacagcaccggcagcaacgtgtttcagacaagagccggctgcctgatcggcgccgagtacgtgaacaacagctacgagtgcgacatccccatcggcgccggcatctgcgctagctatcagacacagaccaagagccacggcagcgctagcagcgtggctagccaaagcatcatcgcctacaccatgagcctgggcgccgagaacagcgtggcctacagcaacaacagcatcgccatccccaccaacttcaccatcagcgtgaccaccgagatcctgcccgtcagcatgaccaagacaagcgtggactgcaccatgtacatctgcggcgacagcaccgagtgcagcaacctgctcctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgatcctagcaagcctagcaagagaagcttcatcgaggacctgctgttcaacaaggtgaccctggccgacgccggcttcatcaagcagtacggcgactgcctgggcgatatcgccgctagagacctgatctgcgctcagaagttcaatggcctgaccgtgctgccccccctgctgaccgacgagatgatcgctcagtacacaagcgccctgctggccggcaccatcacaagcggctggacattcggcgccggcgctgctctgcagatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatcagcagcgtgctgaacgatatcctgagcagactggacccccccgaggccgaggtgcagatcgacagactgattaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgctatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatcgtgaacaacaccgtgtacgaccccctgcaacccgagctggacagcttcaaggaggagctggacaagtactttaagaaccacacaagccccgacgtggacctgggcgacattagcggcatcaacgctagcgtggtgaacattcagaaggagattgacagactgaacgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctcaagggctgctgcagctgcggcagctgctgcaagttcgacgaggacgacagcgagcccgtgctgaagggcgtgaagctgcactacacctga

SEQ ID NO: 77 - protein, 1268 aa, synthetic construct
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATTFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSTVGGNYNYRYRLFRKSKLKPFERDISTEIYQAGNKPCNGVAGVNCYFPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGSASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 78 - DNA, 21 bp, synthetic construct
  source 1..21: mol_type = other DNA
acacgtcaatgggaagtgaaa

SEQ ID NO: 79 - DNA, 21 bp, synthetic construct
  source 1..21: mol_type = other DNA
gctagacgatccagacatgat